SEQUENCE LISTING (28 sequences)

SEQ ID NO: 1 - DNA, 202001 bp, Homo sapiens
  source 1..202001: mol_type = genomic DNA
gcccagcaggtgtcagcctcattttaccccgcccctattcaagatgaagttgttctggttccaacgcctctgacatattagctgcatcattttacatttcttttttttttttccttttaaatggggtcttgctctgtcacccaggctggagtgctgtggtatgatctcggctcactgcaatctccacctccgaggttccagcgattctcttgcctcagcctcccgagtagctgggactacaggcacccaccatcatactgggctaatttttgtgtttttagtagagatggggtttccccatgttgcccaggctgatctcaaactcctgggcttaagcaatacagccgcgttggcctcccaaagtgttgggattacaagcatgagctaccccacccagctcattttacatttccacttgttaaactgaaaactggcccgagaaagcttctgtactgccatccttgcgtccttgcagatgaatcgtaacctagcatagtaggtaggcagactgaaaacctaacttagcagtaggcttctgtaacaacagctgtgtctcagccagttcctgcagccagacttcaaccactcacaggccgcaaactgttcaaactgtgttcggagaaggcgaattcatctggctgttaacgtgcctcacttctgctttctgtggccactttcccttttctgtccataaatttgctttgaccacacagcatccctagagtctccctgaatctgctgtgattctgggacctgcaccatttgtgaattgttttttttttccttgatcagctaaactctgttcaattcaatttgttggaagtttttaacataccaatggtgcaccaaggttccaatttctccacttcctcataaataagtcattttaaatggcttttcagtattccaatatttggaagtattaatgtttctaccaattttctatttttggacattgaggttgtttcattttttttttctttttttgagacagagtctcgctccgtcacccaggctggagtgcagtggcctgatcccggcccactgcaacctccacctccctcctcagcctcctgagtagctgggattacaggtgcatgcaccaccacacccagctaatttttgtatttttagtagagatggggtttcaccatgttggtcaggctggtctcaaactcctgacctcaggtggtccacctgccttggcctcccaaaatgctgggattacaggcctgagccactgcgcctggcctcatcttcttgatattaatgttgctttaacatctttgtccctgtgttttttgtttttttttttgagacggagtctcattcattctgtcacccaggctggagttcagtggcgtgatctcagctcactgcaacctctgtctcctgggttccagtgattctcctgcgtcggtctcctgagtagctgtgttcctgggtctttcgatggttatttaatacttccctacagtaatgccctgtgcgtacatgctaagtgtgatgaaatggttggcacagttaaatcttttgaaagacattgccaagtcactcttcagaaaagtgataggaggtcatagcaattttaagaagtcctcatttctacatttccttactaatctcggttggtgtctcttcaatctttcctcacacttttcttgggtttttcctgaatcatgagtctactacatttacacattttaaagcatctttagaaacaggatctcattttgttgcccaggctagagtttggtggcatgattatagctcctcatactcctgggctcaagtgatccttccacctctgaaaccccaaaatttgagaaaggtctcatttaatttagaaagtttattttgccaaggttgagggtgcacacctgtgatgatatacgagttaaaaagaaattatttaggcagatactgagggtaagaaagtcctcggtaaggttttcttttcaatgaaaagcagcccccaagcattttcttttctaacaaagagcagcctgtaaaatcgagctgcagacatacacaagcaagctggaagcttgcacaggtgaatgctggcagctgtgccaataagaaaaggctacctggggccaggcagatccaacatggcggctccatcttccctttccttgtcaaccatgtgcacagtaaggagcaggcaacatagtgtcccccgagtagagaccaatttgcataataaaaggtgagggtagggtgggcagcttctttgcatgctatgtaaacattatgcctggtccaaccaatctttgggccctgtgtaaattagacaccacctcctcaagcctgtctataaaaccctgtccattctgccgcaggctggaagacccactggggcacccctctctctctataggagacagctattcatttttctctttctttcacctattaaagctccactcttaaccccactccgtgtgtatctatgttcttgatttccttggcatgaggcaatgaaccttgggtattaccccagaaccttgggtattatgccacttcagtgacacagcctcaggaaatcctgatgacatgttcccaagatggtcggggcacagcttggttttatacattttagggagacatgagacgtcaattcatatatgtaagaagtacattggttccgtccagaaaggcggggacaacttgaggcagggagagagcttctaggtcacaggtagacaaatggttgcattcttttgaatctccgataagcctttccaaaggaggcaatcagaatatgcgtctattgactgggcgcagtggctcatgcctgtaatgccagcactttgggaggcggaggtgggtggatcacctgaggtcaggagtttgagagcagcccggccaacatggtgaaaccctgtctctactaaaaatacaaaaaattagctgggcgtggtggcgggcgcctgtaatcccagctactcgggaggctgaggcaggagaatagcttgaacccagaaggaagaggttgcagtgagctgagatggtgccattgcactccagcctgggcaacaagagtgaaactccatctcagaaaaaaaaaaaaaaggcctgggcaaagtggctcacgcctgtaatcccagcactttgggaagccgaggcgggcaggtcacaaagtcaggagattgagaccatcctggctaacatgatgaaaccccatctctactaaaaaatacaaaaaactagctgggtgtggtggcgagcacctgtagtcccagctactcggcaggctgaggcaggagaatggcgtgaaccggggaggcggagcttgcagtgagccgagatcacaccactgcactccagcccggacgacagggcaagactctatctcaaattaaaaaaaaaaaaaaaaaaaaaaaaaaagagagagagaatatgcatctatctcagtgagcagaaggatgactttgaatggaatgggagcagttcctagcttgaacttcccctttagcttcagtgatttgggggctcaaggtatgttcctttcacatacctcagcctcccaagtagctgggaccacaagtgcatgccaccacacgtggctaatgttttattttttttgtaggaatagggtctcactatgtgtccaggctggtctaaaacccctgagctcaaatggtcctcccgcctcagcctcccgaaatgctgggattacaggcatgagccagcatgcccggcctagtctacatttttataaattgctaattcaaagttccctctccaaaacctcatggttttccctgttctcatcccctgcaccctcccttcccctggagtactcacctggccttggaggtctggtgtgagcccggacttcgattctaggcacagcatgtgatgagcgcccccaggtcaaacacctcccctctgcggcctgtgcttcaccgccttgacagtgagaaaggtctcccttcggctcattctcgaagtctcaaacttcacttctcctgtgcgctgattctgaattcagcccccgtccaaggtcctggcccctttctcttctgcttggcgtgttgttcatcaccactgtgcactgctgagggtaagtgcggttctctggacctctgctttatcattagaacagactcttgcggtttcccacgacattcctttcacttctcacttggaagatgagccgtgaggaaatcctgtgttgtgtggtatgtgggctgtgcttctgcttgacttgagggccaagcagcattgcaagccatggttttaaataagaaagaacatttctaaccttcatcttctagtaaggaaacaagtgggctttagagttcttgctcaggaaagacctatgtcccagtccaaccggaccttttactaaagagatcttcctgatcctcctccccaggccaggggaggggtcctccctggggttggagcctttagtagggggtcggagacacgacgtagccttcatgacattcatagtctagttacacgatccctgtaagggtcagttgaagtaagtgctacaaaggaagggaggtgctcagtggagagggctctcttttatgtattatatttctttcatggggagggatatggatcagggatcagcagaggtgtttcagtcccgagggaaagaaagtcagcgtggcttgggagttgggagcagcaagacagtggctcaagatatcttaagactagtggagtacaccttgcatgttaaaagccttgctcagggctgcctggttcttgtaggacgacagagatggcctagctctgcatactgcacccccaggggctcagaacagtgcaaatgtcagtctatctgtcagtggcagagccagccttggagcaggggtgcaaggaggtctctgcactggccaggcatgcagaacattctgttcagtagcactggacagaaggccccatctagatgagacagagctggtggggcaggacaaagactcctggcagctcaaacggcctggcagatgcttggagagagggggcttcttgagacagcaccatttctgggaagagagtcacctgggagggatgaggccacgctccggcttggaggtgaagagaggggctgctgcaagaaagaattagagacatgccagcctttgctgtgttgcccaggctggtcatgaactcttggcctcaagcaatcttcccacctcagcctccccaagcgctgggattatagacatgagcccccatgctggccaataaaagatgattttatggaggggatggtggtgaaggttgtgggtggtatgaaatagtaagaaatatatattggtctgcacccagttcctgccacagagctcctaaaatcctgagaacttcctgggtgagcatcttttgttctaatgaggtgactcttggtggctcctggataggagtgaatcaccagaaagatcaagccagagttagaagcagaaagtgctggctataacacaggaaagctgtaacacaaataataaagtttttttttttttttttgagatggagcctcactctgttgcccaggctggagtgcaatggtgcaatctcagctcactacaagctctgcctcccaggttcaagtgattctcctgcctcagcctcctgagcagttgggactacaggtgtgtgccaccacatctggctaatttttgtatttttagcagagacggggtttcaccatattaaccaggctggcctcaaactccttaccttgtgatccgcctgcctcagcctcccaaagtgctgggattacaggcatgagccaccgtgcctggccaaaagacattgttcttaaaagaatcaactaactaaccaaataaataaaaatctaacctaattaagaaactaaaaatacacaaaaattaatttcaaggggagaaaaatcatgtaaagagagaaagataatgaatactttgcagaaatttatgaacataaacataaaacttggatgaaatgcatttctaggaaaacataatttatcaaaactaaccacaagtaaaatagaagcctaaataggatattttcaagagaagaagtaaagttgtcaaagtgctacccttcaaaaaaacaccaggctcaaacaatctgacatgggaatgttagcacaccttagagagcaaataaaactttgaatgggcttgaaatattccagactctagaaaaacaaaacttcccaattctttttataaagcaagtataaattgataccaaaatcttataaagaccttatacaaaacttcataccaatctcttttatgaatacaaaacccttaataaagtattaccagacagaacccaacaatacataaaaatgtcacatcataacatagtggggtttatttcaataatgcatggatggttcaatacaaggaaattcagtaacacaatataatagatcatgtgaatatacccaaagaaaaaatagattattttcatagatgctgtaaaggcatttgaccaaattcaacacctactttttaggtggtcaataaaataaattagttactccttctttagcatgataaaatatatttatcagcccagaaggcatcattttacccgataagggcacacgctggagggaataatgttaaaattaggaataagaggatagctagtttctttcttcttttttttttttgagacggagtcttgctctgttgccaggctggagtgcagtggtgcaatgttggctcactgcacgccccccgcctcccaggttcaagcgattctcctgcctcagcctcccgagtagctgggactacaggcgcgcaccaccatgcccggctaatttttttttgtattttagtagagatggggtttcaccatgttggtcaggctggtcttgaactcccaacctcacgtactgggattaccggtgtgagccaccacgccagcccaactactttcaacattatccttaatactgatgcttattgacttactatggggttacctctagataaatccataataagttgaaaatataagtaaaaaatgcccttaatacacctaacctaccaaacatcatagctgagcccagcctgccttagctatgctcagacactgacgtcagcctacaattggcaaaatcacacagcagcacagtctactgcagagcatctgctgtttgcccttgtgactgcgtggctgcctgggagcttcccagcttcacaagacagtattacgtagcacatcactagcctggggaaagatcaaagttgaaaatttgaagtgtggtttccattgaatgtgtactgcttttgcaccatcatcaagtcaaaaaattttagttgaaccagcctaagtttgggaccatctttattttcaggaggaacttccatgtacattgatgacggacgatagaatccgtttctatcatcctaatgaacataatgaataaatccagacaaacataaacattaacagagtaagcagctttcggggctggaagccagaagagggtgggagcgcagagagagaggccaaacaccagggctgcttctgctttgcgggtatttgctgatctggacaaggtatctggaaggctgagctaagcctcctttttttttgaggtggcgtctcactctgttgccaggctggagtgcaatggtgcgatctcagctcactgcaacctccacctccctggttcaagcgattctcctgcctcagcctcccgagtagctgggattacaggctcccgccactacacccagctgatttttgtaattttagtagagacggggtttcaccatgttggccaggatggtctcgatctcttgacgtcatgatctgtccacctcggcctcccaaagtgctgggattataggcgtgacccaccgtgccccgtctgagctaagcctcttgagcataggggactaaaaatgaaatctagcgcatgccaagtttagggtcccaggcaattcctttccactttggggtccactttggggtccaccccacccaagaagaaggatgacttggaagtaaaccagctctgaaatatggatggtcctctgggaccataccaatcccttcatatcaaccacatccagttcctcaaaactggaacttggattaagatggcctaggacttctagtgtcccaggagcctggcattgcaaacaaaaatcctctccggaagaagataataccttaagcttcaaatgactctctaataaatttcaaatacaatgtccagcacacaaacacaaattaccaggaacgtgatatgaggcctgatggatgggaattagcagaaacttcaggcatgagaaacataccctcagaggcctagaatctatctagtgtctagataatggagatatgaaatacagacacttaaacaactatgtttcccatgttcaaagaggaaatttgcaaaacttgaaagtgttggcaggaaatcagaaactataaaatgtgacaacagcatactttagagtcagtataaattacggtcccgaaaactgcagaattccagaacttaatggtaaagcaagggtttaacagcagaatagaaatagccagagagaactaggaagtaagtcagatgacactacccagaataaggcactgagaggccaaggaatggaaaatgcagaagaaaggatatggtgagaggatctaatatacatttatttggagtaccagggagagagagaaggagaagaacagaagccgtgtttcaaggacggtgactgagaggcttcgaaactgatgaaagccatcagttcacaaattcaaagcccagtgaattccaaggagaaaaaaagaaatccatactgtgaaagcaagtccagacaatgacaaacaccatcaacaatacacaggacaggcataagatgcatttaatggggacactcagaggcagagggttatcagaaggaggcacttctctcccaagttctcatcatcccagggccagggacagctggtcacaccttagggagttcactaggagagggatctggcttcttgtcattctgggtatttgtagggaaattggaagggaaccgagagcacctagccaatcgcatagcaatgggagatttcaggctgtggggaatgtctttgctggtgaaaagaacatcctgaccttagaaatctttcaccgagggggatctgcgttccagaacttctggagctggtataggtaaggctttgagctttcctactgagccagcctgttgctaggttaccaaaggggacctcgagggccatctggccaacaagcagacttgtctctccttacacccccagacgtatcactgcaaaactacagaaaaccaaagacagagaaaatcttaaaagcagccagatttaaaaaatggcatattagtttcaaagcagcagccatgaaattgacagctgatgtctcaacagcaagaatgaaaagtggaagacaggccaggtgtggtggctcaggcctgtaatcccagcactttgggaggccgaggcgggtggatcacgaggtcaggagaccaagaccatcctggctaacatggtgaaaccccgtctctactaaaaatacaaaaaaattagtcgggcatggtggtgggtgcctgtagtcccagctactcgggaggctgaggcaggagaatggcgtgaacccgggaggcggagcttgcagtgagccgagattgtgccactgcactccagcctgggtgacagagcaagactctgtctcaaaaaaaaaaaaaaaaaaaaaaaaaaaagggtgacgaagcttcaatctcctgaaaggaagcaactgccgcctttgattcgatacccaccaaaatccgtgaagaaggaaggcaaaataaaaacacttcctgattgaactggaaagatttccgcaatagaagacccactgtccaaggaattctaaaggatgctttccaggcagaagaaaatgaccccagaggaagatcagagattcaggaaagaaatggagagtgataaaaatggaaaattcgggggccaatttaaacaaaagctgactgctctacaactgttgtgtctctatcttttgtaacatatatgtgtgtgtagcttttttttttttttttgtcaagatggattctcactctgtcgcccaggctacagtgaaatggcacggtctcggctcactgcaacctctgccccttgggctcaaatgattctcttgcctcagcctcctgagtagctgagattacaggtgcctggcacaatgcctggctaatttttgtatttttactagagatgggatttctccatgttggccaggctggtcttgaacacctgacctcaggtgatccacctgcctgggcctcccaaagtgctaggattacaggcgcgagccactgcatctggcctatgtgtgtgtttatatggaattaaaacacatggcaataataccctccaaattgggagaaaccaaaaatagcatttaaatgttgtaagctccctgcataatcaagaagagaatagatttacgttagattttgatacctggaggatgaatgttgtaatttctagggtgaccatgaaaagaggagacaacggtgtatgtttttttttttttgagatggagtctcactttgtcacccaggctggagtgttgtggtgtgatcttggctcactgcaacctcctcctcttgggttcaggccatcctcccacctaggcctccagagtaggtgggatcacaggcacctgccaccacacctggctaatttttttttttttttaaatatttagtagagatggggtttcaccatgttggccaggctggtcttgaactcctgacctcaggcgatctgcctacctctgcctctcaaagtgctgggattacaggtgtgagccatcgcgcccggccaacagtgatcactttcaaactaacagaggttcaaaaataaaatcagacttaaccaaaaaccaggtaacagagctggtaggatatacagaaagactgacctcacgtatatcaacgattacagttaatattaatgaaggaaatgctctagtttaaaaacgagggttgtcaaagaccccacataagaagctccttaccagcggtgcacctagaacctaaggaaacaggacagatgaaggaggacgcgcccccgccgctgtcctgcgcctcagccatcctatgagacgggaaaggtttctgtctgcagctgggcccgtgctctttaccagctcctggctttcttctctggaaggttcctgcctgttttgccctcacacctgctcctctctcagccctctcaggggtggggctggaggccaccaaagagcctcctctgctctccagttgctcgactgctcctcatttccccctggggtctgcgtcagggtttccttcttttccagccccaccccgcgtgcatcccacctggtctcgggtcggggctgctcccgcttactgccccctgcccaggctggtgtgcaccccctctggctgctttcaaggcctcttctctcttctcggcaggacaggcacaggcaggtggccaggtgtcatgcttagctccccgcccagtgagattctttcatttaacaatcttcccctgaatagttcatgttcattgctgaaaatttgaaaaatatggaaaagcacaaagattaagatataaaccgccctcaattcccctgcccagagagagtcactgctatgacttggtgactaggaaccttatttctctctcgctcttttttttttttttgagacagagtcttgctctgtcacccaggctggagtgcagtggctcgatctcagctcactgcaacctccgcctcctgggttcaagcgattctcctgcctcagcctcttgagtagctgggattacaggcacctgccaccatgcccggctaatttttgtatttttagttgagagagggtttcatcttgttggtcaggcggacttgaactcctgacctcaggtgatcagcccacctcggcctcccaaagtgctgggattacaggtgtgagccactgcgccttcatctctcttctgtgtatgtgtacgctgttttttctttagaatgggggacgttatcaggctctacatggtgtgtagtcggctagcatgttgtaagcctttccctgtgtcacaagtgctcatctggaacaggattctaatgactgcctgtggctatgttgggattcctttaactcagctccttctgcccagcatctatcttttttccatcttttgtcctaagtgttgctataataaatcattgatcacacatgcctgactgtttgcataggataaattacgggaaatgtttttgctgttcagggactgtgcccatttttaggcctcagagacaccatgccagactgcccagtattgatctttactctttttagatgatgccaaacttttctgtgaactttaaaaacctgtgtcttgacagtccatttctgtaagtctttcacattagatttcctgtcaggatgatagtcaattctaggcagatgatgttttctcagccatggctgaagcagttgtgatttgttgtggccatgtaaagtcccgatgatccattgcctccctggatgggttggaataatttggtttgggagcatataacagaatgacctggagtcacagcagctcagacggaagtgtatttctcccttacagatgaaagaattccaggccaggctggaatgacaactgcacacagtcatctgggccccctccttccagctcccatcaccccaggatgtggcttttatgcagatgatccaaaatggctgctcaagtcccagccaacacatcccattccagggagcaggaaaaaggtgtgtctttcccttcattttatgtgattcctttctagaagtactactcattacttctgcttgcatctccctggctagcacttacttagttatatggccatagctagctgaaggaaggacagggactgtcatacactagctaagaggcaaactgcttagataaaaaggtctctaaagaaggtcagagcggctgctagggtgcaactctattacttattgttatgggacgaactgtgtccctcattcaggttgatgtcctaagccccagaacctcagaatgggattgtatttggagacaggttctttaaggaggtaaggaggctaaaatgagatcattagggtgggccataatccgactgatgtcttacaagaagagattaggacacggacatgctcagagggacggccacgtgaggacaccaagaaaggcagctgtctgcaagtcaaggacagggctcaggggaaaccaaccttgccaacaccttcatctcggacttctagcctctaggaccatgagaagatacatttctgttgtttaagctgcccggtctgtggtactttgttatggcagcccaagtaaacaaatacagtcatctgctgctggaacaaatcaccccagcactgtggcttggcagcacacatgtctagtcatagagttatatgtagttacgtgtagagccatatgtatcgtcacacgttctgtgggtcaggaatttggacccagcttaaccagctccacttctcgccagggttcagtcaaataccagctgcctcccacctgagagctcagccggggaagggtccctttccaatctcacgtggtgttggcaggatccagttcctcatggcctgctggactgagaacctcagttctcactgcctgttggccagaggccgcctttatgtcctcgccatgtgggcctctccaacatggcagctgacttcatcagagcatccatgccaagaaggcaacagagagggccagggagactgaagtcatacccttttgcgacctagtcatggggtgacattccatcacctttgcccattggttagaagcaggccaccaggtacagcccaagctcacggggaggggtcatacaagggtgtcaataccaggaggtgaggggtgctggggccatcttatgagtctgcccactgaggtaactaacaaccttgaggcctgacacagtggacaaaggcccttattaacagcagagaactgggaactttatttatttatttatttttgagacagagtctcactcttgtcacccaggctggagtgcaatggcatgatcttggctcactgcaacctccacctcccaggttcaagcaattctgcctcagcctccggaatagctgggactacaggcatgcaccactacacccggctaatttttgtatttttagtagagacagggtttcgccatgttggccaggctggtctcgaactcctgacctctggtgatctgcctgccttggcctcccaaagtgctgggattacaggcgtgagccaccgcacctcgctggaacttaatttttttagagacagtgtcgctctatcacccaagctggagtgcagtggtgcaatcctagctcacttgcagcctcaaattcctgggttcaggtgatcctcccacatcagcctcccaagaactgggaactaacagctgtttctctgctgtccttctcaagaaaagggaggctactgctaccccactggggacaatgctgggtttccctttaggacaggctctgagacaaggcggaggtgctgtttgtggccacagagcaggggactctgggttgcaggtgtggcctggctaaagtaggctttactgggctcctctctgcctgcatcaccccccggctgggcggttgtctctgaggccaaccttactccctgctgggcaggctggacagctgccctctccgtttgcccctctaccacccaaaaggcaggaggctctggagaccaggaccctgcccgccacggcctgtgtcccaggcgtgagggggtgccccacagacctctgctgagctgctgctgaatgacgccccttgggggtcctgccggaaggtcagagcaggggtgcactcccataaagaaacgcccccaggtcgggactcattcctgtgggcggcatcttgtggccatagctgcttctcgctgcactaatcacagtgcctctgtgggcagcaggcgctgaccacccaggcctgccccagaccctctcctcccttccggggcgctgcgctgggaccgatggggggcgccaggcctgtggacaccgccctgcaggggcctctccagctcactgggggtggggtgggggtcacacttggggtcctcaggtcgtgccgaccacgcgcattctctgcgctctgcgcaggagctcgcccaccctctccccgtgcagagagccccgcagctggctccccgcagggctgtccgggtgagtatggctctggccacgggccagtgtggcgggagggcaaaccccaaggccacctcggctcagagtccacggccggctgtcgccccgctccaggcgtcggcgggggatcctttccgcatgggcctgcgcccgcgctcggcgccccctccacggccccgccccgtccatggccccgtccttcatgggcgagcccctccatggccctgcccctccgcgccccacccctccctcgccccacctctcaccttcctgccccgcccccagcctccccacccctcaccggccagtcccctcccctatcccgctccgcccctcagccgccccgcccctcagccggcctgcctaatgtccccgtccccagcatcgccccgccccgcccccgtctcgccccgcccctcaggcggcctccctgctgtgccccgccccggcctcgccacgcccctacctcaccacgccccccgcatcgccacgccccccgcatcgccacgcctcccttaccatgcagtcccgccccgtcccttcctcgtcccgcctcgccgcgacacttcacacacagcttcgcctcaccccattacagtctcaccacgccccgtcccctctccgttgagccccgcgccttcgcccgggtggggcgctgcgctgtcagcggccttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcagcgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtcccggcgggtcccaggctacggcggggatggcggtaaccctgcagcctgcgggccggcgacacgaacccccggccccgcagagacagagtgacccagcaacccagagcccatgagggacacccgccccctcctggggcgaggccttcccccacttcagccccgctccctcacttgggtcttcccttgtcctctcgcgaggggaggcagagccttgttggggcctgtcctgaattcaccgaggggagtcacggcctcagccctctcgcccttcgcaggatgcgaagagttggggcgagaacttgtttctttttatttgcgagaaaccagggcgggggttcttttaactgcgttgtgaagagaacttggaggagccgagatttgctcagtgccacttccctcttctagtctgagagggaagagggctgggggcgcgggacacttcgagaggaggcggggtttggagctggagagatgtgggggcagtggatgacataatgcttttaggacgcctcggcgggagtggcggggcagggggggggcggggagtgagggcgcgtccaatgggagatttcttttcctagtggcacttaaaacagcctgagatttgaggctcttcctacattgtcaggacatttcatttagttcatgatcacggtggtagtaacacgattttaagcaccacctaagagatctgctcatctaagcctaagttggtctgcaggcgtttgaatgagttgtggttgccaagtaaagtggtgaacttacgtggtgattaatgaaattatcttaaatattaggaagagttgattgaagttttttgcctatgtgtgttgggaataaaaccaacacgttgctgatggggaggttaattgccgagggatgaatgaggtgtacattttaccagtattccagtcaggcttgccagaatacggggggtccgcagactccgtgggcatctcagatgtgccagtgaaagggtttctgtttgcttcattgctgacagcttgttactttttggaagctaggggtttctgttgcttgttcttggggagaatttttgaaacaggaaaagagagaccattaaaacatctagcggaaccccaggactttccctggaagtctgtgtgtcgagtgtacagtaggagttaggaagtactctggtgcagttcaggcctttctcttacctctcagtattctatttccgatctggatgtgtcccagatggcatttggtaagaatatctctgttaagactgattaatttttagtaatatttcttgttctttgtttctgttatgatccttgtctcgtcttcaaagtttaattagaaaatgattcggagagcagtgttagcttatttgttggaataaaatttaggaataaattattctaaaggatggaaaaactttttggatatttggagaaattttaaaacaatttggcttatctcttcagtaagtaatttctcatccagaaatttactgtagtgcttttctaggaggtaggtgtcataaaagttcacacattgcatgtatcttgtgtaaacactaaacagggctcctgatgggaaggaagacctttctgctgggctgcttcagacacttgatcattctaaaaatatgccttctctttcttatgctgatttgacagaacctgcatttgcttatcttcaaaatatgggtatcaagaaatttcctttgctgccttgacaaaggagatagattttgtttcattactttaaggtaatatatgattaccttatttaaaaaatttaatcaggactggcaaggtggcttacacctttaatccgagcactttgggaggcctaggtggacgaatcacctgaggtcaggagtttgagaccagcctggctaacatggtgaaaccctgtctctactaaaaatacaaaaattagctggtcatggtggcacgtgcctgtaatccaagctacctgggaggctgaggcaggaaaatcgcttgaacccgggaggcagagtctgcagtgagttgagatcacgccactgcactccagcctgggtgacagagcgagactctatctcaaaaaaaattttttttaatgtattatttttgcataagtaatacattgacatgatacaaattctgtaattacaaaagggcaataattaaaatatcttccttccacccctttcctctgagtacctaactttgtccccaagaacaagcactatttcagttcctcatgtatcctgccagatataacctgttcatattgtaagatagatttaaaatgctctaaaaacaaaagtagtttagaataatatatatctatatattttttgagatgtagtctcacattgtcacccaggctggagtgcagtgatacaatctcggctcactgcagtctctgcctcccaggttcaaatgcttctcctgcctcagccttctgagtagctgggattacaggcgcccaccaccatgtccagctaatttttgtatttttagtagagatggggtttcaccatgttggccaggctggtcttgaactcctgaccttgtgatctgtccacctcggcctcccaaagtgctgggattacaggtgtgagccaccatgcctggctagaataataacttttaaaggttcttagcatgctctgaaatcaactgcattaggtttatttatagttttatagttattttaaataaaatgcatatttgtcatatttctctgtattttgctgttgagaaaggaggtattcactaattttgagtaacaaacactgctcacaaagtttggattttggcagttctgttcacgtgcttcagccaaaaaatcctcttctcaaagtaagattgatgaaagcaatttagaaagtatctgttctgtttttatggctcttgctctttggtgtggaactgtggtgtcacgccatgcatgggcctcagtttatgagtgtttgtgctctgctcagcatacaggatgcaggagttccttatggggctggctgcaggctcagcaaatctagcatgcttgggagggtcctcacagtaattaggaggcaattaatacttgcttctggcagtttcttattctccttcagattcctatctggtgtttccctgactttattcattcatcagtaaatatttactaaacatgtactatgtgcctggcactgttataggtgcagggctcagcagtgagcagacaaagctctgccctcgtgaagctttcattctaatgaaggacatagacagtaagcaagatagataagtaaaatatacagtacgttaatacgtggaggaacttcaaagcagggaaggggatagggaaatgtcagggttaatcgagtgttaacttatttttatttttaaaaaaattgttaagggctttccagcaaaacccagaaagcctgctagacaaattccaaaagagctgtagcactaagtgttgacatttttattttattttgttttgttttgttttttttgagacagttcttgctctatcagccaggctggagtgcactagtgtgatcttggctcactgcaacctctgcctcttgggttcaagtgattctcatgcctcagcctcctgtttagctgggattatagacatgcactgccatgcctgggtaattttttttttttcccccgagacggagtcttgctctgtcgcccaggctggagtgcagtggcgcgatctcagctcactgcaagctccgcttcccgagttcacgccattctcctgcctcagtctcccaagtagctgggactacaggcgcctgccaccacgtccagctaatttttttgtatttttaatagagacggggtttcaccgtgttagccaggatgatcttgatctcctgacctcgtcatccgccgaccttgtgatccgcccacctcggcctcccaaagtgctgggattacaggcatgagccactgtgcccggccacgcctgggtaatttttgtatttttagtagagatggggttttgccatgatgagcaggctggtctcgaactcccggcctcatgtgatctgcctgccttggcctcccaaagtgctaggattacaggcatgagccaccatacctggccagtgttgatattttaaatacggtgttcagggaaggtccactgagaagacagctttttttttttttttttttggggttggggggcaaggtcttgctctttaacccaggctggaatgcagtatcactatcgtagctcacttcagccttgaactcctgggctcaagtgatcctcccacctcaacctcacaatgtgttgggactataggtgtgagccatcacacctggccagatgatggcttttgagtaaagacctcaagcgagttaagagtctagtgtaagggtgtatgaagtagtggtattccagatggggggaacaggtccaaaatcttcctgtttcaggaatagcaaggatgtcattttagttgggtgaattgagtgagggggacatttgtagtaagaagtaaggtccaagaggtcaagggagtgccatatcagaccaatactacttgccttgtagatggaataaagatattggcatttatgtgagtgagatgggatgtcactggaggattagagcagaggagtagcatgatctgaatttcaatcttaagtgaactctggctgacaacagagtgaaggggaacaccggcaaaagcagaaaccagttaggaagccactgcagtgctcagataagcatggtgggttctgtcagggtaccggctgtcggctgtgggcagtgtgaggaatgactgactggattttgaatgcggaaccaactgcacttgttgaactctgctaagtataacaatttagcagtagcttgcgttatcaggtttgtattcagctgcaagtaacagaaaatcctgctgcaatagcttaaactggtaacaagcaagagcttatcagaagacaaaaataagtctggggaaattcaacaataagttaaggaacccaggctctttcttttttttttttttgaaacggagtttcgctcttgtcacccgggctggagtgcaatgatgtgatctcagctcactaaaacctctacctcctgggttcaagtgattcttctgcctcagcctcccaagtaactgggattacaggcgtataccaccatgcccagctaatttttgtgtttttagtagagatggggtttcaccatgttggccaggctggtctcgaacttctgacctcaggtgatccactcgcctcagcctgccaaagtgctgggattacaggtttgggccactgcacccggtcagaacccaggctctttcttatacttaccttgcaaacccttgttctcattttttccctttgtatttttattgttgaattgtaatagttctttatatattctggatactggattcttatcagatagatgatttgtaaaaactctcccttcctttggattgtctttttactttcttgatagtgtcttttgaagtgtaaaagtttttaattttgatgaagtcgagtttatctattttgtctttggttgctgtgcttcaagtgtcatatctaagaaatcattgtctaatccaaagtcaaaaaggtttactcctatgttttcttctaagaattttagagttttacatttaagtctgatccattttgagttaatttttatatatggttcaggtagaagtccaactttattcttttccatgtggttattcagttgtcccagcactgtttgttgaagagactattctttccccatggaattatcttagtacccttgttgaaaattaatcgtccttaattgtataaatttatttctagactgtcagttctacctgttggtctttatgtcgatcctgtgccagtaccatacagtcttgattactgaagtttgtgtcacagtttaaattcatgaaatgtgagttctccaactttgttccttttcaagattgatttggccatgctgggtcccttgcatttccgtacgaattgtaggatcagcttgtcagtttcaacaaagaagccaagtaggattctgagagggattgtgttgaatctgtagatcaacttggggagtattcgcatcttaacaatattgtcttccacctatgaacatgggcaaactttgtgtaaatggtcagattgtaagtatttcgggctgtgtgggcacagtgtctctgtcacagctacgcggctctgccattgtagcatgaaagtagccataagcaatatgtatgagtgtctgtgttccaatagaattttattaatgacaaggaagtttgaatttcatataattttcacctgtcatgagatagtatttgattattttggtcaaccatttaaaaatgtaaaaacatttcttagcttgtgaactagccaaaaatatgcaggttatagttttcccactcctaggttaaaatatgataggaccacatttggaaagcatttctttttttttttttttttttttttttgagacggagtttcactcttgttgcccaggctggagtgcagtggcgcgatctcggctcactgcaacctctgcctcccaggttcaagacattctcctgcacggcctccctagtagctgggattacaggcatgcgccaccacacccagctaattttgtatttttagtagagacggggtttctccatgttggtcaggctggtcttgaactcctgacctcaggtgatccacccgcctcagcctcccaaagtgctgggattacagggtgtgagccaccacaccctgctggaaagcatttcttttttggctgtttttgttttttttttaaactagttttgaaaattataaaagttacacatatacattataaaaatatcttcaagcagcacagatgaaaaacaaagcccttcttgcaagtctgtcatctttgtctaacttcctaagaacaaaagtgtttcttgtgtcttcttcccagattttaatatgcatatacaagcatttaaatgtgtcattttttgtttgcttgactgagatcacattacatatgtatttttttacttaacaatgtgtcatagatattgttccatagcagtacctgtaattcttattaattgctatgtaatattttagaatttctttttaaaagaggacttttggagatgtaaaggcaaaggtctcacatttttgtggctgtagaatgtgctggtgacatattctctctaccttgagaagtccccatccccatcacctccatttcctgtaaataagtcaaccacttgataaactacctttgaatggatccacactcaaaacatttagtcttattcagacaacaaggaggaaaaataaaataccttataaagcactgtttaatattgtattaaattggatcaatttgggggctagaatgtatgttagagacatgatatgtccataggtccttgctatcacagtgaggtctcagggacagtcgtttggtatcatttgggatctcataagcagactctctctgcttgacctgacaaatcagagtctgtgttttaacaggttcagtgagtgacttacatgcacattggagtttgggaagctccactgtaggtgcttagaccttacctttgttgttgctaataacaatgcaagcatttgggaggaagacctgtgttgctcatatgtgtccaggtgtagctgaggtggccttgcttatctgctgtagggccgttgagcatttctgtagctgtgatgagtgagctgaggtgagcctgcggagagctcccagccattggtagtgggactcgcttagatgaactggaaggaccctttcatctgagcagccactatggagaaaaacaaccgaatgaggggagagacaatgtgcaattttatttagggcacaaaggagagctgtggttagaaggtgacatttgagtggaaagggggcaagccatgtgtatagcgggagaagagaggtccaggcagagttaacagaaggcagaaatgctttccatgtttgagaaccagtaaggaggccagtggctgaagtaaggtgaagggcagaaataaggatgaggctgcgagagatgagaggttagagacgagcgtcttgtgcaccaagataagcttgtgtggtcaaaacaagtagtttaatttatgtttttaaaagatcattttggctgggcacaatggttcatgcctgtaataccagtagtttgagacggtgtggtgggaggattgcctgaggccagacgaccagcatagccaacatagcagcacctataaggtctctacaaaaaactttaaaaaattagctgggcatagtggtgtgtgcctgtagtcccagctactcaggaggctgaggaggctggaggattgcttgagtccaggagtttgaggctgcagtgagctatgattatgccactacactacaacctgggcaagagagtgagaccctgtctctaaatatacacacacacacacacacacacacacacacacacacacacacacacacacacacacatatatatgtatatatatgcatttagatgaaaagatcactttgacaataccacatgctggtgaggatttagaaaaactaggtcacttattgctggtgggaatataatatagtacggccactctggaaaacagtttggcagtttgtcataaaactgaacataccgttagtatacagcccagcagcaactacaatcctgggcattaatcctagagaaatgaaaccttaatgttcacataaaaacctatactcaagtatgcatagcagctttacccataatatctaagaactggaatcagctcagatgtccttcaacaggtgaatggttaaactactcagtaataaaaaggaatgagctactgatagcatgcaacagtttaggtgaagttatgctaatgaaaaaagccaatcccaaaaggttatacatactgtatgattctatgtttttttgcaatggcacagttttagggatggagaatagattagtggttgcctggggttagagatggggtagtagagtaggttagtggtggcagaggagagaaaagagagggaggtgaatgtggttataaaaggacaacacaggggaatacttgtaatggaaatgctttgtctttttttttttttttttttttttggcgacagagtcttgctctgttgcccaggctggagtgcagtggcatgatcttttctcactgcaacctctgcctcctgggttcaagtgatacttgtgtctcagtctcccatgttcagagtgaaacaaaccagaggtaatgttcatccaaataatccaacacacatgacattaaaacatcaagatcaggtcggacgtggtggctcatgcctgtaatcccagcacttttgggaggccaaggtgggcagatcacttgaggtcaggagttcgagaccagccgggccaacatgatgaaaccccatcttgactaaaaatacaaaaattagccgggcatggtggtgtgcacctgtagtcccagctacttgggaggctgaggcaagagaactgcttgaacccgaggggcagaggttgcagtgagctgagagtgcgccattgcacttcagcctgtgtgacagagtaagactccatctccaaaaaaaaaaaaccaagatcaattaaaatacagcattactgggccgggtgtggtggctcacacctgtaatcccagcactttgggaggccgagatgggcagatcacgaggtcaggagatccagaccatcccggctaacacggtgaaaccccgtctctactaaaaaatacaaaaaattagccgggtatagtggtgggtgcctgtagtcccagctacttgggaggctgaagcaggagaatggtgtgaacccgggaggcagagctggcagtgagctgagatcgcgccactgcactccagcctgggcgacagagcaagactccgtctcgggggaaaaaaaaaaataaataaatagaatgctgtagtgtccttgagtttacatgcccctccttacgcttgtgtgcccgtgcagattgcttgattacacaattagaggaggctggcggaggattgttttaatttttttttttttgagacagtctggctctgttccccaggctagagtgcaatggcgcaatcttggtgcactgcaacctctgcctcctgggttcaagcagttcttctgccgcagcctcccgagtagctgggattataggcgcccgccaccacgcccaactattttttgtatttttagtagagcagcgtttcaccatgctggccaggctggtctcgaactcctgacctcagatgatctgctgccccagcctcccaaagtgctgggattacaggcgtgagccacacctggccgtttgttttaattttgaaggtgaagtgaaagtgactacatttaccaaaagtgattgaaaagccaggactgttcttaccctgtttttccagttcttgctcagagcaaggtggtttctttttcacttaatcaccatacttacttttcatgtagaacaagtcagtttgagttatcagttcatcatcttaactaaattccatgggggaaggaattagttttagtttcttaaacttccaggtttgcttattggacaaaatgagatagcaaggcagtgtttttaagttagattttttatttctttggtaatacaattttctcagaaacttagtagtcttttagtttagttgtttttagttggtcctatgttttggatcacccctctctactttattttgatagtgccaactgtgaagacatctgaagccataggtttggatgggaaggaggcatctttagcctgatcatcttcgccaggctgtttatctccttttgcttggctgagaagtcttaataggaggcttattcccagctatttggggacatagaagcagttagccattgcttatattttactgaggtctgtgtggtatgttgattgtagtcagttaacgattttgagaactgaaggcagcctggtatatatagagtaggtattagactgtgtttcttctaattgaatttcccatctcttgtaatctatgccatcatcttctgtactgctgagaaagaaagaaagtttctaatcaaactataccactggttgtaagatgcagtttggctttagtgatgttaacacatgattcaaacgtgaaattgattgagtattggtgaaatacagaggagatttaaagccagaagacctgggtttaaatgctggctgtatgacttcatatctgtgtgatcttgggcatgtcatggttggcacttcaatttcttctctctataatgggggaagtgaggccagtcatggtggctcatacctataatcccagtgctttgggaggccaagatgggaagatcgcttgaggccaggagtttgagcaattgggcaacatcgtgaggccccgtctctacaaaatattttgaaaaaattagccaggcccagtggtgcgtgcctgtggtccgcgccactcaggaggctgagacgggaggatcctttcagcctaggagtttaaggctaaagtgagccatgattgtgctatcgtactccagcctgggcagcagagcaagatcctgactctaaaaaaaagtaaaataaagtaaaatgggggaaatgaactgctttagtaacatcatctgttttttctgtgagcagcgtagcttgacagccattggtgaactcgtgccctgtgcttccctgtccagatccccattctgcccgcaacatggagtataacggtttattcatagtagtcgagaaacactcactgaatgaatgaatgaggtgtagaactaagtggagtgggtaattcaacacatattaatttccttcttttttttatttttagaaagaaagaactttcagctaccaagaaagaccgtgtgaatcattgtctgacaatatgtgaaaacatagtggcacagtctgtcaggtaattgcactttgaactgtctagagaaaataagaactttgtatattttcagtcttaatgggctagaatattctttgtgtcccagctattttaaatggattcagaaatccatttaagatgaagaaggacccttttcccatatttctggctatatacaaggatatccagacactgaaatgaataatgttccctttttgtaatcttttatgcaaaaattaaaaccattatggtaattgaacaacatgtttatgtttagttaacacccttagcaactatagttattttaaaaccatctatggtttgatatttttgcatttgttgcaatagtaggaacagcacaagacagttcagtttgtctctcttatttgctttttcttggcagtttgctgtcctattgtacctctgctcctagcagtggctggagcccactcctctgtgcttcgggattagtggggatcgtggggcattgactgtaggtcagctttccttgcttgatctttctcactgggatgaactagcagcaccttcttttgtagctgctttgcttttgactatctttctgaccgttgttcctagtagctgtagatggtaaatatatttaggcctgtttccaatggctcagtaggagacatattcacctatgatatctgaattctgttacccacatgggcatgcgtgaaatagttgccttgccttactttcccttggaataaataattcatgttattctcctggtagaagctagaaaaagcctttatagtcagtcagaaaaaaatttttagacaaataatcttgattttagtactgacaaaaacgtgtggtgattctttttttaatttttttttgagacggagtttcactcttgttgcccaggctggagtgcaatggcgtgatctcggctcactgcaacctctgcctcctgggttcaagtgattctcctgcctcagcctcccaagtagctggagttacaggcatgtgctactgtgcccagctaattttgtatttttagtagagatgttggtcaggctgatctcgaactcccaaccttaggtgatctgcccgcctcagcctcccaaagtgctgggattacaggcgtgagccagggcgcccggtgattcatttgttttttcaaaaaatttcctcttggccattgcttttcacttttgttttttttttttttttgagacggagtcacgatctgtcacccaggctggagtgcagtggcatgatcttggcttactgcaagctctgcctcccaggttcacgccattctcctgcttcagcctggcgagtagctgggactacaggtgctcgccaccacacccggctaattttttgtatttttagtagagatggggtttcaccgtggtcttgatctcctgacctcatgacccgctcaactcagcctcccaaagtgctgggattacaggcgtgagccaccgcgcccggccctctcttgtctttttattgtggtaaaatgcacataaaattgactgtcttaaccatttttaggggtacagttcagtatatatattcgtaatgttgtacagccatcactgccatctacttcataagtttttcttctgtcaaaactgaacatctgtcttcattaaactccctatcatccattctttcctgtagtccctttctactttctgtctgtatgagtgtaactgctctggagacctcatgtaagtggattcctacaggatttgtgttttttttttggtgatctgcttatttttaatgcctctgtgcatttgtattatatactttcaaagtgatttcacaaaaccgtttcattttaggttaactcatttctgttgtttgtgaaatactgtgtatgattctgttctgtttctgtctaatttgtggaaatgttgtgggaagaaaatgaaataacaaatgagcatatgtcctgaaaataaaaatataaaaattctaagttagcatgctattgtagaatacaacgctatgataaaagtaggaaaaaaaaaggtttgaattctatctctgctacctgtgtaagctgggtgactttagataagctgtaacgtgtttgagccttactggctcatttttgaaatgtaatccctagttacacagttcttgtgggatcagatggtacatgtgaaacactgtgaaaaagcaactgcatagatatgttcattagccacctgagcgggaagcgtatcccattgcgatgcccatcatccaaagctatatgttatctttacttttttttttttgagacagagtcttgctctgttgcccaggctagagtgcagtggtgcaatctcagctcactgcaagctccacctcccgggttcacgctattctcctgccccagcctcccaagtagctgggactacaggcacccgccaccatgcctggctaaatttttgtatttttagtagagatggggtttcaccgtgttagccaggatggtcttgatctcctgacctcgtgatccgcccgcctcggcctcccaaagtgctgggattacaggcgtgagccactgcccctggccatctttactttttttgtgaaatgactttaaatacttggcaaacatttggtcattgttcatctgatctccaccatccaggtctcagagaacataatttctctctgaaagcttattgacccaggaaataagatctctttcaatctgagtgcgtcaggctttattcttgtcattttgtcttttgataattttcaaatggaattcatggaatgttggcttatattcatatattagtaaagtatgttgagacatcttaagattgatttgtggttctatatgccatattaaatcaaaataatagctgttaatggttttcacattagtctgtctcttgtttttatggagtaatgctgagagttcattatgcttgttctacagaagagcatgttaaaaggagtttttggagtcagagaggttattcttggtttcataggatacactctatactttttagggatttcagagtatatagctgaaggtgatattttatgtaaatatgttttatggaaacttattgctcatcgctgtttcctgttaactctcctaaaatataattaaacttttggaacttttttatagcttttgtgctagactaatttttgtctctaatgaggttatataaatggcagcttctgacgttttcaatgtaggaagtcatttaaaacttcatgtatattgtgaaaatgtagtctgctttaagctctctaaagtggtctaagttactggttcctaagtatggatgagcatcaaaatcatctggaaaatttgttaaaaatacagtaatgaaggcacctcactgtcctttttcccaaacatacttctgcattctgtttgagtaggtagggactacacatttttcacaagtatcctcttgggaatacccaggaatgcttacttgagcaacctcttactaatatgtaccttgataaggtggctaggtaaacataaatatacaaaaatccatagatctcccatatattagcataaatcagctagaaaatataacgtttaaagatctagttcacagtagcaccaatatatcgaactctaaggaatcgataaatatgcaaaaactttataaaaacttctgttaatgtttctgaaagatataggtgaccactttctagataggaagattttatattactaagttgaattttctctaaattaacacagaaatttaaaataatcttgatcaaaattctagtagaggtatttttgaacttgttcactgcaagaataaatacataattgcaaagaatatctcaaaatcatcaccaggcctggtgtggtggcccatgcctgtaatcccagcactttgggaggctgaggcaggcagatcacctgaggtcaagagtttgagaccagctggaccagtgcggtgaaacactgcctctactaaaaatacaaaaattagctgggtgtggtggtgcatgcctgtagtcccagctacttgggaggctgaggcaggagaattgcttgaacccaggaggtacaggttgcggtgagcctagatcgcaccactgcattccagcctgggcgacaagagcaaaattctgtctcaagaaaaaagagaaaaaagaaaaagaaatcaacactaatatggtgagacttaatgtatgtgacattaaaatagtgattggatgttaaaacaggtatagaacagaaagaagagtgtatgtgtgtatctgtatgaatttatgatgggtgtaacatatatgtattagggaaatgagggaaatgatacatttctctgactttgggagaacattatatctctacctcatattgcaaacaaacataaagttcagattaattacctaaatgtgaaaaaatgaaataatttctttaaaaaatgtaatcttagtttgaggaaggttaacattataaaggaaaaaactgttttgagtggaatatagttcaatatgtcaaaatccaccttcaacaaaattgaaagtaaattgaacttggggaaagtattgacagcatatagatcaaaggttactagcctgtgtaaagagcagttataaatatcgttaagaaaaacactgtcgacctgtcggcaccttgttctccgactcccagcctccagaactgtgacgagtaagtgcttattgtttaaaccacccagtctgtatgtggtattttgttatagaaactcaagctgattaggacactagtaatcagtagactgaaactgaaacaaaaataagaaccttttttacctgtcaaattggcaaacattaagaatattcagatttttgtcagaggtgatacaaccttctaagaaggcaatttgggaaaatataaagctttagattattatatgtctgacctagcagttttacctctagggtgcttacccctaggaaagtgtgtaatgatattggtgcagtgcccttcatcccattagaaaattaaaaataaccttaatggcctaccactaaaaggggattgaaaatttaagatatatttatttatgtgtttattgagatggagtcttgcactgtccgcctgggccagagtgcaatggtgcgatctcggctcactgcaacctctgcttcccgggttcatgtgattctcctgcctcagcctcctgagtagctgggattacaggctcacaccaccgcacccggctaattttttgtatttttagtagagatggggtttcactgtgttggccagactggtctcgaactcctgacctcatgatccgcgcccctcggcctcccagtgttgggattacaggtgtgagccactgcgcctggccagatacatttatacaagagaatgttagttaacattcatagatatttatattttgtttactttttattaaaaaaattttttttagagacaggatcttactctgtcacccaggcaggatgcagttgcacaatcatagcccactgcagcctgaactcctgggcttaagtgatccttctgcctcagccttttgagtacctgggggactttaggcagtgctactatacctggctaatttttaaatgttttatagatgagatcttgctgtattgcccaggctggtctagaattcctgggcccaagtgatcctcccaccttggcctcccaaagcgctgagattacaggcatgagccaccacttctgaccaatagatatttatatttgtgactggaaaatatattaacaatgtgttaaaaaattcagttaaaaaataatgaaagatttttgcttctggctaagatagaataacaaggacagcatttatcttcttgccttgaaatagttgaaaacggaagaaatatatgtaacagtggttttcaagttattgggcatcaggcaaagaagaatagttatcccaggaaaatgaatgtggagagccctacaatttccttacattactgcctggtcatggcaagaggaaaaactgagaggagactgaggctgagccagtggtttgctgggttgaggaggcagagctgggagtgcagagatgcaaggtggtgagagcccatatggaagaataccagggaagagagctgcagagggagctccggagacctgcaccctgccctctcagtaccctgtcatgtgtgtagctgagtactgacgagcacttgcttgtgcggaaatgacccagggctggaggtagagccacctgaaaggattagaaggaacagttgctgaaagtcacacagggccaggaagaatttctaatcacaccagttggagtggaaaacctcagctctcatagagcaggtagggtactcagaagggtttgcccacctagccccagactaagtttcgttactctgaccctacctaatattaaaaagagattaattaaattgttcgcaacaaaaataatatatttcagtgtttgtaacacgtagaagtgaattgtatgacaatagcataaaggctggaagagcagaaattgacatgtatttgcgctgggcagaataatgctcccctctttccccaaaagatatcaagtcctaatccctggagcctgtaaatattactttatatggaaaattgttttatgatgtgattaaattcaggatcttgagatgagggggctatcttggatgatctgggtaggcactaaatgcaatcacatatatataaaaaggaggcagagggagattttacacacagagagaaggccctgtgaagatggaacagaaagatttgaaggtgctggccttgaaaattggagtgatgaagctataagccaaggaatgcagcagccaccaaagctggaagaggcacggagcagttctcatttagagcctactccagagggaatgtggtgctgccaattccttttttttttttttttttaagatatcatttacccctttaagttggttttttttttttttttttttttttagtatttattgatcattcttgggtgtttcttggagagggggatttggcagggtcataggacaatagtggagggaaggtcagcagataaacatgtaaacaaaggtctctggttttcctaggcagagggccctgccacgttctgcagtgtttgtgtccctgggtacttgagattagggagtggtgatgactcttaacgagtatgctgccttcaagcatctgtttaacaaagcacatcttgcaccgcccttaatccatttaacccttagtggacacagcacatgtttcagagagcacggggttgggggtaaggttatagattaacagcatcccaaggcagaagaatttttcttagtacagaacaaaatggagtgtcctatgtctacttctttctacgcagacacagtaacaatctgatctctctttcttttcccacatttcctccttttctattcgacaaaactgccaccgtcatcatggactgttctcaatgagctattgggtacacctcccagatggggtggcggccgggcagaggggctcctcacttcccagatggggcggccgggcagaggcgccccccaacctcccagacggggcggcggctgggcgggggctgccccccacctcccggacggggcgggtggccgggcgggggctgcccaccacctcccggacggggcggctggccgggcgggggctgccccccacctcccggacggggcgggtggccgggcgggggctgccccccacctcccggacggggcggctggccgggcgggggctgccccccacctcccggacggagcggctgccgggcggaggggctcctcacttcccggacggggcggctgctgggcggaggggctcctcacttctcagacggggcggctggtcagagacgctcctcacctcccagacggggtggcagtggggcagagacattcttaagttcccagacggagtcacggccgggcagaggtgctcttcacatctcagacggggcggcggggcagaggtgctccccacttcccagacgatgggcggccgggcagagatgctcctcacttcctagatgggatgacagccgggaagaggcgctcctcacttcccagactgggcagccaggcagaggggctcctcacatcccagacgatgggcggccaggcagaaacgctcctcacttcctagacggggtggcggctgggcagaggccgcaatcttggcactttgggaggccaaggcaggcggctgggaggtgaaggttgtagtgacccgagatcacgccactgcactccagcctgggcaacactgagcactgagtgagcgagactccgtctgcaatcccggcacctcgggaggccgaggctggcagatcacttgcagtcaggagctggagaccagcccggccaacacggcgaaaccccgtctccaccaaaaaacacgaaaaccagtcagacatggcggtgcgtgcctgcaatcccaggcacttggcaggctgaggcaggagaatcaggtagggaggttgcagtgagtagagatggtggcagtacagtccagccttggctcggcatcagagggagactgtgcgagggcgagggcgagggcgagggaattccttaatttcagtttagtgatactaattttggactctggcctctaaaactgtgaaagaaaaaattttttgtttgtttgtttcttttaagccacatagtttgtggtaatttgttacagcagctgcaggaaactaatttatgctgcatgtgaaatggtgtaataaggtagattgtgatgaagatacatagtataaacaattaagcaacaactaaaagcacaacaaggaattatagctaatgaaccaaaaaaggagattagaataataaaaatggtgaatcccaaagaagccagaaataggggaagaggcaaataaaggaaagaaagagcttgatggtagatttcaacctaactatgtcaaaaaggacattacatgtaaaaggcagcgatttttcagattgaatggaaaagtaagactcggtatatgctgctgcctgcaagaaacacattctaaatataaaggcaaaaataacctacaggtaacagaacggaaagaagttcactgtgcttacaagaattagatgcaagctagactggttctgttaatatcagacaaagtggatttcaaagcaaaggctcttgcccaggatgagatggtcatttcataatgatgaaggggattcgttcatcagcctggcatagcaagctgaaatgtttatgcaccggactacagagctaaaatacatgaagcaaagcctgacagaactacaagtagaaacagacaaatccacagtgatagagatttcagtagccgctctcaatgatttgtagaacacgtagccataatatctggatctagaacacttgaccaacactgtcccctgtgcaacctcattggcatttacaggacactccacccagcaccagcagaagagacactctctcaagtgctcacagaatgtttgccaagatagagcagatgctgggccataaaacaagtctctaaattaaaagcattcaaattattcagagtatgttttctgacctcagtatcattaagttggaatatattataggaagataacctggaaaagcctcagatatgtggaaaaacccatttccacatggcccatgggtcagaagtgaagtcaaaagggaaatttgaaagtcttttggattgactgatataaaaacaatagatttctaaacttgtggggtgctgttacagcatagtaaatggaaatttctagcattaaatgcctgttttaggaaagaaagatttcaaatcaatgacctcagcttctacctttggaaacttgaaaatgacaagcaaatggaatccagagttaccagaagggccaggtacggtggcttatgcctgcagttctgccactttgggaggccgaggcaggtggattgtttgagactggcagttgaagaccagcctgggcagcctagggagaccccatatctacaaaaaacaaaaaaattagccaggtgtggtggcatgtgcctgtagtcccagctaaccaggagtctaaggtgggaggattgcttgagtctgggaggttgaggctgcagtgaactgtgattgtgccactgtgttccatcctgggcaacagaatgagaccctgtctcaaaaacaaaaacagttactagaagaatggacatcataaagataggagcagaagtcagtaaaatagaaaacaaaaatacataggaaatcaataaaaccaaaagctggttcatcaagaacatcaataaattggtaaagctgataggaaaaacagtgaagtcacaaattagcaatatcaggaatgagggagatgacagtagtatagattatatagatattaaaaggactgtatgaggcaggtgtggtggttcacgcctgtaatcccagcaccttgggaggccgaggtggacagatcacctgaggtcaggagtttgggaccagcctggccaacatggtgaaactctgtctctactaaaaatacaaaaattagttggtcgtggtgctgtgtgcctgtaatcccagctacttgggaggctgaggcaggagaattgcttgaacctgggaggcggaggttgcagtgagctgagattgtgccgttgcactccagcctgggtgacagagcaagactccatctcaaaacaaataaataaataaaaaggactatatggtaatattatgaacaactttatgccaataaatttgacaacttatagatgaaatggatgagttccttgaaagacacagaaactattaaagctctctcaagaagatatagataagctgattagccctatatctattttattgaatttaaatgtaaaaatcaatatttagttactggaaaacttttaagtgtggttggaaatggtatacgaactttttcaactgaattttatgaagtctaatcacaggtaaaggttttctgatgaaaatttagtgtctgaattgagatatactgtaaaaaatgttatatatcttaattatttcttcacattaattacatgttgaaataatactttgggtgtattgggttaaattaaatattatgaaaatcttgcctgttttctttttacttttgatgcgtcagctaggaaatataaaagtgtagctcacattctgtttctgttgacagtactgctttggagcacagtgtttgaatgatctatcatttcaaagacctttcctcagttcgttattcatggctgtctgtattccacatagataaggtctgaaatactgctaagtggcatgttttgttttatgcttttataagtttgttgatcattactgatgtggacttttggtgcctcttaggctcattgctatcttccaaccattgtttgcaatttttacctagagataaagagaaagagacatttggtttcagagtagttagattgggatcatgaaagagcaacctcattttgatgcttcaaaaatagcacatcccccgtattactgggatttgctattcttgggattacttcaagaacatccttgtgttactggtttggatgcttctgaatgctgtgaagtcagtttcatgtacatggctcatcagtttagctctctcttggctttgtttagacagttggagcatgatggcctaaacagcttctttcaattaaacattttaaaatagtttacaaatagtaaacaaactccagtttttgtgactctttgtctcgcacaacaaaaacacaatctgaccatgatcatctggcatcttagggtgaaatatggttatactttggcccataccgaaagcaagattaaaaaggggcaggagagatagactgctgaactgattttcaaggttccaagaatattgtaggttaagagtaaaagtaaacttttggtagaaagcagtgggttgtctaggattgaagtatctgaagtttttaaacgaaaatttaaaaagaaaaatgagaattgccttacaagtacaatctcttcttttttaaaaaataaactttattttgaaatagttttagatttatagaaaaaaattagatagggtaggaagttttcatataccctacatccagttaccccagttattatcatcctaatttagtgtgagacattttcatgtttaatgaatcaatattgatatgctattaacttaagtccagactttattcagattttcttaatttctatgtaatgtcctttttctgttccagaattccatgcaggacaccggatacctcattacatttcattgtcatgtcaccttaggctcctcttgacagtttctcttctttttttgcttagaaattctccagaatttcagaaacttctgggcatcgctatggaactttttctgctgtgcagtgatgacgcagagtcagatgtcaggatggtggctgacgaatgcctcaacaaagttatcaaagtaagaaccgtgtggatgatgttctcctcagagctatcattgttgtaggctgagagaagaagcgatcattgagtgttcttctgttttgagtccctgaggatgtctgcacttttttcctttctgatgtatggtttggaggtgctctgttgtatggtttggaggtgctctgttgtatggtttggaggtgctctattgtatggtttggaggtgctctgttgtatggtttggaggtgctcttgtatggtttggaggtgctcttgtatggtttggaggtgctctgttgtatggtttggaggtggtcttgtatggtttgcaggtgctctattgcatggtttgcaggtgctctattgtatggtttggaagtgctcttgtatggtttggaggtgctcttgtatggtttggagatgctctattgtatggtttgcaggtgctctattgtatggtttggaagtgctcttgtatggtttggaggtgctcttgtatggtttggaggtgctctgttgtatggtttggaggtgctctgttgtatggtttggaggtgctcttgtatggtttggaggtgctctattgtatggtttggagatgctctggtatctgcctgcattgcttgccacacctgcccggtcagaaggcgctatgttgacaattgtgcctgcacggtgcctaggtcaatgaagggaaccgatggtagccactggatgctcctgggaaaatgtcactacaggcaccagagaagccagagctatgcccaaatttctatgagtctcagttttcttaaccataaaatgggatcaatgtttttgtggcatgtgtatgagtgtgtgtctgtgtatgtgtgaggattaaattgtgtatgtgtgaggactaattgccactactggatcctcaaagtggtaagaagtgttcttattaataatgacatccttacactcttacccagcaagattgatgggtgtggcactgcttctctttttccatcacatggtttccatggtatccttttgcccagggaatctttgctttgtggctagcactttgttgtttggctaatcacgctttctgtggtcaggacgctggcttctctggagccatgggattctagctccctgtcttgtccctagagtggtcactgtcttctctctccgcttgcaattcctgctttgctcgcatctcacttatgcagtgacgtatatcagtttcaccttgttctccgtgcctgctgatcattggcaccacttgcatggtgccatttagggcctgcttccagttaagcttgcttctccacaggcctaaatatccttgcttgcttcttttattctcactggcaggaccagggcggtctgtctttgcatgagacagggtctcgctcagtcacccaggctggagtgcagtggctgatcacggctcattgcagccttgagctaccgggctcaagctatcctcctggcttggccccttgagtagctgggactacaggcgtgcaccaccatgcccagctaatttttaaaattatttgtagagatgggatctcgccaggttgcccaggctggtcttgaacgcctgggctcaagtgatcctccctccttggtttcccaaagtgctgggatcacaggtgtgagccactgtgcctggcccttgatgtttcagttcttgatatttgatcctcagagtcagaaaatctaaaaagagggctatcccaggttgccttggttcatggcaaatgggacgttaagagggcagagagaatatgaacagaaactgttctaatattggtcatttaatgtgtaagtattgttcttttttaaacctccttcattttttttccaggaattgctggacacagtggcttggtgtgtgtctgaggactgtaggccatggccctaggttgtggttttaggtctcaggtgctcttcctggctgtctccttgcttctttcccatgtcctcttctttgtttccagccatttctcccttatgcttaagtttggtgcagcagggtttggctgctctcagattcctgcttcctcagatgctgtagttgtcaggcccagcgggctggcagcgggatcaggatctggctaggtttgctctcactgtggcagagtagggggaggcgtgggagagcacgtgtgaccccaggccagctgtagggagcataggcatggtcacgtagccttcaggtcctagactttgtcttctcatgagtatggctgtgtgtgtatggtgaaaactaggttctacttagcccaagaaaatgggcacattttgcatgtggtttctgtagagaaatgcactgggtatctgacatagcctggcagcatgcctccctcaggtaggttagtctcaggcggtgaagcacgtgtgtccagcaagaacttcatatgtggcataaagtctccgttctgtgaggtgctggcaaatcaccaccaccgtcaagaggctgaagtgatttttgtctagggaggcaggaaaggcttcctggagtcagcagccagtaggtgaaagagtagattggagaccttcttaatcatcaccgcctcttgtctcaaggggtgccaggaagctgtggaggctgaacccatcttatgctgccagagagtgggacaccatgagggtcaggtcaaggggttgtaccttgtttggtagagaattaggggctcttgaagactttggatgtggtcaggggagtgtatcatttaggaagagtgacccggtgaggacgtggggtagaggaggacaggtgggagggagtccaggtgggagtgagtagacccagcaggagtgcagggcctcgagccaggatggtggcagggctgtgaggagaggcagccacctgtgtgtctgcggaagcaggggcaagagggaagaggccagcagcgtgctgccatcacccagcgactggcgtagattgtgagagaccattccctgctcttaggaggggctgagttttagttttctcttgttatacaataagcttggtatttgtttacaaaacatttgtaaagctaaatcaaggtttgataaggcttctagttttatttaagaagtaatgttgaaataaatgtttgtccaattcgctttgctcatttaaggactttcagtacaaactgcaacaacaggattaggatttaaacgtttctgagatgtttttactcctcagaatttcccagaatgtgatctggttttgattttcaagcttgctgacccaataggttaacccacaagttttacgaagaccatctcagtccacttacatcaactgcccatgccacggttaaagagatcatcgactgatgtttggcacagcttcctccctcttgggtgggcaagcatttggaagagaaggctcctatgggtgagagtggggcaccaaagtcttccctgtcccatcccctagcttgagaagcccttctctaatgtggactttgtgccgttagcatcgttactagcttgaagttgaccatctggacgtactttctggtttagcctcacaagtgagcaaggagggttgagagatgtgctgtgaggaatgtggggccccagctggcagcaggctctgggtcaggggggcagggaccacgggcatacctgacagtgaggaggggccacacctgcagaaaaggatgcaggactccgccttgggaagtgttctaggccagagcgagggtctgtggtttataagtacacccacagtgctcgggaccctgcagatgtccagggtgccgtctgagcccgtatcatccaacagaatgttctgctagtgaagattaaagatttactccaggggctttaggatttattatatatatataaatcctatatatataattttttttttttttttttttgagatggagtttcgctcttgttgcccaggctggagtgcaatggcgtgatcttggctcactgcaacctccgcctcccgggttcaaactattctcctgcctcagcctctcgagtagctgggattacaggcgcccaccaccacacccggctaatttttgtattttttagtagagacggagtttctccatgttggtcaggctggtcttgaactcctgacctcaggtgatctgcccgccttggcctcccaaagtgctgggattacaggcatgagccaccccacctggccaggatttattgtatttgaaccatctaccattttaattttgatgttatgtagtatttgatgataatgaaagttaaattgtttttctttccatttttctgtttaagtgaatgacctgtatctagtttattcagtaacttcctgcatatatttgtttctttcattcttaatgaatatattcttaatttagttgctattatgttttgctttgccccaaaattgaaatcttagtttccttttagctcgttttagaactagtgatgggatgtgtcttccataaatctcttgtgatttgttgtaggctttgatggattctaatcttccaaggttacagctcgagctctataaggaaattaaaaaggtgggccttgcttttcttttttaaaaatgttttaaattttaaatttttataggtacacgtattttgtaggtacatgtaaatgtatatatttatggggtacatgagatattttgatacaggtatacaatacataataatcacaccatggaaagttggatatccatgccctcaagcatttatcctttgtgttacaaacaatccagttacatgctttacttattttattttatttttgagacagagtcttgctttcacccatgctagagtacagtggcatgaccttggctcactgcaacctccgcctcccgggttcaaccgaactttgggctggtctcaaactcctgacctcaggtgatccgcccgcctcggcctcccaaagtgttgggattacaggcgtgagccactgtgccgggcctgattgtacattttaaaataactaaaacagtcagggcacagtggctcatgcctgtaatcccagcattttgggaggctgaggcaggtgatcacctgagatcaggagttcgagaccagcctggccaacatggagaaaccctgtctctactaaaaatacaaaaattagccaagtgtggtggcgggcgcctgtaatcctggctactcgggaggctgaggtaggggaatcgcttgaacctgggggtggaggttgcagtgagccgagatcacgccactgcattccagcctgagcgacagagtgagactttgtctcaaaaaataaaaatgaaataaaattgggccgggtgtggtggctcacaccttagtcccagcactttgggaacctgaggcaggtggatgcttgagaccaggagtttgagaccagcatgggcaacatggcaaaacgctgtctgtacagaaattagctgggtgtggtggtgcacaactatagtctcagctacttgggagattgaggtgggaggattaattgagcctggaaggttgaatctataggtagctgagattgtgccactgcccttcagcctgggcgaccaagtgagaccctgtctcaaaagaaaaacaaaaaaacaaaaaacaaaccactattatcgactatatattattgtctatgatccctctgctgtgctgtcgaataccaggtcttgggcccttatttccatcactgagcaaacttcactctgttaagcagcaggtgtgggatttcatcgttattcagtaattcacaatgttagaaggaaatgctgtttggtagacgattgctttacttttcttcaaaaggttactctttattagatgagatgagaattaaaaatggtaacttactttatatctttataattgaagcccactagaccttaaagtagttaccagatgttttatgcatttaaatggccttttctctaaaattagaaagtaacaaggaaagaaaatgcttcgtttctatgcaaccctcttggtgactagtatgtgactcttaatgcaaccctcattgcaccccctcagaatggtgcccctcggagtttgcgtgctgccctgtggaggtttgctgagctggctcacctggttcggcctcagaaatgcaggtaagttgtacactctggatgttggtttttgtcgggggccagctgctactgatcctttatgtctcagctcagatgtcatttcaaaagtctgctctgccctctccaaattgcagtcgaccttgccctgtttatgtttccctcatagcactaatccatgtcagaaattgtcacgtacagtctatctgtgtgcttgtttattttctatcccacccttccgcaagagacttatgggatgtgtgccccaggacagcaggggtcttactgtcttatgctctgttgcagcccagcagcgataacagtgtctgcacatagtacttgcttaaaagatacttgccaaattgttgaaggttgaggtaccaatttcattattgctgactataggagttatagcaaaatatccatttgtctgttacatgagttaaaaatatggttgttgcactgtgaatagtttggtttagtcaaaacagttgtatcttaacggattgagaaacaaaagcaggaccacttttcatcagctccctccttctccttaaccagcaatacatgctgatgctgatatcccatagaccctcagctccatcctgagtcactgggaatgtggtctaaaccctcactattaatatgaactgagtttcaataagaatcttatatgggtcgggcatagtggctcatacctttgatcccagcacttcaggaggccaaggcaggtggattgcttgacccagactaggcaacatggtgaaacgccgcctctacaaaaaatacaaaacttagccaggcatggtggtgcgtgcctgtggtcacagccactcgagaggctgaggtgggaggatcacttgagcctgggaggtggaggtcgtgttgagccaagatcgcaccactgcactccagcctgggcaacagagtgagacctgtctcaaaaaaaccaaaatccagaaaagaacttatatggctgcagaggtataatcactaaggaaatttccttttgtataatcttttttcttttactatcatttaaaaaaatgtgttatatttctgaagcaacacatccaggttctgcacatagcagccaaagtgaccttaaagaatataactgggtcttgtcattcccttatttaaactcttgtacccatttcccagtgccgtttagatagagattccagactcgtcaatggctctgtcacctcagacaccctgcattgactcattagtctgattagagtcaggtttttcttcctcctgatggtttttttttcccccttagttctcagcggaacagtcacttccttagggaggtttccccagccaccctctgaggccgtgcttgttgccagactctgccactagagggcagggctgcaccactcctggcacctcgcacccggcctgccctgtcactctgtgtgttgggtgaattcctgtgatctgtgactcactgctctgtgtcctacacattcggcttttcttctctccccacaaccccattttataattctcctttttcaggaaagctttattcccatttaaaaatttttgtttttaaaatggtattttcttacacttattttctaattaaaaatgagtgttttaagaagtattatgatttactgcaaataatttttaaacccagccttttagatcctctgtgatcataagagaaatgaaggatgtctcccaacacttgagcttcatccacatttcatcctcctgttctttcagctgagttttccccatcccattagggactgttggaatataaaactggcttttccctaacagggaatgaattgcttctgtttctcctgaaggagagctggaagaatgacttgcgttcttttgcatacacaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggtatgattgttgcctcaggtcacaaacatgcgagtgatgctgtgagtgagtctgtggagggtgagggcttctgaacagggagtcctgtgggagtgcttcttggggtatgttgtatgtcgtaatttagactaccatcatttgtgttatttttgaggcacctaaggacttctttccacttctcatttcttactgtggggtgaagagttgaattgggagatggtttctagatgcaaattgaaaaggcatttttccagagcagatttgttttcggcgtactagagtgactctttaacctagctgcgggaagatgactgtgccaagactgcaggtaggagaaagctcactgacgaggccttgtgggtctgaacgtcctgcagctatcagagcctgttggcttcctgttgtgcattccaacaaatcatcttcaaacccactttagtgttttgtttataatgtccagaaatagtgaccctgtcacatgctctacagattacaggattcttagcctcttcctttttggtaggtcagtcctgggtttgagcccaagtgaccctcctgggaggtgatgatacacactgggtagagtggaatcagatggacttggattagaattctgtcctctttactagttattttcctctaggcaaactgcccaacagctctaagctatttccttcgtattctgaaaaataagccttaatgggacccatatagggcaactctgagagtaaaataaaggaatatgtgttagagtgtagcatagtcacccacgggaagggcttagatgttagctgctactgctcttattagctgaatgatttggaataaactgttagcctctctcatgttttttctcttgagcttcgaagttttcttgttaatactaaggagatattcaaactagtcatggggttttggaatgacgaagggagatgatgaatctaaagaatttagtgtaatatttcttcatgctcagtaaatggtagtttctgctgctgttatttttattaccatctctttggaatgggagtaggtgctcctttgtggtcagaggctgtgagagctccacagcgccagtttgcccatctgtacactggggtctgttgaaggcagtcccctctgtgatatctctggctgtcagagctcagatgatagatggtatttttgtactcttagttctcatcattttcatgatttcgatcaccatttgagtatgatgatgctaacactttgttgaacgtagaatccgttaattacttccttcctgaacctttggcattaaaaaaaatctattctgctacctctctgctcatttatggttattcaaatttattatcaagagcctggtacagtggcttgtgcctataattgtagctacttgggaggctgaggtaggaggattgcttgaggccaggagtttgagaccagcctgggcaagatagtgagaccctatctctaaaaaaactgaaaaaaaattagctggacatgatggcatgtgcctgtggtcctagctactcaggaggctgagacaggaggctcggttgagcccaggagttggagttcgaggctacactgagctgtgattgtgccaccacactccagcatgggtggtaaaacaagatgccatttcttaaaaaaaaaaaatatatatatatatattatcaatgaaattcagtagtaccaacaggattataaacaaagatagtagttcccttcctactttttctcttaatccttgtgtctcacaggcaaacataactcttagtatttcttccaatatttactttcatgtttctttctttctttctttttttttctttgagatggagttttgctcttgttgccaaggctggagtgcaatgacgcaatcttggctcaccacaacctctgtctcccgggttcaagcgattctcctgcctcagcctcctagtagctgggattacaggcatgcatcaccacgctcggctaattttgtacttttagtagagatggggtttctccgggttggtcaggctggtctcgaactcctgacctcaggtgatcctcccacctcagcctcccaaagtgctgggattacaggcgtgagccactgcgcccagcaacttccacatttctaaataacatgcttctactgctattttttttttcaattttagacatttttttactttcactatagttctatcagaattcagtgtgtacgttattatgcctaagtaaatagtcatggttgcttacgtattatatttctttgattgtgtttcttatttgatgagaaagctgtgttttttgctctgggttgaaactggagagaggacctggggaggaggaggaggacagatgaagttggtgactgtaccttcatggccatagctgggttctcagcacccggggatctgctgatcacctactcataggccaggcccctatcgaagttctaggtgacccagtgctggggacgggggggccacctgcaaggtctaatcatggaggtgggggctacagtgttggcttgtgctggggccagcatccttaggaaggcatcttggaggtggaggagacagccgcccacttcttgattggggccttcagcagcaccagcttcttgggcaggctggtgctggctttcatcaccatgtcgtgttcaatcttcttccagatcctgacttctaggttcagctttcctcagaccctggttcctttcagaggccattgctgctgccttgctctttgctggcttgtgccttgattatatgtctttgtacaactttttgttttcctggagttaatcttcacatctgttttcttggagttaatcgttacctctatatcgcttgcttattattctttggcctttttgtcttctcacaccttccaacttctttgtaatatgtgtttagtacaatttttcatgacaggtagtttactgaatcagtttttccccagtgtggtcatccaacttgagttatccagctctctgccccagtctgggcaggttgatcttcaggtctgtagtacacttgtatcctaggacttctctttgccattagcctggaatttcctttgcagttctcccgttggatgcccagttcctagatgccatatgtttttctatcgtctagtagcttcctgagagaagatgaatgggagggaaattgtatgaggttttgcattcataaaaatgccattttttttcctgtacacttggctgggtatggtgttctggggtagaaatcattttccctcagaaatgcaaagtctttgccctgttgtcttaaaatctccaacgtgacccgattccttaacctatgaatgtacttttctttggaagctttccatttttggggaggtgaagtgctaggtacttagtaggccttttaatttggaaacttacatcccttcagttctgggaaaattttcttaacatttctctgagaagttcttgccttttattttctgtgttctctcctgaaattggttagttggatgttggtcctcctagattgactcacatcttacctttttcttttctttttctggtactttttagatatccatctcaaactcttctattcattgttatgtttttaacttctttcttttctttgtctcttgatggggtcttgccctgttgcccaggttgtggtgcagtggtgcgatcatagctcactgcagcctcaaattcctgggctcaagcagctgttctgcctcaccctcccaagtagttgggactacaggtatgcaccaccacgtccagctattttctttacttttttttttttttttttgagatggagtcctactctgtcgcccaggctagagtgcggtggtgggattttggctcacttaagcctctgcctcccaggttcaagcagttctcctgcctcagcctctcaagtagctgggattacaggtgtgcaccaccatgcccggctaatttttgtatttttagtagagccagagtttcaccatgttggccaggctggtctcgaacgcctgacctcaggtgatccgcctgccttggcctccgaaagtgccgggattacaggcgtgagcccatcattagatctttaaataccagtatctataagtcttttcctcttgagtcagctagtatccctggaaggaaattactcattttcctgcttggaggctataagcttggctatgtttatcctgcaaccggggactggaagggaggggactgacagtgttgctggtcagggtgccctcttactttttgttttctgtgtgcatctcacgtctgtcctcagcctatgtaaacacctcttgagattatccctctcaatctttgccggaggtgggggaggggctgcttcctgggctgccttggattggagggaagacctcaggtgagtgggtgggaatttgcccaaggagccatgagaccagccactatttcaccctctccatccctccactttcagatgtatgtggcgcctccaaagcccgagctcttcttggcgtctgtggcttcaataagcttgctttttgctggtatccctcctaccctcccctgtccccagcaaagcttgcatttgaacttcttcctacgggctaacaaatcagtcagttatgtagctcttgttactttttagcttccgaagttttgttgacacccgtagtctgctaatgtccctgttctgttctttctgttcgtgtaaatatatgctttatacaacttctttacatgatttttgtggggtttctgggtagcagagcttcacaagttcaatccagcgtgttggattagaaatctcccaccctctggtttattcttattctcaaaattacctgccaaacactgatactcccttgtttttccttttcctgacaggaaatgtacataccatacaggacagaaatcattagtgtatcccttggtgaataaccacaaagtgaacttaacccttgtaaccgccacccaggtcaagacagaatattaccaagcactcagaagcctctcccctattcccccgtcactgctcctgccttcctccccaaggtcatgactgctggcttctaattccagagtctgtttttaaattctgtgtacatagaccatggattaagtgttctttttgtctggtttattttggtcgacattaagttcatgagagtcttctatattatcgtgtgtattagtattcctgtagttttaggagcttcatagcattccattgtagggatataccacagtttattcattgtattatcactgggttgtttctagttcttggctattgcgagcagtgctactgtgaccactcttaggtgtgtcttttggagtacatgtgcaggtttccatcttgcacagctagaggtggagttgttgggtgatagggtgtgtgcatctcagctgcagtagaaactgccaaatagctttccttgagtgcttgtaccagctcacccttttgccactgtgtatggggattccaggagctctggtcctcgctagcacttggaattgctgatgcttttactcttagccttcctgatgggtgttttctggaatcacattatgattttaatttccattccttaaagtacccttggctctgaagtttaatgattcatgcatctcttcccttttgaagtactcttacaggtatgttgtgcatgtgttgaaaagtggcactatctattctaaaatacagtatgcctcctctgtgtttgaacagttgtagcgtggccttggggcctcctgttagctggcttggagaagggattcttgggattgtagagattagacctgaggaggccccttggagctctctgactaaattttattctttattattccaaactatttaagctcaccgtgtgctgactcatcataataatgagtagctctcattgtgcttgtctatttggactcatacaatgattttttttttttctttgagacagagtcttgctctgttgcctaggctggagtgcagtggcacaatctcggctcactgcagcctccacctcccaggttcaagtgattcttgtgcctcagcttctcaagtagctgagactgcaggtgcgtaccaccatgcctggctaatgtttgtatttttagtagagacggggtttcaccatgttggccaggttggtctcaaactcctgacctcaagtgatctgccttcttcagcctcccaaagtgctgggattacaggtgtgagccactgagcttggccaaagtagttttttaagatgttagtatcttttcttgcagctaaaaaagtttgtcagagatgattctactttgttctccaggtgttttctcagggagaaattggaggcagtaagccactgggggagtcctgtggctggggggtggggtagtcctgtggctccttgtcagggagtcctgtggctggcaaggagagaagtcctgtggctgggttgggagggagtcctgtggctggggtctcatcctgtgcctaacagtgtccagaggtgccgagaccagctcagtcggggagaccctaacccagcagcgctagaggaattaaagacacacacacagaaatatagaggtgtgaagtgggaaatcaggggtctcacagcctttagagctgagagccctgaacagagatttacccacatatttattaatagcaaaccagtcattagcattgtttctatagatgttaaattaactaaaagtatcccttatgggaaacgaggggatgggccgaattaaaagaagaggttgggctagttaaccgcagcaggagcatgtccttaaggcacagatcgctcatgctattgtttgtggcttaagaatgcctttaagcggttttccaccctgggtgggccaggtgttccttgccctcattcctgtcaacccacaaccttccagtgtgggcattagggccattatgaacatgttacagtgcttcagagattttgtttatggccagttttggggccagtttatggccagattttggggggcctgctcccaatacagaggtctcgtgtaaattccctgggaggcgataagcctctgagaaacagactatgctaaccacgccatgaaagagaaacttatttataaatcagatgccagttactagtttactgcttatttgcccaggcgtagctctgacagagtccccgactcatagtgcttgctcagtgcatgctgaacaatgattggaatcaagtcatggctcagagcatagttttgaataatgggaaatggatgttcttaagtaacatagtcaccaagataatgcgactagctgggtcaccccttttcaattttaggatatttttatcaagatttaaatggccatcattagagttatagcactttctcctttggattgtcctagaggcccatgagaaagtattccctaatttcttaggagaacagtttgtgggtagtatgcggtcatgtccagttaaattgcagatatttccgatcgaagatgttccagtcctgagaacttcgtgacattagcaggacttctacaagccatctcttagggtggggcatttactgcagttggctagtactcttttctccttaactttgtcatttgttgatttttttttaactgtccccaaatactgtgggcagagtgtatctagaattgaggcctccaccattgcggagaggacatggatgctgagcagtcccctgagtgaaggttataaagaagcaaatagactacacatgtctgtaaactgctcttgagtgtcccaaatttggggtacttcagttcagctgtaggaaaagcctcaaactgtttatactttgcaagaattggaaacttctaattcacgttaagttttatgtaatacatgataagcttcataggagcttcatcttttatctacttggacttttgcttccgtaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggtaaggtggaggcatatgagtggaagagtctccagcatgtactcaagatagacctttgaaataaataaaaccagatgatccctcagcttctagaccaggctatttggcactggttgattgaatgtgaactgcactggggctgctgtgagcccgcatgggtctctgtgaccctgcagatgcagccgtgcccagggactgggcagtgggtgtgggctggtgtgagccctgtctgccacccagggcctggccctctgtctgtgtcggccatgactatggtgagtcttgtaggcttgagactgtgcctcgggttcctgcgggttctctgtaggtcagttgacagtttctcctgttgtttgggtaactgtggaaacgaacactggcaagtgctgaagcgagcatgtggacgtgcgatatgaaataacgacctggctttcaaaggcagtgaggctctctggaaaggaccttgctgagctagggatgtgggtgtgtagccattcccagtgggcctcatggcgtactcgttcatgatcatgtttgtgccatcttgatctctcaggatctcttcttttttaacagattaagccgggaatctccaaacagtgagtcagatgttaagatgtcttgcttccacccccacaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtaggagcacagggtttactctaggccctgcatgtgaatgactgacattcaaagaaccgattaatttggaagagaagcggcagaaccgagagttagaggtgtggactctggagctgcgctgctcgtttccaaccctaggtgctgacctctagctgtcttccctctgtatgtccctgtcaccgtgagtcaaatgcgggtgatgcctcctcaggtgccgtgttacctaagcctctcagagaccactgctaccctgtttctaaaaccagaggtcacgatatgtgttcatccacccagtaaatactgattgagcacccactgtgtgctaggctctgggataggggctgggtatacaatggtgagtatttcagctgcagcttctgccccgtggaggctgtggcctagcacactggtctaggcacggtggtatatgctcactcaaggagatagggacgtggtcgtttggggtgtcggaacaaaatgtcggaacttctctttccaatgcagagaaaccttgcagtaattctaatgtactgtgattggcagttgacttcagttctttgtagcacgcttactcaggttatttcactaactatgtaaccatgcagcctcattttaagcaattggattttttgaactttacttaaaatgttatgtcagggtttttattgtgcttaatgtgtgccatttagctaagttttgtaggatacgaaattgtaagtggcttaaaatgattcttaatagaatcatgaattgaagataatgctaataatttaagcactgagttaggtagtgtttgtaaaatgcttagaatgcttcctggcacatgttaaggccatgtaagtgctgcgtgttgataaacagctgagcaaaagtggactcttaagaaagtattggggctgagagttctgttccaaccagctgccctttggttatttttcagaataaaagcagagtctcatgggatatgacatttatatttccttcacaaaaaacactgctgagtgttttgttgagtaaaaagggtgtagccatggtaataatacatttaaaatatagtttatttcatctttaccttgccttgttttttttttaagctagctttttattgagaattccacacatacaaaagtatcaactcatgaccagttatatttcatttataatcctacttctccctttttttattatttgaaagcaaaccccaattatcctcttatttcatctataagtatttcagtatctctatagatgaggactcttctttatttttaaaactttatttttaaaatgatggtcagatgcagtgttcatgcctgtaatcccagaactttgggaggccaagctgggcggatcacttgaacctgggagtttgagaccagcccgggaaacatggcgaaaccccatgtcttaaagaaaaaaatcagccaagtgtggtgatgcatgcctgtagtcccagctacttgggaggctgagatgggagggtcacatgagcctggaagatcaaggctgcagtgatccatgattgtaccactgcactccatcctgggtgatggagcaagattctgtctcaaaaaaacaaaactgcaaaacaacgtcacaaaacagtgccattgttagacctgaaaatattaaacatttcctacatcaaatacccaccaactcattatcaatttttctctctactcttttggaatcagcatctaaataaaattggtcgataaggattgtaaatctctttgatgaactggttcccctccatcccagtttttttcccttagagttcatttattgagaaaccagattgtttgtcttctaagttttcctgtggtctgatatactgcttccatctccactgtgtaaattaacacctttttctcttctctgtatttcctgtaaatcaataattggaggaaaagccttgtcagatttagtgtatattttatatctgagtccagtatttcttatataatattttaagataagtgtactcttttaaaaagtattgaaactatatgctcaattttttttaactgatgcttttaagaaggctgcttgatcataaaagtttagagatcattggtctgatgggaaaagcaaataattactaaaccgtttagcaaggttgaggtgcacatggtggggcctggagaagttcagtcatgagccgtcacttatgggcacgtggaatctgacccggcacagagttgggagaagacaggagctttatagacagaaaatgtggtctttgctaagtcccaggagtgaaagggtgagacagtgctcacagcacacgagtgtgggtgcgtagacagagcaagggtgggtcctgaaaaggcctgcaggctttctcatagattagcaagagtgctggttacggaggtttctaacatttgtgaacagatcgaaactgtgttaaattgggattgcagtaatcctggaaggacagggatagagggtgaaggggaaaaaagggtatggatgtgagacttaattgctgattttcttaagacctttctccaaagtaaataaatgatgtggcacatttttgaactggcaaattctaaactctagatatgattatctctataacatatcttactccatcttcttttgactaaaaactgttcttaattaaattaccatgagacgttcaattcagcaaatgtagtttggctaaccatatttaattagaatttaatataatcctaggcctggccaaactattaagcaagtgtgggcaaaatattgataattttagatatgcaggaacttagtttgctttccatgtgtgcttttcgaaaaaggaataaattgaaaaatagaggaagccctgaaatccaagaagcaaactctctcacctaggcatgcagtaaaagcaattctaggatgattgctgtttggcgcgtagttcgtattagaaaccattcttcttgaataaatagtatgtttaagaagctgggcagagggaaggcatatgcatatattatcaacaaggagggagaaaaaggcaattagtaaccatccataggagggtcagcaagatttataaaggaaatttgtgatccaagtatgaagcaaaataaggtgcagaataaattttaagcaagtaatagattagagtaagagaacccatttgaccattaaccttgggacattctctttcaaatgacatggagtagtactgaaatctttctttctttctgagtctaggttattgtgactggactcagaaagaaatatttcattattgcagtgaataacatttgtgaacattattgttcataaattatgcagtgaataacatttatgaacacgtgatgtgtaagatacatactgtttatttttagttaagttttttggctcaacttctaggcagagaacattaaatgtaaatagtgttacctaggagcatgtaaatggaaatctccatagtatgaaagcagtgctgttgctaacagaatttaggagggggcagatgaggtgaaggaaatgtgggtgctgatttccttattacattgagaggagccaggagattctttgttcaaaatggatggcttaagaagtcaaagtataagctgattacgtagagcaggtacccaaaaatgttttgtgtaaggggccagatagtaaatattttcagtcttgcaggccatcccaagtctgtggcagctactcaacactacctttgtagcatgaaagcagccacaggcagcccataaatgtggctctgttccggtgaaactttaggtacaaaagcaggtgcaggccagacctgacctgtgcactgtggtttgctgacctgggattcaggggtatagaagttaccatcagaagagctaaaagtgagactttttactttatactcttctacactgtctgattttgaaaaaaagaaacatgtattttataatattaaagatagggttggcaaatagcaaataaaaatacagaataccagtgaaatttgaacttcagatacattatgagtaattttatggtgtaagtatattccaaatcatgtgggacatacttacactacaaaattatttgttgtttgtttacagtttaaatttgagtgccttgtattttatctggcaactgtaattaaagggaaaaagaataaattcattatgttcatataatgtgatatagcaggggtccccaacccccaggctgcagagtggtactggtccatgggtccccaacccccaggctgcagagcggtattggtccatggcctgttaggaaccaggctgcccagcaggaagtgagcagcaggtgagctggcattcccacctgagcaccgcctcctgtcagatcagtggcagcattagattcccataggagtgcaaaccctattgtgaactgcacatgtgaggggtctaggttgtgcgctccttatgagaatctaatgcctgatgatctgaggtggaacagtctcgtcttgaaaccatcccctggccctgtggaaaaattgtctcccatgaaaccagtctctggtgccagaaaggttgggtagcactgtgatatagtattaaaagtgctaataaatatggcatactgcctttaaaatgtctggtagctctttctcagtggcactcataatagtgttttttgatttttaaatgtgtgtcaagctgactctcccctccgtgtatgctgggctttattttccctttcctagtcaccagttttgggaaatagagatcttcattctcatgctgctcctctagtgcaagtgctccatttatttttaaggaattaatataacaaaaaatcatgggaatttagaaaacaacatggaagctaatgatcacattggtggaagtgatagggaaatatttagggggagaagttaaggtataaactttgtcaatgaagtcctattaaaaacaacaaaaaagtgaagcttaggatgcattttataaactctgaccagaacacctgtgtttctctgtttctaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttataggcaagttattagcaaggtctactcttacaattaactttgcagtaatactagttacactctattgattatgggcctgccctgtgctaagcagtctgcattccatcttccttgccaaaacttataatacaaatttcatctttattttataaataggggagttgggctgggtgtggtggctcacgcctgtaatttcagcactttggaaggatcgcttcagcccaggagtttgagacaacctggccaagtgagaccctgtctctacaaaaaaaaaaaaaaaaaaaaaattagctgggcatggtggcacatgcctgtagtcccagctgctttggaggctgaggtggtaggattgcttaagcccaagaggttgaggctgcagtgaatcttgatggcagctgcactgagcctggtgacagagcaagatgctgtctcaaaataaatttaaaaataaaataagagaattaaagtttagcaggttgggtggcaaaatgaggccacacatttaaagcccctcctcctgattcttttctctgccttggctgcctcctgtggcattttaggtgctgagaaatgaaaacagtagggaaaatagttccaggatcctcatgttaatttgccagaaatggcatcttcaagtcgtcagagggatctgagagttccttcctggcctgacttgagaaaatccgtctgtccccagctctgcgtctgcctccactgcccagtcacctcctctccatgctcttggggctgggccctaccccaccatgcagtgctgccctggagcagtgagcttggtgggtcctgtctggcatgagagctgcctttgggagctggatcccagcctctaccactgggtctggtgcctagcaggctatggataaacttctgctgactccggcctctcctaagccactgcaacgtggtcggtgtagtgcacagtgtgtgtgcagcgtggccttactcacagcctccacattagagagaatctgactgaagtcttactgctgcctcgtgtgaacataaatgtttgccagaaccatgagcaggaaatgttaatctgccttgtttcctgtcctttacacggaagaatttttttctgtatggaatgcgtgccttacaaataatgagtggaaatacccatcgctaatgaaaagttatacttgactgttagtcagctaaataatctgagatttctaatacttttaatttggcttttacaatgcaatttatcttagcttttttgatttcttaggtcatatctttagaactatatatttgaatgttaatgtaattttcatattgaaattaaaatgttgaactgcgatgttaagtgtttcctgtggaaaaacgttcacattttctctagttttaaagttgaatcaagctgtttgaagattttcacatttcttctagattttatcagcttgttactttatctgtcactttctgtgatttgcagctggagggggttcctcatgcagccctgtcctttcaagaaaacaaaaaggtgattatttcagaaatcagagtcttgtgttgaatcttactgattttcttgtatttctgtaatgtaatgtatcttgtatttcttgtaatactgtattggactctgtgtatatctcttctcagatgagtgattatatgtgtgaatgttgctggaatctgataaccaggcctgaatagttttgtagggtggcttttaaaaattactttcatatcagaattgctttgtcataaattttgaacgcatcataaatttctaatgttcggggtcagcagactttttttgtaaagggacagagtgtaaacatcttagctttatgggccatatggtctcttttgcaacattcagctctgccctgtgacaggaatgcagttgtaaagacatgagctactggccagctatgttccagtagaactttacttacagaaacagacaggctgtagtttgccaatacctgccttagggaatgtgttgttatattttgtgagttaccttctcagtaaattttatttagtattagtcaggaatattattaagtagcttcttttccagcctggtcaacatagtgagacccggtctctaccaaaacaaaacaaaacaaaaaaacagccacgcatgtggcatgtgcctgtagcctcagctgctgctcagggggctgaggcaagaggattgtttgagcccaggagtttgaggtcacagtgagctgtagtcatgccactgcactccagcctaggcaacagaatgagaccttgtgtcttaaaaaaaaaaagtttcctttgttgggttattttaatttggacctggttatcatttttcagccatatttaactttgtacatatcagaatgttctgataaaacttaacttttattaaagtgtttgtgatataatctgctagttttggtacacattatcttttgcaatgccagttattttcttttccagtgtgggtttgcataggaaaagaattgctgtcactttctattttgaaatcttaaaagactgatccttttttgtgtcatgatttgagtatttaattgagagcctaatgcctaatattatttgcagtattaaatgggatcttaacaggaatagcattctagccttcattgaattaagtaaacatttcttaagagaacttggaatctataatatttgcgtcatcatagtatgagatacttaatcaagtttgagattttagtgaaacattgtttagaagccaaaaggattctaggaaaaattaatgtctatattcttgaattaggagagattttgggacgtgtgactaagttacgctgacacttgtttgtttcttagtcgctttttccagtggcggtgagaacgaagatgactgattcacattgctcagatgagtttatcctcttctggctgggacatgggatatatcctgtctcttttaagcctttttggtatttttcccccattgagagctgtgtcttcaaactcttctgttatagctggaaaatcctttttaagtgaaatctgcccaaattataagacagatgaaggtagagttgtgttggatataggattagggtgaaagtagtgggggtgtcctggagcctctcttctggtggcagcctagctcttgtgcctttgaggaaattaccctggggacggctctgtggaacatatttgcaaaccactgatttggaagatagagatggcttttgttaagatctgaattcacctttttggcattttatttgatttctcaaggtaaagaacttattttgtaataaagtttcctattatttagtagataggccaagttgctgtgttaattccatgtagattttgggtttcctttgctcattttttcactcttaatctcacatcattgtaagtttatggaagttatcatacttctgactttttctttgaagagcagaaattagaaattcccaataattattttgatagtgtcatttaatgacactcacatgtgatgtagccacaaagatttaatgagttcagttttaaatcatattaagactgttggtttcatttgttctcattaatgtaattctgaagatgaacaataaaatgtatttttagaactttcaaatgaaatattatttcatccttccagatcatataatgcttaagttctgattgttaatcataaagtctagaaaattaaaagataataaaatgaaagtgacttttaggtattagagttttattataaattctggtgtgtcattggagctatgacatgaatatttcaaaggccaatagcattggatctttacagttataacttaccatttttaagtttaagtagtaatatagattatttaataatcaaaatcaataaatattaattattaaaatgttttgtggtatagtttgagaatcattgcttttaactttttccatataggtttattgactttaatagcattctaaacataacatctctacattctttgtgtttaatactgtggaggtataaaaatacttatatatgatgataaactatattagagtaaattaaatattcttatgagtttcattttagagtgcatttacttaattttgaagtccttatttttagcaaactaaaaggaatgttggtacattatttactaggcaaagtgctcttaggagaagaagaagccttggaggatgactctgaatcgagatcggatgtcagcagctctgccttaacaggtagttctcactagttagccgctggtgtggaccttcactgtctgccttccaccccttgcccttcctgctcgtccccctgcacctggtggacagcacgactgggggcagcagtggagccaggttgcttaaatggggcatattcgggcttcttttataatacttactctgaagcttgtgtgtctgtggtgtttgcatcatatatttgttgttttccatggtttaggctgttttaaaattaggtttatggcttgagcatagggctttgtgagtaggggatggcaggtcgaaacatctcatgagttggatgggttatgctgggggttgggaaatgggatgaaaaattatgggatgaaaaattgcctatggatagtttaacttgaaagaatctgcctttgtttacagatagttatcttttttcttttttgagatagagtctcacactgtcacccagtgcagatacccagtgtcactggagtgcagtggtgtgctcttggtgcactgcagcctccgccttctgggttccagcgattctcctgcctcagcctcccaagtagctgggactacaggtgcccgccaccacgcttggctaatttttgtatttttttgtggagacgggtttttgccatgttggtcaggctggtcttgaactcctgacctcaagtgatctgcctgcctcagcctcccacagtgccgggattacaggagtgagccactgtgcccggccagttacagatacttatctaatgaaattctctgtgtactttataaaagatgaggattaactgaaggtactaataactggattatatgagggtggttttggttgtataatcctatctaaaagaatattttagctataactgaaagtaagacttaaatatttagagaggaaaatctgaataattctagtagtaattatttatttacaaaataaaaatagatttttttttgattacacaaattaaacaacaataaaacatcacagcaatccggatactataaagctcacatgcttaccgacccaactgccccaggagtgaccactgccaacagcttcatgtcgacctttttgccataatttttatatagccttttttgtttttaaatggtaatttagaaagtcaactaggaaaatgtgttacaggtttatcttccaggagaataggactggagtcgagatcttgaatgtggcttggaagaaggcaagcccaccccagagagatgagttgacagttgtttctgaccactgcttgcttagagggcctgcgtgtctgtgaccgcctagctttgcgcccctgactaggctgccccttaattacaaatgtctttatatattgctccagctaaggcttggagtagtcggttaagaacttgaacttcggtttttgcagtgaaacagcatttgagaatatcaccttctgataagccttattttataaggtgggtactgtagtgggaggcagtgtgagagatgcttgaaggatgcactgctgtcctgcatttcagcatcttcaggatgctgtgcagctgaaacatttgataacggtggaactgttcgttattttgcaagcctgtgattccctattgaatgttttctctcgccatttgacaaatgagtgtttctctgtcttcagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtaagtgggcagaggggcctgacatctttttttttattttttatttgagacagagtctcactccatagtgcagtggaggccgggcacaggggctcatgcctgtaatcccagcactttgggagactgaggcaggcggatcacttgaggtcaggagttcgagaccagcctggccaacatggtgaaaccctgtctctactaaaaatacaaaaattagttgggcgtggtggcacatgtctgtagtcccagctgttagggaggctgaggcaggagaattgcttgagcctgggaggcagaggttgcaatgagccgagatcgtgacactgcactccagcccgggcaacagagcaagactccatttcaaaaaaaataaaaaaataaagtgcagtggctcgttctcagcccactgcaacttctgcctcccaggctcgagcgattctcccgcctcagcctcctgagtaggtgggattacaggtgggcaccaccacactcagctaatgtttgtattttcagtagagacagggtttcaccatgttggccaggctggtctcaaactcctgaccttagatgatccacccaccttggcctcctaaagtattgggattatagttgtgagccaccatgcccggccctgccacctgccatcttttgagttcttccctggagacctagacctgaaccctcctgcttgttctcttgttatctaatacccctattgacagcgcagcttagatcattaatggagagcttgacctcatctgataccttcactgaaggaaacaacttagtgtcttttgtgttgaacactgaggtaaaaaattggaatagttgattatatgaactctgctaaaattgagtgcattttacattttttaaggccttgttgggccctggttaaataattatttttaaaaatccttaaggagcctattataaacagatctgtggtcttaatgaaatgtgattaatactgtgcattattttaagaacttttgacttttcaaaaaacttttacaacatttcccatttgatagcggcataggtttaagcacttctcatctctaagttagtggacaaaaaaccctcatggatagtctaataatgtttgctacaagtccatgttgagttttatactccattttattttcagttttaaaaactgtggttaaatatgtgtaacataaaatttatgttcttaaccattttttgcgtatacagttcgctggtattaaatacatttaaataatgtcatggaatcattgctaccacccatctctgtaaccttttgatcatgtaacactgaagctctgttcccattgaactctattcctcctttcccgccaagtccctggcaaccacgattcttctttctgtcttctgaatttgactactttgggttctcatatactttaggagtcacacagtatttgttttacttagcataatgtccccaaagctcatgcatgttgtagcctatgttagaacttcctaatgtttcaggccaaatactattccattgtatggataggccacattttgcttttccattcctctgtccatggacacttgtattgcttcatgttttagccattgtgaatcatgctgttatgaacgtgggtgtacagatagctcctggagactctgctttccatttttttggctaaatacccagaaatggagttgcttttacattccaattttaatttaaaacattcatatcattgagtgttttacttaatagtatagtagttaacaaacttaataaaatagtattttggtaataatttgctggtagtccattgttcagtttttttaggtaaattacacaggacatttcaagtggacatgaaacatcttgtgatgtggaatcatgccccaagctgatggctaaacatatgaaataccataccctaaatttagtagatttagtctttgcaatttaggagataacctgttatattgttaggtttttgtcgaaaagctttgtcctcatatttccaacttgctgtaaaatttgtttgtgaagacaaatatttttgtatgggttttttctttttcatattaaaaagaaatgtccacattggaatttttttggagtttttagagctaatagagcttttcataatgtagtgggaatgagtgatcagtaagctcttagcagtttccatgcgtgcatttctgtgccttgaaataaatgacagatgagtacatttgtgttctgtgtgtaaaatgtgctctttcctcattgcacttccatgttggagggcttgtctcttggtgatcacacttcaaaattctcacagccccccttgaaccgtttaggtgttagacggtaccgacaaccagtatttgggcctgcagattggacagccccaggatgaagatgaggaagccacaggtattcttcctgatgaagcctcggaggccttcaggaactcttccatgggtatgtggactacaggtgatgcgctacaaagtggtttgtattcagacctggacatcttaattatatctttgcttccaagaagaagtcctttgatactgttttctgagttctgaatagctgatgaaaatgaccaattgaggaataatcatactttttcttgatctaaatcttatacttttgagttatcttagcataaatgtataattgtattttaagtggaaatttgtcacttaatcttgatttctctgtttttaaagcccttcaacaggcacatttattgaaaaacatgagtcactgcaggcagccttctgacagcagtgttgataaatttgtgttgagagatgaagctactgaaccgggtgatcaagaaaacaaggtgagggacataggcttgagacgacttggtgtttctgagcttgtgtgaggatttaaaatcgccctggctactgtctactttattgctttcccatccctgggcctttaaatttcccctttaaataccagctcttcccaggcctgttgttttctgcctttccaggtactacccacagccttgagaattgcctgagttctgcctcctttgagagtgtgccccagacaaatctattctgtactgaatgtttccttgtctgatttcttggatcattcatttgatggttgcgtatggcctgcaacgtttcttgttttggttctactgaactgttctaaaagtctctcttcatattatctttttacatgtaaatgtaactgtcttcacttttaattcctcaaggacaaggaatagcgtttcacagttcgtcccatcaatcagaattatagcctttggcatctccctatctaccaggcccacttcctcttagatttgggcttccccaggctgttgcctttccccaagtagcttctgcttgtcctgtagaagacctttcatgctttgcttctgcagcagccgttcctgaatgcctagtgtcaactgccttcttaccacgcccaccctccctgcatgctgcatttatcccctgccacagccctgtgaccctgtgtcctgctgcctctgacttgtctgtttctgcttggccatggtctctgtgaggtcaggtgtgcatatgggcacaaaccagggcatctctttatccccagcacctggcttaagtgctgctctggaactatctgttgaatgaactaatgcatgaatgtattgttgagtatgagacaaacaagtgtcattgtctcctttctagccttgccgcatcaaaggtgacattggacagtccactgatgatgactctgcacctcttgtccattgtgtccgccttttatctgcttcgtttttgctaacagggggaaaaaatggtgagtacaaaaggggatgtgcacagttgaaggaaataactaggtttcagaggtcagcttggtggcctgtttttgccttgcgtgcagcagaggaagtagaatctgaggatgagtttggttttcactagccgaggggagggaggaaatgatgggagcaggtaggttattgggtctggttttgttcatttgaaaacaatctgttgtttgaggctgaaggtggcttgggtgatttcttggcagtgctggttccggacagggatgtgagggtcagcgtgaaggccctggccctcagctgtgtgggagcagctgtggccctccacccggaatctttcttcagcaaactctataaagttcctcttgacaccacggaataccctggtatgttaaaagttcacatcttattttctcagatttaatcattattgtaaaaactatttcagtattgactattttagttttagagcagtaagtgttttgagttcatttgggatatttgacctgcgttgtagctcttcagaaaacacatgaatagtgaagttctttgtttcatgggttccctttagatgaaacccatagaggagaaaagtagaaacctcagcacgtaagagccaacatatatacacatcggatttaaacctaaagcacaaattgtgcctggtcgcagtggcgctgagtcgcactcagccaggccaggcattcacactcagggtgagtgggaaccaggactggctgaggcagcagtggacccaagtctccatcgcgcccatgcttactatggagccttctcgttctctctttttctttgggtgagagggtacacttgtgtttttgaatttatatgaggtaagtgtgtaatagggttttttctaatcttttttaagtggaatctggaattttaatcagatttattatctgacaacctagaattataatccagaaagtctgtggtattgaggacatattggcaatatgatgaatctctaattcttaaatcctgaaacttttttttttttaatcacttagggttattatagtgaagtcatttctgaatttggatcttctcttcacacctctttttctctttcctgagaattaagcttttgtttcgagttagaaagttgatagtagggaattgttccatggctgagcaatttatctccacagaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggtaacggccagtttttcagctgtgttttttctagttatgcttactaaggtttaagtttagatgatgatgtttgttgcttgttcttctggttaggaaatacattttctttggcggattgcattcctttgctgcggaaaacactgaaggatgagtcttctgttacttgcaagttagcttgtacagctgtgagggtgagcataatcttctgtggaaccatttcttcacttagtggacattttatcattgctacaattaaaattggagcttaataggaaatatttccatgcactctaaagctgtaaccagtaatacccaccatgtatccatctctcagctttagaaagaaaacgttgccagtaaagttaatgcttcataaacttcagtttaagttctaattctcagaatatttgtttgaaatagacctcttcctaaaggatatatttagaaataacctatcattaagtgtaaagtctgttgaatatgctgggcacggtgactcacacctgtaatctgaccactttgggaggccaaggtggaaggattgcttgagcccaggagttcaagactatgggcaacatagttgaccctgtccctacagaaaattaaaaaaaaaaaaaaaaaaagtagctgggtatggtggtgcatacctgtagtctcagctactcgggaagctgaggtggaggggggattgcttgagccccagagatcaaggctgcagtaaggcgtggttacaccactgccctctagcctgggcaacagagtgagactgtctcaaaaataatagtaataataatcagttgaattaaaaaaaaaaaaaaaaaaaccactgtgctaggcccatagtatggtaagagttaaagtgagccttagggattatttactcaacctctgtttctgtataaagtggaataggctcaattctttaagtgatagcatgttgaacctttccataccaactggctcataagtcacaactggccagtcaacaagagtaaaaattaactggtaaaaatcaaagcaaaaaacctacaattgtcaaatttgtgggataactcccccttttaaaatgtcatgcctgacagtaatttctctctagtttccaggttttcagtcagttgtgtcttttttgagcagaaggaagcatgctaagagctcaatcttgtggctagctgggggtctttgtgtcagccatgcatgtgatggtgcccctgggtgcttggggctgcaggggaggggtacagcagtaggggcctgttctgttctctcgtgctgtggagtacatagtgacatagtggggtggtccttggtgtaggtcccttgttcctacccctgggtctgagatttatttagaagtggtgttggggctgtgcggcaggcccctctgtaactgatcaatgtttgtgaagttgctgtttgagagttgaaaccatgacataagcagaaatggaaggaagaaagaaccagttatgtgaaagggacacatttacttttaagcttgtatttactgagataaagtattcttaatcaatgttcttgagaggtgtgggaaaaatgcaacatcctggttgcagttaaacccagaacattgtgtgttgaagagtgacggttctcaaaccgtcaagacgcgggtactgagtgggactaacctgctgtcctcttgccttggaccttgtgttccagaactgtgtcatgagtctctgcagcagcagctacagtgagttaggactgcagctgatcatcgatgtgctgactctgaggaacagttcctattggctggtgaggacagagcttctggaaacccttgcagagattgacttcaggtaagtgagtcacatccattagatttcatgaactaagctcaattgaaagttctgggatcacttgatgcaaggaatgatgttatcaagtaccctgtccatcagaaatccgagtggtttaggtagatgacagtgattttctcctcccagtggctttttgctgaactttgccctatgcttggaattttattttattttattatttatttagagacaagatcttgctctgtcgcccaggcttgaatgcagtagcacaatcatagctcactgaagctttgaactctaggactcaagtggtcctcctgcctcagcctcccgattagctaggagaataggtgtgtgccgtcacactggctaatattttttgtagaaatggggtcttgctatgttgcccaggctggtctcaaactcctgggcttgattgatcctccatcttggcctcccaaagtgctgggattacaggcatgagccactgtgcctggcctagaattttaaaatataagtagaagagtagatttttttttttggtagtcctcgtcatttaagtattctggatagtgggaataaaagagcttagaatttttcatctttgtcttaaacttttaaaaaaatgtagcttatattaattctgcttgtttaaaaagaatatactcttcattatactgaacctaggtaagacagctggtttatattttgttgcaattaaaaaacgtgagctgtggttgcagtgagccaagattgtggccattgcacttcagcctggcaacagagtgagacttggcctcaaaaaaaaaaaaataacatgagctgtgttggcactttcattttctaagagtagttttggctggagaagttttctttcagtactttcttttagaagggaaattttcctttataatttagggtttgttttttttttttccaagccaccttttatagagcccttgtgggttatttcatttaatccttagaatgtttataaatctgggcttgttctcggctccacccacagatagggacgctgagcgtgcatgagtgggcagcaagatagcaggttatggagggcccagctcaccccttctgtggcttgagccaattttatagggcacttacagagtcttttgaaatagtatttattttgaagaaaaagaaaaacagtttactgagtactgtcttattgagtctggaattgtgagaggaatgccacctctatttatttaaagccattggccttttttgttgttttgagtaagtgctgcccaaggtccttccagggcacctggatgagcctgctctggagcaagctggcggtaagtgtttactgagtaactaaatgatttcattgttaaatgtgctcttttgttaggctggtgagctttttggaggcaaaagcagaaaacttacacagaggggctcatcattatacaggggtaagcggtttatttttgtgagatgctgttttaccttcaagaaggtgaaagtgaggctttccttgtggaatttctctaaatgcattcgtcatgttttagatgtttatttcacagtttatatcatgaaagttataatcttgtcatatggatttaagtctagtaatgttgagttctttctcactagctttccaaaatatcttacctaaaatttagtcaaatacaagattatgtttatttttattatccttctctctaaagcttttaaaactgcaagaacgagtgctcaataatgttgtcatccatttgcttggagatgaagaccccagggtgcgacatgttgccgcagcatcactaattaggtatttaccaatattttatctcttttccttttttggttgaagtactaaaagatacgagaatggaaagagagggaagaattcaaaggatgtagagcagtattcctgaatctgagctcatttcagccattctattcttaaactataatgaaaaaaaaatccaaaaaagtctaaaattataattaaaaaaacaacaaaatactaactgtccattgtaaaaagtaatgcactttcattgtaaaaattttggactatagagaatagtactaagaagaaaaaaaaaatcaccttcaattctgctgccacctggaggtaatcactgttaatattttgctatatactctatgagtttcttgttcaaaatcaggtcaaaattacatgcaattttgtaatctgacaatttccacttaatattttattagcattttcctgttatgaaacagtaattttagttatgggtcgttgttttgctatgcggttgggataaaattttatatactttttttggcaattacttattatacataaatgtttgtgtatagttttctttttctgagaattcctggaagttgagttaccaggcccggctttgaatttttttttttattttttttttgagacagagtcctgctctattgtccaggtgctatctcggctcactgcaacctctgtctccctggttcaagcgattctcctgcctcagcctcccgagtagctgggattacaggggcacaccaccacgcccaattaatttttgtatttttagtagagacagggtttcacgatattggccaggctggtctcgaacttctgaccccgtgatccacctgcattggcctcccaaagtgctgggattacaggcgtgagccatggcgcctggccaggctttaaatttaaaacaaatcttctaatagctttatggaggttataatttacatttcttgaaatgtactcactttgagtgtatagtaaactccaattttatcacatttctgtcaccccaaatgtatccttgtgcccatttgctgtaacctccggttcctgccccaactcctaggcagccactcatctattttctgtcccttaagatttgtgttttcgccaggcgctcatgcctgtaatcccagcactttgggaggccgaggttggtggatcacttgaggtcaggagttcgagaccagcctggccaacatggtgaaaccttgtctctactaaaaatacaaaaattagtcggatgtggtggcacacgcctgtaatcccagctactcgggaggctgaggcaggagaatcacttgaacctgggaggcggaggttgcagtgagcagagatcgcgccactgccttccaacctgggcaacagagagagactgtctcaaaacaaacaaagatttgtattttctggacattttatagtactggggtcatagtatagatggacttttgcatttggcttcttttacttaattgtgagattggttcttgttgtagcatgtatcagtagtttgttcatttttattggcgaaagtattctattatatgaataataccatattttatctatccatcagatggatattatagagttcatgttttggctaatttatgaattatggtactgtgaacatttgcctgcaagattttgtgtagacatgtcttcatttctcttgagtagatcacctagaagtggatttttaaataattttggtacttactgtgaaactgctcttcaaaaacataccattgttccttccttccttccttccttccttccttccttctttccttcctcccttcctccctcccttccctacttccctctccctttccctttcccttccccttttcccttccccttcccgcctgcctgcctgcctgccttccttccttccttccttcgtttctttctacatatacacatttttttaaatttcaatggtttttggggtacaagtggtttttggttacatggctgaattttggttacatggtgaagtctgagattttagtacacctgtcacccgagtagtgtaccttgtacccaatatgtagttttttgtccctcaccttccagccttccgccttgtgagtctccaatgtccattataccacactgtatgcccttgcgtacccacagctcagctcccacttctgagaacatatagcagaaacatgccaaagtatactcccactaccagaatgtgattgtgcctgattcttctcaccagtacaaatatttcaaaaaaagttaaatatgtatcagttttttgggcagaagttgatacttctctttatttatttattttttttgagatagggtctcattctatgatgcccaggctggagtgtggtggtgcgatctcggctcactgcagtctctgcctcccaggttcaagtgattcccacgtcagcctcccaggaagctggaattacaggcgagggccaccactgccagctaatttttgtattttttggtagagatggggtttcaccatgttggccagactggtctcaagctcctgacctcaagtgatccacctgccttggccttccaaagtgctgggattacaggcgtgagctaccacacccggctgatatttctttttaaaataacttaccttcttttgaaagtaatacatgtttaatgaacagaatttaaggaaaatataaaaaaacgaaataatctttgtaatcaaactactgaaaagaaaaccaaagttacattttggtgcatattctttttcattttcatcattgtaatttgcatttctttgattacttgtgagacactcctttcatttacttaataggtttatatgacttgcctattcagagattttgcagctttaccattttctgcaaatgatagcaacttctttttgtttgtttgtttgtggagacagagtctcgctctgtcactcaggcaggaatgcagtggtggaatcttggctcattgcaactattgcctcctgggttcaagcgattttcctgcctcagcctcccaagtagctgggattacaggagtgtgccaccatgcccggctaatttttgtatctttagtagagatggggttttgccatgttggccgggctgatcttgaactcctggcctcaagcggtccccctgtctcggcctcccaaagtgctgggattacaggcgtgagccaccgtacccagccagtagttacttcttatattctagaaaaaattctactcatgatcaagtctccatgaggaaagagactttaattgaagatcatggggcttgcagaccaatatgataaaatagttcattgtttctaaaagtattactgagtgttgatggcagatatgaacccttttgtttttgtaggaaaatgttacccgtattctccatttgaattcagtttagatttgttaggaatcgcagcttaagctttgccatctgggagtgtttgggacagttttgcagacaaaattgcaaaagtgcctaaggaatgcagctggcattcagacctgctctgtgctcagtactctgtggacagacactgttcagcacttgttgatcagaaggtttagaaagagaactttcaaagttggtttttaattaaagcatttaatagtgtaaatagaaagggattaaattttatgacagacaaaagaaagtacagcacccagctgggcgtgggggctcacgcctgtaatccagcactatggggggctgaggtgggtggatcacgaggtcaggagttcaagagttcaagaacagcctggccaaggtgatgaaaccctgtctctactaaaactacaaaaattagccgggcgcggtggcaggcgcctgtaatcccagctactcaggaggctgaggcaggagaatcacttgaacctggacggcagaggttgcagtgagccaagattgcaccattgtactccggcctgggccacagagtgacattctgtctcaaaaaaaaaaaaaaaagaaaaaaagaaagtacagcacccagttatgtccgagtgggtgcatgagagtgaccctgagattggagacaacgctgtcacgtgcttgaagaacgccacctgagaaagggggcgagaagtggtgtccgctggtaaccagaggtgttggcttagccatctgcagggaggagggtggtctatcacaggtgagtttcatctactttcttaagcaaattaaccttacttttgtgttaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccaggtatgctgacccagtggcatcttcacattgtcgggaaaatgccctttcctgatgcctttctttaggctttaattgaaaacattttattttctagaaaaaagcttcagctcaggatgtttgagtgtaggtcagtcctttgataggatattatcattttgaggattgaccacaccacctctgtatttaagctctgccacaatcactcagctgtgacactgtaaatctcttaatagtttattacattccatgtgctgacagttgtatttttgtttgtgacacttacgtattatctgttaaaacattttcactttagttgtgttacctttaaagaggattgtattctatcatgcctgttgattttttggtgagcgggctattaaagtcagtgttatttagggttatccactagttcagtgatttgcgagattatcattcacatttattgtggagcttttgaatatcgtgtcaaatggccacatatatcccattcttatctgcttcttaggtgagtgggacacagtgctttaatgaagctataatcttcagaattctagcttgcagagaagattgcagaagtgataagacttgtgctttttaattttgtcttttaaatgttattttaaaaattggctttatatgatactctttttttctgctgagtaacagtgttttacaaaacttggactaaatgacttctaagcttaaatgatcacttgatgctttttttctgaattaggaactcagcttatcaaatatcaaagtcataattcctgaataaataacgtcttttttcatgtaaagactgctttaaaaaacacatggaaggctgggtgcggtggctcacgcctgtaatcctaacactttgggaggcccaggtgggcaggtcgcttgagctcaggggttcaagaccacccagggcaacatggcaaaacccacctctactcaaatacaaaaaattagccaggcgtggtggcgggcccctgtaatcccagctactcgggaggctgagggatgagaatcacttgagccccggaggcagaggttgcagtgagccaagattgtgccattgcactcccagcttgggctacagagtgagactctgtctcaaaaaaagacacacacacaaacaaaaaaaacatggagacatttttttggccaccttaatatttcccctcagataatttcctttgtttaaactcagaactggcattttctctcttggagaagattcaggacaaatactcctttaagataagtagaagcagtgaaagaggatttgattatcaggaatttgataagcttagaataaattgttgcttcttaatgtcatttcagaagatgaatatttattaatagatgccaactgagatatcattaaaattgattactaactactacttggaaaagtctcccagttccaaacttcagcaggcctcttgacaattcagctgtggtcaattgggtcttgcgtgatagatacaatgaccaattgtgcagcagagtgtgctgcttagctgcctattctgttagcattcatgtgttaacttaaaatcataatctccttagttttgttgagtgtctccgtggacaagacactgtgagggatacaaaatcagattggctttattcaaaccactggggtattataattcatttataatttattttattttttgccttttttccatgtgttctaaaggaattagagtttgtatataactataatgggggatagaaattgacatgtgccatgaagggaatgcaaaaaagtgccgtgggagatgagaagtggagaaaggaatttcttttttcttggaagcaggaataacttcatgaagcatgtatttcaacttaaacagatagtaggcaacgctgtaaggggagtatggctgcagcaaaagtgttcggggcagactgggaggaagggagggaataaattcagccattgttatggaataatgatcaaaatttattttcagcccgtttcacttaaaagttgagactgcttaactttttttaatctttaatcttaaacttttaaatgccatttgatctttaaaaatatatgttttaatagtgtattttaagtctctatatttttgttattagaatatatagaggctataacctactaccaagcataacagacgtcactatggaaaataacctttcaagagttattgcagcagtttctcatgaactaatcacatcaaccaccagagcactcacagtaagtctctttcttgatcggtcttactgacattgtaatagtttttggtagcttgtatggccagttagttgtatggtcatcttacggtgaggtgcttgtcttacagctcttacttatccatgaggcttgctaagaaattgtgcttctgtgaaaagaatctcagcttactccaggaatgtaaatgactatgttttttctgattattaaagtaatacacgcccaaaataaaaaaattcagccaatttaggaagacacaacaattaaaataagccaggcatggtggctcatgcctgtaatcccagcactttgggaggccaaggttgggggctcacttgaggtcaggagtcggataccagcctggccaacgtggtgaaaccccatctctactaaaaatacaaaaattagctgggcgtggtggcgggcgcctgtaatcccagctactcaggaggctgaggcaggagaatcgcttgaacctgggaggtagaggttgcagtgagctgaggtcaagccactgcactccagcctgtgcaatagagcgagactctgtctcaaaaaaaaaaaaaaaaaaagaaaagaaaaaagtaaactactgtcacctgcattggtaatgtatcagaagtttaaaatgtctagattataattaactcagtgacctggtaatatatactaagggaaaaatatttataatttacatttttacatttttatttttttaattttattattttttttttgagacagagttttgctcttgttgcccaggctggagtgcaatggcatgatctcagctcaccacaacctccacctcccgggttcaagcaattctcctgcctcagcctcctgagtagctgggattacaggcatgcaccaccatgcccggctaattttgtatttttagtagagacagggtttctccatgttggtcaggctggtctcaaactcccaacctcaggtgatccgccctcctcgaccccccaaagtgctgggattacaggtgtgagccaccatgcctggccttacatttttataataagaatttatgttgctgacattagaaaagaaccataatatccaagaatccaagaataattaaattatgtacatatgctagtatatagtgtgatgctttggagaatttttaacaatatggagatgtataatctggattgtaatattgagtgaaaaaaggcagaatacaaacctggtgggggtatagtcggatttcagttaagaaaaataatatttacatatatacatttctcacactggcagataatcaccaagataaattttgggattgtggatgatttttttcttctttatatttttcagatattctcaaattttctaaaatgagcaagtataacttttgttatcagaaaaaaataatatacaaaagtaatgttaatttgctggtgaccaggttaaacctttttatttttattttttgagatggaatctcactctgttgcccaggctagagcacagtggcatgatcttggctcactgcagcctccgcttcctgggttcaaatgattctctggccccagcctcctgagtggctggaattacaggcgtgtggcaccacacctggctaatttttgtatttttagtagaggtagggtttcaccaggttggtcaggctggtctcgaactcctgacctcgtgatccacccacctcggcctcccaaagtgctgggattacaggcgtgagctactgcgcccagccagacctttttattttatttgacaaaagaaatacttccatgttatagaagactaaatattgtttgggctgtctgcagtatggtcttcccttgatttgttcaaaatatcgtaaactttgcttatttatttttattgtggccgactgtgtcgggcactgttgtaggcttgggatggaaaaacaggattcctgcccttagggtttctgcaggctggtcagggagacgatgtggtaagctggagctcagctcctaaggatgtgcaggggcagttgagaggcggaagggtgggagatcattccagggtgtgggcagcacaggaacctctcttcattgggatataattgccattctgataacacgtgtttgaggtgtctaaagtaggaagttgtaccatggtgggacagatatcctgtggttatcatacacagatctcagttttcttctcattgtttgtactttttataaagggtaacaggagatataattcaataaacctttgtggtgtttgggtgtgattttattgtttctttcttctcagtttggatgctgtgaagctttgtgtcttctttccactgccttcccagtttgcatttggagtttaggttggcactgtgggtatgtattttcctcagtatatattaatagttgtctacaacagtatgacataaacatagttattaggatgccctttttctttctttttaagtcttttatcaatttggctttttggaaaaatatctgatggaatacttgtttctgctatattagctgtgtgagactagtgacaggagctgtgggaaatgaatgccaaatgttcttaggcattgatgggaatttcagggtgtggtcttcaagttcatttaagggaattttcatatgctggcaaaaggcttttctcattagcttgactctttccaaaattatttgctgtgaattagaagtttaggaaccttttttcacttaattgtgacctagcatacgaaatggtgatgatttaggaactactgttcttgtattaacagcttttatttaaaaatgattttcctccagtagatggccctactagcatctgggaaataatttcaagtcttctccagcattcaggaataggctttcattttgtgtatcaattactgagaatgattttggtgactcacatcacatttgagaagtaaacctgcagatttcttgtgtgtgtcagcaaatgaccaactgatatttgcttgaagtggattacattatctgctctagaatgattgctttcccaccttcctcacatacagactgagcagctacggtttctaatcataggtctggcactagacttcacttctgggcaactttggcattggagtaaaatgtattaatttaaagaaagttaaaaatccgttcaagtaaacatacagttctaatactttttacaatttaaaatatagatttaaatgataaaataaaaaagaaaatatgggtagacaccataatcctcgtttctgcatctgttcacaaggggttgatatttatgagttctattctccatatccattctatgttctcttaatgctcagtcagcacctcaggtggttggagttcaatgcttggtagtttgacttacactgtcttttctaggggattgagccctgggtagtcctgcttatttgaggttgcaatttgtctttcaataacttttactacaagatatggcgtgttaaaggataccattggggaaccaacataataatatcaggaaaactaaccacgtcagacctgccccattgtgtatcaagtacactatttttccatagtaataaagagttcaccccagccaattctcttttattttgtgcctgtttactcaatggcattaacatgcccaaatgtctgggtagctgtctcatctccagttcagcagaaccattgtcatatgccctagtaaaagcattccttcattggacacttaggccccaatactttcattcagatctactacctgatttcatttctcaaatgatttttatggagctctgatttataggaaagatgttagttgattaaaaataaaacaatttctgagctggtataaaatgtattgtgacatgccttcctcttggaattgcaagagaaaggaagactgttgtttgcttaaaaattgtctataatttgactttgcaaatgtctgcttccagagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcaggtactggtactgagttgaaacagggactccaggacttggattttgatttccttagggggaatgggggtggtgagcatatgaggggaaaatactataaggtcattgccagtgatggcttgtccctttagtcaaatttcagatgttacctatatgcataaacacatgcagttggcagctgttctgtgctgagtattttaaagtagcctcttcccaatatagcccctcagttaactacaagtaaactcattttgaatttcattttaatgggcaccatatgccagtactccctcgggcactgggatgttaagaaagtataatgtatggacttcattctcaagttagttttagattagagggggatacacgtaaacaaaagtgcagtggtcacacagagtggccctaatcactctccttgggcagatttatgggctggtaggaaagagcacaacacggagagggtgtagcaccttggcgatgataatggaggatgtggccagcaaggaagacggagtccattgaaattgattttgggagaagttgccaatctccatgaaagaattggggcctgtgctatttgcttcagggggctataggagagtttcgtgaaagggactaaaagatgagtattttaataagatcattcatccaacttgaacatgggctggaggagaaggtagggagactcaggagattaatgttgatgctaaggcaagataatggctttgggactgtagggaagacactgattgtaagagaatgaaggaggcagaattgccaggcctggttcaccaactgaacttcggttgtgaagacaaagaaacctgggatgacttcacatcctgggcaggtgtgtggtggtgacagtcatggaaattgggaacacagatttgtgcgggaaacatcagtttcagtttgagtttggcttatcagttgaatatcaggcacagatgtctggccaactctcaacatagggtcttaaatgacttcagttccccaagcaatttgtccttcccatgctattggggtggagaggtaatgtctgtgcccatatcacagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggtaatgtcccacttgggtgctggattcatacagccttaatgactatgggtttccagactacctttgtttagtaatctgtcccttctttattctctttttgctttaaatgaacaaaattgctcagattgtgacactaaatttaacatcaaaatgtgaccatgtggatgggtgcagtggctcgtgcctgttattccagcactttgggagactgaggcaagtggatcacttgaggccaagagttcgagaccagcctgggcaacatcacgaaaccccctctctactaaaaatacaaaaaattagatgggttgggccgggcgtggtggctcaagcctgtaatcccagcactttgggaggccgaggtgggcggatcacgaggtcaagagatcaagaccatcctggctaacacagtgaaaccccgtctctactaaaaatacaaaaaaattatctgagcatggtggcgggcgcctgtagtcccagctgctcgggaggctgaggcaggagaatggcgtgaatccgggaggcggagcttgcagtgagccgagatcgtgccactgcactccagcctgggtgacagagcgagactccgtctcaaaaaaaaaattagatgggcatggtggtgcgtgcctgtaatcccagctacttgggaggctgaggcaagagagttgcttgaacctgggaggcggagtttgcagtaagccttgattgtgccgctgcactccagcctgggtgacagagtcagactctttccaaaagaagaaaaaaatgtgaccatgtgttttatagctcttttagtatcatcagtcactgttatccctaagagggaaatacctagctttagttttaggtttccagcattagccaagaaagctcagaattgatgttcctggccaagtacctcattgctgtctccttaaatcttggttaatggctactgtcctggctagcatagttatggagcatttccatggttgtagaatgttctgccaatctcagggacagttttgcttttctgtgaagcaataaaatcaacttcaaaacaaatgttaactatttgtacaatggatttaagatagaccagttcacatacttttttttttttttttttttgagatggagtttcattcttgttgcctgggctggagtgcaatggtgtgatctcagctcactgcaacttctgcctcctgggttcaaacgattcttctgcctcagcctctcgaggcagattacagctgggattacaggcatgcaccaccacacccagctaatttttttgtagttttagtagagacggggtttcaccatgttggtcaggttggtctcaaactcctgacctgaagtgatctatccgcttcggcctcccaaagtgttgggattacgggcatgagccaccacgcccagcctaagatagaccagttcacttactgtttatatctgattactctctctttgccttgtcttctacctttaaaaatctccctactaacttcccattctcctttagctgccatcagtcttctcccttctctgcaaacatctctggagagtcccagcctcagcccacagagcttcccactgctctgaggtggaccttgtttgcaaggcttctttggctctcttggcctggaccctgtctactacttcagccatccttccttaacccctgctggtggtttctgttgccacactccatagcagcgtttcccgcccagatcatgtctttacatctctgggcactgctctggtcctgcctgcctttccctctttgtatcctgcaggctgctacccccatcttgagtgtcctcttcagttggctttcagagggcctcctgggtgttcccttacccacttgccactccccagtcactgggttcagtccttcctgcccaccagcacatgctttctaggctctgtcctaggccgtcttctctctttgtagtctctgggccagtgctgttctagagagtggcagaattttctataaccatggcagtgctccatagctatgccaggcaagacagtagccactaaacacatatagctgttgagcccttgaaatgcagctagtgtgactgaagaactgaaccccgattcggtttaattttcattaaatttaaatttaaataaccttatgtgggtagtggctccagtattgggcagggcagcctgagagtcggggctgttctcctgtcttcagtgtctagatgagggacctcagaggacctgtctctggagctgcagttcaatgtagccagctgccccgtgacacttacatatagctgatttgtggatatgtcagacacggtgtgatgagctcagctttctgtcctcctccccacatctgcccctgccccatttaccccactttgtgtcttatcaagctagaaacaggtcaccacaagtcttcatttccactcaccaagtcttttgtttcccctactaaatattttgcgagaagaaagtgtgtacctttgtattcacatacatgtacatgcacatatacatgcacatatgcaggggtccccaacctctgttaaaaaccggactgcaggccgtgcgtggtggctcacgcctgtaattccagaactttgggaggccgagaccagtgcatcacaaggtcaggagatcgagaccattccggctcacacggtgaaaccccgtctctactaaaaatacaaaaaaaaattagccgggtgtggtggcgggcgcccatagtcccagctacctgggaggctgatgcaggagaacggcgtgaacctgggaggcggagcttgcagtgagccgagattgtgccattgcactccagcctgggcgacagagcgagactctgtctcaaaaacaaaacaaaacaaaaaaaaaaaaaaccaggctgcacaggaagaagtgagcaagcattaccatctgagctctatctcctctcaggccagtggtggcattagattctcataggagcgtgtatgagttcgttctcacacttctgtaaagacatacctgagacatataaagaaaagaggtttaattggctcacagttctgcaggctgtacaggcttctgtttctgggaaggcctcaggaaacttgcagtcatggcagaaggtgaaggggaagtaggcacatcttcacatggcccacaggaaaaagagagaaggagagagagagagagacagagagagagagagaaaaagaaagattgagagggagagaggagggagaaaggagagtgcctgtagggggagttgctacacaaaggagcaccagggggatggtgctcaaccattagaaactacccccatgatccaatcacctcccaccaggccccacctccgacactggagattacaattcagcatgagatttgggtggggacacagagccaaaccatatcagagcatgaaccctattgtgaactgcacatttgagggatctaggttgcatgctccttatgagaatctaatgcctgatgatgatttgaggtggaacagtttcatcccgaaaccatcccccgccaaccctggtttgtggaaaaattgtcttccacagaaccggtccctggtgccaaaaagtttggggacctctgcacatatgcatgcacctgtacatggacacataatacatgtacatatgcatactttatattctctgccacttctggtccagactgatatactatctcatttggattactgcactagccttttgttttggaaacagcattttttaaaaaatttaatttaatttttttgagatagggtgtcattctgttgcccagcttggagtgcagtgtcatgatcatagctcactgcggcctcgatctcccaggctcaagtgatccttctgcctcagccttctcagtagttgggactacaggcatacccaccatgcccagctaattttttgatttttttttttttttgagacagagtctcagcctgtcgcccaggctggagtgggttggcgcgatctcagctcactgcaacttctgcctcccaggttcaagtgattctcctgcctcagcctcccgagtagttgggattacaggcgcctgccaccacacccagctaactttttgtatttttagtagagacggggtttcaccatgttggccaggctggtctcgaacttgtgacctcgtgattagcccgcctcggcctcccaaagtgctgggattacaggcgtgagctaccgctcccagccaggaaacagcattcttgagataattcatataattcacccatttaaagtatataattcattctctttagtatgcccacagagttgtacagccatcaccagaatcagttttagaacccataaaggaactctgtactctttacccaaaacctccatgcctccagctgcaggcagccactaacctgccttctgtctctgtgactctacgtcttctggacattactgtggatgggctcatacagtcagtgagcttgtgactggtgccttctaccaagcagggttttcagtgtagcagcctctctgtttttcttttttttttaaattgtgacggaacttctgcctcccgggttcaagcgattctcctgcctcagcctcccgagtggctgggactacaggcccatgtcaccatgcctggctaattttttttttttttttttttagtagagatgggtttcaacatgttagccagggtggtctcgatctcctgacttcatgatccgcctgcctcggcctcccaaagtgctgggattacaggcgtgagccaccatgcccggctaacctttcatttactgtctgcatttcttccctgatgccttccagtccatgcacccgattgtagccattcatcctattatggtttaaggtgactgtcttagtcagcatgggttgccataacaaaataccatagcctgggtggcttcaacaacagaatttacttctcacacttctggaggttgggaagtccaagatccaggactttcgccttgccctcatgtggtgagggggtgaggaagctctgtggggcctcttatatatggatgctaatctcattcatgaggggtctgccctcatgacccagtcacctcccaaaggccccacctcctaataccatcaccctggtaattaagtttcagtgtataaatttgggggactatagacattgaaaccataacaagcacttttctaagatcagggagtgagtaagtagcagagctaggacctcaattccacatgtcagtcatcttgccttcactctgctccatgatggctgcctcctagagcattgggagtctcgatgttctatatgctctcatgtgttgtgtattggagatagttgaggctttatgaatacatctggatttgttgacttctagctttgctggtaaccagctgtgaccttgaataagttacttcatctctgagcctgtttcctcttttagaaacaggagtttaaaatgctgctttgggttgggcacggtggctcatgcctgtaattccagcactttgggaggctgagatgggaggatcactggagcttggagttcgagaccagcctgggcatcatagtgtgagatcctgtctcctcaagaaattaaaaaattagctgggtgatgtggcgtgtgcctgtggtcccatctactctggaggctgaggtgggaggattgcttgagcccaggaggttgaggctacaatgaaatatgattgcaccccatcctgggtgacgagtgagaccctgtctcaaaaaagaaaaaaaaaatgctgctttgtacccctttcatgtcatggcgtcatggccaacatagaatgccctggttgtttgctgttggagggcatgggcctgggggctccctgagggctccttccatcttcaactcattctctgtgcacctgttaggaagttgtgggccagtccctaccatgtatcattgtgtgggtaaaagtaaataaaatgtgtacagtgtctgaactgtacatatcagggtccaagaacaaaatgagtgacatgggttagctctttttaataaatggtaaaaccaaatattctaattttcagttttgttatacttccatcacatgtttttgtttttttgttttttgtttttgtttttctattttaggcagccttgccttctctaacaaaccccccttctctaagtcccatccgacgaaaggggaaggagaaagaaccaggagaacaagcatctgtaccgttgagtcccaagaaaggcagtgaggccagtgcaggtaggaaacagcgtggggaagggagggacatgagtgcagcatctgtcatgtagaaacataggatttaagtaacttggtgttttagagaaataaatataatacacatcagtaaagtgagagaaagtttctccaggtgcggttcaagatattagaaactaatgactgatgtacacagaccaccttttggtctgaagcatttctaagtgccactggctgacatgcagcccctacagcctccaggcttccagccctagcatggagcatcactctcctatgcttccctggttgcaggtgatggctggagaggcctcctgattttcagtaagggaagtggtgtagatgcttaggaatagatgtagtgagtgaaaaaactgattctgatatgtcaaaaattctgattggaaatggaatatttacatttggaagagctaaaggcgagagaaagtggggataaagtcatctgagttggaggagcttaaaccattcacaagtttggaggacctttttttacccatgaaaaggtcagaacagaaggggctaggatttaggtgtgactgcagtttattgaattcccatccatactgctctcggtgggcagtggcaggggcaggagaggagcctggcaaagcatgaagtgactgctgctgcctctgctatctgggacgcctggccacctgtctgtacagtctccctccagacccattctcacgctgtctcttggcacccaggggccagtgatggttctcccatttgttttgtgtatatagcatttatatcaaggctatttatttatttatttattttatttatttatttttttgagacagagtctcactctgtcacccaggctggagtgcagtggtgcaatctcggctcagtgcaagctctgcctcctgggttcaagcaattctcctgcctcagcctcctgagtagctgggactacaggtgtgcaccaccacacctggctaattttttgtattttttattagtggagacggggtttcaccttgttggccaggatggtcttgatctcctgacctcgtgatccgtccacctcagcctctcaaagtgctgggattacaggcatgagtcactgtacccggcctatttatttatttttaattgacaaaattgtatatatctgtaatatacaacatgatgtttgaaatatgtgtacattggccaggcgtggtggctcacacctgtaatcccagcactttgggaggctgaggtgggcggatcacgaggtcgggagttcaagaccaaactggccagcatggtgaaatcctgtctctactaaaaataccacaaaaaaaaaaaaaaaaaaaaaaagccgggcatggtggctcgcgccagtcgtcccagctacttgggaggctgaggcaggagaattgcttgaatctggcaggtggaggttgcagtgagctgagttcatgccactgcactctagcctgggcgatagagcgagactccgtctcaaaaaaaaaaaaaaaagaagaaatacatatgcattgtggaatggctaattaacctgtgcatcacctcacgtatcattgttttgtggtgagaacacttaaaatctactctttcagtgattttcttgcatatggtacattgctattaactgcagtcaccatgctatacagtagatctcttgaactcattcctcctgtctataaatgaaattttgtatccttgaccaacacattcaaggttttttttgagatggagtcttcttcacccaggctggagtaccatggcacgatctcatctcactgcaacctccgcctcccaggttcaagcaattctcctgcctcagcctcctgagtagctgggattacaggcacatgctactgcacctggctaatttttgtatttttagtagaagtggagtttcaccatgttggccaggctggtctcgaactcctgacctcaagtgatccgcctgccttggcctgccaaagtgctgggattacaggtgtgagccactgcacccggcctcaagcgttttaaaagatgctcttttctaaggattgactgtagtacaggaggaagattgacctgttgaaaagcctcagcctttacaagtgtaaaattatcagtatattactatcatctttctgatgaattaaataaactaaggactccaagtcaaaagtcttcaaactgaagtagaatagttgtatatagtgcttggcactttaatatttagtatcggtttaatgataatgtttgtgcctttgccgtctttaaaacatttttacatcatccctgtttgattacttggtgtgctcatgaagttgttggccactaaggaatcttaggctcagagaggttctggaattggccagtggtccttgaatcagctgctcctatgattctctaactgatttctcacaaagcaaacaagcaatcataacaaaacaactgtgcacactgctcttcttattttgttatttaaaaagtacttaggctctacttatgtttgttagtcaatttctcattacttctagttaatcaaaaggtcagaggaaatacttgaatattttcatactagaatactttaaaaaatcatgatttccagtaatctctttaaaacttggcaagttattttgatctaaaagtttatcttttgtgtgcatatttttaaagcttctagacaatctgatacctcaggtcctgttacaacaagtaaatcctcatcactggggagtttctatcatcttccttcatacctcaaactgcatgatgtcctgaaagctacacacgctaactacaaggtatgggcctctgcatcttttaaaaatatatatgcacacatacttacgtctaatggatagttgatgtttttcttatgatttgtaggatgtataagccctttgagatatgagttacatttagttttttcaagtttgtttgtctttcagctttgtttatgatagcttctatcatacaggtgttttggattttcatattgtttgtactcacagctaagattgattacagtgacagagctaggatgtgcagccaggttatagggggaagtggccctggtggagtctggagggatccgtgtacaggcttccttccctcccgtgaggctcacacaaaaatacagcaacatgctggtcctgcaggtaccctctgcctaacatgagccacaattccagactcacagaagaaaagcaggtgttcggcataaaccatgtgtttcaaatagtctgggcatggtgagccacttgttatcagctagggaaagtttatgtcagcgtaagaaactgttcaccagatacccccaagagccagcctttctgtctagggatgttttagttttttagttcatttttttttttaactttaaaattttctgttcatctgcaatttgttagatatgaagtatgtgtctaatttaatttttgtttttggttgtccccaataatgtttacagaagaatttttctgcactaattggcttgagttacttacattctcatagttctctagtttcagtagtttcatttattattttgttatatcaatctatctgtctgctcatctattagaagcatccttgttttttttttttcttttttagacagagtcttgctctgtccccaggttggagtgcagtggtgcaaccatgcctccctgcagtctcagggctcaagtgatcctcccacctcagctcctgagtacctgggactaccggcatgtgccaccacacccagctaatttttacattttttgtagagacagggtctccctaagttgcctgggctggtctcaagctcctggcttaagtaatcctccctccttggcctcccaaagtgctgggattacaggtgtgagcaactgcacccggctacaagtatacttcttaattattgtagcttaatggtatttatgaggggatcagttcccctgttgttctttagaattttctggatattcttctttattgattttgggatgtgaacaatagaatcaacttctacttgtagattgatttagggagaacttatacctcagatgttaagtcaccctgtccagaatgtgggatgctttcctatttgttcagaactttttaaattacctcagaagcacatgaaatttaaaggattttaaaaaaaacttaaagattatttcacatagctcttgcacatttcttgataaatgaatcctcaggtattcctctgtttttgttactaatagttacttcttatgggttttttttcccctgaaaatcatttatcaaacgtatgtggcttattttctgaaggatgtttgataattttggaagatatgaaagtcttcatattttacaaggtttgaggtctctttaagctgcatggttctcatgtcagctcccaaagcagaagacggcatgttgaaaaatgccgtagagaagatacttcttttccacctgttttcaactcatatcatcttgaatttcagggcacctttccatgctcctagtgcttgctatctgtttattattttccttcctgaataccctgaactccagcatgttctgctgtaattctggcctccctggcatcttggactcctgtttcctttgctctgtcatccccgcggtcagctcctgctgcgcagcttctcagctgaagtgcgtttggagtgcctggcgtgtcttgctggatctttgagtattgcctctggtttccttggttccttctgctgagttgctcagcgtctccactccccatttcttgtgtggcccttcctgcactcctctgattccttttgtcttccctggtttcttgctttggtttcgagtctccacagaacttttgcagctcttctgaagacctggaagctttttcatcttaattctcatctcatgacctcttttcccttctttgagagctagaacttcccatggtgaacttctctttccagaattccatgccttcttttccctcccacttacctgttgtccaggagaggtcagattgctgtgcatattggaggagaaccctttcttccctgggctcttcatctcacatgacatcaccacatcacctcgttccttggaccctcagtggtgtcactgctggatttttctttcctttggctggccttagggcacacccaggttgactagcgtagtcatggtatttagatccactcacattttcagtttctgtgtctgtctcttgcctgcttctgacttcgcccagagaaagcttctctttcacaagggttcttagatttatgttcactgagcaccttcttttctgaggcagtgttttaccaatatttattttcctagtcagtctcgccttacctttcttgttatgcatgtctttggtcctgacccattctctgagtctgtaaaatagaattgctgtataatttaattacatgaaatcctttagaatcttaacacatcttacacctgatttaatattttattgtatccaaattgaaccaaccctatgtgaatttgacagtgatttctcccagggatcctagtgtataaggaataggacttagtattttctattttttgatataccacataccagatactgattatgatggacatttaacccttttttctcattatgaaagaaagttaggaattatttcttccagtagcgccagtgtaacctgaaagcctttgaaagagtagtttttgtatagctatctgaaaggaatttctttccaaaatatttttccagtgctgacaacaaacacgcagacacaccctgcaaggtgagtgtacggcgccgcacagtggaggcatctgctgcagccgtcgatgtttgtgtctttggttgtacattatgagatcgtgacagggccagtaaccgtgtgttctctccttcaccttcccaaggtcacgctggatcttcagaacagcacggaaaagtttggagggtttctccgctcagccttggatgttctttctcagatactagagctggccacactgcaggacattgggaaggtttgtgtcttgttttttctccttgggttgtggctggcacacttgatgtgcgtcttctgggctgagttcatctaggatggagcctggttctccagggtgcctccgggagactcctccctgccccacgtgcttgcgtcacaggacccaagtctgactctgccttagccatgaagtttagggggaagtttctatttgtattctatttttgtctgttatcatgtattagcttagacccagtttagtttggaaaatcagtgggtttcaaaatgtgtttgtagagtcctttatttcttaacttgaccttttcaagtggaaaggggcaaaacagacgggtaagggggcggggcgggaggtgtgacttgctcttttgtgcctgaggaagtaacagagctggggttgacagtcatattctctgacacagatagtctctgacttatctcacagaaagtcagcggcagagcctgagttaaaagtctcgtagattttctttttcttttttttggtggctaatttcagttttatttatatttgtttatttatttattatactttaagttctgggttacatgtgcagaatgtgcagttttgttacataggtatacacgtgccatgatggtttgctgcacccatcaacccatcacctacattaggtatttctcctaatgttatccctcccccagtcccctcactccccatgggccccggtgtgtgatgttctcctccctgtgcccatgtgttctcattgttcaatttccacttgtgagtgagaacatgcggtgtttggttttctgatcttgtgatagtttgctgagaatgatggtttccagcatcatccatgtgcctgcaaaggacatgaactcatccttttttatggctgtatagtattccatggtgtatatgtgccacattttcttaatccagtctatcattgatggacattcgggttggttccaagtctttgctattgtgactagtgccacaataaacatacatgtgcatgtgtctttatcgtagaatgatttataatcctttgggtatatgcccagtaatgggattgctgggtcaaatggtatttctagttctagacctttgaggaatcgccagactgtcttccacaatagttgaactaatttacactcccaccaacagtgtaaaagtgttcctatttttccacaacctctccagcatctgttgtttcgtgactttttaacgatcgccatcctaactggcgtgagatggtatctcattgtgattttgatctgcatttctctaatgaccagtggtgatgagcattttttcgtatgtctgttggctgcataaatgtcttcttttgcgaagtgtctgttcatatcctttgtccattttttgatggggttgtttgcttttttttcgtaaatttgtttaagttctttgtagattctggatgttaatcttttgtcagatgggtagattgcaaaaattttatcccattctgtaggttgcctgttcactctgatgatagtttcttttgctatgcagaagctctttagtttaattagatcccgtttgtcaattttggcttttgttgccattgcttttggtgttttagacatgaagtctttgcctatgcctatgtcctgaatgttatggcccaggttttcttctaggatttttatggtcctaggtcttatgtttaagtctttgatccatcttgagttgatttttgtgtaaggtataaggaaggggtccagtttcagttttctgcatgtggctagccagttttcccaacaccatttattaaatagggaatcttttccccattgcttatgtgtgtcaggtttgtcaaagatcagatgattgtagatgtgtggtggtatttctgaggcctctgttctgttccattggtctatatatctgttttggtaccagtaccatgcagttttggttactgtagtgttgtagtatagtttgaagtcaggtagtgtgatgcctccagctttgttcttctagcccaggattgtcttggctatgcaggctcttttttggttccatatgaagtttaaaatagttttttccaattctgtgaagaaagtcagtgatagcttgatggggggatagcattgaatctataaattactttgggcagcaaggccattttcacgatattgattcgtcctatccatgaacatggaatgtttttctatttgtttgtgtcctctcttatttccttgagcagtggtttgtagttctccttgaagaggtccttcacatcccttgtaagttgtcttcctaggtgtttcattcccttagtagcatttgtgaatgggagttcactcatgatttggctctctgtttgtctgttattggtgtataggaatgcttgtgatttttgcacattgattttgtatcctgagactttgctgaagttgctaatcagcttaaggagattttgagctgaaccaatagggttttctaaatatacaatcatgtcatctgcaaacagggacagttttacttcctctcttcctatttgaataccctttattgctttctcttgcctgattgcgctggccagaacttccaatactatgttgaataggagtggtgagagagggcatccttgtcttgtgccggttttcgaagggaatgcttccagtttttgcccattcagtatgatattagctgtgggtttgtcataaatagctcttactatgttgagatacgttccatcgatacctagtttattgagagtttttagcatgaaaggctgttgaattttgtcaaaggccttttctgcatctgttgagataatcatatggtttttgttgttggttctgtttatgtgatggattacgtttattgatttgcgtatgttgaaccagccttgcattccagggatgaagctgacttgattgtggtggataagctttttgatgtgctgctggattcagtttgccagtattttattgaggattttcacatcgatgttcatcagggatattggcctaaaattctctttttttgttgtgtctctgccaggctttggtatcaggatgatgctggcctcataaaatgagttagggaggattctctctttttctattgattggaatagtttcagaaggaatggtaccatctcctctttgtacctctggtagaattcggctgtgaatccatcctggactttttttggttagtaggctattaactattgcctcaagtttagaacctgttatcagtctattcagagattcagcttttttctggtttagtcttgggagggtgtatgtgtccaggaatttatccatttcttctagattttctagtttatttgggtagagatgtttatagtattctctgatggtagtttgtatttctgtgggatcggtggtgatatcccctttatcgtttttattgagtctatttgattcttctctcttttcttctttattagtcttgctagcggtctacctattttattgatcttttcaaaaaaccagcacctggattcattgattttttttggagggttttttttcgtgtctctatctccttcagttctgctctgatcttagttattttttgtcttctgctagcttttgaatttgtttgctcttgcttttctagttcttttaattgtgatgttagggtgttaattttagatcttttctgctttctcttgtgggcatttagtgctataaatttccctctacacactgctttaaatgtgtcccagagattctggtatgttgtgtcttcgttctcattggtttccaagaaaatttttatttctgccttcatttcgttatttacccagtagtcattcaagagcaggttgttcagtttccatgtagttgtgtggttttgagtgagattctcaatcctgagttctaatttgattgcactgtggtctgacagacagtttgttgtgatttctgttcttttacatttgctgaggagtgttttacttccaactatgtggtcagttttagaataagtgcaatgtggtgctgagaagaatgtatgttctgttgatttggggtgcagagttctgtagatgtctattaggtccgcttggtccagtgctgagttcaagtcctggatatccttgttaattttctggctcattgatctgcctaatattgacagtggggtgttaaagtctcccactattaccgggtgggagtctctttgtaggtctctaagaacttgcttcatgaatctgggtgctcctgtattgggggcgtgtatatttaggatagttagctcttcttgttgaattgatccctttaccattatgtaatggccttctttgtctcctttgaactttgttgatttaaagtctgttttatcagagactaggattgcaatccctgctttttttttgctttccatttgcttgttagatcttcctccatccctttattttgagccaatgagtgtctttgcatgtgagatgggtctcctgaatacagcacaccaatgggtcttgactctttatccaatttgccagtctgtgtcttttaattggggcatttagcccatttacatttaaggttaatattgctatgtgtgaatttgatcctgtcattatgatcctagttggttattttgcccgttaactgatgcagtttcttcatagcgtcagtagtctttacaatttggcatgtttttgcagtggctggtactggttgttcctttccatgtttagtgcttccttcaggagctcttgtaaggcaggcctggtggtgacaaaatctctgcatttgcttgtctgtaaaggattttatttctcgttcacttatgaagcttagtttggctggatatgaaattctgggttgaaaatactttttttaaagaatgttgaatattggctcccactcttttctggcttgtaggatttctgcagagagatctgctgttagtctgatgggcttccctttgtgggtaacccgacctttctctctggctgccctttccttcatttcaatcttggtggatctgatgattatgtgtcttggggttgctcttctcgaggagtatctttgtggtgttctctgtatttcctgaatttgaatgttggtctgccttgctaggttggggaagttctcctggataatatcctgaagagtgttttctaacttggttctattctccccatcactttcaggtacaccaatcaaacgtagatttggtcttttcacatagtcccatatttcttggaggcttggttcatttcttttcactcttttttctctaatcttgtcttctcgctttatttcattaatttgatcttcaatcactgatatcctttcttctgcttgattgaatcggctgtcgaagcttgtgtatacttcacaaaattctcgttctgtggtttttagctccatcaggtcatttaagctcttctctacactggttattctagccattagtctaacatttttttcaaggtttttagcttccttgtgatgggttagaacatgctcctttagctcggagaagtttgttattaccgaccttctgaagcctacttctgtcaattcatcaaactcattctccatccagttttgttcccttgctggtgaggagttgtgatcctttggaggagaagaggtgttctggtttttggaattttcagcctttctgctatggtttctccccatcattgtggttttatctacctttggtctttgatgttggtgacctacggatggggttttggtgtgggtgtcctttttgttgatgttgatgctattcctttctgtttgttagttttccttctaacagacaggcccctcagctgcaggtctgttggagtttgctggaggtccactccaggccctgtttgcctgggcatcaccagcagaggctgcagaacagcaaatattgctgcctgatccttcctctggaaacatcgtcccagagcacgaaggtgtctgcctgtatgaggtgtttgttggcccctactgggaggtgtctcccagtcaggctacatgggggtcagggacccacttgaggcagtctgttcattatcggagcttgaatgccgtaccgggagaaccactgctctcttcagagctgtcaggcacgtatgtttaaatctggagaagctgtctgctgccttttgttcagatgtgcccttcccccagaggtggaatctagagaggcagtaggccttgctgagctgcagtgggctctgcccagttcgagcttccctgctgctttgtttacactgtgagcatagaaccacctactctagcctcagcagtggtggacacccctcccccagccaagctcctgcatcccaggtcgatttcagagtgctgcgctagcagtgagcaaggccccatgggcgtgggacccgctgagccaggcacaggagagaatctcctggtctgctggttgtgaagactgtgggaaaagtgcagtatttgggcaggagtgtactgctccttcaggtacagtcactcatggcttcctttggcttggaaagggaagtcccccgaccccttgtgcttcccaggtgaggcaacaccccgccctgcttcggcttgccctccgtgggctgcacccactgtccagcaagtcccagtgagatgaactaggtacctcagttggaaatgcagaaatcacctgtcttctgtgtcgatctcactgggagctgtagactggagctgttcctattcggccattttggaagcatcccttgttttttgaggtggagtcttgctctgtcgcccaggctgacgtgcatcggcacaatctcggcccactgcaacctttgcctcctggtttcaagcgattctcctacctcagcctccggagtagctgggattacaggcacctgccaccatgcctggctaattttttgtatttttagtggagatggggtttcaccacattggccaggctagtctcgaactcctgaccttgtgatccacccacctcagcctcctagagtgctgggatcacaggtgtcagccaccacgcccagccatattttcagatctccctctctttgccctaaaccactgtgcttaataagtagtttttagtggccagcagtctccatgtataacacattttagcaaaatggaaaatactatatgttttaaatttgaacgtgagattatactgaaataaaaatcatctaactgggattctttaaatagtaagattttcttttttgtatgtgggtttttttttaaccttattattatgactgtcatatatagaaatggctgtttttcagttacagtcagtgaatgtatcaaatgctgccttatccaaataataaaagtaaattattaataagtcacaatttaatgaagattgatgttagttgatctttatattcttgaaatcagccatatggttgtgtgtgtatgtatatatttttaaaggtacataaagataataagctcatctctgaaaatttttacatttggcataagaataactggataattaagcatcttattctctggcctgtgtctttacagttaaaggtagatttactcacctctccttttttgtttttctaagttcatcttttttgctgtttcaagacagaggcccattttagctttctcgcatatccttttgtttgtactttggaagcctcacctgcttaattgttgagtttttatccgtggtcttttagagggggatatgtagggtagaagctttcacaggttcttgtttgcacttggcccctgactgttttgaggaatctccctcactgactcacagcatggcaaggtttcagatctctttctgccacacagcagttctgaggcagctggaaagatatccagatgcttagattgtcaggccaggcttgagatatacaaactattgagccttatctgtgaccttgcttaggtgaaggcatcagagcccctgcaccaacatgcataggcctctgcatgtgtgcggggctgggtgttgaggtctgagcacaagtgtagctggagaggtgagcttgatgtggcgacgggtatgagcaggttttcttcagacttctgtgagtttacctagttccaggatttaaaggcacagagactttagaattaaaatagaatcattttctttttctaaatagcaacactaggaataaaaaataataattccacattcttgacaggtaatgttttttcttgtcttctaatccttatttattccatactcatttttatacataattgaaatgtattatgcattggatttttcttttgcattatattatagacgatttttcatgtaactccttactgttccattttatatgttttgtctggtttaagactttatctgcaaaccgggaaactgtctctacaaaaagaaaaacaaaaatagttggccgcagtggcatgcgtctgtggtcccagctactcggggctgaggtgggaggattgcttgagccttgggaggttgaggctgcaaagagccatgatcatgccattgcactccagcatgggtgacagactttatactgtctgttttgggtgatttgataatgatatgccctgatgtagtttttttatatcttgtgtttcttgtgcctgggtttattgaggttgggtctgtggcttcatagtatttttaaagtttggaaaattttaggccattctttctttctttctttcttttttttttttttgagacagtgtctcgctctgtcgcctgcgttggagtgcagtgacactatcttggctcactgcaagctctgcctcctgggttcacgccattctcctgcctcagcctcctgagtagctgggactacaggcgcctgccaccacgcctggctaattttttgtatttttagtagagacgaggtttcactgtgttagccaggatggtctcaatctcctgacctcgtgatctgcccgcctgggcctcccaaagtgctgggattacaggcgtgagccactgcacccagctaggccattatttcttcaaagattttttttctgccctgcctccctccttttttccctctcttaaaggggctgtgatttcctgaatgattgcttagtgttgtcccatagcttactgatgctcttttcagtgtttgattgttttatgtgttttctgttttgtatagtttctattattgtgttttcaagttctctgatcttttcttctacagtgtctactctgttgttaatctgttaatctgttgttaatcctgtccagcgtatttttttttttgtttttgaaacagtctcactctgttgcccaggctggagtttagtggtgcgatatcagctcactgcaacctccacctcccaggctcaagcaattcttctgcctcagcctcccgagtagctgggactataggcacgtgccaccacacctggctaatttgtgtatttttattagagatggggtttcaccatgttggccaaactggccttgaactcctgacctcaggtgattcatccgcctcggtctcccaaagtgttgggattataggcatgagccaccgtgtctggcccctgttcagtgtatatcactaattttgtttttatctctagaagtttgatttaggtcttttaaaaatgtctccctgtgtttctgtttagctttgtgaacacaattgtaataactgttttaatatccttctctgctagttctaagatcttctaataacttcccagttcttggtgtttctcattggttgattgatactcctcgttttgggttgtattttcctgcctctttgtatggctgccaattttttattggatgcccaaccttgtgaattttactttgttggatgctatatatttttgtgttcccatagatcttcttgagctttgttctgaggttagttgagttacatatagatggtttactcttttgggtcttgctttataatttgtcagatgggttggagcagtgcttagtttaggactaattttttttttggactaattattcctctttaggaataattaggtaccatgcttaggaggcaagaccatcctgagtactctacctaatgaaccagaaagtttgggttttccagtccgcctgctgagaacagtgactttctagccctgtgtgagcgctgagctctgctccttctaatcctttccaatgcttctttccctggcctcagggagttttctcacacacatatctctgctgagtactcgagagggaccttccccagatctccagagctctctctgtcttgttttctcttctctggtgctctgtcttatgaactgtggctgtcttggtctccttagattctcagcacctcttcaattcagagggttgcctgtccctcctccttgtgccacagcctaggaactctctcaaagcagcgagttggggcagccatagggctgacttagtctctcgtctcccagggatcactgtccttcattgctcatgtccagtgtcttgaggactctgggttttgtctgttttgttttttggtttgctttggttgtctcaggcaggagggtaaacccagtccctcaccctcattgtgctcagtagtggaagtctcactctattacattagatattagtatttgtagcagagccctggttccctggtacttggggagctcttgaaaggccagaaacagcatgctttctcaccttttccagggcttcagtttctggtgcacatcaagcattccatacacatttgttaaagtcctttgttagacaagtagtgattcacaggttctatttgtaattttttcagttaacatgtattgggtatctgctgggagctagtaaaaacaaaaagtggtgtgtgacaaattcaattctgacaagaacaaccttaaacacttagaatatactttgagcatatcagaattttaaaaatgtgtggcccttgagtatttgaaaccaacaagaatctattgcttattagtagaggatattttgttaaacaagtggagagagaggcattttcagtctaattggtgttggcttttagcagctgatggaaaccagttcgtgattagccaggcagtggtgaaacaggctgtgcattctgaatgcctaggtatctaggcattcagaatggtggcgctctttgagttagcatcttcttctttcttgattcttttttttttttttttgagatggactttcgctcttgttgcccaggtaacaactccagtgcaatggcgccatctcggctcactgtaacctctgcctccctggttcaagcgattctcctgcctcagcctctcaagtagctgggattacaggtgtgcgccaccacgcctggctaattttgtatttttggtagagatggggtttcactatattggtcaggctggtcttgaactcctgacctcaagtgatgcacctgcctcgatctcccaaaatgctgggattacaggcgtgagccaccactcccagccccttcttgattcttgaaaaggacattgggtgctgtacatctcgttatagatgttgataaaaatgcttgtgagaagagtaacattaaggtagttatttggtcatttttgcagattattttaagacaattctaggactgatttgtggtaaatcacacattgctgtatcatagttgtgttcactgaacatattcaggggctctacagatgcagggctcttagctgctttgcacacttctgaattcctgccctgcgaacaggactggatacctaatagacaacaggtacttgataacagtttattgaattaatgagtgaatgaacagatacataaatgcatgaaagaatggttgtaatgtatataacttggatttcaagactttttactgactgttcaaaataagaaattgaaaactttcctctgattttcctctactatttacacaatttaaatggaagttatcttgtaccttcaatttctgtctaggattcgtacaataacgggtcatctctgagtcgcttaatgtctcacttgtctttctacagtgtgttgaagagatcctaggatacctgaaatcctgctttagtcgagaaccaatgatggcaactgtttgtgttcaacaagtaagagcttcattcttttcctcttctgttaagacgttcgggtatgacagcaaaacgctgctactccttaagaggcaggcgctgttggcataatcagctgggaggattgtggggtccagcgcagcactttttggctcagtccatgattgagccaagaggccatccttcccttcactccccaggaggacgaggtctgtcactgtggagggcagaggacaccagaagctcctctgcaacctcgctagttaacttccagtccctcggagtttctgtttagaatgctcaatctcatttagaattgcaaggaaacccaaaacgcctatttaaggtacaaacagcacttcatacaatatctcatgaggtattaatagtgattcacaggaagaatttcacgctgtgagtctttgctaacatatccagttatttacagatggatttgatatttgtgtgggagattcttaaaagtgttgttcacgccacattgttgatgcctcatttttttcactgtagttgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcggggtaacagttgtggcaagaatgctgtcgttggtggaagcacgaaagagcaagcaggaaatactttgtaaaagaataaaaacgaaaaatgttagcgaacatcttctaatagtctgctgtattcagagaactctaggagatatatatggttgatgcaaagatgatttaaggcatagcccggccttccaagaagtgtgtggccagtgagtgagatgggcttgggacttacacatctcagaggtgggggtagaggaggaggaacactgagtgggctgagaagcagccagctctcattgccaaagtgtgtcagcaaaccagaatgcagttcataatgtccccacccattcaaagcacaggacctgtagagtggtgtggcatgtgttggtggcacttttcaggcctgtaacaaggatgaaagaacagcttcatagcagcacagtagtgctggtgttcagaggtgtgtgaaggccatagaagcatcttggatatattaccttgtgttttgtcagctttatgactagaagtctcttttcacttaaatttgttttttttttttttgagacggagtcttgctctgtcgcccaggctggagtgcagtggtgcaatctcagctcactgcaagctctgcatcctgggttcatgccattctcctgcctcagcctcccgagtagctgggactacaggcgcctgccatcacgcctggctaacttttttttgtatttttagtagagacggggtttcaccatgttagccaggatggtctcgatctcctgacctcgtgatctgcccgtcccggcctcccaaagtgctgggattacaggcgtgagccaccgcgcccggcctcttttcacttaaatttatgtttgtgtttttaatgcctagtatacaggacttcttaaattgccttaagtatgaacaggtatttgagttgctaatctgtatagtagcaataatagaatcccttgtttttccttttataaatttagcgattaaatagctacaattaaaacactagagtcaggagtcaaggaaaatacccatgttccaggctgtatgttagtgatgtacttactatatattggagtttcaggagtaagtctgtttcaatgctttctgtaaccatttggggtattaataagcatgtgagtgtgtgcatgtttgggttaatttcatatatgtttcttagaagggatatcattgatgtaaatattttaaaggcttgtcctccaaaaaaatcatgtaatttcttctaaattactgatcttttaaatgaccttcacctttctctcaaatctcacttaagactgggctgagtagtcagtttcctgtagcagaaaaaagctcagacttgagtagccttctgcgagtgaggagacttgatggctgtcaggcagctgtaaactctaaatagagtgtcattatctgaagagggcgatgctgccacactgagtggcctttcaagttgtttctcaatctgacacgttctgatcgtgtgaatgtgaaattggtttgagcaggagtatatctgagtgcagaggagattatttaaagatattctcattctctgcttcccttttattcccatttggcagatggtttgatgtcctccagaaagtgtctacccagttgaagacaaacctcacgagtgtcacaaagaaccgtgcagataaggtaaatggtgccgtttgtggcatgtgaactcaggcgtgtcagtgctagagaggaaactggagctgagactttccaggtattttgcttgaagcttttagttgaaggcttacttatggattctttctttctttttttcttttttatagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtttgtcacttttatctttcatccatcatacctgttcctaatttagtacaaattaccctaaaagacactgaaatctactttaaagaaatgtggtctgcatgtttccctcatcagttgctgctgcttatctttttcatgcacctagctggtgcagaaggcctggggcatagccagcctcagcaagtcagcatccttgccccagctccctggactcaaggctaacctggggttggctgttagggatttccaaaggtttgtcccatccacttgcctcccctccaaaataagtttgaatttaaattgtgagatacaattaagatttattgtttggggaacatttttgcaaaatctagagttagtttaaacagattatcaattattaccataattgatcatctgcagtttcaagctatctaacaggttcacttacctctttaaaaaggaatggaatttagcaggacagtaactgagacccgtgctcctggagtccatgtgggagctgtgtggctctgcacaagcatttgcacgcttcccctcttgactgcattaccttcctcctatagttgctgtgggcaccagattctggctagtcctgtcccttcatgatgcacattttcctcaagattcgtcccagttaaatcactgcagatgaaactgccttttcatcgtcaaaatttaactgtcatttttgagccgtgatcttgggctactttcttatgtggggtaggaatatttgtgagttagaaatattacacttctctatttccttctagacgtaaatctgttaatcctgtcagcactgttactcacctgaaagggtctgtttccctaggagaactgagggcactcggtcaacactgattttccacagtgggtattggggtggtatctgcttgttttttttgttgttgttgtttgtttttttttgttttttttttgagatggagtctcgctctgtcacccaggctggagtgcaggggtgcgatctcggctcactgccagctccgcctcagaggttcacgccattctcctgcctcagcctcccgagtagctgggactacaggcacccaccactacgccaggctaattttttgtatttttagtagagacgaggtttcactgtgttagccaggatggtctccatctcctgacctcgtgatctgcccgcctcggcctcccaaagtgctgggatgacaggcgtgagccaccgcgcccggcctggggtctgcttttaatgaaggaggcatcaaggggtgggctttgcgttggcctgatgctttcatctttctttcacaaaacctgtccgaagaaaatccgtctaaatgggccattgctctcctcaggaaatagtcattgggaacttcttttcctttcctttgacactaggaggctgactggggagaagccctggtctatggctgtgggcagcaggggctgagaggagcaggctctcaggggggcacgggtaccccaagggaagccagagccctgatttgttccattctagtaagaacaaagactgctctggtttcatgtttgttctgattgcctttcatcaaccggtcccctttctcccagttcttaagattcagtacagtgacagttttatgaacaagaatagaacactagaacagacaaaccattgaactctatgctgataaagatttattgagctcctgctgtatgtttgcattctgcccagaggctctgagaaaaccaggccatatgctccatgctttatccatggaagctccccgtcaggttgggaaagctgacagctgcagggaatacagtgtgacacaaaactggctcccatgcagcccttacgtgtcgcctctcagatggttgggggacgaaggtcgactcctttgggtatcttattactaaaccagtttcagggaatctgtgccaccctatctgccattaacgtgaacagatgagtccccaaggtgtaattttgggtattgtctgatgtctcttggaatttattatttgtttttccaatgagatttcacctcagggtatagtaaagttgttgaggggattcctggatgtgttctgcaattatctaggctgatttcagaatagagttatgcttatagtcaaatttatcagctgtcaagaattttatttaaaatttatgcagataagcaggaggaaaagaagcctggtttttacattttaatcctattattgatgtgaaattttattttccttcctgtaggtgtttattggctttgtattgaaacagtttgaatacattgaagtgggccagttcaggtaatagcattttattattttagatttttttcttcttcttgtgtacttacatgtaatttaggttattaagtgaatgtttaaactactgttaggcatttttgctgttttctttaaatggaaatctgactaacatactgtgcatttttgcttctcttaaaaattaatgtatatctcaagacttgtttggaagtagttatgtatctgaaaattccatatgttgtcagtattcattgcacatttcaaagcatttaattgtgttgacagatggtggaatgaaatcttgtggtggagcactagtttttaaatcttcttagagaaagcagttttatataatgttgtctttagtaattattatgcatttgtattctctgcagctttttcttgctagatgttgaggttttaatacttcttgctagtccattacaggtttataattattaaaagttaaaattcttttagtacctaaaatgcttaataaacattgtaattaggaaaatttagtgcagaaggaaagtgttcccagattccctggggtctggaaacatagtgtttattctaattacatgacacctccactgtgttttggggcaagttactgtttctcttttgagtttcaatttcttcaagagcaaagaggcagaggagagctaggaagatcgtagctgctgtgcccctgtgccgtcgggtgccttctacctgctgcctccgaacctttacacatgtccctgctctgcgcgagggcacagatgggatgcactgtggcaggggtggggttagagtagatcacggacacctgttagcttgatgtgtgcttgctgtcaaggttgaatcatgaattattttatgttgcttatattgatatgtatcttaattttaaaagaaaggtctaaatggatgtttttgtttttagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatggtaacgggacacacctttcactgtcgtcttcggtgtcgtgatgtgcttggcagtgttcgttttcatatacccactttgaacgttgtcagtggcagccatgtgcttctcaggctctgcatgtgtgtctgtgtatgtgaaggtactggttagagacgtttcaaaagagaagagagcatattctttactctcagcaatttgtaatcttctcagggaaaaaaattcaagaaacagtaagataacctaaggtacagatagattctgaatataaagttcctgttcattcacatgaaacgctaaaagttcttcacttgatcttagccaaaaggccaagaagcgatgcaacactaaaaattcttaaatcgaacttgccgtgaattaaattttgatctctcatccagtggtattggagatatagtttgacttgggttcagggctttctgttttgcctgatgattttgctggagcttaaataaggaacccaggagatggccagctgtgcaagcccccagcctgtggaaggagctagtgtggttttatgaatgagttgcaaatctttctttgagctttttgaactgatcttccagcattgccctattgacccctccctgactcctttgctggaatctgtaggcttttgaactttgacagggacacatcctaagacccttgcaaactcccagatgtgagaatggcactactacttagagtcttttcgactcagcgtgtgtgcagaagagcatcaaccgggctgtgttgcgaggcagggccttggctgacctctcagtgtttacatagctaagccagttagtgtttgccacggcctcacaagggcttcagattcacacagccaaagtatagattattaaaggcataggtgtttggtttcctggacttggagggtctttggacagaaaatcagtaggcaaccacacccagtactttgtgctgggaagcttggtcatctgtgagagggtcagagagtatacccatgcgtgcatgccaccgaagggtcagtgagtattcctgtgtgtgcatgtctcagggccggagagagtatgtgtcactgagaggtcagagtgtttgtgtgtgtgtcaaagagggttgcattgtgcccttcactgaggggtcagagggtgcctcgcgtgtgtgtgtgtgtacgtgtgtgtgtgtcactgaggggtcagagtgtgcctgtgtgtgtgcttgtgtgtgcgtacatgtcactgaggggtcagagtgtgcctctgtgtgtgtgctcatgtgtgtgcatacgtgtcactgaggggtcagagtgtgcctctgtgtgtgctcatttgtgagcgtatgtgtcactgagggggtcagagtgtgcctctgtgtgtgtgctcatgtgtgagcgtatgtgtcactgagggggtcagagtgtgcctctgtgtgtgtgctcatgtgtgagcgtatgtgtcactgaggggtcagtgttcctatgtgctcatgacattgagggtcagagtgtgcctgtgtgccaatgaaaggcatttcttatatttttttatatgtggtcatagtagaccagttaatttattttgactcctgtgttagaccaaaataagacttgggggaaagtcccttatctatctaatgacagagtgagtttacttaaaaaagcataataatccagtggctttgactaaatgtattatgtggaagtctttattgtcttttcagatgaatcaagtagattattcttgagaccaggaatgttgctgttttggttatttggaaagttttatcattttcaaattgacttttgaatttgagtcaccttttttcagaagtggtgttaaattataggagccctaggttttttttctttttttagaagtcatcacaaaatgatcagtgttcagaggaagagctttgaccttccacatggtataatgattgataaccttaattcatctcttaccataaaccaagtatgtgtaagggttttctttatttcttgaaagcattttgtagatgttgagagcagttttccaaatgtaatttccatgaaatgcctgataagggtacccttttgtccccacagccataccggctctgcagcccatagtccacgacctctttgtattaagaggaacaaataaagctgatgcaggaaaagagcttgaaacccaaaaagaggtggtggtgtcaatgttactgagactcatccagtaccatcaggtaagaggaatgtatgttggaactgtcgtggatactttattgacccgtgcagatggaaggaagtgccatgtggtaacgctcactgttaactgtgttactttgaaccaggtttgggctttctggggcctgggtagatgccggtgcagggggatggggagggaggcggggggtgggggggtgtggtggagttggggaggtgcagtggcaggaggtgttgttggtgtgtatcctttttttttttttgagatggagtctctctccgtcgcccaggctggagtgtggtggcacgatcttggctcattgcaagctccacctcccgggtttaagcaattctcctgcctccacctcccgagtagctgggattacaggcatgcaccaccatgcccagcaaatttttttttttgtatttttagtagagatggggtttcaccatgatggccaagctgtttcgaactcctgacctcaagtgatcctcctgccttggcctcccaaagtgctaggattacaggcgtgagccaccatgcccagcctggtgtttatctttaaagtgggcacagccacaggagttcacctgactcctggtctgagagtcacgagatcgttcaagatagtgaggccctcttttccaaaacgaggaccaaaaatcaattgacagtgttggtcaagatggtagaaaccttaaaatgatagaaatctcaactctgaaataaaaactttatttgtatatttatttaccactattttgacatagggctaaggtctttttctttgagctgatttctggttttgttttcttaaagtggcataagaattcaaagacattttgaggaaggctgagtgcagaaatctctctttttaaatgacttctcctttcttttaacttgcactgttgtctagccctcacttattttgtcaattctttttagctgtttgtctttgaatcttcataaagccatagcttttctcataagaagcagcactttctttgttcattcatattttaatgaacccctgtagtatttaattaaatacttaatgcctaattaaatcacataattgcaatgcaaaagtacatgtatcataaagaggtctgaaaatgagcaactggcaagcaggtggtggcaggcagagctgcttgggtgggtgggtgtcatggagaggagttcatcagccacatgttcagtgagctctggatatgtctgtttagaaatgatcactaataaacttgtgctcaaccatgtatacctctgggaagcaggtgctcttcagtagattgcctctgcagagaacacagaattgaagtgaatgtccacaaaggcaatgagccacctgcagaatagtttagtcaaggctgtgtttgaagtttgccaaagattaatatacatttgattttcatgttgtgccttttctctgattgtgaaatattacaaattctatacaaataacaatgatggcaaatcctcctgagcaaagtgtgcaccttgtatgtgccctagaggaacttgtgtttcgttctgattcccctacatttctcatgtcatagagtgggggttgcattagtgtccccctgtcctcgctgggatcacatctgtttggatcctagagtcttccagctgaactgggacaagtataacagacggacacgtaggggtggaaaggcgtctcttggcagcagactttctaattgtgcacgctcttataggtgttggagatgttcattcttgtcctgcagcagtgccacaaggagaatgaagacaagtggaagcgactgtctcgacagatagctgacatcatcctcccaatgttagccaaacagcaggtttgtccccgcagccttggcttgttgttgcatagtgatggtagcttaaggtccttgtgaaaggtgggtggctggaatcagctcttccttcagtcctaatctgtgccttgatagcagttctccgtgctagtcatgggacagctgacttcatttcttctcacaatgccatctcaggttggtattgcccacctactttacaggggggatcccacagctccgagaggttatggaggtgatcaggcagcacacagctttagagtgctggggtgagggcgggccaaggctaactctaaagcccgaacccttacctcctacactgcctcctgcattctggtcaacccagtgttttatttggtggttagatttttgtttttgttaccttactgcttgtaatttagcagttttcctttcctttcccttcctttcctttccgacagggtctcactctgtcacccaggctagagtgcagtcgtgtaatctcactgcaacaacctctgcctcccaggttcaaccaattctcccacctcagcctcctgagtagcaaggaccacaggtgtgcaccactacgcctggctagttttttgtatttttagtagagatgaggtctcgctgtgttgcccaggctggttttaaactcctgggcgcaagtgatccaccaaccttggcctgccaaagtgctggcattacaggtgtgagccacctcgcctggcctattcatcactaatcagaatttctatgatcaaatgacatgaatcattgtttccacaactgcagtggaaggaaatggcctggcagtgccagtttcagaagcagcctgcccccagtcaggcacaggccactgtgcccccagtgtagcagcacctctgtagctcacagagaagggtggtggggacctccttgaggcagctctgccagaaaatctcatgagctgcctggcacagcttgaggttgccttttaagtggactcagcaaatacatgtttgttcatcttgattatacacaataaacaactactctgtatagtacgagtagtccgtggtttttggcatttgatttaaacttagaggcatgtgatattgatgttactgccttcatgactgcacccccattctgatttcataatggaatgttatcttgagaccagttagacaacaggacagggatcttggcttctggtgagattgacagcagttttagtgtggtcagggtctccctgcctacagatggttttagaatggtgccctggaagctttatcccattcttttctgtgcgtaatctgagtagagtggagatcgaaggcctgaatacatagtaaatacctgacttaatatctgccgcaatggaaattgtgtgatacaacatttatgaaacgcttagtgcagcacctgccaggtagctcaccacaggtgcatgttgcattcagaagtagtgctagatactatcctgttactggcagtgcatacatcagtgatcaaagcagattaaagaaagaccccctgccttcttggagtgaagattttgttgggatgcgggtaaggggacagacaatagaaaagcaagtgagtgaagtctataccatggcggctgatcaggaacaccgtacagaagaatccaggagggaagagagttaggtggtgtctgcggtgggagtggcattgttcagctggtgatgagaagaagctttggtgatctggtgacatttgagtgaatttgcagaaaggaaagatacaagcctaggagatacctggggaaggaacattccaggcagagcaaatagcagtgcaaaggccctggcggggggcggacatgctgttagggtacaagcaatgagggtggaggagtggggcagccatggggagggaagggagtgaggcctggtggggtgaggccagtgtggaggagccttgagagggtttgcgctgatgtggtgtaggttttagcaggatcattcttattcctgagttgagaatagccttgagggggaggtgagggcagagcagggccacccatgtgagacccggcactggagtggaatggcccaagtcagcatcccttggcagcatgaaagcaaaaccagcaaggtttgctggtggcttagatgtggcatgtgagagagagcagggctttgggggtgatttcagggtgaggacagggtggctgtggacaaggtagggcagacattgggggcagcaggaggtcagagcctgtctggatgtagcagttgagaccccataggtgcctaatgaggtgaggccagcatcaggtgtatgagcctggagttgtcgagagactgtggggcagggggtcagcatctgagatgtccactcacagtggacccagactggctggagaggaggaggagcttgaataccgagcctgctgagtcccagctccaaggtcaggtaggtgaggggagccagtgctggggcagggggagtaggcaggtgtggggttcctaaagccaagattttttttaaggcattttgtgcaggagggcgacatctgctgtcagcaccttgggaacttggcccaggtttggcagcaccgagggcactgatgagtgcttttggaggagcaaagggagccaaaccctaatgggaatgtgttcctgaaaggacaggagagagacttgggaaaaggttttacttgaagagggaacggagaaatagggcagtagccagaggaggagaggagtcggcaatgggttaagttggcagaaatgaaggcctgtttacgcactgagggcagaagcaacagggaggatcagttcatgacacaggagacacaaatcgccgttgtggtgttcacagacatgggttaggattggctgcatggatgacagagcactgtgggttctcccagagttgctggggaggaggcagagttggtgagcacaggcgagggtccaggatgcaggaatcctggagctcaagtcagttgttcccttgttgtaagatgtggccagtgttgtgagcttcacatctgtgccttgaaaaacaccacatctgtttgcagagttgtttactatgtatacacactcagtagaaacaaaaattggaaacagtcagtgcccaccatcaataagtaatggttgaacacactgtggtataagcttagactattttagcttgggctattttgcatgattaaaaatgttctggccaggtgtggtggctcatgcctgtaatcccagcactttgggaggccaaggcaggcagattgcttgagctcaggagtttgagaccagcctgggcaacatggtgaaaccctgtctctactagaaatacaaaaagtagctgggtgtggtggtgtgcgcctgtagtcctggctaactcaggaggctgaggtgggaggatcacttgagcccattcgtgcgccactgcactcctggggcacagagtgagactctgttagaaagagagagagagaaagaagagagagggagggaggaaggaaggaaggaaataaatggaagaaatggaagggaggaaggggagggaggaaggaagaaaggaagttcagccagttgccttgggagttctccattgcactgggttaagtgagaagagcagagacgtttatgatttttcaaaacaactaaaacaaaacctctgtgggtgagggggcaaggatatggctataggaacatggggcagattaagaaagggatatacacacaccacttagcatttgttacaactgttgtgggagggatggagtgcagaaaaagaaaaaaaaaagtgcacaccatcccatgtatgtgtatacaaagggacgcttggaagactggtccccaaaatgttggtaatgattgtgtcagggtgctgcagtgctagttgattttttttcacacttttgtatatttgagtcttttacagaaagcatttattatttatgtaataaaaatctaaatgacaagatttctgttatgggaaaaatgtagctatacagtgttgttgtaaaaatgtttgcttggttcaccactgaacttaaaatgcttttaaatgagggaaggtgacgatgagatgattatgatgatttgcccttgagttacatagctggtgtacaggaagctgtcgtttcttttggcttacgtagaaatgtttgtggtgtctaattccacagatgcacattgactctcatgaagcccttggagtgttaaatacattatttgagattttggccccttcctccctccgtccggtagacatgcttttacggagtatgttcgtcactccaaacacaatggtgagtctctcgcctggctcagcagatgaatctggacggcttgttcaggctctgattactgggaccacccccagaatgtctgagtcagtcagtttgggtagggcttcttgagagtttgcttttttttttttttttttttttggtgtgggggtggtgcggaacagagtctcactctgtcgcccaggctggagtacagtgtcatgatctcggctcactgcaagctctgccttccagcttcacaccattctcctgcctcagcctcccgagttgctgggactacaagcgcccaccaccacgcccggctaatttttttgtatttttagtagagatggggtttcaccgtgttagccaggatggtcttgatctcctgacctcgtgacccgcccatctcagcctcccaaagtgctgggattacaggcgtgagccaccgcacccggcctttttattttttttggagatggagccttgctctgtcacccaggctggagtacagtggcgctacctcgactcactgcaacctccgcctcccgggttcaagcaattttcctgcctcagcctcccgagtagctgggactacaggtgcgtgccactgtgcccggctaattttttgtatttttagtagagacggggtttcactgtgttagccaggatggtcgcgatctcctgaccttgtgatccgcccgcctcggcctcccaaagtgttgggattacaggtggctctcgcaccaagccaagagtttgcatttttagcaaattcccaggtgaaactaatgcctgcttttctgggagcacactttgggactcagtgatagagaggtttattggtaggatagtaaaataggagttattttctttcacaaaattggcaattgggggaaatttaatcttccttttttcttcagctgtgacttatgtattatgtttattttaggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtatgctttctatctgagcctataactaacccatgccttttgggaagtcacgtgatgtttcacagtcagtaagtctggaataatacctggtcttgcttcacttctgagttgggtaaagaagtctgtatcagtgtaattttctaatccgtcctgcattatctatggctcttggttcatacctgtcttgaagttctgtcatgttctgtctcttgtcctcagtagagatgctacagcagtggctcgcctcaggcagggcagggcagtggggtggctgtcctgggggcaggcagtaggggcacgctgacgtcagggaagttgaaacccaagagaagccagtaaaagtgagtctcagattgtcaccatgtgctggcagttttacacgctgtcagtaataaaagtcttctccctgcagggcagcctgcctccaataaatacgtgtagtatcaaatcctgtcttccctcataaattgtttggaagctccccaaggacagtgatgaggcactcgtaagtgcttgctgcctagatgggtccctctccacctttgctagattctgagcattcactgagttagagctgcttctgcaaatgtgctgcttctgctaagtggctgtgacttcatgcagccttcacttggtttgtcatcagtggagatgccctgtgttgtcgaaggagataagcccagtaagcctgctgggcaccttttggtttgcaggttcagcaggcagcccatggctttccctgtgtcgcattgaagcagctggctaaaattgatgatacattaaattcctgtgacagatgatcagcttgtatttgtgtaatggtgtacagttcacaaagcttaaaaaaatgctacctgccatttcatcctcagtgaggaaggtgatacacagagagaccaagtgactgtgtccacggcgacggcgctctgcatttcactttagcggttaatgtactctacctatatttttactttatatttaccatatatcttttcatgtatacttggcgtaagtgctttatagtagtcacctaattcactgtcatcttttttgtttcttggaaggtttctattacaactggttggtattcttttagaagacattgttacaaaacagctgaaggtggaaatgagtgagcagcaacatactttctattgccaggaactaggcacactgctaatgtgtctgatccacatcttcaagtctggtaggtgaatcacattagtcttcctggagtgtctcgttccccattctgcactatacactctcagagtgtaggagctgtgctgcccggtagaaactctgccttgcccagtgtgccagttgaaaatatttgttgctgtaagagtacacctgataccatgtgacccagcagttccactcttgggtatatacccaaaagaatggaaagcagggtggtgaaaagatatttgcatgccagcattcatagcagcattattcacgatagctaaaatgtggaaccaactgaagtgtccctcgatggatgaatggataagcaaaatctggtgtatatttacagtggaatattattcagccttaaaaaaaggacattctgacacatgctacaacatgggtgacccttaaggacattatgctaaatgaaataagccagtcacaaaaggacaaatactatgtgattccacttacatgagggacctggagtagttaattcatagatatagaaagtagaatggtggttgccaggggctgcaggggaggggagttatttttacaagatgaagagagttattctagaaatgaatggtggtgatggttgtataacattatgaatgtacttaatgctactgaactgtacagttaaaaatagttaagaggaccaggtgtcatggctcatgcctgaaatccaagcactttgagaggccaaggcaggaggattgcttgagccaaggagtttgagaccagcctcagcaacatggtaggaccccatctgtacaaacaaactagccggggatagtggtgtgcatgtggtcccagctactcaggagactgaggctggaggatcgcttgagcccaggaggttaagtctctagtgagatgtgttcatgccactgcactccagcctcggctatagagtaagaccctgcctcaaaaaaacaaaacaaaacaagacaagagccaaaaatggttaagatgggccaatcacagtggcttatgcctgtaatcccaacactttgggaggtcaaggtaaaaggatcacttgaagccaggagcttgggaccagcctgagcaacatatcgagacccctatctctacaaagaaaatcaaaaactagctagatatggtgggcacatgcctgtagtcccagctacttgggaggctgaggtgggaggatctcttgagctcaggagttcgaggctgcagggagctattattgcactccagcctgggctacagaatgataccctgcctcttattaaaaaaaaatccaaaaaaaaaaaaaagtaaacctgagagcttcctcctcctgtgttaaatttggaggccaagatgtttttgttacttttacaaatgatcaaggacggtgaaggttgggcatggtagctcacacctgaaatcccagcactttgggaggctgaggcggggtgatcgcttgagcttgagaccagcctggacaacatagcaagagaccccatctccacaaaaataaaaaaataaaaaaaaatagccaggagtagtggcatgagcctgagcccaggaggtcaagctgtagtgagccatgatcatgccactgcactccagcctgggcgagatcgagaccatgtctctagagaaagaaaatgacaaggacagtgaacccaagaaagtcataagatgccagctgtgcagcaagcatggaaagcagccagtccaaattaggacagtgtgttttccaagaagaacgatcgtttgtaatgagaatgctttgctttaaataaatgactaaatagctagaagcctagttctaggggataggcacgtctttcttctctcaagaaaatagaaaggcaattctaatttctagtaacagcaaacagcattaagtcatggtccaaatatgaggcaaaccaaaatgtggcttgattgttcagcagttgatctgttggaagcccttgatattaaaaaggttctcctttaagcggcttaggagtcacgatcaaagacctatagaaagagatgccatccttctaggatccttggctctcttgggaactagattcagatagtcataatgtaaatactgcttgagctttctttctttctttctttctttctttttttttttgagacagagtttcactcttgttgcccatcctggagtgcaatggtgccatctcggctcaccgcaacctctgcctcccaggttcaagcaattctcctgcctcagcctcccgagtagctgggattacgggcatgcaccaccacgcctggctaattttttgtatttttagtagagacagggtttctccatgttgaggctggtctcgaactcctgacctcaggtgatccacccgcctcggcctcccaaagtgctgggattacaggtgtgagccaccgcacccggcccgagctttcatttttgaaatcaatgtatgactgaaacactgaagacttactgacttaattatggtttcagaacagaatgaaaatgtcttcggttctgatgaatataaaaggaaaactaaccaagttaatttggcaagtagatggtagagatagaggtggggagtggaaggggaactaaaatcttcacctagcattgttgggattatatggttacatcatctgaagttgacagaccaaaatatagaggcttcagaggtctccaaatagaactaaacatgtaattcagattgttaggaggtagtataaatgagctaaatctcatctttattacggtagagttaatgggtgatgtctaaagttgtctgaagtctataaatcatgacaaattatgatgtggtgattgtattcaacagtctttcagttgcagggataaaaccccagtttaaactagagtaagagaaagaatgtgttggtttaagctcctggaaagtgcaggcaagggtagttggtaggactgcatctagtgttgtaattctgtggtctgcattgtatatttatgcatctcagctctgctttcttcttttcatttatataatttttaaattttattttaaagatagggtctcactttgtcgcctaggctgaagtgcagtggcatgaagtgcagtgcgaggctcactctagcctcgaactcctgggctctagagttcttcctgcctcagccttctaagtagctgagacaataggcatgtaccaacatgcctggataggttttaaaatttttttgtagaaatggaagtcttgctgtgttgcccaggcgggtctttaactcttagcttcaggcgatcctcctgcctctgcctcccaaaatgctgaggttataggtgtcacccaccacgcccagtctcatctctgcttcctgtgttagttttgttctctggtgggctgttttcacatgaccgaagatgacctctagcaggctgtgttctcagcccctcaagtaggcctatgtgattggccttgcatgagtaatatgggtgaccataaacccctgaatgctctggtccacatgggccaaatgggagactggacagcattccattgatgaggaggtggggctggtctccgggagtaagggagaggagcacatgcagtaactgatggtctgctgcaagggatagcagcacagcagttagaattttggaggtaactaccagaactgaaaacagaaatgataacaagtagttgccttaaaaagggatgggagcagggtgcttttgtgatcaaagctcctttctcttactggatttttgtacacattttgcatacatatcttagagtaaaagatagcattttcagccttggtccatttgaggatactcttggcgtggcccgcctccatgctagcaggctctggttgtgccaagttcagttgagcatcctggctcttgcctgcacggaacttccagtcagtgcgtcagtatcacaagtcttgatatttcctatgaagaagaacagtagtgcagtgacagacgaaatgggtgggcaggcagaggcaggatttctgagggagagaagtagctagctttttgcagagaagagttccggcacccaagagagcagctgagagtacaggcaggcaggcaggatgccggtagggcccggccgcacggcgccacagaatcctggagaaaggggcctcttcatggcctctgcattcagctgctgtcaccctccgcacaggccatggccaaaatttaattttcatagtggactctagtttttgagccttacttgctattattgaaataattttcttgtttctttttaaagatcttcggattatgcttcactgaccactgtaataagtttaaagttgagaaaatatggcttgttaatgaatgataggtcaattttagtatgttggtcattttaatattttgccaccagttggtttggatttgatgccaggaggagacagcctcatttctaaggactagtcttgcctttgtgggataagggtggtgtgttctgtgtccttctacatgtccgagcgatctctgtgcagctcaaatgtggtcactgtcttattgcgctgatttcctctccttccatctcacaattgaggcaaaatattgttactgttgaagtgttgtccaataggacttccagcagagacaggatgtctgcactgtctaatttagttgcctttagccacatgtggtgttctgtacctgaaatgtggctggtctgattggatagcttaatttataattttatttaattttaattaacttaaatttaaacagctctgtgtggatagtggctcctgtatgagacagtgcaggtctgttgagaagcagctttactggtgggagtggagggcttggagagggcacgtgggtttcctgctggtatcttttgaccttatttaatctgcccaacatttgcaagtaagttgtgtgtgtgtgtatatataaatgtgtgtttctgtcttcttgtttcctttgactgcatttatttgaaagacactaggtggcagaattactgtatttgattggtttcaagataagagttgaaataattcatctcgtgtttttatataagtaaggtgtgtttagcatgtaaaattggtaatatgtattcacgtactgcttaaacaaaggctatgaattccacccataaaccgaaaatgaagacctttaaatttgtccatttcaggcgtgggtacttcttaaataatacctggttcaggaactagtcagaatggcacccttgactttttgtttcctgcttttcctcttgttgggagaggagggtattcatcccaaagtggtttgcctatttcacattccatctaggataagcagaatagccaagaaagatagctgtcctcctgtttacaacatttggggtaaccagcatccctctcttttggtccaagatagactggtttagaaacagatgatggcaccagaggcccaggaggtggaaacatcagctttgtttgttgtccatgtggctgaattagagctgtctggccttgtagcctcaacacggccttccagctttgctcaccgtgattttcaaggacacatcttgtgctcttccctgcctgccatccagactatacccagtcagggtggcaggagctgctgccccttcctccctgagtcctggtcgtgggtggtggagatgtgccatgacgctcacggaggcatgctcaccccttcctctgtggcagaggggatggctgcacgacagctcttccctgtcctttccaaagcgtctgtggttccactttttggggcaaagcaggaatactggaagagagagaaagtggtcctttctatagtaataaagttgacattgattcaagttcatgcttggggaaaggacagggctactaacaattataatgctgggagcaatggaattttctcatgggtatgtggtaggtttaattttaattatcccagttaattcttagaactgctctgtgaagtatttcccgctttgtgcttaagttctaaaagatcctgtgccaaaaccaagaatgaaaacccaagcattctttcttgcccatcgatctttctctcatcaggccacttcttgggttgatagtggtgagtgtagccgctgccactttcagaatacccaccatgggccccagtcactgtgtggcgtggagaagagatggttctctctgtgtcatagctgaacaagcccagcccagagaggtttctgccctaggagctctcgatggtggaattgggatgcgatcccacatcctgcctgttttgaaaacagcattctttatttccaattcctgcttccattgttccttttaatatttctttgtttagctcacaaaaacacggcttgcggagctgctgcgtgcagctgtagctgtttctctgggtgcagcctgcatccgccttcctgcccgcctcctttcctgcactgccatcgtggtctccgggcacttggtccctttctcttcccctgagtccctttggctcccctgtgccacccttgtgatccacaggctctgccttctttctgtctcagactgctgctcatcactactcgggaccctaggaagggaggttccaccgagaagcatcttctcatctcagccacgttctcagtgccactgttgtctttgttaggtaatggtagctactgtaacaaataaaccaacatttccatggcttcacaccagagaaggttgtttcttggttttatgacaatgtattgagggtgttcttggttcacggatggttttcctccatgtgggaattcggggacccaggctcctttccttcttttggttctgttctccaggccttcacatcctctgtgtctggttggggacaaggagagggaaggtaaagaaggctttgtggccttggataagtgacaggcatgcctttgctggtgttctctcgtggtgacaggtcacagccccaccctgtaaaaggggactgagagacgtcgtcctgctgcttcccagcagcagcactgtggtctctgatgtgttttctgtgaggataaaaacaggtgattccaggatgaggaaagtcagggaaacccttggaaggaggggaccaggcgggtgtcaccatgggattagtggtggcttcagaatgagctgcagcgagtgccatgccttctaaagcttttgctattctgatatgcccacaccatgcccagcaggtgtctgccttgctctccgcagagagagtgatgaatccttctcatgagcctctgtccagttgttcctccctccacctggaagggaccctgggttcctcataacatcccagcggaacaggggaccttctatcctgtccccaagttcatcctcatcctcctgccggcttcctggcccctcttatgtctgcttcctgacgccacatccttctggattctctggaattgaattttgcctttgatgcttatttaaaaatatccattgcaggccaggtgtggtggctcacacctgtaatcctgtgcactttgggaagccaaggtgggcagattgcttgagcccaggagtttgagattagcctgagcaacatgttgaaatcctgtttctatagaaaatacaaaaattagctgggcatggtggcgcacacctatactcccagctactcaggaacctgagacaggaggatcaattgagccccggaggccaaagctacagtgggctgtgatcgtgccactgtactccagtctggtcaaacagagtgagaccctgtctgaaaaaaaaaaaaaaatccattgcatacttcaccgtagcgaaacatgtatgtcttacctttcctttcctgcctgtagctgctcttttacacttaacagccacactaagccagccttaaatgaaaaacaaaccagcacttcctgtgccctcctgcttccttcatgaggggtccctccctctgtgtacactccattctcattgcccatggtggtttgtttccctcttgtttctcaagccatggcagcctgcctcttgccctctttactaaaaaggcctttgcagaggctgcctgtgttctttctttctaggtctctctcatcctaggccctccagcttgattctgtggagctgccctcttgtcactcagtagcttgtggggtcttctctgtctagccacttaattgattgtgttcctcgagttgctgtccatggtctctcgttactgttttctctgtgtttctgcctctctccttggccttggtaggtccatcccctttgtgaccttggctgttgctctcatggacaactttctcttgctggtccttgtagtcctggcatccagcttctcgacacgggacttgtcctgccagtacctcagacttgcacttaaaattgaactagcaccactgtcactctccagggcctcttcttgttaattagatcattagggatgttcagaatcccagcatcatagtatgttcctcctcccgctaccccaggaaccctaaccttacctcctcctctctatctactaggaggtggccctcagagtccgtctcatcttccacctgaacttccctaataggctccagcagctgccaccccgggggctgagtacttcctccatgccttgtgcagtgctgagccctttacctgggttctcctgtttgctccttattacagccctgcgaacagatactgctcttaattccatcttacacctaaggaagctgaggccccaggtaaggtgcatccaaggtcacccaggtagtagacagtagagccacgatctgaaccaggcagtctgattcagagcctgtgttgacactcagccacctagaacacagcttggattgtgggtttctattacctgttcaaaacccctacatcccgggtctgtccctgcacgtgctctgtggcctggctgcatcttccttgaaggcagtgcatgcctcttcactcagggggcccatgcaggaacagagggccccacagaaggatgaggccagtgcagaatgggctggaggggacaatgctgaccaggaagcaagtgtagagaaatcccaggaaacctggaggagccagagacaaggcattagaactcctcgtcgtgacctggtctgcattctctgagtgtgctgcttctgttagctcgcttccttggtctcaggttatagtttaaggcattgtggagccctaaaaagcctgtactctgtttttacctgttttaggaccctttcactttggggatgtgttgattttttttttttttttttttttttttttgagatagagtctcgctccattgcccaggctagagtgcagtggcacgatcttggccactgctgcccctgcctcctgggttcaagcaattcttgtgctcccgcctcccaaatacctgggattacaggcacccgccaccacactcggccaatttttgtatttttagtggagacagggttttaccatgttggtcaggctggtctcgaactcctgacctcaagtgatctgcccaccttggcctcccaaagtgctgtgattataggcgtgagccaccacacccggcctgaaatttaaatcagaaataaaattttgatcccaacagtgatgccaggcagcccagatctgggggagagggtggccttggccagctgggcctttctctgtttcccaagtcttgctgcctctccctgctgggctttgcagcctgtgcatgtctctgtgcctttgaccttgtttatccaaaggagaggatagaatgaagtcatgattcctggagccctgagaaggatgctgtggagaaatttgccggtagaatctagctgagtgtgttgctgaggtgccagcattgtgtgtggggaggctgaccgcttggcctgcctaggcccaggatgctccatggccgggcacagaggccacttggctgtcaggtgtcaggagcctgcagagggcacacagagcctggaccgcaggggggtcctgctttctcacctggcctccttcagcatttctgtccctcagtccttagcaagcccaggagctgttgagtttggcaggtgccgagtgctgttcctgcctgtgtagctgtggctcagtcctgtgggggccccgctgtggcccgagtgcagtgattcgaggcgctgagtgttccctgactccttctccaggagctgtgttcagactttcgcagctcttggcttggagctcctggagggcttggcattgccgaccaatgtggaggtcgacagtgagagaggaggaatgctagctttcttgaccagtccattaaataagtgggatattggccaggcacggcggctcacgccttaatcccagcactttgggaggctgaggcgggtggatcacgagctcaggagttcaagaccagcctggccaacatggtgaaaccccctctatactaaaaatacaaatattagctgggcgtggtggcaggcgcctgtaatcctagctacttgggaggctgaggcaggagaacagcttgaaaccggaaggtggagtttgcagtgagccaagattgcgccactgcactccaacctgggcaacaagagcaaaactctatctcaaaaaaaaaaaaaaaagtaggatatctgtttctgcttagaaaaatcagaattttctaaatgccaggtgttctgaatacgtaagtatgggagacgactcagcctgtttcatttttatgtaaaatcttcgcgtagccatgtggcactggaccgagatgaaagcaaagacatttctccttaactttgtttctaggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaagtaggttcataatgccccacagcccagggcgccagcccagcaccctgtcctgagactcccagtaacctgagctttggccaccgttaaagcattttcattttccattttttgtgagggcttgtgaaatttctgctgcatattaatattcctttcatggacagcatattattgggacaaacatgcggtccagctaaaggcattcaaaatagcagttgctttctaaatgcgattttctttggcaggttctttgacaccattgcatcttgtgggatatgcttgtcatgctctgtggctcctactaagttctagtccttaaattggttccatagccagacatgttgcaatgtcttaacctcattataaagtaaatgtggttctggttatccttagataatgaagtaacagtgtagcaaatttcaaaacctcttggaaatgttattttaccattcaaaaaggcttactaaggttctcgttatgggtggccctctttttgcaaaaggttttcaggcttaagctccatttctaggtgctccaacactccattatttgtatatgtatggaaataaaagctgtgaccacccccaaccctggcccccgcccagctgaatcctcagcacagtatttctggaaggctcaagatcccacgctggggaaaagaagttctggagacaaaagagggcaggtgctgccgtgcctctctgctcagtatggatactggaccttgtgctgccagggctcccagtagggccagttcatggcactcagctggaaagtccactgttgggaggcattcttaaccatccactctgtgccgtatgtagtggggtctggtcattctgttggaggagacagaccagtgacgacatttgaaatgcttggtggatgtcttaggcctgttacgatgactgagcactgtgggggcaggagacagaaagtcagtgtctcctagttctgtgctgctttaacgtgcatagaaatcagctgcggattcagcagatcactccttttctgacagatgggcctgcttactctgatgttatatcagaaagctctgaatctgggaattgtgtcccctgaattggagtaacagaaatgcttagatgatgagtgtttaaaagaaataaaccaaaggtaaatttagtttggaattcagcaagcgtcttcattcagccctctgagggcaaactacagctttttgtaaatgtaggtaaattctgtgactgtttcgtgaccccctctgatccagttttcctttataaccttctgtattgttccttctattatcctgaaataacattaatagattaggctgggcgtggtggctcatgcctataatcccagcaccttgggaagccaaggcgggcagatcacctgaggccaggacttcgagaccagcctggccaacatgatgaaatgctgtctctactgaaaataacaaaaattagccgagcatggtgacaggtgcctgtagtccctgctactcagaaggctgaggcgggagaatcgcttgaacctaggaggaaaaggttgcagtgagctgagatcgcgccactgcactctagcctgggtgacagagtgagactccatctcaaaaaaaaaaaaaaaaaaaaaaaattaatggatcaatggatttttaacctaataattaaatttcaaaaaatatcgttctttaatggtaatgtaaaggtaaaattaagataatatgtaacaagcatgtgagtgtctaaggtgtccccgtggtggaaggaaaaaataaatccccataagtgtccaagatgcccatagagagcagagctgttctggtttaaacccctgctcttagcactgtgtttttccagctgtgggtggtgggggatgagtatctttttatttccatgagatgagaaaaatgaattactagaagtgtgaaatacaaaacacagctgctctttttttagccatagactcagcagccataaaattgctgtatccagttgcagaaattcctgctgcttactcttgaccctctctcggtttgtgtgcatctcctctcaggctggctcccagatgggagctggctccaggcgacactgggtgctctgctccaggaggtccttatgtgggtcctgccctagcctagcccctctcttatggactctgtcactgtgggtttatgattcactctcaatctgtcttacctcttggtgaactgttagagtcctgcctatactttggcgcttgtgggtgtgttgtggtacacatgatgtgttggtcacttcccagctcatcttgttctgagtcaccctagatttgggacattcattcgccaccagtaccgggcggtgtatggcctgagatttgggggggcttgtgctgctacaaattggggctgaatttgagttgacagtggaccttctttatgtctactgctcatatttgaattgcaaatactgcctcttctctttcagaggctcattaccctatagctgtattattgcaaagtgcacaattacagcttgagtgtaagtcacactgcgctggcaggacggcccactgagaaagggcacgtttcctgttcgttagttttcacattgacacataatttacaatacagtaaaatgtacttttctatcaactgtagtcagtaacagcccccctcccccaaccacatcaagatatagaggagtgctgtcacttcaaacagttccctcttcctctgccacatcctgcccctccccaggtctaaccaccaatccgtgctctgtccctctgttcagcccattgcagaaggccatagaaatagaatctataggctaggtgtggtggctcatgcctgtaatcccagtattttgagaggctgaagtgggaggatgacttgaggctgggagttcaagactagcctgggctgcctagcaagaccccatctccagaaaaaaaaaatttaaaaattacaatcacgtccctgtagttcagctgcttgggaggctgaggcaggaggatcacttgagctcaggagttagaggttacagtgagctatgatcgtgccactgtgctccagcctaggtgacacagcaagacgttgtctctggggaaaaaagaaagaaacggaaccacgcggtgtgcagccttctgagtctggcccctttcggtgagcagtgtctaaagttctgtcgcgtgttgcccacgcgtcggtggctcgctccttgcaactgctgagcattgtatggctaggctgtagtttgttttcacttcaccagttgggaaacagagaaaaggcactttttaaaaagtttaaatctgtagaattttggtttttaccagttctcttctaaatcctgagggattacaggaaaagttgttgtatttcagaatattcttagcttgatgtgacctctgtccccgttaaggccctttgccgcaatgggaaggacgtcgctcggtcagaccctgaaggtcagaggggcagtttgggagtgtgtcaacattttaactgtatggactagagccaagagtctcaaggtttataattcccacgtattcaaaaagaaaaaaacaataaagtgagaagtcagtgtagagtgaaataacctgtgttagtggggaagaagtgtttttaaacaggatttccataacgtataacatcaacatgtttagagtggtgatgtttcattgggaaacgaacagtaaaacatgaaagcagggaggttttcattctggcagttggcaactttcacggcagatggagaatttcaaaagcaattgctcaattatcaaacatagccagtgtgagttctgaaataaaggtgctgattgaatgtgcagctttatggtggattttgctattcaggcaagcattttaattttctgcctgttaaattctgttttctttagtttttcatatgtggtttattgtagcttaggaatagataactgagagtatatattacacatacaacattctgatatggcaatatttaaaacaacttgtctgttttagaactagaattaaacataatcatcttcagtattttgcaaataagctcactgccatccagaaacattgtcaatgcatctgttgctccttctagaagacacagtctgtccagcacaaagttacttagtccccagatgtctggagaagaggaggattctgacttggcagccaaacttggaatgtgcaatagagaaatagtacgaagaggggctctcattctcttctgtgattatgtcgtaagtttgaaatgcctgtaaacggggttgagggaggtggggaccaggagaacatcctgtgtagatgacacttgcatggaccctctggaacccagaccgcccggtgtcctgccaagctccatcgaaactaaatctagaatgaatgtttacttctgctgtgacatataattggagaccaggcctggccttccagtcactggattctaagttggactgtgagagtttttgcagctgactcatttatcaaatgcccggctattggctcacgcctacatgatgctgggtatgtttgttaatttgagggaagcaatggaataataataactaatgatttaaaaaacaaagtaagtgcattgactgtagtggggttctgattttaaatttttttaaaaattaataccaggagcagtggcttatgcctaaattccagcaactcgagaggctgaggtaggaagatcacttgagcccaggagtttgagacaagcctgggctatggtgtgagacacccatctctaaaaaaataaaaaataaaaaattatccaagtgtggtggctcgtgcctgtaatcacagctctttgagaagctgagggcggaggatggcttgagcctgggagttcgagaccagcctggcaacacagagaaaccctgcctctaccaaaaaaagaaagagaggaagaaagaaaaattagcctggcgtggtggtgcatgcctgtggtcccagccacctgagagactgagaagggaggattgcttgagcccagaagtttgaggctgcagtgagctgtgactgtgtcactgcactccggcctgggtgacaaggcgagacccctgctctaaaataatttttttaagttaatttgtagaaaaggtgttagatgttctttgtcacattttatgatggattcctgtttaaatgccgttctctttaaagaaaaaaaaataacttgtgggagtttttaaccataaaactagcatcacatatttaccatggagaatttacaaaaaaacaaataaacggaggaaaataaaacctcctgtaatcatactactcagagataacttgctgttagattttggtctagatttaatactttttctatatttatattaaaaatatttaaaacatatgcatttctttgtcacaaacatggtatcttatagatactactgtcacatagcaaaacagtgttaaatattctgaatcagaaaaggaagccgactctccaactgaaagaggtgttatcctagagactttttctggtgatgacaatttattaatagtcactttttgctttactttctctattgaagtagtttttctattttgttctacttttaaggataatataatttataatgctgtttttcacagaaatataagaaaaaagatactaattttataagttaataaagtttgatcatcccaaatccaaaaatctgaaatccaaaatgctccaaattctgaagctttttgagtgctgacattatgttcaaaggaaatgttcattggaaggtttcagattttcggatttagggagctcaacaaataagtataatgcacatatttcaaaacctgaaaaaaatcctaaattcagaatacttctgatcccaaacatttcagataagggttattcaacctgtactgtcagatgatcccaaatgaaaaatattaatcgttaaccaaatatcaaggaattgatcacattttacagtttctgcctaggattatgaatcaagatgaaaaggctctgcatgtttaaaaatatatatttttattttcttataaatcttaaatatctacacttaagatttatttgatatgtgggatccattcatattttggattcaacagttctgtcaaaactgtggcagtgataggggattctttttttcccactgaactatcacaaaattggaaaaagagtaattggagaaccccactggcttagccggcccgaagcccgggagagggcaggcagtgctgtggatggggtcatcccagcgcaacgctgcccctgctacctgcggatctcgctgaggcctgcctttgtcctttgacccttggccatttgttagtgtctctgagagctggactgctgtaccctacttccccagggggcctaacttcacacagcctctgccgcagtgcgtggttggaggtgacggccttggtaaatcgagtttcctacctcctcaattatttgtgctcatacactgtatatttttagtgaggtttatatttgggatgtgttttctccttcttaccctttctggcctttctatggcattaatacctggtctcttcttgtgtacttgaaaatgaatctctcatcatatttttccttagtgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactgtacgtcttcatcctgccgactattgccagttgcagttttccctgccttaaaaatggagtattgaaatttttaactttaatttctgatttgcaaaatagtcatcttttgttcttttccttcttgctgttagccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacaggtattgggaagagaaaccctgatattgatttatattgaaaatttagcaggccaagcaaaacaggtggctggctttttcctccgtaagtatggtcttgacatggtcaccgatagaaacatggaaacatctgcaaacttgccgttactcgtgtgtccgatctgactgtttcttgtatttttttctagtctgcccttactaggatgaactgtacacatcagttcatcctttttaaatgagcatgaggttattttgggttgttaggtgttacaaacacactaatgtgtttttgtctattagagcagcatggcccagttgccaatggaagaactcaacagaatccaggaataccttcagagcagcgggctcgctcagaggtaatgctggaaacacaggtcgtccttgtgttaggacaacccaggatataaaggatatagatttgtacgggaataaattcacaggacaagaaatcgatgtgccttataggtgggtttactgcagaagtgccataatagaaccttcctacttttaaaacaaccagatctcactttctaaagagtaaaggatgaccggcaggatcacgtctgtgacgtgagtggaggcagtttgcactcctggtggctgtttgagaggtagcatttagaatgcctgtattcactgtcctgtgatgagtgggaaaataggttatcaggtttatcttagcaaaatcaaagcatgtcatctaattgctaaacaagagttggcaaatctgagagacattactcaatccttggcatgcaggacttacatctgcatcctgttgccattttatgtcttcaaagcatttaatcatttagttgtgtttgcaaagtctttgagaagcctttgtcagaaatccctacatctcctatgtgagtgtatttccatgactgcagaataagttaaacttttacctttttccttcccttgcggggcggggtggggggcagggattgtgtgtgtgagagggagagagagacagcagagaaggagaatataattatcatgctgtgtactttgagctgaaactgcaaaaaaggaaaaacacacaaaaattattatgcttttcagtctttagagtaccttgtctattatgcttttcagtctttagagtaccttgttgatggtgtttttaaatgggattgggcacaattaggtggacagtttgggatgatttttcagtctgtagggccaagctcttttgtaatttgcattatgaagttgtcactctcatagcagatggcgggagataaactattattactttttgaccctagacttagtcttcagtccagatgagggagattaaaagattataaatatcttgtgccagatgaggtgattttattttgaaatgaccatgaattcctatcagttgtcttactgggatatttgatagtggaatttgtgcatttgagtcttagatgatctgttttacatttattaagaaagcctttattagcttttatactgtgtattgcctgttgcagtgtttgagtataaatgaaatttctggaaaatattaatggagtacaaactgtgatacttaaaagtaaactagggcctgcatttgtatcatgacctgtttgagtattgatgagaagatagctgtgaagaaaaaggtttaaacaagtgtattttcctttaagaagccactaatagtgcatctccttagagtgtatatttctagaatcctagtgtgcagagtttagactaagactaaaaaaaaaaaaaaacaaattatactgtaatttcatttttatttgtattttagacaccaaaggctctattccctgctggacaggtttcgtctctccaccatgcaagactcacttagtccctctcctccagtctcttcccacccgctggacggggatgggcacgtgtcactggaaacagtgagtccggacaaagtaagtgtccagcgtgtctgcatgggaggcacagggcgctgagtgcctctgtcacctgtggcagatacagagagtgcagaggaggtgccgtggacccaaggagttctggcgctcggctcggctcagtgaagctgtggttagagacgtggggggccatcaaggtctgagggagccaagcagtgctgatgtgggacccttttggtaggagtgtggggtgagtagttagtgggtgaatcaaggaatagtcggccgtggcctgcaggcccctgactgcacaggccttcaagcacatgtcaatgccgttagcctccctccatctcctcataccttctggccacctgtgagttgcactgccactgccagccattctggtatgttgtcagcacctccactgctcatacctcatggttagggaccacctggagccttggtagagccttggtagagccttggtactctactttcctggacaaagttcagcttatgaatatgaatttagatttcaaaaaccagcagcccaagtataagaaagcgaaggttcagtcctgccttcttaggctctattcgctaagcacctgccctgccctggttgctggggagagatgagtaaagcagacaacccaggagaggatggcaaaggggccgctaacccttagtggtttagctatatttggaaggcctattggaagttcaccaggtgaagggggaggctgtgagggtgcccaggcaggtaacagaagtccaaaggggaaaacctgtggtgtggtgagccgtatagccacagcctgccggccggcagccctctcagcctagtgcggtgttcccaagcactggcctaggcctgtagctccagggatgtgaagtccccttgaacgccgcccatcatgttccccttatccatttttttcttcccaggactggtacgttcatcttgtcaaatcccagtgttggaccaggtcagattctgcactgctggaaggtgcagagctggtgaatcggattcctgctgaagatatgaatgccttcatgatgaactcggtacggggggagcagtggaggcaaggaatcctcagcttttcttgtgacttccaagtgggatttgtctcatcatcatgtgacccacttgttgacaacacatgttggggactccagtctgggcagggacgggatgtcggagagactccactctgaatggggccgggaagtggggaggactccatttcagatggggtcgggacatgggggttatgctgatcgagacagaaaagcacattgtttcagccacattagaatccacggaggtgttgttttgaaatccagctggccccaaggctgggtgtatggtttgggatgagaactatctggcctccactggaggaacaaacacaggatgttatcatctaagctccatggccaagacagaatggaagtcaaggttgcgtatttgccgtagacttcaacacagtgtcgtaatgcgtgacgtcaataacttgtttctagtgtcttggaagttgatctttagtcgtaaaagagacccttggatgcagcgagatttcctctactcacacctctgttagatgtagtgaggttcttcaccccccaaccccagatgtcagagggcaccctgcgcagagctaggaggccatgcaaagccttggtgtccctgtccctcacccgtgggcaggtcctgtgagcagtgggggggccacctcttgggtatggtgcagccatggcccaagcagggcttcttctcagacctactaggacgggagaaacctcctggtgctttagccctgcgttgatatgcagcaaatgggagggaagtgggcacctgggaggacaaatgcctgtagaggccgggagtgacggcaggtgttcatgaaaagagaccttgtggggagggcaacacaacagtgtgttctgatgtactgaagagctcaactgaaaacaacaggagaattagcccaaaatccatttactaaaattgtttatctttttttttttttttgagacaaagtctcgctgttgtcccccaggctggagtgcaatggcgctatcttggctcactgcaacctccgcctcctgggttcatacgattctcctgcctcagcctcccaaatagctggtattaacaggcatgcaccaccacgcccggctaatttttgtatttttagtagagacgggatttcaccatgttggccaggctggtctcaaactcctgacctcaggtgatccgcccacctcggcctcccaaagtgctgggattataggcctgagccaccacgcccggcctaaaattgtttatcttaagattcatgcagtgaaagctaacttactgagtgataaatttgcttagtgatctgtttattaggttttccaaatttgctaattgggctttgaacagctgtaaaagttctgactgtaaaagaaagcttcaacttttggcattcatgatgcttttctgagtattaaactaagatagatgttttacctgaaggatcggccaccaatctttaaatggctaaacaaaagggttgctaaaacataatccaaattgacataagaaataccatttttccaaccaaaattttggcattcatatggctacttttacgtatttcagctgcatttgaacatctttttcaaactttagggtggttggtgtatcactgaggtcttggatgacactttagctttgattttgtttttatgaattaaaattgtcataccaaaatttttatttcaagcaaatccaagagcataaaaaattaaaatattacttaaaatactaagagagaacagatatatattttactaagcatatgttgaatgaaattgttcaaatatttataacaggcatagagtagaattttcttaaaaatatttttgatggtataccaatttgtattttctcagaaacatttgccttattcttttttctgttgtgtttttcttacctgattgaaagctcataatctgttgttattgtttgttaacctttaatgctctgatttcaggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggtaattaaaattaaaatttatcttatttttaaaaagcattccagggccagtatagtactttgcaccaagtaaatgtacaataaaggcagtggatctaatacattgaaagcgtttacagaggtagctaaagagcagcacgggtgtcctcggctcagaatttcttcctgtgtgtttgccactttgccattcattgacatggtcatggacatagggctctaagcccttgaggaaggctgggccagacctcaggggagatgcagccccaaaccacgtgcagtcctgtggacggatgtgtagatgtgccactgaggaacaatgtcttgagctttcatcagattctcagagaattgcttgactgcctttcgaagttgatgcatctgtgctcacgtttgcacccacccacgaggtccttctgtttcaggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccggtgagtccccgtccatgaacggtgggttcctatcatagttcctgtctgcttcaccatgtttttattttgtgctgcctgtttgccaggtactaagctaggaattggggatggagaggtagataaaatatgcatcaggaagggctgggccccatctcttactctccaatatattggagtctacactggaatttaactggaatttgcttttttagtcattttatttagattttgaagtttcagctttcatcaaaaatacctctaaactttatgtctctgtgatctttggtcttagctgttttatgtatttagtcttatatgatcataagattaataacattacattcagaagattatttgttttctgtcagagttaaaatgtttgtttttatactgcattgtaatattaacgtactgtaaaataaaagtggcttgttcttttcaaggaacagtatcctcaacaagggtcattagccacaatttttaaaaaattggacgtcatagtttacatgttagagggcgttttgaagctttgtatttttaaattaaatgttatagagtgatgttttcatgtttcataattgttttcatctgtgcatttgtagccaacttgaaaacaaagatccagggattactacttaaaagccagacttcttggaggttatagtgatgattttgatagtatcttgagccgtctcataataacctcagggtgagagatggccaacaggagacagtcgagggacttagaaatctgaatgaaatctgaagttcaaatcttcagacatataccactaaccaagagattggtacctcagtctagtattgtctgtttgtctaaaattggttctaaggaatctaggctagtctgtctatccctttcaacttttgtgaggctgcacaaatgtaaaatgttgaataaaaagcactgatggaagtgtgtagaaattcttctctttgttctgttgtaattttagttgcagtgcagcctggagagcagcttcttagtccagaaagaaggacaaataccccaaaagccatcagcgaggaggaggaggaagtagatccaaacacacagagtaagtctcaggacccatttttttcttacatgttgttcctccaggacttaaaaatcattcacagagacgtgcaccgcggtgagtgtggactcctggaagcgcaccgtagctccgctgtgtcctgctgctcctccctagctgtcagggaggctgtagtccattgctttgccagctcttttgtttccgagtgaacaccttatccgtacacatgcggctgtctctgaccctacagaccagctgggatgccactgggggagcgctcccttccccccgcacttcccacactctgcagttattctgagatccttgagggcagggaacaggtttgtcttctttgtgttctcagaaattaatgctcggcctctggtcagcaagcaacaaccttttgttgagtgataatgaataaataaatgtttcccacatgagtattcagtaacctcagtgtcaggttcagccatctgttttggtggatatttaaaagaaaattccgcttttcctacagaaaaaaaaaaaaatccaaatcccagtgatttaagccagttatagacttagacatatactacggcttttcatgcactttcctcccaattctagagtaggtattttactaggaaaatggtggcagtgcctgttgggaggaagattctttggccaagtgtcttttgttcttgccagggcccctaggctgctggggtgcttcagcttctttagcccagtgtctggtggggaatggcccctgttgcctgtcccacagaggtgggggtgcctcacctggagcctgtccacacattttacacagcacgcttacctggagcatcaggcatcttttccatgctctgtggctcaggaaacacgccttttcaatcatgagtgcaccagtgcttttgggctttttctccccgcttttgtgcaatcctggttgtggatggagttttcctgtctttagtcttctgcatagtacttttctcttctggttcccggttcaaggttttgtaattagagaatgacccagaagcaatggcattttaatgcacagccaaggacttctctgaatttgtatctcaaacctctgtgggtccttcaggcttcagtttgtgatttcatgatttcttgttgctacctaaggaatatgaaaacacccacctccctactctgcatcttccagccgagtggcacctcaggctgtggatcctgtgcttctgtggtgaggataagaatagtgccaaccgtgtggattgaaatcaatcagttaatccctccatgtaaagcacctggaacggatgacagtcttgttatgaatactcaacaaatgctatcatgatttttagttagatttccattgctttaaaacagttgagacatcttggcggtttgagttagagcaacgggccctgaagtgggttctgtttgggtgaagatgattatgcttattccccatggccctctttaggcaagagtgggaagctttctttgtttttttaatcacctcgataggacgttacttcttaaaggtcatccaataaatattaataggccgggcgcggtggctcacgcctgtaatcccagcactttgggaggccgaggcgggcggatcacgaggtcaggagatcgagaccatcccagctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcaagactccgtctcaaaaaaaaaaaaaaatattaataaagccaactcgttagcgtggggcttaattgcttaagtccaatgagaagtccttctctatcctaggaagttgcccaaactgtagaatctcgtggcctgtgggtaatagccacgtaatacacactcactgcctcaacaaatcatattttagtaggtatgatattctagactcaagacaccattctgtggatcttcccaagggtgtgaagtgtccacagcgtctgccttgggagtttccatgcccaccagaaccatgccccaagcccctcaagcactctgacctaggaaagccagtgaagcaaggatgacaacatggccctttgatactagctgagggacagacacaggtcctgggagaccagagaaagacgaggggcagaggaggtgtcctaaaggaagtctgaggctgaggagccacaggatggcttccagctgtcacaggctgctgctggccttatcacagagagtgggccagagggctgggaaccaaggccagagctcaggttcaggaccattccagcaatcccagcagaaaatggggagaattgtatggtataggcggatatgaaggtagaatctgcaggccttcagtggccaactcagagtctaagtggattccacagttacagcttgagcagctggttgtaggtcatgctttctacactgggcatataggatgtgttttttaaaaagtcctctcttaaccgttgcttgtttagatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgagtctgctcgttccttgcagaagaccaagtacggtgaaaggcaccggtaggccctgggctgggcacacgtgagagggcgggacagaatccccgcagcccagaggctgcctgctgtggttctggtgcccactgtggttctggtgccaggctgctttcctcaggcaccacgtgtggaggtcgctagtagaaatactgggttttctaaaatgaactgaggccctacatccctaagagattagtgttagacctgattctagagcaactagaccactttgcttaatagcagaccagaaaccacaccccctcgagtgagtgagattttcctttggagataattcatgtttttctacacagttttgcagttgtcttcagaattggtttaaagtaggtgttattgccaggcgcagtagctcatgcctgtaatcccagcactttgggaagccaaggtgggcggatcacttgaggtcaggatttcgagaccagcctggccaacatggtgaaaccccatctctactaaaaatataaaaattagccaggtgtggtggtgtacgcctgtaatcccagctactcaggagactgagacaggagaatcgcttgaacccaggaggcgaaggttgcagtaagccgagatcgcgccactgcactctagcctgggcaacagagcaagactccgtctcaaaaaaaaaaaaggtaggtgttattgatcagaacccttgtttcagataacatgaggagcttagcttgaggagagtgagggttgatggagggggactgacttctgcccagtgaaatggcatcatctcccaccagcccgctgaaataagatgatggggcctgttccttagggcctgcagcatcctcaggcaggaaagaaaggccgacctggcagggtgtgagccagcaggtgtaggtcagggagaatggagccaggtcccagggaagaggcttgtggctgcctgagaagggtgcgtgcctgcctgtgtgtgtgtgtgcacgtgtgtgtatgtatgctggagagtctagggaggcttgctccaaggacgcagtattgtttgatcctgagagataaggattctgccgcagggaatgaaggtattccagatggcgggcttattccgaagaagaggccagtgcctggcggtgctggaagcagttgcagaacagggagttgtaggctttcctgggaagagagcagcaggggtgctggagaagcaggccacacttgctgcatggggttgctctcggccccactcttggtgcacagcgagtcactgtgggttcattagcatctggttatgagacagtaactgctcctttggaggggctcgtggagaccatgcaggagggcacggtcttgaggtcatgccgtccagagcacacctgaggataggccaggacgggctgcacgctgtaggtaaaattcctccagcaagctcttcactggcattgaggagttccctgagtgcggtcatctggaaggcagctgtaacaggcactgcagtctctccctgggtgggtaccagagaggagcataggggagcataaccgatttaaagagagggctttcctgtggtgaggtaagagattagctggtcattatcatagagccccctctgcctttgtgcagatgggctgtgggaatcctggggttccgttgggtcctttgtcacctcactgaaggcatgtaagctgagctggccagaccgtgagctgatcctgccacttgaacagcatcaagcctgcctctggattcttctgtgcatggcacttgtctgagcacctcacgcacagagaactggacttcagagtttacagaaataagctgtatggttcattttcatgcctgcttgccaataaacatatctgagctgaacctcattgaacgcctgcctttattctagcacagcacctgctgtttgtgggcgaggggtgctgtctctaactcctgcctgcttctcccagcactccctgagtggggtgtgccagcagcctcaggatgaggacaggaagtgggagggcagagcagatttgggagggccacttgatggggaaggaagtcccaggaagcagttggagctgttttctgggggagaaggtgccagctctgggacagtgttggggtagtgaggagggagcccagtggagagaagtcgggcttcctgcttcctcacagtatgtctgtcctgactcaactcggatgatgtcacttccttttcatcttctcaggtgtggaagcttggatggtcacccaaaccgggaggggattttggcacagcattccctgagatccccgtggagttcctccaggaaaaggaagtctttaaggagttcatctaccgcatcaacacactaggtactcttggggcctctccttcaggtcaccattgtcggacatctaccgggaggaaatccagagcccccagtactgggatcttctcatttgactccagaaaagatttaagcatgataataatacaaacctatgtgaatacattttgcagtgttggcaaaactccttttatactgagaaaatagatcccagttcctgtgttttgtggcttgaatcccagctttgtgtattccgggcttgtttgaagtcaggaaaggttcatgtgtagtggacaacgtgagaccaaattctgccttagattttgcatttaggctaaacagtggcagcacttgtctcagaatgttttcttgtgttcaccagtctgatcctgttgtgtctcagtggtccattttctcatatgggaacaagcagacgggagcagatggagtcaggtttcttggcactcgccttccccagagcctagaggcagcatggggagaaagcaggcttggggctcagacagtcctggtctgcttccagccctcctacctgagcagcgcagggcaagtccgtctaacctctagagaccctcagttttgtcatatgtaaaatgggggtcgtgtctatttcatagaattgttgcagatttagaaattacatttctaaacaaatgttaccccttatttctaaataagtgtctaaatgaataagtcaccacttttgcccctatttgatggcaagaggtgtgatcttgtggtgggactgtaatcagtcagttctcagtgactgtgccctgctgtggtgtttcctggaatgttcctgtcttgtcctagaaagtctggcaggggcaccctgactccactgtccagtcctctccccagtccctcgggcttctgcagatttgaggcttgtttggatcccagaaggttgtggcaggagacaccttgcctctactttcccctttataattcaatgtccaaagagagccctgagcaggtacctcacgccagctgcctcacggagctcctcctcttcctggctgtgaggatcggtatcagtggcctcctgctctctcccccttgcctaacacgagcacctttgcttacttgggtgcccttgctcttgaactgcccatcggacgtgcgtgacccaagactgtgccgcagtccttgccttgtctgtgctcattttctttgttcatttttttccctgtaacgtaaattgttatatttgtctgtatctgtgtctgaatcagtcctgcacgctctccttctctctgtctcttgttctttctttaccccgtttatcacggggaccccgatgtccattgctctagttctcctgtcctaagcaccccatcccgtctctctggccttaccacaagtggcgtggctgcctcagacatcatgatggggacatgaagcacagctgtcagaaacaactgttcgttagatacactcgaatgcagctcatcaatagggatggagggtctgtcggatgtattttcactgaatccccgttcctaccttgatacactctttttaatctattcttctagacaggtcagaggaaccattactttgacttttaaatttttagcagctttattgaggtagaattcacatactacagatttcacccactctaagcggacagcttggtggccattagttttatccacagagttgtgcagccagctgcacagtctcagggctggactccagggaagattttagcccatttagtgagtggggcagaagtggccctggccctgcacgaggttgcctgcatgggcgtccctgccctgtccctgtgtctgctccactgggggttgaccaggctgccagggccgacttgggcctgtgccacctgcctctcatgtgtctcggacagtgcagccgatgtctatacttcggtttcctcaatgatgaaatggaggggatagtgttccccgcatcatagaactgtgtgaggtttaagggactcactgcccttggcgtggagccttctccaggggccgtgctgtgtcggcgtagctgtcagctctccgttacaggcttgagaagggttgacactctctcatgtaacatttatatttctaggctggaccagtcgtactcagtttgaagaaacttgggccaccctccttggtgtcctggtgacgcagcccctcgtgatggagcaggaggagagcccaccagaagtaaggccacaccctgtgctggttggcacatgggcagttatggccgcttgcaggcctttggtggggaataaaataaggcagcaagctggtgttctttttttctcttaccttatttttgaaagagtagctgaatggtgtcttgactgatattccagagcagggacaaagcctgctgaggtctgggggctgcgattaccaatggctggaatgcattttattacggtgcattccatgttaaggatcaatacgattgtgccctttctggaaaatatcttttagtttatcaatattcagaggagtgtaggttgaattaaaatgaaaaggcactttataaaggccatgagtagtacctggtttcatttttctaatgtcttgcagagattttatcaggcttcttgaagtgttcacgtacattacgctaacacgatattaataataactgtgctctggtacagcggagccagcagaatgggaagttgtggaatgcaggcccttgattctgatagaaggtgtggtttgaactcacagaaatgacagtttggagggtagacatatgtcacaagtcatcaagattgtctttaaattcatgcatagaagctaacagggtgtcataagcaaggcctgtaaaatgtatgagggaattcaaagataatttattaaaaagtaattcatgtttggagttttgtgcccaaaggagtccttgatttgaaaaatgggcttttgcccatcagattgtttcagggcccgtgtgtgcggaggccctgccttgtgccccgtgagctcagcctgacagaaatcctttggtagcacttaaggctcctcttcctcccattgaggcagggaagactctgggttctgcaggcagaggtggttgtgggtgtcttgctgctcttgttgacatgtgggctctccttccaggaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgcttgagttcccacgtgtctctgggacatagcaggtgctggggacagtgggttccccgctgaagcgtccagcagcttcaaccaggccgttttccttcattgctagaattgaaaacaccgtccgtgtggcctgtgcaggagatgcagacccaaaggtggcctcctggtcagtgagaagctggaaacgtgacaggaactgacgtggggttattgagcatttaggggaagacgttagcagagcaggaatgagcaggcaactagtagaacacccacttaagggctcacggacaggtgctcacttaggaagtgagtttcatttggtattacaccaggttcctttaggcaaagcggagggaaagttctggtgtttttcacttgtaagattttgaaggaaacaaaacactctttaccttttttctaaaatgtaggtttgggaggaagctgagcattatcagagggattgtggagcaagagattcaagcaatggtttcaaagagagagaatattgccacccatcatttatatcaggcatgggatcctgtcccttctctgtctccggctactacaggtacctgagggaaagggtgcgggggagcggttgtacttgggctagaatgagagaagactggcatgctcaccacaccagtgatgcgggaagacctgagtgtggtctgagttggaggctgtggtgctaaatacgctgcccctttcataagcaggagtcttagtcaggcccagggaggaagtaaaatctggaaatgaatgagaagcattctctcctgccagtcaagaaatgagaagcgaaagaattctcacgggctgtaagaccagcaggatttaaaagttgaattagttgcttatgttaagaactcaaccaagttcatctacacaagctgaatctccagcttttcctaagaaaccatgtgtggcagtggctgcagggcagggcacagctgggcctgagcaccccgctccctgcacctctcccctccctgggccctgcctgtcactgcccactctcccaccaagccttccggttgtgtgcctgccctatcacaggcatcggagcttgtcacctggtttaaaagaagagagttgtgtggggatttgggatgcacgtttttcactcaaaagtattttagcgtagagctctgtgattccgtagctatttaggagtttaagcaccttgaaggctttaattgcagaaagttctatgtggacgtgcaatgtgttatacgcagtgtctatgagactcaaatgtttattagggcgttgaagtaaactgagcacttggagggccatggatccagccttcaaggagctcataagtcaggaggacccaggagcaatgacctgtcatagaaggcagaaaagaggggcacagaggtgggtgggaggcatacacaggcagctcctggagctccaaggggagcaagtgcttccagggaagggggcgtggaggcccctttggaggaggcaagttgatctggggtctggcagagggttagctggggacatttagcgggaggctggtgcccgggaattggggggatgcccagcagaaagacatgaggaggctggcctggggcgtgggggggtgtgaaaggttaagtgggggcattatcctgctcccgctcctgccggctgtatctggtcagcctgggcaccgaggtggggttctggaaggcactgttcaccaaaatgcttatctgggtcccccagagagcttgcctgcctggactgtcggctcgcctgcaactgctgactcctaagcttttgcagctcagcccacaaccagttcctattcacagaggtgggagctgaggggtgacaagtgactgctgcagtcttatttgtcatagagaaaaagtgacagagtccagcttgcccactggccctgccagcttaactggttataaagtgacaaatccccaagacccacagggctctgcacaacctgggccctcctgccagtggcggcgagggcaggtggctcacggctgggtgcctgtctgggcaggagctgggctggtatggggtgggcctgcggccctgcccccctgtgcagatcaagactcagggtgctggtgttcacaggtgccctcatcagccacgagaagctgctgctacagatcaaccccgagcgggagctggggagcatgagctacaaactcggccaggtcagtctcgcgcccccgccgcctggcctctgtccgtttctgtcctcagactttggcgcttgacacacccaggagaaaagctcagtgcactttttaaatgaaaggaagttttccttttttttaaaaaaaaatttaatgttcattgtttttatctgttttattcctaggtcccgcaagcagaggaagcattagttttgtttttatttatgttctgtattccagaaagtagttaagagacctcacatgtagcgatagagatgtgtgtaagagacagtgagagggcgtgacttggacttaagcaaggaccgtgagacacaaaaaggggggtgaggacagagtggagtcagctgaaatgctcaggaggaagtagacgccatgaagggccatggtatggggggccgcaggcgtggccgtgagtgtccctggggccagctcttggggggctccctgagtgtccctgtccctgtggccagttctgggtgggagccccgtgtgcaggcagacagctcggccacttcctagcaggtcacattggtctgtgcttctgtttcctcctcagataagtgaagggattcaagggtctgggtgtggtggctaacacctgtaatctataacattttaggaggctgaggcaggaggcttacctgagctcaggaggttgaggctgcagtgagccatgattgcaccactgcactccagcctgggcaacagaccagtactctgtcccttaaaaaaaaatgtaaacagaaacgtagggccatttgcatatgatggcacatggcgtggagccctacaggtgtatgctgggcggggcccggctgtgctggccgacttgcacctttccctccaccccggtgctgtgtctttcgctcaccgggttcctgatttagtgaaagcagttgtgcaggacagttctctttgtagcttttgtttctgtggaaatgggtcagaatatggtgtttagaaacacttatgagctctgagagtttcctcttctgagttcctggcctgcagccttcacagcagaaaccctgtgatgtcacaagcctgtttctgttccctgctctctgcctgtactgtcctgttttgtgcctgccggtttcagtgacaggaagcagggagctactggaccagcctgtatttttctagacatagttggaaaaagaagtcccactcttctgtcctttcacctttgacagatgtttccaccccaagataagtgaaaatgaccaataggatgcactgtatttttcatgaaagtgtttctgaagggcaggctgagagtgagaggcctggggctcactgggtgcctctggccttgtcctgggcccagggacactggtctgtgcccgaggtattccctatccccccaaccccgctgcatttggccacatccttcaatgtttgcgttgtgtccagcgtccgcaaaccaactgtcatgggatcatactggggctgaagtacggtcccacccctgccctgtctggggctgaagtacagtgccacccctgccctgtctggggctgaaggacagtgccacccctgccctgtctggggctgaagtacagtgccacccctgccctgtctggggctgaaggacagtgccaccccttccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctgggatgtttagcccctagatgccactggactgagccgctacttgcttttgggaaagaggggtgggggttaggggtctgggcgaggggagtgcaggggctcctccttggcctgagagctgttcatacagactcctcgcccactccctgcagggtgctgggtcccaggggggaaatggcccttggtgccaagaacgtgagttggggctagtgccagtgatgatggagaacagctttttatgggcacacagcccacagcactgtgccaagtgctcgaggcttcccgagaaccaggcagaaaggaggacagtcgaggtgtgctgactgcgtggtggctgcgtgatctagagcgcgggtcacaaaggcgcgagggagctctggccttgggtttaccgcaatgactgccagtgcgggagactggaaaaggaatctcacgtattggttccgtgttttggggactccattcagatgtcacttaggagtgaaagcatcccttcgtagagcctctttctgtgtcaccctcctcagctgctcctggggttgactggcccctgattcatgcctttagcatgtgctggagcttcccagcagctgtccagcccctgccccaccctctctgtgggctcccttgcccgtaacctggggtgtctgaacgacccttgctaaggggcagactgttagacggtaggcatgtgctgagtcccagtggccacacccacccaccaggagcctggcactgtggccgcagcactgagcagtgccccgtttctgtggcaggtgtccatacactccgtgtggctggggaacagcatcacacccctgagggaggaggaatgggacgaggaagaggaggaggaggccgacgcccctgcaccttcgtcaccacccacgtctccagtcaactccaggttttccaatggcctttttctttttaacagaaatttgaaatttcttatcagtcatttgatttgtttgaggtgcttcttgaaatgagcctctcatctcatgtacttggaaaatacccatctcgcatattccacaggaaacaccgggctggagttgacatccactcctgttcgcagtttttgcttgagttgtacagccgctggatcctgccgtccagctcagccaggaggaccccggccatcctgatcagtgaggtggtcagatccgtaagtgagccttcccattcccctcacacctgcacgtgccacacgcaccacacacgccacacaccccacacacacacaccgcccacacacatgccacttgcacacacacccctcatgcatgcaacacacacacaggccacacgcaccatagacaccacacacacatgccacatgcacacacatacacggcatgcaccatacacacaacacacacagcacacatgccacacacacacgccacaccacatgcaccacacacatgccacatgcacacacactccacatgcatgcaccacacacacacacacacaccacacacaccacatgcaccacaccacacaggttacatgcacacaacacacacatgccacgtgcacacaccccacacaccacatgtatgtgccacacacagcacacaaccacacacatgcaccacacacatgccacatgtgcatgcaccagacacatggcacacactacacacacgccacgtgcacacaccccacacacatgtacgcaccacacacatgccacacacacatgcaccacacacatgccacatgtacacacatgtatatacacaccccacaccacacacacaccacttgcacaccacgcacacacaccacatgcgcacacacacaccacatacgccacatgtacacaccatacacacaccatacatgcaccacgtgtaccacgcacccacacagacacagcacacgcatacaccacacacacacgcacacatgcgtcccgcacagtaatgtctcttgggtgtaagaacacgacttgccagtagtagcgttctggatgcgttgcctggattctaacagcgcgattctccccttgccctcctggttttccacatctccagcttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggtaagtgacaggtggcacagaggtttctgtgctgaagccacgggggcccatctgccttgggacctggtgttggccagaggtgccgggtgcggctgcctccttccaagagttgacccgaaccggactccacggcccacgtgagctgcagtgcttctcagatggagggggttcagcgacggtcagtgccattcacaggtcactgtgatgtgggttgtggcggccaagccatggtttggggtcccgtatccctgggcttatgacatcattgtagtagcccatccccacagaaccacggtgtgtggtggcgctgaggcatcgtagatggtggaaatgctactggcttccccatgctctgccctgaggcctgactgcctcactccccttctcagttatgttccaggccccccgagcttcctggctggacagcttctctcctgggggccgttttgtcacagtgaccctgtgtttctagtcccaaatctgggtgctatagtctctttttagcgtggtggttgtcttagtcttttttggctgctaccacaagttaccttagactgggtaatttataaacagtggaaatttacttctcaccgttctgggggctggaagttttcatggtcaaggtgccagcagatttggtgtgtgatgagggctgctctctgcttcatagatggcatcttctggctgggtcctcacggtggaaggagtgaacaagctccctcaggccttttagaagggccccaatccacaagggctctcccatcatgacctcatcacctcccaaggccccaccttcttgtactgtggcactgcaaattaggtgtcagtgtaggagtttcaggagggatagaaacattcagaccatcccagcggtcaagtgttcatcctcttgagttcctccttattctgcttctggtttatcaggattcagccagtgcagcatggtacctgtattctgtggcacatcaccacatggtatttgccaagtatccatcacctgcacacgtgaaatcattgcccgtgggtcccgacatctggcgaagcatattcaaggatggcagaactgtcagagctggcacctctggttccttgtcatgtggcattacctagtaatccattttatgatagcaatggaaactcatttcttcaacaaacacctgagtggctgccgtgtgccagccgtctggggcccttggtgagaatggcatggtggtgcccatcagggcctgcctagcccgtgctctggacgggctcctgtgtgtcaggaacgacaatgctgtcatgacggtgaatgatttttttttttgccatcactccagccgctaacatttgcggagctcttcctcccgcacccccacctgacaaggccaagggtgaccttggccccaccctaggcggccaaggtcagaggttagctggcttgtctgggtcacacaaaatgcagcagaggttgaggtgagcacatgtccgtgacctggagcctgactccctctctgcgagtcttgactgctcttgcctagactctgtcctccccgagcccaaacgccagtcatcttcccttgtgggtgtccttcagcctggtgccatgctggtgactcagcagccgtccagggagtggaaacaattgagtgtgtgggttccctgtgtgggcatctctcttcacggcgaacaccctctgggtgttgcccacacgatgtcaaagcggctcttggaaggggtccttctcctttgtgggaagtttcagctgctgggctaacttgaattgtaactgtggttttgtgctcaggcccagatccccctaggcaagtgttgtgccatcagtaatcaaatgagaaataatcattttgaaaagcagatcctaaggcaggatggtcatggacactcactcccagctctttgtgcactcatgctttctggaagatggccatcctctgtgaaggttttcagcgcgtcatgcttggtacccacgtatccagagcatgtcgttttgaggtatttgcccaccgttgtgaaatccgtgccacccgagagcaggtcctgatgtggggctttcagaagtgggacctggggccgtacgcagtccttagggaggggccgtgtggcgttgtgcgtgtgaggggatagcacagggtgaggtgggggcccaagaaggaagtgacccacaaagaacagcctcctcttttggtccttgttcctgggatggctgggagtggcttctgtgtcgtccggccatttcccctgcggagaggctcctaccactgccgagaacctcatcattccacaaaaacaagaggccgcctggccatccagcgctccatgggaattctgtgtccccatagtcttgggctgaaggagggtgacattccttgctgacttctgcaggggtctcctcactgttaaagagcagattgaaagtgaagaacgtgggctaagtgtttaggtcgatatttaaccctgctaggttttggatactaagtgaaattgaggccattttggttgaagttgacagaaaccactatcagggatccccaagactaccccaggcttttctagaaagactctcagctaagatgtgttatggtaaaagcacacaaaacaaaatcagcaaagaaaattagcaagggcagaggcccatggggcgatgtcccgaggacaccaggcttgagcttccagaatcctctcccagcggggtcgtgcaggacgcacttaactccccgcacagtgagccgtgacagcgcgtgtgcagtgtcgtcgccaggaaagcacactagagactcggtgccagggtttttactgggggctgggcacatgggcaccctctgcctgcctcgtgcccagactctggactcccggagggaaggcaagttctcagcaccaaccctggtgcccacacaagcagctgagcacagggagcccctcctcagtgaggatggtgggcaccgtcccaacaccagccaggggccagccttgcacacaggcctctcaggatggtctccggcctgctgtgtagtctcttctgcacacaagcgtgagggcagcgcccccgcctcggctgtggggaggagccactgggacgtgagctctggtggcatgcagcagcttttgtctgtgtgtgcctaggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccagtgagtgggagcctggctggggctggggcgggggtctcagaatgagctgtgaaggaagcagcatcaccctctccaagtgcccaggctcctggccagatggcaggccaggtatcagtgggaacccaggtgggtgccatggctgaggtcagtgagacgcaagagcacaggtgcgtcctagaggcttcctcgggcacctccagcgagctggagctctcgcctctgctgctgtctcatgtggcgcttagcacactctcccacgtgcccattcctgactctgctctcgaggccatcggctctcattctctgctcccagaaccctgttattacccaggctagcctcctctctgcaccttccccgccctggcccagtacctccctcttgtttccactgtgattccgacctcaccttatcttaaagctgctggacggcaggttctgtacacacgtgtccttgacaaagcacggctggtgccgcaacccctcagcgagcaagtcaagctcttcacagcgatgtcttacaagcgcagagggctctgtgacaccctggtctcaccgccactcttccaaagtcgcagaggctttagcagagatgggcccagcctctctgagtcataggcttctgcacacgggagctgtctttagagggagggtggaatttcatcagccacccacatgggggagttgagggcaagaattaggagcaaagatgggaaggggtctgggaggaatggccagtgatcccctttgacaagtgggcaggaaacgggggctaggtcaaagttgagtggaagacctggagggagacgggaaggtctctgtaggcacagttcagacaggagggaggtgtgagccagggcacatgccggtggccgtctggcaggatttgggacatgctggagcagggacagcggctcatcaggggccattgccctcatccaggccagagtgtcacaagcccgtggggaggcccttctcgcctgtcatccttgctgggcagtgggtgctgtgctagcaggacaggcggacggctggcaactgtctctgcatccctggagcctggcatagggccaagtcacacggggcacaggcctgcaaatcaggcacatatgttggtgcagtgacgtgattttggggggcagccccagaacaggccccagacacaggccaaagccctgcctgtgctggtgtgttgggctgttctatggctcttgctgtgggcatggaggactcagggaaggagagttgaggtggtccaggagttgcgtttgggatgcagagagcttgtggcatccaggtagaaatggtgcgtggggctgacctcagcaccatgggcagaggggccgtgtcacgtgcctccgaggtggaggtgggaccacgtggtgacagatatacgcatcactgggcacgtttttgtgggtgttggggggcatcgtattggctcctctgttcacagtggccactcattcagtccctggctaccaggtcctcactgtgccatggggaaggccggcgctgtcgggggatcacagaaggcagcacgtcatgatggcatgtgccatgaaggaaaagcacagggcactcaggaagtagaggggactggcctggggtgtgggaatctagggcctcgttgagggacagagagaggaagtgtgtggtggccagcatggaggtggccacaggggaggctgagttaggccgagagggcagggcgttggggaggtagacgggctcagccactcagggagtggtcaagcagaggctgaagggtcaggccaggttgcaggggcctgggggagccactcagggtaggcgctcccgggagcccgcctggcccatagctctacactcccgcgtggggccggacatgctgtgaagccctctccacgttggatgggggtggctgagcctggatgctgtctcccgttttcagctgcgtgaacattcacagccagcagcacgtactggtcatgtgtgccactgcgttttacctcattgagaactatcctctggacgtagggccggaattttcagcatcaataatacaggtgagtgggccctggctgtcttcctctgcacacggggagtgggcttcccttctcttttccttgcaggatcataccagtgggccagttttgacttggtcgggaggaggcatgaacacctgagactgtgcagcgattctttgacacagaggcctttctccctgtgcagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggtgagcatgtacacggtgcccataaggccagcccaagtcctgttcaagggaggcaggagcatgctcactcaagggacctcgactaggtgccctctgatttcacacttctggtgttgccccaagccggccccatcaccttgcaagaaaggctctggagcccccagggctggagtacctggtcagggttgaccgtccctgtggtcactcatcccatgtggctgagctgggctgggtcctgggcaagcaaggggctgatatcacctgctttcagatctccagggactcactggacccctgtgtacaaagcactgtctacagagcctattgggttgtatagaggtaaccttcgtactgaacacttttgttacaggaaaggagaaagtcagtccgggtagaacttcagaccctaatcctgcagcccccgacagcgagtcagtgattgttgctatggagcgggtatctgttctttttgataggtaagaagcgaagccccatccctcagccgttagcttccctagaactttggcctgaagctgtgcttttgtgtgtgtctgctgatcccctggcgctgttgctggagtcctgccagtgattccccaccacagcctgaccatgggctgccttggctcagggttccactggcgagctggtggtccttggaccccagcactcaggtgtagcgttgaccagttccaaggttgtcccagtgcctgcccatctctcctgagggctcagggacagtacctggcagttgggggtgtggcagggggcaggaatgaccagcctctgggagggtggggcagaagcctgtacagtgaggaggagctggctcagcctggctgcctatcgtgagaggggagcccacggggctgtgggaggggggccgtggtgcctgtgagcagggtgaggagcagcggcaggaggatgaaggtggaacccacacatgcatctttgagacccgtgtggtcagtggcttctgccccccaccaccccccactgctgtgcgtgcatagaattggcttccctcacctgctctggaagtgggttaggagcttggtagggctttttctcaaggacaagggcccctgatttgctctcaggcctcagtcctggcgacatggtggatctggagccttgttgcactgccttgcctgtgctctccaatcagggtggccagtggggagccatttggcttttctcaagagcatactcaggtggaccttgctccactgtttgaccagatgaggcattctgaacagccaagcctgtgctggtctgttttcatgttgatttttttttttcttttctttttgagatggagtttttcccttgtcacccaggctggagtgcaatggtgtgatctcggctcactgcaacctccgcctcccgggttcaagtgattctcctgcctcagcctccctagtagctgggattacaggcacacaccaccatgcccagctaatttttgtgtttttagtagagacggggtttcaccgtgttggctgggctggtctcgaactcctgaactcaagtgatccaccctccttggcctcccaaagtgctgggattgcaggcgtgagccactgcgcccggcccccatgtcgatttttaaatgcacctctgcatcgttcttcagtccccatatgctcactgagcaccactgcgactggcagacgggcacagggaggcgccacgaccagtcctggccttcaaggggcttgtggtctagtgggcccaatgctaggtggcgagtgctccaaagagtgtggtgcacgccttccgcttgaccgctctccagacgccacagggaggcacctcgcagctgaccacagatttctctctgtggagcagtgtcttcagagcggctgccatgccactgctgggcgagggtctgcgggcgggtagagccaggagcacctgtgaggaagtgcactgccattttcgtagctgcttcccgtgtgtctcagttacacacggctggcatgtgtgcactgatgagacgggaacgtgatggttgcttttcagcactgaaagggatactgctcagggggcgtgtttcaggatctggttagggaagaagcagcgagagcacagatggggccctgtgtggtaacaagaaaaaagtcctggttgacaacagtgccacgaagcgttagaacacatagggatgtttgtggagcatttgcatgtggaaagcagcaaaaacataatgggaacgggttcttttgttatgatttttaaaaatctcttttgtaacatccttcccgctgcgccgtttctgcatattcctttatgtagctttcaaactcctcttaggagttctggtccctacagggcgtgggagcccaggctttacgtagctttcaaactcctcttaggagttctggtccctacagggtgtgggagcccagggcctgtgccgagcagcctgcctccacgagctagacagaggaagggctggggttttgcctttttagtctcaaaattcgtactccagttgcttaggctctgactttccccacttggaaagtccctcacggccgagggtccctcccagccctgatttcacatcggcattttccccagtattagagccaaggccctccgcgggcaggtggggcagctgtgggagctggtgccagtctctgacctgcgtccctcctcccaggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgaggttgcatgtgggatggggatggagtgggaaagcctggaggtggagttgcctccgacttcccagcagattcgccagcagagcccagctcctccgctttaaagcagcaatgcctctggcccccaccccacccccgccacccaggcgcagcaggtgcttcccgtccccccagccctgacactcaggcacctgcttgctccttgcaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatgtatcctctctgggtccctggtgctggccccgtttcccttgtcaacaccgaggctcatgtttcatgataaggttttgaaacctaacctttgcaaaaaccccacagatgccagggtgacaggccctcagccccagggaagtaaaatgctgacaggggtacagaaaggagcacgtccagacatttgctgaccagggcctctcagaggggccggtgtatggcaggagggtcgcagctgaggggcctttctgtggagggcctgggtgaggggagcgagggtgggcggtggtctctgcagacgtcccgcccactcgcgggctctgtgtggctgggcttctcctgacactgcttctcattagctttggtcattgtgcctcgatcgccctctcggggaaaggcttaagtaaagatccagttcccacccccagatgctggctgccaggagtttccctttccacagcccttccccaagacagaccacaagagcctccaagcagcacagttgtcctggtgctgacagcacagccttgcccggcgtgcctggcacggctctgccctcactgcattggagcagggctagtggaggccagcggaagcaccggccaccagcgctgcacaggagccaggccaggtgagtgctgccgagtgggtgccctgcctgcagggcatccagccagccaagggttgcaggaatggaggtggaggcgctgatgcagctggaggcatccaggtggcccttccggggctctgctcgctctccaggctccctggacccctttgtagactgtttcaggagaggaactcccaggtgaggacagggaggcagcattcccctcatttgccggcctttttccttaactcctgcaccagcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcagttgttgctgtgtgaggcttgctttgcttcatgagaacctagaccttgctgagctggagtcttaggaagcagtctcctaagtgcttctccagcaggggcagaaactgtcccaccagctaacatctggcattatggagggtcccccaggcagctgccagcagggacaggccccgtgttttctgtagccagggatgaggaagtggccccagggcatgggcctggctgggtgcttctgcaagggccttcccaaaccacagtacaggtggtcttcctgccctgcagatgggagctgtgggagctgctggagctgctggagccttcatggtcaagtgacatcataagcttatatgacatacacaagcctcaggacttggcccatggcactgaagcaggtcatcaggcccagcacagagactagagctgtgttctcacagggcccaccacccttccacctccttggccattgacacctgcgtccctggcccagctgctcccaggtaacccccaaagcagctggcacatcccacctctggtgtggccggggctgctgtgtgtccgcagggcctgccccgtctattctagcttgtttgtcctgtctgaaccagcgcctactccaagaagcctctgctcagcccagcggggatgcttctaagctccggacgagcctctcggaagccttggtgattggtggtgtagtcatcttgggatgcagatgtcttaccaacctgcaagaacaaaaaccctgtggcttcctctggtgcagggtatttagtcaatgtttgctgaggtcccgtctggttctggctaattggcaggggtcgtccacccattctttccctgctctgctgtctgtgccaggagagacgggggccagtcggccaaggggccagctcctgctgcctgctcctcttgggcacgtgcgggggccccctttctctgagcagggatagggatcagtctgccggagggatgtggtggacaggcctaaagcatttggggcggggcatgccacttgagctccctaaatctgtctcctcataggtgacaccgctccagggccccccagtggcctctcctttcagagctacctaaattctggtcacttcagagaaatggagcacccccttctccctggtccaggtgtggacagcctggcacactgagcacacctggcatggctggtaatttcagaaagaagaggggccggggtccagtgggaagcagcggtgaacccctcgtgagtgggctttgcagtccctccccatgccacggcagagctgccctcaacacagccttcctcttcctcatcggagagcacaccctgtccccttgccgagctgtgccctgtgccttcggtggtatttgattttggctgctactggctttgttgggatctggaagtcgcttcccctgcgtggtgcgtggagcactgtaagtcagatgagggaagtagccagggtgaggtgagtaccgggtggagccgccactgaagggactgggtaggggggccttgcctctacatgatgtgacacagccaaccgaggacagaggaagccccgttcctgggggtgtggggtgcacccctcagggaagcctgcagtggggcctgaggaaaggcatcctccgcgagcccacgagtctggtccatgagcaccgtgacagtgtctgtgggtagaggtggacccggccttgtgtcatcaccaggacctcttttgggaaaccatgtggacatcgcttgcgggtcccccaggctctgcagccccagcagcctggctgccttttgggcaagtggcttgagccacagaggacccagtcctgttgcagccacatcctctgggggggcccgccagtgtggccggctttctccaccctacaccaggcctccaggtgtcctggtcgggggtgtctgggccctgggtgggccctgtggacctgtgaggtcagggtcagggcatcactggaggcagagggctgaagttgtgggtctgggttccccttgtgtgcacaggcccctgccctccatgcttggtcaggcagctacccccaaaactgctaggacaggctggtcctgaggtggatcctggcccctgtaccctctggacagcccacccgcccaaccttctaccctgccccagcggcggcagtgttggccacatccttcccctcctggccccaattgctctggggaagtccaggctccggagcctgcccaggggccccccgtgatttgggcccaggactccacgtggttctctgccttcacccaagccctgaactcctcagctgccaaatccccacccatctgcacaggctgtgctcaccactgctgctcctggaaggtgcccctcagtgggacgcccacctcctctctgggcttctgtgtttgggagccctgctgcccccacccttggtcagtccccatgtcctgctggcctgtcaggcagggcagaaaatccacccagaaatgctgagcaggatgagagtctagttgggcccagcctcattatttagaagggatggaggcctagggagcatgcttctagcctgagcccagcagggccccgcccatgtcccaggtctgcaccagggacagctcctgccgaggcctgacctgccccttctccctcaggtgctgctggttgaccagcctctggccctaggagaccccgtagcgactgagggtcccagcaggccatgcagctttgccaaggtacgagcccctccccagcaggggacagatgtggggaccctcccaggcaggagcagctgggtgcctggtgctgccatctgctgcctgcctggttcttgtcctcacattggaggtcagtgtgagggctctgcctcgggaaaggccatggagcttgccctgtccagggcctcccatgtgcactgagcctgggaagagagggttggagttgagccttttaccctgggaatgctgcctggaggatggtgcgggtgtggggtggcaccctgccaggcagggccctgcctccctgcgcccactggaactcgggcaggcaggggtgtaggtgcctcctctagagccgtccggtgggggcccccggcagtggtggtggtgtccactggccagcagctgccccttcagccaggacagtaggcctgacgctgtccccagcagctccaaggtggatttgtggaagggggtagagggcacgtagaggccccatgacctccccagggttctgggagggctgtgcccccttagccagcaccatgctgggtgatatagtcagatcctgttacccctgttgtggaggtgaggaaacaggttagtggggaggacatgactaaggtccatgctgagtcgctagagctgcacccagaaccactgctgggaccccatgcctttctgcttaccccttgtgccgggagatgccaagagatgctgggagccagccccacctctgcccttggagtcatggctacggaaagggcattcggaccggtccctgacctcaccggggagggccgaaccctgttcctgaggagccagggcttcctagaggaggtaggccttctagtcactccttcatctgcaggcactccacagagctctctgtgccagcccccagcacggagggctgaccttagtcgagtggagatgccccagtgccaggcagtagggatgatgtctcctgaggcccagatggaagggactggactagtctcatggggctgatggtggggccaggccttgaccagggacccagtgtagggggtgcagagacccctctgagttcctcacacatccctggggccctccccatacacttcctatcctgactgcgggcaagagggagccccagttcgccttccctatgctgggcacccacagtggggctgggcacccccgccatgcccctgccctgtccttcccctgagagcctcggtcccacctccaaggtgcctcagaggacagcaggggcagcgggcagaggccgagatgcctcctcattccaggctcagctgcccttcttggggcagcccacacctgagagtctcctgcagttggtcaggcctgaggagggcaggggggtgcctgctgtccctctgctgaccacagtggcatttagcctgggcaccgcgcccagcacagtccatgctgcacaggtgccgtgggctccacagagccctgcctgacatgcatgtgttacgtttcgggtgccgatgcccttgggcggcacttctccgggcagaacccccaggccaccgctccggttccggttccgctgcatctggggctctcggcaggctgtggtcctccggccagcctgggggcatctcagtccctcagccccacaggggcctgccccgcagcctgggcctcgagccccgtctccgcacgctgtgccgaatctggctgcccatcagctccctgcgtacccagactgtgccctgccatgcccgtggctcttcccaggagtgccctgtggcctccccctggcttgctgggctgattccctcctgtgtctcaaacagagctcacctttgccatcactgctgtcctcaccggccggtgccagaggcccgtgtctgtgtaccctgtgtctgcacctctgggcagggcctggctctgaccaacccgggcttccagtgtccacagacctaaggcccagggcgcctgggggctggagcaagagaagcaaaaggagccaagggtgggggtttggggttcttgtgagggcccagccccaggaccccaggaccaggacacccaggagccccagggcccagccccagttcagaaggcaggggccttctgagggagcttaagggtcccacagcccaggacccccaccagggccagtggccagcgttgggggactcagcctcctcgtcgctcgtcctctctgtttctcccaccttttgccccctttctccttgcctgttcccacccgaggccccctcttggcctgcgtgagccggggcggcactgaactgggggccgatccgcctgggcggcggtgagaggcagggccgggagccgggccgctgggtttgggcctggcccgctcgccgcaatattgatggcccgtcagtgcagccctgattcctgtgctttcagttaaaaggtttctgttgttgtagcttatgcagttgctctgttgctatggaaacgtgacatcaaaatgacgtttcccgtttaaaagcttttaactaaattcctgcctgtcagatgtaggccccattttgagcgtggagctgccttcgagcgagcgtgagcggcgcctcccgcccatggtgcgtggggccgggccggggccctcgctgagcgcgctctctcaccccacaggcgcctccggcatggcggcggccgaggggcccggctacctcgtgtctccccaggcggagaagcaccggcgggcccgcaactggacggacgccgagatgcgcggcctcatgctggtctgggaggagttcttcgacgagctcaagcagaccaagcgcaacgccaaggtgtacgagaagatggccagcaagctcttcgagatgaccggcgagcgcaggctgggcgaggagatcaagatcaagatcaccaacatgaccttccagtacaggtgggcgagcgggcagtgtgggccccaccaggacgggcgggcccgggcgtggcgggccgctcctgactttcttggagctctgagtcgggacgatgtgtgggtcgtggcctgcctgtcggtctcctctggccgggtatgggcagaaccccacggggtgagacggggcccacggaaaccgtgtgtgcagccttccattggggaagtggggaaactgaggcccagcaagggcaggaaaccagtctaagagctgaggggtagcaggggtggggctggtgctgggcagaggccaggatggctcccaggacgtatgggcggtctgggcactgtccctcggaggcagcaacactcatggtggtgcccactgacctcacaccctgctcccccatagggaggcggcggctgccagtgccctccccaccaccaagctcccaagctcagcaggggtttcaggggcctactgcgtcattggggaaattgagactgcaagtgagaaggaggctcagtgctctgcgacttggagcatccactgagcctctgccatgagccggtgagccccactggggctggccctagggtcacggtggggtatttccagaaatcaccaggtgaggtgcaggaccagccagcgcatgggtggggcttacggtgcgaagaagaaagaggtggaggcctgccctggcccaggactcccagcgtgggggctcccggcctggccccacctctgctcctgctacatggcaggtgggcccttcctgccctggcaacctgcagggaaggccggaggggaccacccagccagggagatgttggcgtctaggaggggacaggtgtggtcccacacacccagcatcttaaagtgcgtgggtccccagcccattaggacagggtcccgggtgggcaggggtcatggtggggtgaaggtctcaggcacaggcaaggtcacaggtgcggtgagggtcttgcagggtgtgaaggtcataggtgtgcggtgaaggtcacaggtgtggggtgatggttttgggtgtggggagggtcttgcacggagcgagggtggcagcaagagctggaagctgcagggggagaatggcagcagagagcacccggccctgtgggcggcctggacagggctgggcctggggctgccggagagcctgtcagcttccaggatgggagtggcctcactcagctgctccacctccgggtcaggcaggtgagcctggggcagagaggctgagagcacctgagccacttgtgggagaggccacccccactgcccccctcaggcgaggagccggcctccagcacagcagaagggaacccccagtccccagccctagtgggagtggggaagaggcccagcaaggccccggacagaccgccagcctgtgaggtctccgctttcagttgcgttgatttgattttttctgagccttgaaggaggggtccggggcctggccctgcccaaaggcccctaggcaggccccaaagccgggacctagggtgctgagcatgacggatgttgggtttgagcggctggcttgcgacgtgagggctgaggtgtgagcctgggtatcttcagaggttcggtggacacaggcagctgcccgcggccccactgttcccgtggcctcctagtcctgctcaggcacctggtgaggaagggacgcagagggcagtgggaggtggccacgactgttccagcaggctcccctctgactcaggaattcacgggcaccacctccctggctggctctggttggtgtctggccaggttattcattatttatgctgaaagcctcttcagagtcccaggggagggtttctgtctccattcctggaggctgagagatgagggtgcagcagagtgggggcctccactccagaccctgcagtctgggctggccaagggctgcaccggtgcactgcacgtcatggctgatgaagcacttccacaccgcagcccctcagagctgccacagtcagccttagttcaccgagggggaagctgaggcccagagcatgagagggacttgcccagggccacatagtccttagcagaggaagctgtggctgggtgactcgatctttgtcctttttctttatacccgcagtctccccatagcagaggcttttcttttttttttctttttcttttttttttttttacaagaactctttatatattaaggctgttgggctgaagaagcctgagagggtggctggttctgtggagcatggtttgttgaagtacagtttgggggcctcctacactgagaataggccttttctcgtttctccaaagagtgggctggctcaagtagggcagagagagaagcctggggcagaggttagggatgggcacccagcgcctgccctcacacgctctgtgctggtgtcttcacagccacgtgccaccctgggcagcatcccctgctcaccatctggctgtgcctgtttgctgggggcacctcattcagaatccagcttattgtttccaacggccaatggccacaccctggcaggtagcaagagtaggagagaggagacacccactccgagcacaggttgggtttggagcccggccttggggcactctgtcactcaaaggcagagtggggagtgggcactgggccttaggaggtactgggtccagtgaggcagagatgcccctgccccacccccaccttgtggcttcttccctggcctggccagagctgtctggccgccatggggccctgtgtctcctgccttgacctcccagagggcagccgaggcccaggggaggcctggggacttagcctctcagggcaggacctgtctgcaggagtaggtgggtgctgggggtcccagtggtaatgaggcatcaggcagtgtgggaaggggcccatccggcccaccccagggcctctgggcaggttgcaggttgtagcgctggatctaggctcctgcccagactgtaggttcaaccaagaatggcatgggagcccagcctgctgtttgctttattaaatctgccctgtagctgggggaggggcttactttgatcatcactatgtcattgatataaaaatagaggctcagagaggtgaatgaacctgcccaaagtcacacagcaaagtgtggagatgagatactgactcagggctgtggacactgaagcctgtgctctaacgccagtggctgtcgctccctgaggcattctctcccgaacaacacagttattatattacaaaatattatcactatatttatatatcttataataccttattattacaataaaaccttattactctacctttcaaaatgaattatttaaaaagcagtatttgctcattgcagagagtctagaaactatagaaaagcaagggaaaagcaataggaccagccccaaggtcccagcatgcacagataaccttagtaatactgggacgtgtgcttcctttttaacatctgagcccgtgtaggtcctgaagcccagcttctttctaagtccattgtcatcttgaccctggagcctggccgattttgctggggaggcccttgccagccgagagcggctcctgcctgtgccggcgtggcgcgcccctctgctgaggctgggcaggacaggggctgggccagctctgtttctcacccttggctcttgtgtctctcgtttcaggaaattaaaatgcatgacagatagcgagtccgccccgcccgactggccctattacctagccattgatgggattctggccaaggtccccgagtcctgtgatggcaaactgccggacagccagccgccggggccctccacgtcccagaccgaggcgtccctgtcgccgcccgctaagtccacccctctgtacttcccgtataaccagtgctcctacgaaggccgcttcgaggatgatcgctccgacagctcctccagcttactgtcccttaagttcaggtagtgtgtctgcttgtccttcccctgccctggggtatctcagcccccaccatttagagaaagggactgggagtggcaaggccggcggcggcggccacagtggttgcagaggccgtggctgcgggcagcgcctccagggacaggcggcctcagaccagggagggctttagtgtccacaggcagaccgagtttgtctcccagctccatcacttttgagctgcacggaaagttccttgacttctctggcctcagtctccctcctataaaatgggggtaaatcagtacctttctcagagggtggctgggagcatcacaggagagaagacgcagcatggggcccggcacacggagggagaccaagccccagaccccagaatgcgccccctggcctcccttagcccacacagaccccaccctcacaggctagctgccctctcagcactggggagggtgtcgggctgcacctcatcacgtgttgccgtgggcatgacccgtcccctctgccatccatcccacacctcagacccgtcccgtgctggccacgtgactgtgcctgcaagatgctcacagggcagccgggagccaggcagcatgcaggacagacacctgcggggtgggcctggggagcccagagaaggtgcttttgaggaggggacatttggggtgggctttcaaggtaaaatagaagttggccatttggaggcaagaacaggaagattgtggatttgagtcacagcttctcccctgccctggtcttcaagtctttctgacaggaggtgtcagaaaagtatctttagtagagaaggcgtctccgaggagggtccctctcatgccgggggccgctgcttgactcaggatttctcattgaagacctgagacaaaaacgcttttgctggcagctagaaggaaccagcaggaggcctgagatttgtggctgttgttcccgtggactgagcccagttctcagactcagctgcctggggccttgcacaggactggggcgtgggggctgccctccctgatcaggcccaaagcgcggatctcacgcccctgaggttggctgtaccctctcagctcagagcagagtgtgggccagggatgagcaggcactggagcagggccctggggtctgtgggttttggcagctccctgcccttcagggaggtctgctgagaccacgggtggcccctaccccagcagcagagctctcaggaggcgcccacagggctggactgcctttactcaccacctctaccagagctctgaggtcctggggagagagcccaggcctcttgtgggccccacaccctctaggtgcctgtccttctgcctctctaccaaggtgtgccggccccatttctaggccgccgggagataagggggctcacatctcaggcccttccttctgggacctcagtttccccatctgcctaaggccgggtggggctggtggtcttggcttccctacaggggtcctgagtactctgcactacccagcaccccccacccctgccttcatctctccctgggggtggtctctccacccctggcccccaactggggctgagcccccacctgcccagtttggtgggtgaagggtgctccctggcaggatatgcccctctgcagcccagaacatcccaccctttccagaccgaaggggtgtggattgtcctgggaccctggtcattggggtcatccgctagtcgcaaaggacggcaatgcctgtggcctctctttctttctttttctttttttttttttttgagacggagtctcgctcttgtgcagagagcagtggcgcgatcttggctcactgcaacctccgcctcgtgggttcaagcgattctcctgcctcagcctcccgagtagctgggattacaggcacccgccacaacgcctggctaatttttgtatttttagtagagatggggtttcaccatgttggccaggctggtcttgaactcctgacctcaggtgatccacctgcctctgcctcccaaagtgctgggattacaggcataagcctccacacccggccacccctgttactttctgtcaaaggcggtgggttctggcccctcctttgcacatggaatatgagaccctgagtaagtgacctgactccctggggcctcagtttccccatttgcccagtaggattgtcgggagggtccggtgaggcccctggtgtgcccaggctctgtggccagcacgtccacagccggcactgtccttccaggtcggaggagcggccggtgaagaagcgcaaggtgcagagctgccacctgcagaagaagcagctgcggctgctggaggccatggtggaggagcagcgccggctgagccgcgccgtggaggagacctgccgcgaggtgcgccgcgtgctggaccagcagcacatcctgcaggtgcagagcctgcagctgcaggagcgcatgatgagtctgctggagaggatcatcaccaagtccagcgtctaggccagcaggcggcggcggcggcggggccgggcggctggtggtactgctcaggccacccagggcaggccactcaggccaggcgggcaagggggccgccccgcgagcggagaccgccttccacctggcctctggcaggatgtcccttctgaggggtattttgaggaacccccaggccctggggaccgtgaggctccagtctccagcatgaatgcccttcctcggacacaggccagggcctctggggttcactccgagtaagaacgtcctagagccactctccagtgtcgttactatcaatgatacttgacgtggctttgatattaaacgtatactttttcattcttgcctggaacgcacagtttgctgttgctggcttggtgaggatgccctgattgatggatcccgaaaatgaaagcagatggaaacgggttggggcaggctggagctgggggagctctctcctgaagggaaccctgtgtcctccctcaccaggacctctgcgtctctccttaaatggcctctgacgcctgatgaaaaccccagcgaccttccaggaggcttttattcagctctgtttggagcatcaggtgtttccactgcctccttagcaatgacactaataaaagtcgtaacacctgttcacatgcacagccctgttgagtgttctgggtgctggagatatcatggtggatgacacaaaggccctggcctcttggagcttatgctcccatgcggggaagacacatgggtcagtagagaaatggttgcaggttgtgataagtgctggaagggaggggttggcctgaggacacggaggcagacatacgtggagctgggaacagtggccacacagggaacggccagtgcgaaggcccagaggcagaggacactggagcaagcccaggagcagctaggaggctggtggccagcagccaggccacggaagcccgtgcagcccgtggggaggagtgttcatgcttttcaagcttagtgggagtcttttggccagtgcagctctgggtctgacatcggtgggggacagaggggtggtggagcggccacagctgcaagctcacctcactgccggcccttccaccagtttcaaactctttctagaagctccagctttcccaaagctgaattctctatgagcctccttggccgggactcgggcgtctggttgccctggctgcaaaggaggctggggccaggtgtgtttgagtcacctcctggaattaggcaagttgctgcccaaatagaaggttgttggcaggtgggtcagcaggtgaacagcatggtttgactcagggttcagaaaaatctccctctggctgccaagcgagcaggccgtggagacaggtgcagaggcaggtgtggcagcaggcatcctgccaggcagtgctgcagtcatcctgcgacaagcagcagcagctcatcctaccctctagggggtcttgaggtcagccaggcaagagagcagcttggactccactgggtgtgggaccagcctgtggaccatggtggtgtggagggtgccctcggcctgcctgtgtgaaggagaggccggcgtgttctgtggagcccaaaggggagctgggcaagcaggattcacttcactctgagggtcctggagctcccaccctcctcagccatctccccagagcctgtgtgccgaggactcggcccatgttgctgtgggatgagaggcagagtgtcgtgagggtgtaaggagcggcggcagtggtgggaggagggagcagcagccagcgctacggtgccagtttccagctgccagatgacgccgctgaccctgtggttgagaagagatgcacagagccagctcttgcaagccagtgtggctgccatagcacctgccgagaagcagaaggaagggtggccccaggaggacagaggatgcgggcacatctgatgcgggcctgagttttgggagcttttgctctagccagtttccagctccgggacccacccgcctcgtaggcaagacaccacccaagaaatcatttgcttaacaaacacactgggctccaactggacacctgtgccaccctagatgctgggaacccagccatgacacaggcacctgcccccagctgctgaccactgaggctggctagcagctcccatggggccagtgtggggttccccagcctcctaacagggagccagtcacaagccctcgagagggaagggtgcccgcggccctggcaggaaggttaggctggacgctcccacaagacataacagatggaggttctaaatgatgtagcaacttcttcaccctgaaactgctgtagagtcagccatgacgcaccggtacttcagtaactgccaggcatccgggacagcacaccgcgagtcgctgctgtgcttgggttagaagtggtttggtctgttttcttctcgccctctctaatcagagtcagtgattcatgcccttccatcaccttagagaaggggcaggcgctgcccgaccttctccaggctggagcagcatcgcctcatgtcagcagaactcagctgtagaatatcgtggggttggtgcctttcatcagcagcatgtccttaacaactttctgatttcttccttagttgttggtccattaaggagaaaaaaaatgatctcagccattgctaaaatatttgataagattcagcaaagcagcatgttaacattgaaaactagaatcaggagccaggcagatgtgcttgcttttcacctgtagtatttcatgttgttttgacgtttttagctaatgcattaagataaataaacaaaagccgggcacggtggttcacgcctgtaatcccagcactttgggaggctgaggcgggaggatcctctgaggtcaggagttcaagaccagcctgaccaacatggagaaacctcgtcattactaaaaatacaaaattagctgggcgtggtggtgcatgcctgtaatcccagctacttgggaggctgaggcaggagaatcgcttgaacccgggaggcggaggttgcagtgagctgagattgcaccactgcactccagcctgggtgacagtgaaactcggtctcaaaaaaaaaaaaaaattaaaaaaagataaataaaataagcaggataagaaatgaagaaagtagagttacctttgttttcagatttcatttttgtatacccagaaagccaaatgtacaaaagactgggagctctttaaaccagcttaaacttgttgaaaatgaggatgaagaaatatcccattcagagttggaatgaatttaacccagaaggaacaggacctctactgaagagaactatgcagtcttactgaaaaatctaaataatacctgagcgctggagaaacttcgcacactcctgaaagctccaaagtcaatgtcatcattttattaatgtcattccaaacatagtctcaataatatcacttcttggttttgacatggacgcgatgatgtttaaattcatatgaaaaaagaacggggccaaaagtccaaggccagtcagcgtgagaagaccgctcggcctccctcggagtcggggagttggaaccgcagactgagatcatgtggctgctggaggccaggacgaacgtcgggaaatggagactcctgcgttgctggtgggatgtggtgcagccgcttccaggagcaatttggtgtcccgtcctaaagctgaagaaacgcatttcctctggtcagtgccactcctagacaggccaccctgcggcagccgtcctcaaactggtctgaggacccctcaacgctcttaaaaatcattaaaagtgggccaggtgcggtggctcacacctgtaatcccagcactttgggaggccaagacaggcggatcacgaggtcaggacattgagatcatcctggctaacacggtgaaaccccgtctctactaaaaatacaaaaaattagccgggcgtggtggcgggcgcctgtagtcccagctacttgggaggctgagccaggagaatggcgtgaacccaggaggtggagcttgcagtgagctgagatcactccactgcactccagcctgggcagcagagcgagactctgtctcaaaaaaaaataataaataaataaataaaaataaaataaaataaaattcattaaaagtgccaaagaacttttgcttatgtgagttctaatgaccaatattaatacacattagaatatcttattagaaattaaacctgagacctttagaaaacatgtattcatttcaaaatagcaataaacccatgacatattaacataaataacaattgtatgaaaaatatattttccaaaacaaaaagttttcgggagaagtgtggcatagttttacatggtcgtaaatctctggcttaagagaagcccactggcctctcagcaggctctgggtccgtccactttgggggtgttttggttgtgaagtataggagtgaatggagaagctcattcttacccagatgtgtatttgaaaagaaaaggaacattttaataacctttgcaaataatcggtatattcttccgtgatcctattccaacactggacaggtggtggtttgttttttttttttggagacggagtcccgctctgtcactcaggctggagtgcagtggcgcgatttcagctcactgcaagctccgcctcc

SEQ ID NO: 2 - DNA, 19 bp, synthetic construct
  source 1..19: mol_type = other DNA
gaaggtgaaggtcggagtc

SEQ ID NO: 3 - DNA, 20 bp, synthetic construct
  source 1..20: mol_type = other DNA
gaagatggtgatgggatttc

SEQ ID NO: 4 - DNA, 20 bp, synthetic construct
  source 1..20: mol_type = other DNA
caagcttcccgttctcagcc

SEQ ID NO: 5 - DNA, 20 bp, synthetic construct
  source 1..20: mol_type = other DNA
cactgccccctgttgtcata

SEQ ID NO: 6 - DNA, 19 bp, synthetic construct
  source 1..19: mol_type = other DNA
gagcaaaaccagccatcga

SEQ ID NO: 7 - DNA, 20 bp, synthetic construct
  source 1..20: mol_type = other DNA
tccgttttttgatggcagca

SEQ ID NO: 8 - DNA, 20 bp, synthetic construct
  source 1..20: mol_type = other DNA
ctccgtccggtagacatgct

SEQ ID NO: 9 - DNA, 23 bp, synthetic construct
  source 1..23: mol_type = other DNA
ggaaatcagaaccctcaaaatgg

SEQ ID NO: 10 - DNA, 29 bp, synthetic construct
  source 1..29: mol_type = other DNA
tgagcactgttcaactgtggatatcggga

SEQ ID NO: 11 - DNA, 15 bp, synthetic construct
  source 1..15: mol_type = other DNA
tcgccgcttgctgca

SEQ ID NO: 12 - DNA, 17 bp, synthetic construct
  source 1..17: mol_type = other DNA
atcggccgtgatgtcga

SEQ ID NO: 13 - DNA, 23 bp, synthetic construct
  source 1..23: mol_type = other DNA
ccatggtcaaccccaccgtgttc

SEQ ID NO: 14 - DNA, 17 bp, synthetic construct
  source 1..17: mol_type = other DNA
attgtcatcaccagaaa

SEQ ID NO: 15 - DNA, 15 bp, synthetic construct
  source 1..15: mol_type = other DNA
ttgtcatcaccagaa

SEQ ID NO: 16 - DNA, 20 bp, synthetic construct
  source 1..20: mol_type = other DNA
ctcagtaacattgacaccac

SEQ ID NO: 17 - DNA, 20 bp, synthetic construct
  source 1..20: mol_type = other DNA
tctctattgcacattccaag

SEQ ID NO: 18 - DNA, 17 bp, synthetic construct
  source 1..17: mol_type = other DNA
ttgtcatcaccagaaaa

SEQ ID NO: 19 - DNA, 17 bp, synthetic construct
  source 1..17: mol_type = other DNA
  modified_base 2: mod_base = OTHER; note = uracil
ttgtcatcaccagaaaa

SEQ ID NO: 20 - DNA, 17 bp, synthetic construct
  source 1..17: mol_type = other DNA
  modified_base 3: mod_base = OTHER; note = uracil
attgtcatcaccagaaa

SEQ ID NO: 21 - DNA, 16 bp, synthetic construct
  source 1..16: mol_type = other DNA
attgtcatcaccagaa

SEQ ID NO: 22 - DNA, 15 bp, synthetic construct
  source 1..15: mol_type = other DNA
  modified_base 2: mod_base = OTHER; note = uracil
ttgtcatcaccagaa

SEQ ID NO: 23 - DNA, 16 bp, synthetic construct
  source 1..16: mol_type = other DNA
  modified_base 3: mod_base = OTHER; note = uracil
attgtcatcaccagaa

SEQ ID NO: 24 - DNA, 16 bp, synthetic construct
  source 1..16: mol_type = other DNA
ataaattgtcatcacc

SEQ ID NO: 25 - DNA, 17 bp, synthetic construct
  source 1..17: mol_type = other DNA
  modified_base 1: mod_base = OTHER; note = 2'-O-methoxyethyl adenosine 3'-phosphorothioate
  modified_base 2: mod_base = OTHER; note = constrained ethyl thymidine 3'-phosphorothioate
  modified_base 3: mod_base = OTHER; note = 2'-deoxythymidine 3'-mesyl phosphoramidate
  modified_base 4: mod_base = OTHER; note = 2'-deoxyguanosine 3'-mesyl phosphoramidate
  modified_base 5: mod_base = OTHER; note = 2'-deoxythymidine 3'-phosphorothioate
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11..12: mod_base = OTHER; note = 2'-O-methoxyethyl 5-methylcytidine 3'-phosphorothioate
  modified_base 13: mod_base = OTHER; note = 2'-O-methoxyethyl adenosine 3'-phosphorothioate
  modified_base 14: mod_base = OTHER; note = constrained ethyl guanosine 3'-phosphodiester
  modified_base 15: mod_base = OTHER; note = 2'-O-methoxyethyl adenosine 3'-phosphorothioate
  modified_base 16: mod_base = OTHER; note = constrained ethyl adenosine 3'-phosphorothioate
  modified_base 17: mod_base = OTHER; note = 2'-O-methoxyethyl adenosine
attgtcatcaccagaaa

SEQ ID NO: 26 - DNA, 17 bp, synthetic construct
  source 1..17: mol_type = other DNA
  modified_base 1: mod_base = OTHER; note = 2'-O-methoxyethyl adenosine 3'-phosphorothioate
  modified_base 2: mod_base = OTHER; note = constrained ethyl thymidine 3'-phosphorothioate
  modified_base 3: mod_base = OTHER; note = 2'-deoxythymidine 3'-phosphorothioate
  modified_base 4: mod_base = OTHER; note = 2'-deoxyguanosine 3'-phosphorothioate
  modified_base 5: mod_base = OTHER; note = 2'-deoxythymidine 3'-phosphorothioate
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11: mod_base = OTHER; note = 2'-O-methoxyethyl 5-methylcytidine 3'-phosphorothioate
  modified_base 12: mod_base = OTHER; note = 2'-O-methoxyethyl 5-methylcytidine 3'-mesyl phosphoramidate
  modified_base 13: mod_base = OTHER; note = 2'-O-methoxyethyl adenosine 3'-mesyl phosphoramidate
  modified_base 14: mod_base = OTHER; note = constrained ethyl guanosine 3'-mesyl phosphoramidate
  modified_base 15: mod_base = OTHER; note = 2'-O-methoxyethyl adenosine 3'-phosphorothioate
  modified_base 16: mod_base = OTHER; note = constrained ethyl adenosine 3'-phosphorothioate
  modified_base 17: mod_base = OTHER; note = 2'-O-methoxyethyl adenosine
attgtcatcaccagaaa

SEQ ID NO: 27 - DNA, 17 bp, synthetic construct
  source 1..17: mol_type = other DNA
  modified_base 2: mod_base = OTHER; note = constrained ethyl thymidine 3'-phosphorothioate
  modified_base 3: mod_base = OTHER; note = 2'-deoxythymidine 3'-mesyl phosphoramidate
  modified_base 4: mod_base = OTHER; note = 2'-deoxyguanosine 3'-mesyl phosphoramidate
  modified_base 5: mod_base = OTHER; note = 2'-deoxythymidine 3'-phosphorothioate
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11..12: mod_base = OTHER; note = 2'-O-methoxyethyl 5-methylcytidine 3'-phosphorothioate
  modified_base 13: mod_base = OTHER; note = 2'-O-methoxyethyl adenosine 3'-phosphorothioate
  modified_base 14: mod_base = OTHER; note = constrained ethyl guanosine 3'-phosphodiester
  modified_base 15: mod_base = OTHER; note = 2'-O-methoxyethyl adenosine 3'-phosphorothioate
  modified_base 16: mod_base = OTHER; note = constrained ethyl adenosine 3'-phosphorothioate
  modified_base 1: mod_base = OTHER; note = 2'-O-methoxyethyl-3'-phosphorothioate modified nucleotide wherein the nucleobase is adenine, modified adenine, hypoxanthine, or abasic; or the nucleotide is absent; or is a terminal group
  modified_base 17: mod_base = OTHER; note = 2'-O-methoxyethyl modified nucleoside wherein the nucleobase is adenine, modified adenine, hypoxanthine, or abasic; or the nucleoside is absent; or is a terminal group
nttgtcatcaccagaan

SEQ ID NO: 28 - DNA, 17 bp, synthetic construct
  source 1..17: mol_type = other DNA
  modified_base 2: mod_base = OTHER; note = constrained ethyl thymidine 3'-phosphorothioate
  modified_base 3: mod_base = OTHER; note = 2'-deoxythymidine 3'-phosphorothioate
  modified_base 4: mod_base = OTHER; note = 2'-deoxyguanosine 3'-phosphorothioate
  modified_base 5: mod_base = OTHER; note = 2'-deoxythymidine 3'-phosphorothioate
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11: mod_base = OTHER; note = 2'-O-methoxyethyl 5-methylcytidine 3'-phosphorothioate
  modified_base 12: mod_base = OTHER; note = 2'-O-methoxyethyl 5-methylcytidine 3'-mesyl phosphoramidate
  modified_base 13: mod_base = OTHER; note = 2'-O-methoxyethyl adenosine 3'-mesyl phosphoramidate
  modified_base 14: mod_base = OTHER; note = constrained ethyl guanosine 3'-mesyl phosphoramidate
  modified_base 15: mod_base = OTHER; note = 2'-O-methoxyethyl adenosine 3'-phosphorothioate
  modified_base 16: mod_base = OTHER; note = constrained ethyl adenosine 3'-phosphorothioate
  modified_base 1: mod_base = OTHER; note = 2'-O-methoxyethyl-3'-phosphorothioate modified nucleotide wherein the nucleobase is adenine, modified adenine, hypoxanthine, or abasic; or the nucleotide is absent; or is a terminal group
  modified_base 17: mod_base = OTHER; note = 2'-O-methoxyethyl modified nucleoside wherein the nucleobase is adenine, modified adenine, hypoxanthine, or abasic; or the nucleoside is absent; or is a terminal group
nttgtcatcaccagaan

(19) United States
(12) Patent Application Publication
(10) Pub. No.: US 2024/0325425 A1
(43) Pub. Date: Oct. 3, 2024

(54) ALLELE-SELECTIVE COMPOUNDS AND METHODS FOR MODULATING HUNTINGTIN EXPRESSION

(71) Applicant: Ionis Pharmaceuticals, Inc., Carlsbad, CA (US)

(72) Inventors: Michael Oestergaard, Carlsbad, CA (US); Punit P. Seth, Carlsbad, CA (US)

(73) Assignee: Ionis Pharmaceuticals, Inc., Carlsbad, CA (US)

(21) Appl. No.: 18/443,786

(22) Filed: Feb. 16, 2024

Related U.S. Application Data
(60) Provisional application No. 63/587,714, filed on Oct. 3, 2023.
(60) Provisional application No. 63/485,850, filed on Feb. 17, 2023.

(51) Int. Cl. A61K 31/7125; A61K 47/02; A61K 47/46; C12N 15/11

(52) U.S. Cl. CPC A61K 31/7125; A61K 47/02; A61K 47/46; C12N 15/11; C12N 2310/315; C12N 2310/3341; C12N 2310/3525

(57) ABSTRACT

Provided herein are compounds, pharmaceutical compositions, and methods of use for selectively reducing the amount or activity of HTT RNA comprising SNP rs7685686 in a cell or subject, and in certain instances reducing the amount of mutant HTT protein in a cell or subject. Such compounds, pharmaceutical compositions, and methods of use are useful to ameliorate at least one symptom or hallmark of Huntington's disease.

### SEQUENCE LISTING

The present application is being filed along with a Sequence Listing in electronic format. The Sequence Listing is provided as a file entitled BIOL0471SEQ.xml, created on Jan. 31, 2024, which is 262 KB in size. The information in the electronic format of the sequence listing is incorporated herein by reference in its entirety.

### FIELD

Provided are compounds, pharmaceutical compositions, and methods for selectively reducing the amount or activity of a huntingtin (HTT) RNA comprising SNP rs7685686 in a cell or subject, and in certain instances reducing the amount of a mutant huntingtin (mHTT) protein encoded by HTT comprising SNP rs7685686 in a cell or subject. Such compounds and pharmaceutical compositions are selective over wild-type HTT or non-target nucleic acids such as bone morphogenetic protein receptor 1 (BMPR1). Such compounds, pharmaceutical compositions, and methods are also useful to ameliorate at least one symptom or hallmark of Huntington's disease (HD). Such symptoms or hallmarks of Huntington's disease include, but are not limited to, brain atrophy, muscle atrophy, nerve degeneration, uncontrolled movement, seizure, tremor, anxiety, memory lapse, and depression.

### BACKGROUND

Huntington's disease (HD) is an autosomal dominant disorder caused by the expansion of a cytosine-adenine-guanine (CAG) trinucleotide repeat region in HTT (also known as interesting transcript 15 or IT15), the gene that encodes huntingtin protein (HTT protein). The resulting expanded CAG repeat region encodes an abnormally long polyglutamine (PolyQ) tract in the HTT protein, resulting in the expression of a mutant HTT (mHTT) protein. As a result of excessive polyglutamine length, mHTT protein forms aggregates in the cytoplasm and nucleus of CNS neurons (Davies et al., Cell 1997, 90:537-548). Due to its genomic instability, the expanded CAG repeat region can further expand with age and during meiotic transmission to include additional CAG repeats. Individuals with 27 to 35 CAG repeats typically do not develop HD, but their children are at risk of developing HD. Individuals with 35 to 60 CAG repeats typically experience adult-onset HD. Individuals with greater than 60 CAG repeats generally develop juvenile HD, experiencing symptoms of HD before the age of 20 years. Individuals with a normal number of CAG repeats (<27) are not considered to be at risk of developing HD.

In addition to the presence of an expanded CAG repeat region, the HTT gene may further comprise one or more disease-linked single nucleotide polymorphisms (SNPs). An exemplary disease-linked SNP is rs7685686, which has an A nucleotide instead of a G nucleotide at this position. Additional exemplary disease-linked SNPs include, but are not limited to, rs362271, rs362272, rs362273, rs362307, rs362331, rs363099, rs2798296, rs1263309, rs762855, rs6446723, rs2298969, rs7691627, rs6844859, rs4690073, rs2024115, rs16843804, rs363064, rs363088, and rs4690072 (Carroll, et al., Mol Ther. 2011, 19: 2178-2185; Skotte, et al., PLOS ONE 2014, 9: e107434).

Currently, there is a lack of acceptable options for treating Huntington's disease. It is therefore an objective herein to provide compounds, pharmaceutical compositions, and methods of use for the treatment of HD.

### SUMMARY

Provided herein are compounds, pharmaceutical compositions, and methods of use for selectively reducing the amount or activity of a HTT RNA comprising SNP rs7685686, and in certain embodiments reducing the amount of a mHTT protein encoded by HTT comprising SNP rs7685686 in a cell or subject. In certain embodiments, the subject has HD. Compounds and pharmaceutical compositions provided herein selectively reduce the amount or activity of the HTT variant comprising SNP rs7685686 over wild-type HTT or over a non-target nucleic acid such as BMPR1. In certain embodiments, compounds useful for selectively reducing the amount or activity of a HTT RNA comprising SNP rs7685686 are oligomeric compounds. In certain embodiments, compounds useful for selectively reducing the amount or activity of a HTT RNA comprising SNP rs7685686 over wild-type HTT or over BMPR1 are oligomeric compounds. In certain embodiments, compounds useful for selectively reducing the amount or activity of a HTT RNA comprising SNP rs7685686 are modified oligonucleotides. In certain embodiments, compounds useful for selectively reducing the amount or activity of a HTT RNA comprising SNP rs7685686 over wild-type HTT or over BMPR1 are modified oligonucleotides. In certain embodiments, compounds useful for reducing the amount of a mHTT protein encoded by HTT comprising SNP rs7685686 are oligomeric compounds. In certain embodiments, compounds useful for reducing the amount of a mHTT protein encoded by HTT comprising SNP rs7685686 are modified oligonucleotides.

Also provided are methods useful for ameliorating at least one symptom or hallmark of HD. In certain embodiments, the symptom or hallmark includes brain atrophy, muscle atrophy, nerve degeneration, uncontrolled movement, seizure, tremor, anxiety, memory lapse, or depression. In certain embodiments, amelioration of one or more of these symptoms or hallmarks result in reduced or slowed brain atrophy, reduced or slowed muscle atrophy, slowed nerve degeneration, reduced uncontrolled movement, reduced seizure, reduced tremor, reduced anxiety, improved memory, or reduced depression.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout the disclosure are incorporated by reference herein in their entirety.

Unless otherwise indicated, the following terms have the following meanings: As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” means a sugar moiety with a 2′-OCH₂CH₂OCH₃ group in place of the 2′-OH group of a ribosyl sugar moiety. Unless otherwise indicated, a 2′-MOE sugar moiety is in the β-D configuration. “MOE” means O-methoxyethyl.

As used herein, “2′-MOE nucleoside” or “2′-O(CH₂)₂OCH₃ nucleoside” means a nucleoside comprising a 2′-MOE sugar moiety (or 2′-OCH₂CH₂OCH₃ ribosyl sugar moiety).

As used herein, “2′-OMe” means a 2′-OCH₃ group in place of the 2′-OH group of a ribosyl sugar moiety. A “2′-O-methyl sugar moiety” means a sugar moiety with a 2′-OCH₃ group in place of the 2′-OH group of a ribosyl sugar moiety. Unless otherwise indicated, a 2′-OMe has the β-D ribosyl stereochemical configuration.

As used herein, “2′-OMe nucleoside” means a nucleoside comprising a 2′-OMe sugar moiety.

As used herein, “2′-F” means a 2′-fluoro group in place of the 2′-OH group of a furanosyl sugar moiety. A “2′-F sugar moiety” means a sugar moiety with a 2′-F group in place of the 2′-OH group of a furanosyl sugar moiety. Unless otherwise indicated, a 2′-F sugar moiety is in the β-D-ribosyl configuration.

As used herein, “2′-F nucleoside” means a nucleoside comprising a 2′-F modified sugar moiety.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “5-methylcytosine” means a cytosine modified with a methyl group attached to the 5 position. A 5-methylcytosine is a modified nucleobase.

As used herein, “abasic sugar moiety” means a sugar moiety that is not attached to a nucleobase. Such abasic sugar moieties are sometimes referred to in the art as “abasic nucleosides.”

As used herein, “administration” or “administering” means providing a pharmaceutical agent or composition to a subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is brain atrophy, muscle atrophy, nerve degeneration, uncontrolled movement, seizure, tremor, anxiety, memory lapse, or depression. The progression or severity of indicators may be determined by subjective or objective measures, which are known to those skilled in the art.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide. Sense oligonucleotides include, but are not limited to, sense RNAi oligonucleotides.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl sugar moiety. In certain embodiments, the furanosyl sugar moiety is a ribosyl sugar moiety. In certain embodiments, the bicyclic sugar moiety does not comprise a furanosyl sugar moiety.

As used herein, “blunt” or “blunt ended” in reference to an oligomeric duplex formed by two oligonucleotides means that there are no terminal unpaired nucleotides (i.e. no overhanging nucleotides). One or both ends of a double-stranded RNAi agent can be blunt.

As used herein, “cell-targeting moiety” means a conjugate moiety or portion of a conjugate moiety that is capable of binding to a particular cell type or particular cell types.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides. In certain embodiments, the chiral center is at the phosphorous atom of a phosphorothioate internucleoside linkage. In certain embodiments, the chiral center is at the phosphorous atom of a mesyl phosphoramidate internucleoside linkage.

As used herein, “cleavable moiety” means a bond or group of atoms that is cleaved upon administration to a subject, for example, inside a cell, a subject, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methylcytosine (^{m}C) and guanine (G). Certain modified nucleobases that pair with unmodified nucleobases or with other modified nucleobases are known in the art. For example, inosine can pair with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or nucleic acid at each nucleobase of the shorter of the two oligonucleotides, or at each nucleoside if the oligonucleotides are the same length.

As used herein, “complementary region” in reference to an oligonucleotide is the range of nucleobases of the oligonucleotide that is complementary with a second oligonucleotide or target nucleic acid.

As used herein, “conjugate group” means a group of atoms directly attached to an oligonucleotide that confers at least one property to the resulting conjugated oligonucleotide. Conjugate groups comprise a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that when covalently bound to a molecule modifies one or more properties of such molecule compared to the identical molecule lacking the conjugate moiety, wherein such properties include, but are not limited to pharmacodynamics, pharmacokinetics, stability, binding, absorption, tissue distribution, cellular distribution, cellular uptake, charge, and clearance.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “deoxy region” means a region of 5-12 contiguous nucleotides, wherein at least 70% of the nucleosides comprise a 2′-deoxy sugar moiety. In certain embodiments, each nucleoside is selected from a 2′-β-D-deoxynucleoside, a bicyclic nucleoside, and a 2′-substituted nucleoside. In certain embodiments, a deoxy region supports RNase H activity. In certain embodiments, a deoxy region is the gap or internal region of a gapmer.

As used herein, “diluent” means an ingredient in a composition that lacks pharmacological activity, but is pharmaceutically necessary or desirable. For example, the diluent in an injected composition can be a liquid, e.g., aCSF, PBS, or saline solution.

As used herein, “double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings” or “wing segments.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to reduction of the amount or activity of the target nucleic acid by the action of an oligomeric agent, oligomeric compound, antisense compound, or antisense agent. Hotspot regions comprise at least one portion that is complementary to an active antisense oligonucleotide.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned in opposing directions.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “the nucleobase sequence of” a reference SEQ ID NO, refers only to the nucleobase sequence provided in such SEQ ID NO and therefore, unless otherwise indicated, includes compounds wherein each sugar moiety and each internucleoside linkage, independently, may be modified or unmodified, irrespective of the presence or absence of modifications, indicated in the referenced SEQ ID NO.

As used herein, “nucleoside” means a compound, or fragment of a compound, comprising a nucleobase and a sugar moiety. The nucleobase and sugar moiety are each, independently, unmodified or modified.

As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked).

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a polymer of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. An oligonucleotide may be paired with a second oligonucleotide that is complementary to the oligonucleotide or it may be unpaired. A “single-stranded oligonucleotide” is an unpaired oligonucleotide. A “double-stranded oligonucleotide” is an oligonucleotide that is paired with a second oligonucleotide.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable cater or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means an inactive or less active form of a compound which, when administered to a subject, is metabolized to form the active, or more active, compound. In certain embodiments, a prodrug comprises a cell-targeting moiety and at least one active compound.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNAi), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act principally through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H agents may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “antisense RNase H oligonucleotide” means an oligonucleotide comprising a region that is complementary to a target sequence, and which includes at least one chemical modification suitable for RNase H-mediated nucleic acid reduction.

As used herein, “antisense RNAi oligonucleotide” means an oligonucleotide comprising a region that is complementary to a target sequence, and which includes at least one chemical modification suitable for RNAi-mediated nucleic acid reduction.

As used herein, “self-complementary” in reference to an oligonucleotide means an oligonucleotide that at least partially hybridizes to itself.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard in vitro assay” means the assay described in Example 2, and reasonable variations thereof.

As used herein, “standard in vivo assay” means the assay described in any of Examples 3-7 and 9, and reasonable variations thereof.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). The stereochemical configuration of a chiral center is random when it is the result of a synthetic method that is not designed to control the stereochemical configuration. In certain embodiments, the stereorandom chiral center is at the phosphorous atom of a stereorandom phosphorothioate or mesyl phosphoramidate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) β-D-ribosyl sugar moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, symptoms and hallmarks include brain atrophy, muscle atrophy, nerve degeneration, uncontrolled movement, seizure, tremor, anxiety, memory lapse, or depression.

As used herein, “target nucleic acid” and “target RNA” mean a nucleic acid that an antisense compound is designed to affect. Target RNA means an RNA transcript and includes pre-mRNA and mature mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to a subject. For example, a therapeutically effective amount improves a symptom of a disease.

### CERTAIN EMBODIMENTS

The present disclosure provides the following non-limiting numbered embodiments:

Embodiment 1. A modified oligonucleotide according to the following chemical structure:

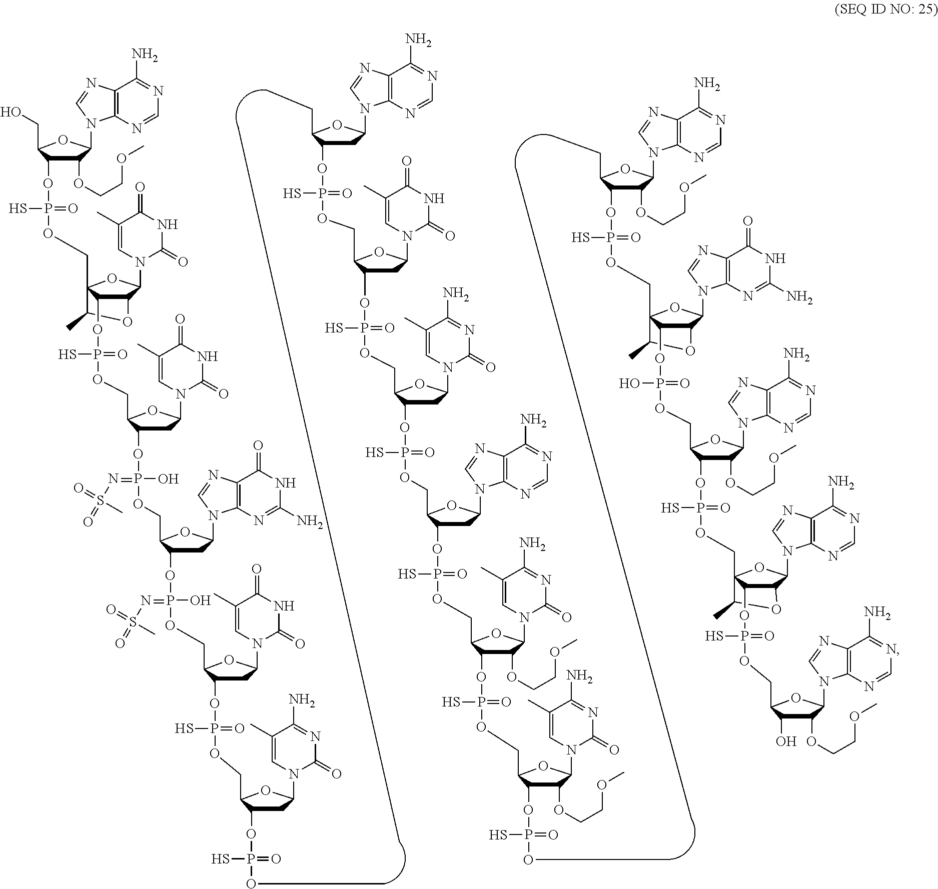

or a pharmaceutically acceptable salt thereof.

Embodiment 2. A modified oligonucleotide according to the following chemical structure:

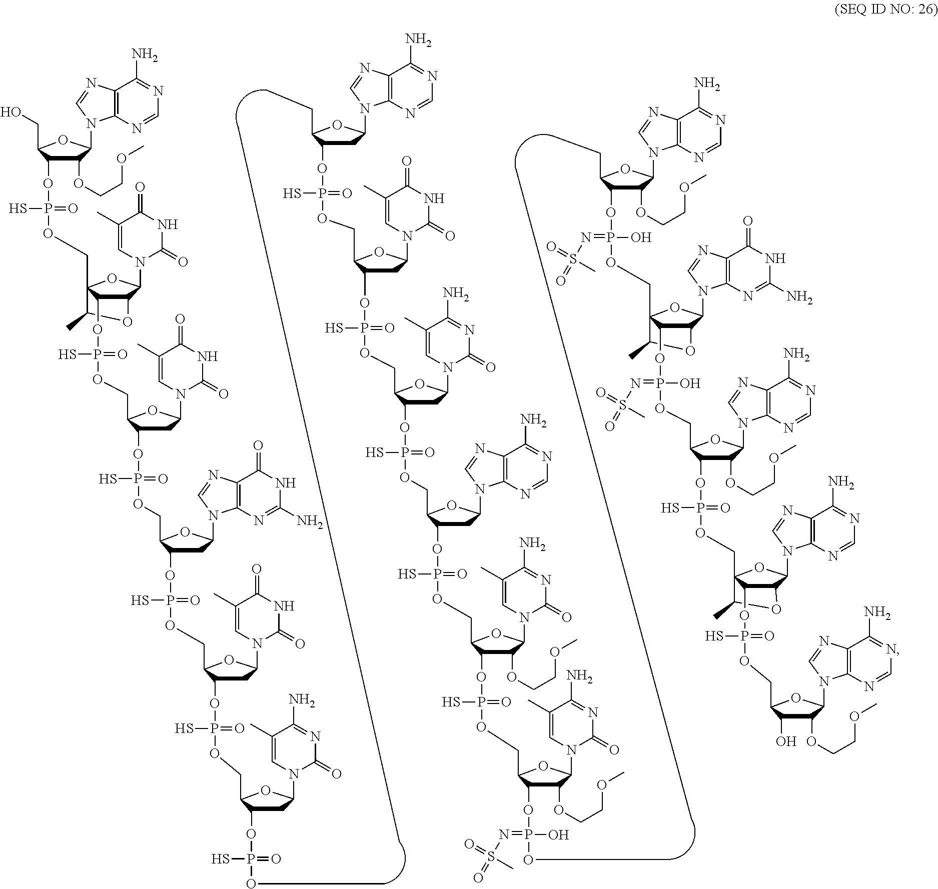

or a pharmaceutically acceptable salt thereof.

Embodiment 3. The modified oligonucleotide of embodiment 1 or embodiment 2, which is a pharmaceutically acceptable salt comprising one or more cations selected from sodium, potassium, calcium, and magnesium.

Embodiment 4. A modified oligonucleotide according to the following chemical structure:

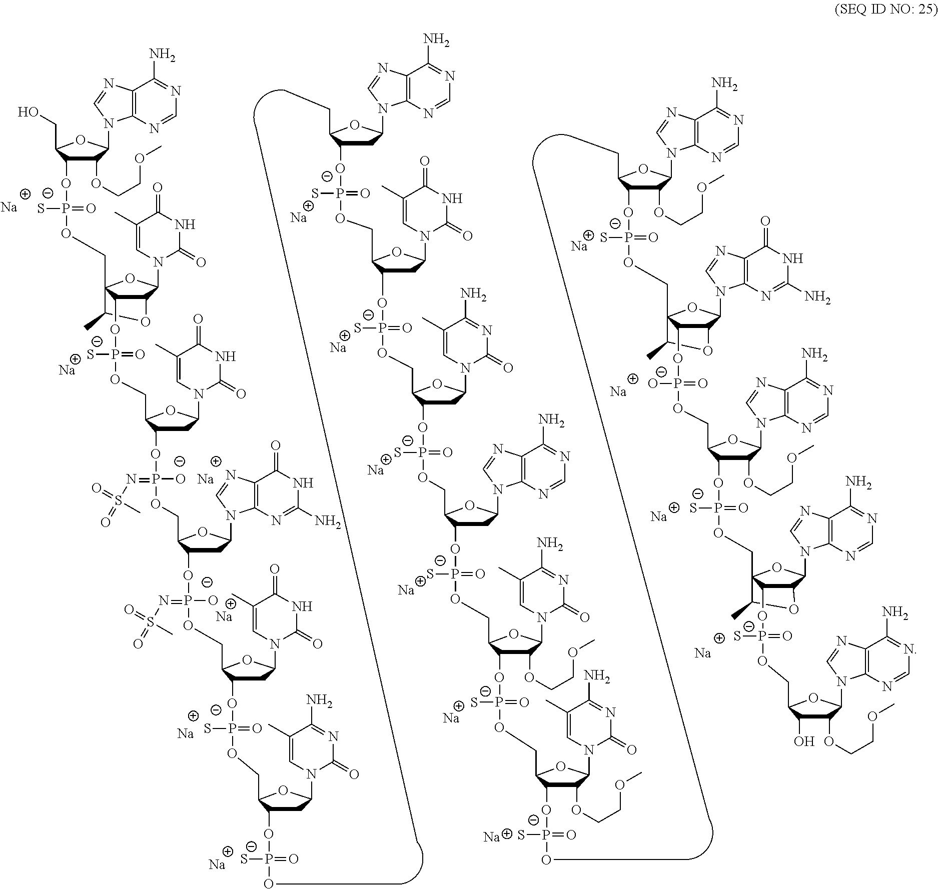

Embodiment 5. A modified oligonucleotide according to the following chemical structure:

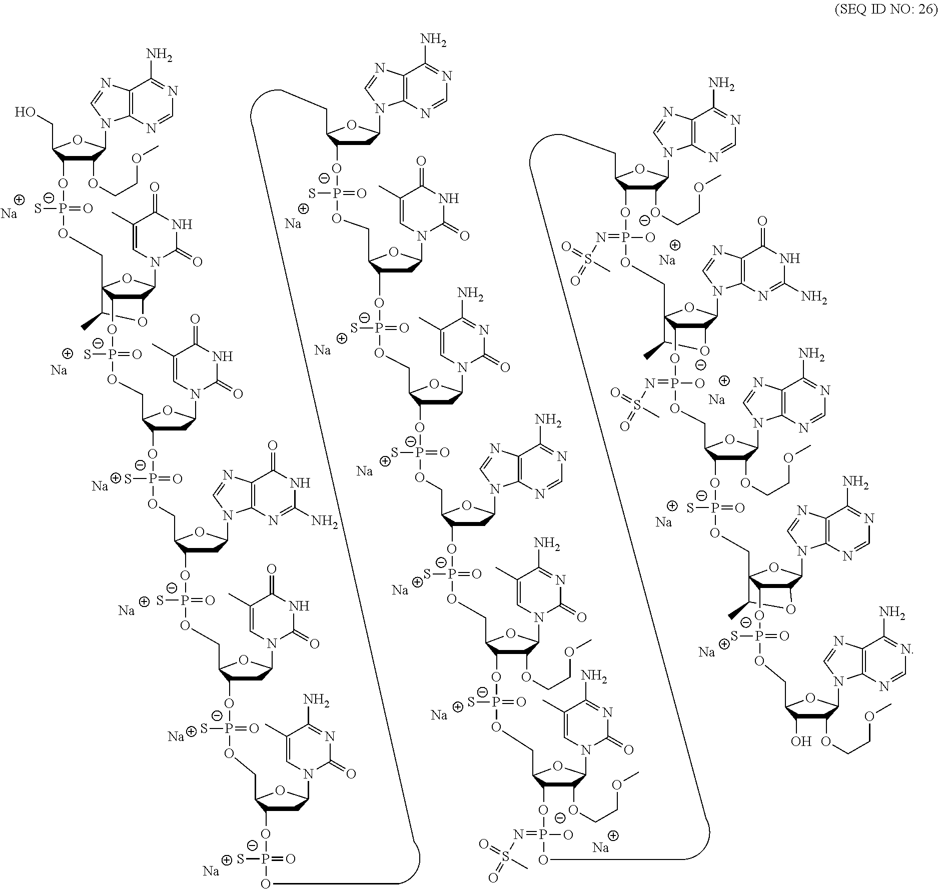

Embodiment 6. An oligomeric compound comprising a modified oligonucleotide according to the following chemical notation: AₑₛTₖₛT_{dz}G_{dz}T_{ds}^{m}C_{ds}A_{ds}T_{ds}^{m}C_{ds}A_{ds}^{m}Cₑₛ^{m}CₑₛAₑₛGₖₒAₑₛAₖₛAₑ (SEQ ID NO: 25), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
z=a mesyl phosphoramidate internucleoside linkage, and
o=a phosphodiester internucleoside linkage, and
   wherein the oligomeric compound optionally comprises a conjugate group or a terminal group.

Embodiment 7. An oligomeric compound comprising a modified oligonucleotide according to the following chemical notation: AₑₛTₖₛT_{ds}G_{ds}T_{ds}^{m}C_{ds}A_{ds}T_{ds}^{m}C_{ds}A_{ds}^{m}Cₑₛ^{m}C_{ez}A_{ez}G_{kz}AₑₛAₖₛAₑ (SEQ ID NO: 26), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage, and
z=a mesyl phosphoramidate internucleoside linkage, and
   wherein the oligomeric compound optionally comprises a conjugate group or a terminal group.

Embodiment 8. The oligomeric compound of embodiment 6 or embodiment 7, wherein the modified oligonucleotide is a pharmaceutically acceptable salt.

Embodiment 9. The oligomeric compound of embodiment 8, wherein the modified oligonucleotide is a pharmaceutically acceptable salt comprising one or more cations selected from sodium, potassium, calcium, and magnesium.

Embodiment 10. A population of modified oligonucleotides of any of embodiments 1-5 or a population of oligomeric compounds of any of embodiments 6-9, wherein all of the phosphorothioate internucleoside linkages of the modified oligonucleotide are stereorandom.

Embodiment 11. A population of modified oligonucleotides of any of embodiments 1-5 or a population of oligomeric compounds of any of embodiments 6-9, wherein all of the mesyl phosphoramidate internucleoside linkages of the modified oligonucleotide are stereorandom.

Embodiment 12. A pharmaceutical composition comprising a modified oligonucleotide of any of embodiments 1-5, an oligomeric compound of any of embodiments 6-9, a population of modified oligonucleotides of embodiment 10 or 11, or a population of oligomeric compounds of embodiment 10 or 11, and a pharmaceutically acceptable diluent.

Embodiment 13. The pharmaceutical composition of embodiment 12, wherein the pharmaceutically acceptable diluent is artificial cerebrospinal fluid (aCSF) or phosphate-buffered saline (PBS).

Embodiment 14. The pharmaceutical composition of embodiment 12, wherein the pharmaceutical composition consists essentially of the modified oligonucleotide of any of embodiments 1-5, the oligomeric compound of any of embodiments 6-9, the population of modified oligonucleotides of embodiment 10 or 11, or the population of oligomeric compounds of embodiment 10 or 11, and aCSF.

Embodiment 15. The pharmaceutical composition of embodiment 12, wherein the pharmaceutical composition consists essentially of the modified oligonucleotide of any of embodiments 1-5, the oligomeric compound of any of embodiments 6-9, the population of modified oligonucleotides of embodiment 10 or 11, or the population of oligomeric compounds of embodiment 10 or 11, and PBS.

Embodiment 16. The pharmaceutical composition of embodiment 12, wherein the pharmaceutical composition consists of the modified oligonucleotide of any of embodiments 1-5, the oligomeric compound of any of embodiments 6-9, the population of modified oligonucleotides of embodiment 10 or 11, or the population of oligomeric compounds of embodiment 10 or 11, and aCSF.

Embodiment 17. The pharmaceutical composition of embodiment 12, wherein the pharmaceutical composition consists of the modified oligonucleotide of any of embodiments 1-5, the oligomeric compound of any of embodiments 6-9, the population of modified oligonucleotides of embodiment 10 or 11, or the population of oligomeric compounds of embodiment 10 or 11, and PBS.

Embodiment 18. An oligomeric compound comprising a modified oligonucleotide consisting of 12 to 30 linked nucleosides and having a nucleobase complementary to huntingtin (HTT) SNP rs7685686, and wherein at least one internucleoside linkage of the modified oligonucleotide is a mesyl phosphoramidate internucleoside linkage.

Embodiment 19. An oligomeric compound comprising a modified oligonucleotide consisting of 12 to 30 linked nucleosides and having a nucleobase sequence comprising the nucleobase sequence of any of SEQ ID NOs: 14, 15, 18, 21, and 24, and wherein the modified oligonucleotide comprises an internucleoside linkage motif selected from: 5′-sssssssssssoooss-3′, 5′-sssssssssssoosss-3′, 5′-ssssssssssssooss-3′, 5′-sssssssssssooss-3′, 5′-sssssssssssssss-3′, 5′-sssoossssssssss-3′, and 5′-sssssssssssoss-3′; wherein each “s” represents a phosphorothioate internucleoside linkage, and each “o” represents a phosphodiester internucleoside linkage.

Embodiment 20. The oligomeric compound of embodiment 18, wherein the modified oligonucleotide comprises one or more internucleoside linkages selected from a phosphorothioate internucleoside linkage and a phosphodiester internucleoside linkage.

Embodiment 21. The oligomeric compound of embodiment 18 or embodiment 20, wherein the modified oligonucleotide comprises an internucleoside linkage motif selected from: 5′-ssssssssssszzzss-3′, 5′-szzsssssssssosss-3′, 5′-zsssssssssssosss-3′, 5′-zssssssssssssszz-3′, 5′-sszzsssssssssoss-3′, 5′-zzsssssssssssoss-3′, 5′-zssssssssssssszz-3′, 5′-ssssssssssszzzss-3′, 5′-ssssssssssszzss-3′, 5′-sszzsssssssssss-3′, 5′-zzsssssssssssss-3′, 5′-zsssssssssssssz-3′, 5′-sssssszzsssssss-3′, 5′-ssszzssssssssss-3′, 5′-sssssszssssssss-3′, 5′-ssssszzssssssss-3′, 5′-ssssszzszssssss-3′, 5′-ssszsszssssssss-3′, 5′-ssszszzssssssss-3′, 5′-sszzsszssssssss-3′, 5′-sszzszzssssssss-3′, 5′-ssszszszsssssss-3′, 5′-sszszszssssssss-3′, 5′-ssssssssssszss-3′, 5′-szzsssssssssss-3′, 5′-zsssssssssssss-3′, and 5′-zssssssssssssz-3′; wherein each “s” represents a phosphorothioate internucleoside linkage, each “o” represents a phosphodiester internucleoside linkage, and each “z” represents a mesyl phosphoramidate internucleoside linkage.

Embodiment 22. The oligomeric compound of any of embodiments 18-21, wherein at least one nucleoside of the modified oligonucleotide is a modified nucleoside.

Embodiment 23. The oligomeric compound of embodiment 22, wherein the modified nucleoside comprises a modified sugar moiety.

Embodiment 24. The oligomeric compound of embodiment 23, wherein the modified sugar moiety comprises a bicyclic sugar moiety.

Embodiment 25. The oligomeric compound of embodiment 24, wherein the bicyclic sugar moiety comprises a 2′-4′ bridge selected from —O—CH₂—; and —O—CH(CH₃)—.

Embodiment 26. The oligomeric compound of any of embodiments 22-25, wherein the modified nucleoside comprises a non-bicyclic modified sugar moiety.

Embodiment 27. The oligomeric compound of embodiment 26, wherein the non-bicyclic modified sugar moiety is a 2′-MOE sugar moiety or a 2′-OMe sugar moiety.

Embodiment 28. The oligomeric compound of any of embodiments 22-27, wherein the modified nucleoside comprises a sugar surrogate.

Embodiment 29. The oligomeric compound of embodiment 28, wherein the sugar surrogate is any of morpholino, modified morpholino, glycol nucleic acid (GNA), six-membered tetrahydropyran (THP), and F-hexitol nucleic acid (F-HNA).

Embodiment 30. The oligomeric compound of any of embodiments 18-27, wherein the modified oligonucleotide comprises a modified sugar motif selected from: 5′-kddddddddeeekekee-3′, 5′-kdddddddddeekekee-3′, 5′-eddddddddeeekekee-3′, 5′-edddddddddeekekee-3′, 5′-kddddddddeeeeeeee-3′, 5′-kdddddddddeeeeeee-3′, 5′-eddddddddeeeeeeee-3′, 5′-edddddddddeeeeeee-3′, 5′-ekddddddddeeekeke-3′, 5′-ekdddddddddeekeke-3′, 5′-eedddddddddeekeke-3′, 5′-eeddddddddeeekeke-3′, 5′-ekdddddddddeeeeee-3′, 5′-ekddddddddeeeeeee-3′, 5′-eedddddddddeeeeee-3′, 5′-eeddddddddeeeeeee-3′, 5′-ekddddddddeeekek-3′, 5′-ekdddddddeeeekek-3′, 5′-eeddddddddeeekek-3′, 5′-eedddddddeeeekek-3′, 5′-ekddddddddeeeeee-3′, 5′-ekdddddddeeeeeee-3′, 5′-eeddddddddeeeeee-3′, 5′-eedddddddeeeeeee-3′, 5′-ekddddddddeeekek-3′, 5′-ekdddddddeeeekek-3′, 5′-ekddddddddeeekek-3′, 5′-ekdddddddeeeekek-3′, 5′-eeeeeedddddddkke-3′, 5′-eeeekkdddddddeee-3′, 5′-eeeekkdddddddkke-3′, 5′-eeeeeedddddddeee-3′, 5′-ekddddddddeeekek-3′, 5′-kdddddddddkeekk-3′, 5′-edddddddddkeekk-3′, 5′-kdddddddddeeeee-3′, and 5′-edddddddddeeeee-3′; wherein each “d” represents a 2′-β-D-deoxyribosyl sugar moiety, each “e” represents a 2′-MOE sugar moiety, and each “k” represents a cEt sugar moiety.

Embodiment 31. An oligomeric compound comprising a modified oligonucleotide consisting of 12 to 30 linked nucleosides and having a nucleobase complementary to huntingtin (HTT) SNP rs7685686, and wherein the modified oligonucleotide comprises a modified sugar moiety selected from a 2′-OMe sugar moiety or a 2′-alpha-L-deoxyribosyl sugar moiety.

Embodiment 32. The oligomeric compound of embodiment 31, wherein the modified oligonucleotide comprises one or more modified sugar moieties selected from a cEt sugar moiety and a 2′-MOE sugar moiety.

Embodiment 33. The oligomeric compound of embodiment 31 or embodiment 32, wherein the modified oligonucleotide comprises a modified sugar motif selected from: 5′-kydddddddeeekekee-3′, 5′-kyddddddddeekekee-3′, 5′-kdyddddddeeekekee-3′, 5′-kdydddddddeekekee-3′, 5′-ekyddddddddeekeke-3′, 5′-ekydddddddeeekeke-3′, 5′-ekdydddddddeekeke-3′, 5′-ekdyddddddeeekeke-3′, 5′-ekydddddddeeekek-3′, 5′-ekdyddddddeeekek-3′, 5′-kyddddddddkeekk-3′, 5′-kdydddddddkeekk-3′, 5′-kddd[aLd]ddddeeekekee-3′, 5′-kddd[aLd]dddddeekekee-3′, 5′-ekddd[aLd]dddddeekeke-3′, 5′-ekddd[aLd]ddddeeekeke-3′, 5′-eeeekkddd[aLd]dddkke-3′, 5′-kddd[aLd]dddddkeekk-3′, 5′-ekddd[aLd]ddddeeekek-3′, and 5′-ekddd[aLd]dddeeeekek-3′; wherein each “d” represents a 2′-β-D-deoxyribosyl sugar moiety, each “e” represents a 2′-MOE sugar moiety, each “k” represents a cEt sugar moiety, each “y” represents a 2′-O-methyl sugar moiety, and each “[aLd]” represents a 2′-α-L-deoxyribosyl sugar moiety.

Embodiment 34. The oligomeric compound of any of embodiments 31-33, wherein the modified oligonucleotide comprises at least one modified internucleoside linkage.

Embodiment 35. The oligomeric compound of embodiment 34, wherein at least one internucleoside linkage is a phosphodiester internucleoside linkage.

Embodiment 36. The oligomeric compound of embodiment 34 or embodiment 35, wherein at least one modified internucleoside linkage is a phosphorothioate internucleoside linkage.

Embodiment 37. The oligomeric compound of any of embodiments 34-36, wherein each internucleoside linkage is independently selected from a phosphodiester internucleoside linkage and a phosphorothioate internucleoside linkage.

Embodiment 38. The oligomeric compound of any of embodiments 34-37, wherein the modified oligonucleotide has an internucleoside linkage motif selected from: 5′-sssssssssssoosss-3′, 5′-ssssssssssssooss-3′, 5′-sssssssssssooss-3′, 5′-sssssssssssssss-3′, and 5′-sssssssssssoss-3′; wherein each “s” represents a phosphorothioate internucleoside linkage, and each “o” represents a phosphodiester internucleoside linkage.

Embodiment 39. The oligomeric compound of any of embodiments 18-38, wherein the nucleobase sequence of the modified oligonucleotide is at least 85%, at least 90%, at least 95%, or 100% complementary to an equal length portion of an HTT nucleic acid, wherein the HTT nucleic acid has the nucleobase sequence of SEQ ID NO: 1.

Embodiment 40. The oligomeric compound of any of embodiments 18-39, wherein the modified oligonucleotide consists of 12 to 20, 12 to 25, 12 to 30, 13 to 20, 13 to 25, 13 to 30, 14 to 20, 14 to 25, 14 to 30, 15 to 20, 15 to 25, 15 to 30, 16 to 18, 16 to 20, 16 to 25, 16 to 30, 17 to 20, 17 to 25, 17 to 30, 18 to 20, 18 to 22, 18 to 25, 18 to 30, 19 to 20, 19 to 25, 19 to 30, 20 to 25, 20 to 30, 21 to 25, 21 to 30, 22 to 25, 22 to 30, 23 to 25, or 23 to 30 linked nucleosides.

Embodiment 41. The oligomeric compound of any of embodiments 18-40, wherein the modified oligonucleotide consists of 15 linked nucleosides.

Embodiment 42. The oligomeric compound of any of embodiments 18-40, wherein the modified oligonucleotide consists of 16 linked nucleosides.

Embodiment 43. The oligomeric compound of any of embodiments 18-40, wherein the modified oligonucleotide consists of 17 linked nucleosides.

Embodiment 44. The oligomeric compound of any of embodiments 18-30, and 39-43, wherein the modified oligonucleotide comprises the nucleobase sequence of any of SEQ ID NOs: 14, 15, 18, 21, and 24.

Embodiment 45. The oligomeric compound of any of embodiments 31-43, wherein the modified oligonucleotide comprises the nucleobase sequence of any of SEQ ID NOs: 14, 15, 18, 19, 20, 21, 22, 23, and 24.

Embodiment 46. The oligomeric compound of any of embodiments 18-45, wherein at least one nucleoside of the modified oligonucleotide comprises a modified nucleobase.

Embodiment 47. The oligomeric compound of embodiment 46, wherein the modified nucleobase is 5-methylcytosine.

Embodiment 48. The oligomeric compound of embodiment 46, wherein each cytosine is a 5-methylcytosine.

Embodiment 49. The oligomeric compound of any of embodiments 18-48, wherein each nucleoside of the modified oligonucleotide is unmodified adenine, unmodified guanine, unmodified thymine, unmodified cytosine, or 5-methylcytosine.

Embodiment 50. The oligomeric compound of any of embodiments 18-49, wherein the modified oligonucleotide comprises a deoxy region.

Embodiment 51. The oligomeric compound of embodiment 50, wherein each nucleoside of the deoxy region is a 2′-β-D-deoxynucleoside.

Embodiment 52. The oligomeric compound of embodiment 50 or embodiment 51, wherein the deoxy region consists of 6, 7, 8, 9, 10, or 6-10 linked nucleosides.

Embodiment 53. The oligomeric compound of any of embodiments 50-52, wherein each nucleoside immediately adjacent to the deoxy region comprises a modified sugar moiety.

Embodiment 54. The oligomeric compound of any of embodiments 50-53, wherein the deoxy region is flanked on the 5′-side by a 5′-region consisting of 1-6 linked 5′-region nucleosides and on the 3′-side by a 3′-region consisting of 1-8 linked 3′-region nucleosides; wherein at least one nucleoside of the 5′-region comprises a modified sugar moiety; and at least one nucleoside of the 3′-region comprises a modified sugar moiety.

Embodiment 55. The oligomeric compound of embodiment 54, wherein each nucleoside of the 5′-region comprises a modified sugar moiety.

Embodiment 56. The oligomeric compound of embodiment 54 or embodiment 55, wherein each nucleoside of the 3′-region comprises a modified sugar moiety.

Embodiment 57. The oligomeric compound of any of embodiments 18-56, consisting of the modified oligonucleotide.

Embodiment 58. The oligomeric compound of any of embodiments 18-56, wherein the oligomeric compound comprises a conjugate group.

Embodiment 59. The oligomeric compound of embodiment 58, wherein the conjugate group comprises a conjugate moiety and a conjugate linker.

Embodiment 60. The oligomeric compound of embodiment 59, wherein the conjugate linker is a phosphodiester linker.

Embodiment 61. The oligomeric compound of embodiment 59, wherein the conjugate linker consists of a single bond.

Embodiment 62. The oligomeric compound of any of embodiments 59-61, wherein the conjugate linker is cleavable.

Embodiment 63. The oligomeric compound of any of embodiments 59, 60, or 62, wherein the conjugate linker comprises 1-3 linker-nucleosides, wherein at least one linker nucleoside is linked to the conjugate moiety, to the modified oligonucleotide, or to another linker-nucleoside by a phosphodiester bond.

Embodiment 64. The oligomeric compound of any of embodiments 58-63, wherein the conjugate group is attached to the modified oligonucleotide at the 5′-end of the modified oligonucleotide.

Embodiment 65. The oligomeric compound of any of embodiments 58-63, wherein the conjugate group is attached to the modified oligonucleotide at the 3′-end of the modified oligonucleotide.

Embodiment 66. The oligomeric compound of any of embodiments 18-58, wherein the oligomeric compound does not comprise linker-nucleosides.

Embodiment 67. The oligomeric compound of any of embodiments 18-66, comprising a terminal group.

Embodiment 68. The oligomeric compound of embodiment 67, wherein the terminal group is an abasic sugar moiety.

Embodiment 69. The oligomeric compound of any of embodiments 18-68, wherein the oligomeric compound is an RNase H agent.

Embodiment 70. An oligomeric compound according to the following chemical notation: N¹ₑₛTₖₛT_{dz}G_{dz}T_{ds}^{m}C_{ds}A_{ds}T_{ds}^{m}C_{ds}A_{ds}^{m}Cₑₛ^{m}CₑₛAₑₛGₖₒAₑₛAₖₛN²ₑ (SEQ ID NO: 27), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar is also absent,
e=a 2′-MOE sugar moiety,
k=a cEt sugar moiety,
d=a 2′-β-D-deoxyribosyl sugar moiety,
s=a phosphorothioate internucleoside linkage,
z=a mesyl phosphoramidate internucleoside linkage, and
o=a phosphodiester internucleoside linkage, and
   wherein the oligomeric compound optionally comprises a conjugate group.

Embodiment 71. An oligomeric compound according to the following chemical notation: N¹ₑₛTₖₛT_{ds}G_{ds}T_{ds}^{m}C_{ds}A_{ds}T_{ds}^{m}C_{ds}A_{ds}^{m}Cₑₛ^{m}C_{ez}A_{ez}G_{kz}AₑₛAₖₛN²ₑ (SEQ ID NO: 28), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar is also absent,
e=a 2′-MOE sugar moiety,
k=a cEt sugar moiety,
d=a 2′-β-D-deoxyribosyl sugar moiety,
s=a phosphorothioate internucleoside linkage, and
z=a mesyl phosphoramidate internucleoside linkage, and
   wherein the oligomeric compound optionally comprises a conjugate group.

Embodiment 72. The oligomeric compound of embodiment 70 or 71, wherein N¹ and N² are each independently an adenine nucleobase.

Embodiment 73. The oligomeric compound of embodiment 70 or 71, wherein N¹ and N² are each independently an unmodified adenine.

Embodiment 74. The oligomeric compound of embodiment 70 or 71, wherein N¹ and N² are each independently a modified adenine.

Embodiment 75. The oligomeric compound of embodiment 70 or 71, wherein N¹ and N² are each independently a hypoxanthine.

Embodiment 76. The oligomeric compound of embodiment 70 or 71, wherein N¹ and N² are each independently an abasic sugar moiety.

Embodiment 77. The oligomeric compound of embodiment 70 or 71, wherein N¹ and N² are each independently a terminal group.

Embodiment 78. The oligomeric compound of embodiment 70 or 71, wherein N¹ and N² are each independently absent.

Embodiment 79. The oligomeric compound of any of embodiments 70-78, wherein N¹ is an adenine nucleobase and N² is an unmodified adenine, a hypoxanthine, an abasic sugar moiety, a terminal group, or is absent.

Embodiment 80. The oligomeric compound of any of embodiments 70-79, wherein N¹ is an adenine nucleobase and N² is an unmodified adenine.

Embodiment 81. The oligomeric compound of any of embodiments 70-79, wherein N¹ is an adenine nucleobase and N² is a hypoxanthine.

Embodiment 82. The oligomeric compound of any of embodiments 70-79, wherein N¹ is an adenine nucleobase and N² is an abasic sugar moiety.

Embodiment 83. The oligomeric compound of any of embodiments 70-79, wherein N¹ is an adenine nucleobase and N² is a terminal group.

Embodiment 84. The oligomeric compound of any of embodiments 70-79, wherein N¹ is an adenine nucleobase and N² is absent.

Embodiment 85. The oligomeric compound of any of embodiments 70-79, wherein N¹ is an unmodified adenine and N² is an adenine nucleobase, an unmodified adenine, a hypoxanthine, an abasic sugar moiety, a terminal group, or is absent.

Embodiment 86. The oligomeric compound of any of embodiments 70-79 or 85, wherein N¹ is an unmodified adenine and N² is an adenine nucleobase.

Embodiment 87. The oligomeric compound of any of embodiments 70-79 or 85, wherein N¹ is an unmodified adenine and N² is an unmodified adenine.

Embodiment 88. The oligomeric compound of any of embodiments 70-79 or 85, wherein N¹ is an unmodified adenine and N² is a hypoxanthine.

Embodiment 89. The oligomeric compound of any of embodiments 70-79 or 85, wherein N¹ is an unmodified adenine and N² is an abasic sugar moiety.

Embodiment 90. The oligomeric compound of any of embodiments 70-79 or 85, wherein N¹ is an unmodified adenine and N² is a terminal group.

Embodiment 91. The oligomeric compound of any of embodiments 70-79 or 85, wherein N¹ is an unmodified adenine and N² is absent.

Embodiment 92. The oligomeric compound of any of embodiments 70-78, wherein N¹ is a hypoxanthine and N² is an adenine nucleobase, an unmodified adenine, a hypoxanthine, an abasic sugar moiety, a terminal group, or is absent.

Embodiment 93. The oligomeric compound of any of embodiments 70-78 or 92, wherein N¹ is a hypoxanthine and N² is an adenine nucleobase.

Embodiment 94. The oligomeric compound of any of embodiments 70-78 or 92, wherein N¹ is a hypoxanthine and N² is an unmodified adenine.

Embodiment 95. The oligomeric compound of any of embodiments 70-78 or 92, wherein N¹ is a hypoxanthine and N² is a hypoxanthine.

Embodiment 96. The oligomeric compound of any of embodiments 70-78 or 92, wherein N¹ is a hypoxanthine and N² is an abasic sugar moiety.

Embodiment 97. The oligomeric compound of any of embodiments 70-78 or 92, wherein N¹ is a hypoxanthine and N² is a terminal group.

Embodiment 98. The oligomeric compound of any of embodiments 70-78 or 92, wherein N¹ is a hypoxanthine and N² is absent.

Embodiment 99. The oligomeric compound of any of embodiments 70-78, wherein N¹ is an abasic sugar moiety and N² is an adenine nucleobase, an unmodified adenine, a hypoxanthine, an abasic sugar moiety, a terminal group, or is absent.

Embodiment 100. The oligomeric compound of any of embodiments 70-78, wherein N¹ is a terminal group and N² is an adenine nucleobase, an unmodified adenine, a hypoxanthine, an abasic sugar moiety, a terminal group, or is absent.

Embodiment 101. The oligomeric compound of any of embodiments 70-78, wherein N¹ is absent and N² is an adenine nucleobase, an unmodified adenine, a hypoxanthine, an abasic sugar moiety, a terminal group, or is absent.

Embodiment 102. The oligomeric compound of any of embodiments 70-78, wherein N¹ is absent and N² is absent.

Embodiment 103. The oligomeric compound of embodiment 70 or 71, wherein the conjugate group comprises a conjugate moiety and a conjugate linker.

Embodiment 104. The oligomeric compound of embodiment 103, wherein the conjugate linker is a phosphodiester linker.

Embodiment 105. The oligomeric compound of embodiment 103, wherein the conjugate linker consists of a single bond.

Embodiment 106. The oligomeric compound of any of embodiments 103-105, wherein the conjugate linker is cleavable.

Embodiment 107. The oligomeric compound of any of embodiments 103, 104, or 106, wherein the conjugate linker comprises 1-3 linker-nucleosides, wherein at least one linker nucleoside is linked to the conjugate moiety, to the oligomeric compound, or to another linker-nucleoside by a phosphodiester bond.

Embodiment 108. The oligomeric compound of any of embodiments 70, 71, or 103-107, wherein the conjugate group is attached to the oligomeric compound at the 5′-end of the oligomeric compound.

Embodiment 109. The oligomeric compound of any of embodiments 70, 71, or 103-107, wherein the conjugate group is attached to the oligomeric compound at the 3′-end of the oligomeric compound.

Embodiment 110. The oligomeric compound of any of embodiments 70-109, wherein the oligomeric compound is a pharmaceutically acceptable salt.

Embodiment 111. The oligomeric compound of embodiment 110, wherein the pharmaceutically acceptable salt comprises one or more cations selected from sodium, potassium, calcium, and magnesium.

Embodiment 112. A population of oligomeric compounds of any of embodiments 18-111, wherein all of the phosphorothioate internucleoside linkages of the modified oligonucleotide are stereorandom.

Embodiment 113. A population of oligomeric compounds of any of embodiments 18-111, wherein all of the mesyl phosphoramidate internucleoside linkages of the modified oligonucleotide are stereorandom.

Embodiment 114. A pharmaceutical composition comprising an oligomeric compound of any of embodiments 18-111, or a population of oligomeric compounds of embodiment 112 or 113, and a pharmaceutically acceptable diluent.

Embodiment 115. The pharmaceutical composition of embodiment 114, wherein the pharmaceutically acceptable diluent is artificial cerebrospinal fluid (aCSF) or phosphate-buffered saline (PBS).

Embodiment 116. The pharmaceutical composition of embodiment 115, wherein the pharmaceutical composition consists essentially of the oligomeric compound of any of embodiments 18-111, or the population of oligomeric compounds of embodiment 112 or 113, and aCSF.

Embodiment 117. The pharmaceutical composition of embodiment 115, wherein the pharmaceutical composition consists essentially of the oligomeric compound of any of embodiments 18-111, or the population of oligomeric compounds of embodiment 112 or 113, and PBS.

Embodiment 118. The pharmaceutical composition of embodiment 115, wherein the pharmaceutical composition consists of the oligomeric compound of any of embodiments 18-111, or the population of oligomeric compounds of embodiment 112 or 113, and aCSF.

Embodiment 119. The pharmaceutical composition of embodiment 115, wherein the pharmaceutical composition consists of the oligomeric compound of any of embodiments 18-111, or the population of oligomeric compounds of embodiment 112 or 113, and PBS.

Embodiment 120. A method comprising administering to a subject a modified oligonucleotide of any of embodiments 1-5, an oligomeric compound of any of embodiments 6-9, or 18-111, a population of modified oligonucleotides of embodiment 10 or 11, a population of oligomeric compounds of any of embodiments 10, 11, 112, or 113, or a pharmaceutical composition of any of embodiments 12-17 or 114-119.

Embodiment 121. The method of embodiment 120, wherein the subject has or is at risk of developing Huntington's disease.

Embodiment 122. A method of treating Huntington's disease comprising administering to a subject having or at risk of developing Huntington's disease a therapeutically effective amount of a modified oligonucleotide of any of embodiments 1-5, an oligomeric compound of any of embodiments 6-9, or 18-111, a population of modified oligonucleotides of embodiment 10 or 11, a population of oligomeric compounds of any of embodiments 10, 11, 112, or 113, or a pharmaceutical composition of any of embodiments 12-17 or 114-119.

Embodiment 123. The method of embodiment 122, wherein at least one symptom or hallmark of Huntington's disease is ameliorated.

Embodiment 124. The method of embodiment 123, wherein the symptom or hallmark is brain atrophy, muscle atrophy, nerve degeneration, uncontrolled movement, seizure, tremor, anxiety, memory lapse, or depression.

Embodiment 125. The method of embodiment 124, wherein administering the modified oligonucleotide of any of embodiments 1-5, the oligomeric compound of any of embodiments 6-9, or 18-111, the population of modified oligonucleotides of embodiment 10 or 11, the population of oligomeric compounds of any of embodiments 10, 11, 112, or 113, or the pharmaceutical composition of any of embodiments 12-17 or 114-119 reduces or delays the onset or progression of brain atrophy, muscle atrophy, nerve degeneration, uncontrolled movement, seizure, tremor, anxiety, memory lapse, or depression.

Embodiment 126. The method of any of embodiments 120-125, wherein the modified oligonucleotide of any of embodiments 1-5, the oligomeric compound of any of embodiments 6-9, or 18-111, the population of modified oligonucleotides of embodiment 10 or 11, the population of oligomeric compounds of any of embodiments 10, 11, 112, or 113, or the pharmaceutical composition of any of embodiments 12-17 or 114-119 is administered to the central nervous system or systemically.

Embodiment 127. The method of any of embodiments 120-126, wherein the modified oligonucleotide of any of embodiments 1-5, the oligomeric compound of any of embodiments 6-9, or 18-111, the population of modified oligonucleotides of embodiment 10 or 11, the population of oligomeric compounds of any of embodiments 10, 11, 112, or 113, or the pharmaceutical composition of any of embodiments 12-17 or 114-119 is administered intrathecally.

Embodiment 128. The method of any of embodiments 120-127, wherein the subject is a human.

Embodiment 129. A method of reducing expression of HTT in a cell comprising contacting the cell with a modified oligonucleotide of any of embodiments 1-5, an oligomeric compound of any of embodiments 6-9, or 18-111, a population of modified oligonucleotides of embodiment 10 or 11, a population of oligomeric compounds of any of embodiments 10, 11, 112, or 113, or a pharmaceutical composition of any of embodiments 12-17 or 114-119.

Embodiment 130. The method of embodiment 129, wherein the cell is a brain cell.

Embodiment 131. The method of embodiment 129, wherein the cell is a neuron or a glial cell.

Embodiment 132. The method of any of embodiments 129-131, wherein the cell is a human cell.

Embodiment 133. Use of a modified oligonucleotide of any of embodiments 1-5, an oligomeric compound of any of embodiments 6-9, or 18-111, a population of modified oligonucleotides of embodiment 10 or 11, a population of oligomeric compounds of any of embodiments 10, 11, 112, or 113, or a pharmaceutical composition of any of embodiments 12-17 or 114-119 for treating Huntington's disease.

Embodiment 134. Use of a modified oligonucleotide of any of embodiments 1-5, an oligomeric compound of any of embodiments 6-9, or 18-111, a population of modified oligonucleotides of embodiment 10 or 11, a population of oligomeric compounds of any of embodiments 10, 11, 112, or 113, or a pharmaceutical composition of any of embodiments 12-17 or 114-119 for the manufacture of a medicament for treating Huntington's disease.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited to, substituents at the 2′, 3′, 4′, and/or 5′ positions. In certain embodiments, the furanosyl sugar moiety is a ribosyl sugar moiety. In certain embodiments, one or more acyclic substituent of non-bicyclic modified sugar moieties is branched.

In certain embodiments, non-bicyclic modified sugar moieties comprise a substituent group at the 2′-position. Examples of substituent groups suitable for the 2′-position of modified sugar moieties include but are not limited to: —F, —OCH₃ (“OMe” or “O-methyl”), and —OCH₂CH₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), or 2′-O(CH₂)₂O(CH₂)₂N(CH₃)₂ (“DMAEOE”). And the 2′-substituent groups described in Cook et al., U.S. Pat. No. 6,531,584; Cook et al., U.S. Pat. No. 5,859,221; and Cook et al., U.S. Pat. No. 6,005,087

Synthetic methods for some of these 2′-substituent groups can be found in, e.g., Cook et al., U.S. Pat. No. 6,531,584; and Cook et al., U.S. Pat. No. 5,859,221. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted sugar moiety of a modified nucleoside comprises 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted sugar moiety of a modified nucleoside comprises 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO2020/072991. A 2′-modified sugar moiety has an additional stereocenter at the 2′-position relative to a 2′-deoxyfuranosyl sugar moiety; therefore, such sugar moieties have a total of sixteen possible isomeric configurations. Modified furanosyl sugar moieties described herein are in the β-D-ribosyl isomeric configuration unless otherwise specified.

In certain embodiments, non-bicyclic modified sugar moieties are stereoisomers of DNA, such as 2′-α-L-deoxyribosyl sugar moiety:

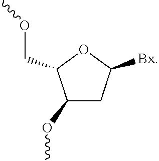

In certain embodiments, non-bicyclic modified sugar moieties comprise a substituent group at the 4′-position. Examples of substituent groups suitable for the 4′-position of modified sugar moieties include, but are not limited to, alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include, but are not limited to, alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include, but are not limited to, vinyl, alkoxy (e.g., methoxy), and alkyl (e.g., methyl (R or S), ethyl).

In certain embodiments, non-bicyclic modified sugar moieties comprise more than one non-bridging sugar substituent, for example, 2′-F-5′-methyl sugar moieties, such as described in Migawa et al., US2010/0190837, or alternative 2′- and 5′-modified sugar moieties as described in Rajeev et al., US2013/0203836.

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′-(CH₂)₂-2′, 4′-(CH₂)₃-2′, 4′-CH₂-0-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′-CH₂—C(H)(CH₃)-2′, 4′-CH₂—C(═CH₂)-2′ and analogs thereof), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl. Representative U.S. patents that teach the preparation of such bicyclic sugar moieties include, but are not limited to: Imanishi et al., U.S. Pat. No. 7,427,672; Swayze et al., U.S. Pat. No. 7,741,457, and Swayze et al., U.S. Pat. No. 8,022,193; Seth et al., U.S. Pat. No. 8,278,283; Prakash et al., U.S. Pat. No. 8,278,425; Seth et al., U.S. Pat. No. 8,278,426).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Wan, et al., J. Medicinal Chemistry, 2016, 59, 9645-9667; Wengel et al., U.S. Pat. No. 8,080,644; Ramasamy et al., U.S. Pat. No. 6,525,191; Seth et al., U.S. Pat. No. 7,547,684; and Seth et al., U.S. Pat. No. 7,666,854.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

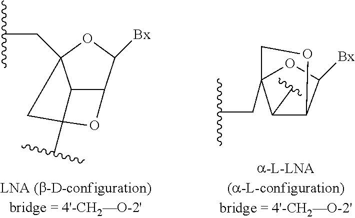

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol. Canc. Ther. 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include, but are not limited to, hexitol nucleic acid (“HNA”), anitol nucleic acid (“ANA”), manitol nucleic acid (“MNA”), fluoro HNA:

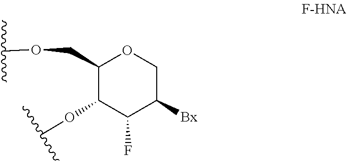

(“F-HNA”, see e.g., Egli, et. al., J Am Chem (2011) 133(41):16642-16649, Swayze et al., U.S. Pat. No. 8,088,904; and Swayze et al., U.S. Pat. No. 8,440,803) F-HNA can also be referred to as a F-THP or 3′-fluoro tetrahydropyran, and nucleosides comprising additional modified THP compounds having the formula:

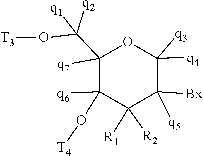

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported. As used here, the term “morpholino” means a sugar surrogate having the following structure:

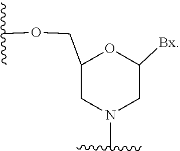

In certain embodiments, morpholinos may be modified, for example, by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid), and nucleosides and oligonucleotides described in Manoharan et al., U.S. Pat. No. 10,913,767. Representative U.S. patents that teach the preparation of PNA compounds include, but are not limited to, U.S. Pat. Nos. 5,539,082; 5,714,331; and 5,719,262.

In certain embodiments, sugar surrogates are the “unlocked” sugar structure of UNA (unlocked nucleic acid) nucleosides. UNA is a nucleoside wherein any of the bonds of the sugar moiety has been removed, forming an unlocked sugar surrogate. A representative U.S. publication that teaches the preparation of UNA includes, but is not limited to, US Patent Publication No 2011/0313020.

In certain embodiments, sugar surrogates are the glycerol as found in GNA (glycol nucleic acid) nucleosides as depicted below:

### (S)-GNA

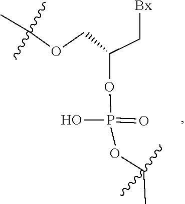

where Bx represents any nucleobase.

Many other bicyclic and tricyclic sugar and sugar surrogates are known in the art that can be used in modified nucleosides.

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase). An “unmodified nucleobase” is adenine (A), thymine (T), cytosine (C), uracil (U), or guanine (G). A modified nucleobase is a group of atoms other than unmodified A, T, C, U, or G capable of pairing with at least one other nucleobase. A 5-methylcytosine is an example of a modified nucleobase. A universal base is a modified nucleobase that can pair with any one of the five unmodified nucleobases.

In certain embodiments, modified adenine has structure (I):

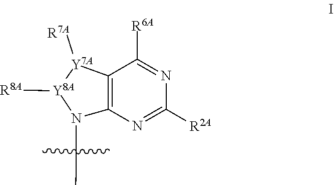

wherein: R^{2A} is H, C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy; R^{6A} is H, N(R^{a})(R^{b}), acetyl, formyl, or O-phenyl; Y^{7A} is N and R^{7A} is absent or is C₁-C₆ alkyl; or Y^{7A} is C and R^{7A} is selected from H, C₁-C₆ alkyl, or CN(R^{a})(R^{b}); Y^{8A} is N and R^{8A} is absent, or Y^{8A} is C and R^{8A} is selected from H, a halogen, OH, C₁-C₆ alkyl, or substituted C₁-C₆ alkyl; R^{a} and R^{b} are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7A} is N; Y^{8A} is C, R^{8A} is H, R² is H, and R^{6A} is NH₂ (unmodified adenine).

In certain embodiments, modified guanine has structure (II):

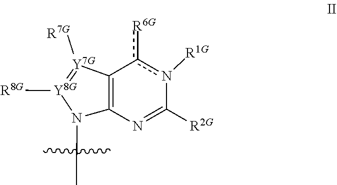

wherein: R^{2G} is N(R^{a})(R^{b}); R^{6G} is oxo and R^{1G} is H, or R^{6G} is selected from O—C₁-C₆ alkyl or S—C₁-C₆ alkyl and R^{1G} is absent; Y^{7G} is N and R^{7A} is absent or is C₁-C₆ alkyl; or Y^{7G} is C and R^{7G} is selected from H, C₁-C₆ alkyl, or CN(R^{a})(R^{b}); Y^{8G} is N and R^{8G} is absent, or Y^{8G} is C and R^{8G} is selected from H, a halogen, OH, C₁-C₆ alkyl, or substituted C₁-C₆ alkyl; R^{a} and R^{b} are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7G} is N; Y^{8G} is C, R^{8G} is H, R^{2G} is NH₂, and R^{6G} is ═O (unmodified guanosine).

In certain embodiments, modified thymine or modified uracil has structure (III):

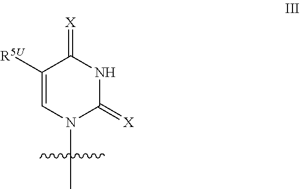

wherein: X is selected from O or S and R^{5U} is selected from H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl; wherein if each X is O, R^{5U} is not H or CH₃ (unmodified uracil and unmodified thymine, respectively).

In certain embodiments, modified cytosine has structure (IV):

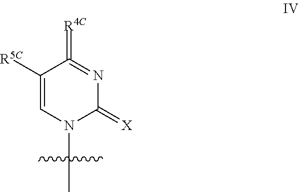

wherein: X is selected from O or S, R^{4C} is N(R^{a})(R^{b}); R^{5C} is selected from H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl; R^{a} and R are independently selected from H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where X is O, R^{4C} is NH₂ and R^{5C} is H (unmodified cytosine).

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 5-methylcytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo (particularly 5-bromo), 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Rogers et al., U.S. Pat. No. 5,134,066; Benner et al., U.S. Pat. No. 5,432,272; Matteucci et al., U.S. Pat. No. 5,502,177; Froehler et al., U.S. Pat. No. 5,594,121; and Cook et al., U.S. Pat. No. 5,681,941.

In certain embodiments, each nucleobase of a modified oligonucleotide of the invention is selected from A, G, C, T, U, and ^{m}C.

In certain embodiments, each nucleobase of a modified oligonucleotide of the invention is selected from A, G, T, and ^{m}C (i.e., unmodified purines and 5-methyl pyrimidines).

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include, but are not limited to, phosphodiesters, which contain a phosphodiester bond (“P═O”) (also referred to as unmodified or naturally occurring linkages), phosphotriesters, methylphosphonates, phosphoramidates, phosphorothioates (“P═S”), and phosphorodithioates (“HS-P═S”). Representative non-phosphorus containing internucleoside linking groups include, but are not limited to, methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO2021/030778, incorporated by reference herein. In certain embodiments, a modified internucleoside linkage comprises the formula:

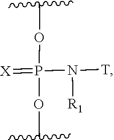

wherein independently for each such internucleoside linking group of a modified oligonucleotide:

X is selected from O or S;

R₁ is selected from H, C₁-C₆ alkyl, and substituted C₁-C₆ alkyl; and

T is selected from SO₂R₂, C(═O)R₃, and P(═O)R₄R₅, wherein:

R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;

R₃ is selected from an aryl, a substituted aryl, CH₃, N(CH₃)₂, OCH₃ and a conjugate group;

R₄ is selected from OCH₃, OH, C₁-C₆ alkyl, substituted C₁-C₆ alkyl and a conjugate group; and

R₅ is selected from OCH₃, OH, C₁-C₆ alkyl, and substituted C₁-C₆ alkyl.

In certain embodiments, a modified internucleoside linkage comprises a mesyl phosphoramidate linking group which has the formula:

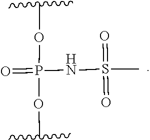 [SMILES: *OP(=O)(NS(C)(=O)=O)O*]

The mesyl phosphoramidate internucleoside linkage comprises a chiral center. In certain embodiments, modified oligonucleotides comprise (Rp) and/or (Sp) mesyl phosphoramidates, which are shown in the following formulas, respectively, wherein “B” indicates a nucleobase:

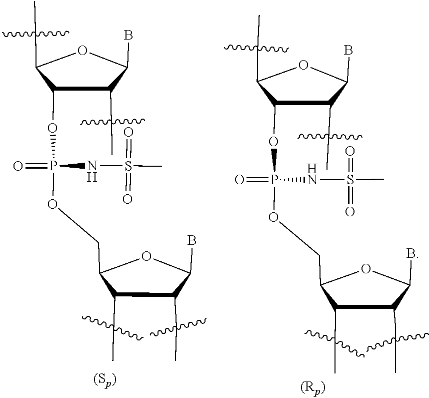

In certain embodiments, a phosphorothioate internucleoside linkage may comprise a chiral center. In certain embodiments, modified oligonucleotides comprising (Rp) and/or (Sp) phosphorothioates comprise one or more of the following formulas, respectively, wherein “B” indicates a nucleobase:

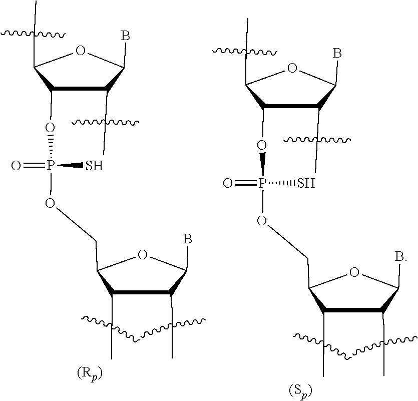

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising such internucleoside linkages in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each internucleoside linkage having a chiral center. Nonetheless, each individual internucleoside linkage having a chiral center of each individual oligonucleotide molecule has a defined stereoconfiguration. In certain embodiments, populations of modified oligonucleotides are enriched for modified oligonucleotides comprising one or more particular phosphorothioate and/or mesyl phosphoramidate internucleoside linkages, each independently in a particular, independently selected stereochemical configuration. In certain embodiments, the particular configuration of the particular phosphorothioate and/or mesyl phosphoramidate linkage is present in at least 65% of the molecules in the population. In certain embodiments, the particular configuration of the particular phosphorothioate and/or mesyl phosphoramidate linkage is present in at least 70% of the molecules in the population. In certain embodiments, the particular configuration of the particular phosphorothioate and/or mesyl phosphoramidate linkage is present in at least 80% of the molecules in the population. In certain embodiments, the particular configuration of the particular phosphorothioate and/or mesyl phosphoramidate linkage is present in at least 90% of the molecules in the population. In certain embodiments, the particular configuration of the particular phosphorothioate and/or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nucleic Acids Res. 42, 13456 (2014), and WO 2017/015555. In certain embodiments, a population of modified oligonucleotides is enriched for modified oligonucleotides having at least one indicated phosphorothioate and/or mesyl phosphoramidate in the (Sp) configuration. In certain embodiments, a population of modified oligonucleotides is enriched for modified oligonucleotides having at least one phosphorothioate and/or mesyl phosphoramidate in the (Rp) configuration. Unless otherwise indicated, internucleoside linkages having chiral centers of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

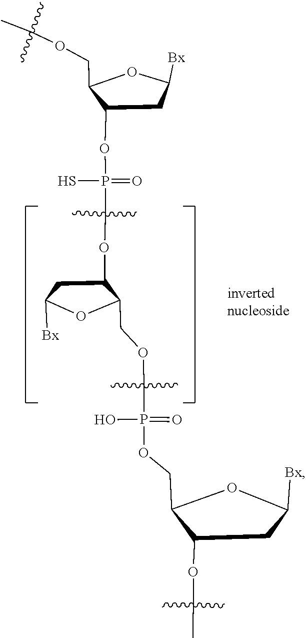

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, nucleic acids can be linked 2′ to 5′ rather than the standard 3′ to 5′ linkage. Such a linkage is illustrated below.

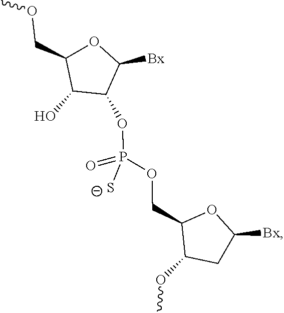

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise a deoxy region. In certain embodiments, each nucleoside of the deoxy region is a 2′-β-D-deoxynucleoside. In certain embodiments, the deoxy region consists of 5-12 linked nucleosides. In certain embodiments, the deoxy region consists of 6, 7, 8, 9, 10, or 6-10 linked nucleosides. In certain embodiments, at least one nucleoside within the deoxy region comprises a modified sugar moiety. In certain embodiments, exactly one nucleoside within the deoxy region comprises a modified sugar moiety. In certain embodiments, two or three nucleosides within the deoxy region comprise a modified sugar moiety.

In certain embodiments, the deoxy region is flanked on the 5′-side by a 5′-region consisting of linked 5′-region nucleosides and on the 3′-side by a 3′-region consisting of linked 3′-region nucleosides; wherein the 3′-most nucleoside of the 5′-region is a modified nucleoside and the 5′-most nucleoside of the 3′-region is a modified nucleoside. At least one nucleoside of the 5′-region comprises a modified sugar moiety; and at least one nucleoside of the 3′-region comprises a modified sugar moiety. The three regions (the 5′-region, the deoxy region, and the 3′-region) form a contiguous sequence of nucleosides. In certain embodiments, the sugar moiety of the 3′-most nucleoside of the 5′-region and the sugar moiety of the 5′-most nucleoside of the 3′-region each differ from the sugar moiety of the respective adjacent nucleoside of the deoxy region, thus defining the boundary between the 5′-region, the deoxy region, and the 3′-region. In certain embodiments, each nucleoside of the 5′-region and each nucleoside of the 3′-region comprises a modified sugar moiety. In certain embodiments, the nucleosides within the 5′-region comprise the same sugar modification. In certain embodiments, the nucleosides within the 5′-region comprise two or more different sugar modifications. In certain embodiments, the nucleosides within the 3′-region comprise the same sugar modification. In certain embodiments, the nucleosides within the 3′-region comprise two or more different sugar modifications.

In certain embodiments, the 5′-region and the 3′-region of a modified oligonucleotide each comprises 1-8 nucleosides. In certain embodiments, the 5′-region comprises 1-7 nucleosides. In certain embodiments, the 5′-region comprises 1-6 nucleosides. In certain embodiments, the 5′-region comprises 1, 2, 3, 4, 5, 6, 7, or 8 nucleosides. In certain embodiments, the 3′-region comprises 1-7 nucleosides. In certain embodiments, the 3′-region comprises 1-6 nucleosides. In certain embodiments, the 3′-region comprises 1, 2, 3, 4, 5, 6, 7, or 8 nucleosides.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-8 nucleosides. In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety. In certain embodiments, at least five nucleosides of each wing of a gapmer comprises a modified sugar moiety. In certain embodiments, at least six nucleosides of each wing of a gapmer comprises a modified sugar moiety. In certain embodiments, at least seven nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-β-D-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 5-8-5 gapmer consists of 5 linked nucleosides comprising a modified sugar moiety in the 5′-wing, 8 linked 2′-β-D-deoxynucleosides in the gap, and 5 linked nucleosides comprising a modified sugar moiety in the 3′-wing. A 5-8-5 mixed gapmer has at least two different modified sugar moieties in the 5′- and/or the 3′-wing, two different modified sugar moieties in the gap region, or a combination thereof.

In certain embodiments, modified oligonucleotides disclosed herein are modified by a specific sugar modification. In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers. In certain embodiments, modified oligonucleotides are 3-10-4 cEt gapmers. In certain embodiments, modified oligonucleotides are 4-10-3 cEt gapmers. In certain embodiments, modified oligonucleotides are 4-10-4 cEt gapmers. In certain embodiments, 5-10-5 cEt gapmers. In certain embodiments, modified oligonucleotides are 6-10-4 MOE gapmers.

In certain embodiments, modified oligonucleotides disclosed herein are modified by two or more sugar modifications. In certain embodiments, modified oligonucleotides are 3-10-3 mixed gapmers, wherein each nucleoside within the 5′ and the 3′ wings comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 3-10-4 mixed gapmers, wherein each nucleoside within the 5′ and the 3′ wings comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 3-10-5 mixed gapmers, wherein each nucleoside within the 5′ and the 3′ wings comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 4-9-4 mixed gapmers, wherein each nucleoside within the 5′ and the 3′ wings comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 5-10-5 mixed gapmers, wherein each nucleoside within the 5′ and the 3′ wings comprises a modified sugar moiety selected from a 2′-MOE sugar moiety and a 2′-cEt sugar moiety, and the gap nucleosides comprise 2′-β-D-deoxyribosyl sugar moieties. In certain embodiments, modified oligonucleotides are 6-10-4 mixed gapmers, wherein each nucleoside within the 5′ and the 3′ wings comprises a modified sugar moiety selected from a 2′-MOE sugar moiety and a 2′-cEt sugar moiety, and the gap nucleosides comprise 2′-β-D-deoxyribosyl sugar moieties.

In certain embodiments, modified oligonucleotides disclosed herein are modified by two or more sugar modifications within the gap region. In certain embodiments, modified oligonucleotides are cEt/MOE mixed gapmers, wherein each nucleoside within the 5′ and the 3′ wings comprises a 2′-cEt sugar moiety or a 2′-MOE sugar moiety and each nucleoside within the gap comprises a sugar moiety selected from a 2′-β-D-deoxyribosyl sugar moiety, a 2′-α-L-deoxyribosyl sugar moiety, and a 2′-OMe sugar moiety. In certain embodiments, modified oligonucleotides are cEt/MOE mixed gapmers, wherein each nucleoside within the 5′ and the 3′ wings comprises a 2′-cEt sugar moiety or a 2′-MOE sugar moiety and each nucleoside within the gap comprises a 2′-β-D-deoxyribosyl sugar moiety. In certain embodiments, modified oligonucleotides are 3-10-3 mixed gapmers, wherein each nucleoside within the 5′ and the 3′ wings comprises a 2′-cEt sugar moiety, and each nucleoside within the gap comprises a sugar moiety selected from a 2′-OMe sugar moiety or a 2′-β-D-deoxyribosyl sugar moiety. In certain embodiments, modified oligonucleotides are 5-10-5 mixed gapmers, wherein each nucleoside within the 5′ and the 3′ wings comprises a 2′-MOE sugar moiety, and each nucleoside within the gap comprises a sugar moiety selected from a 2′-β-D-deoxyxylosyl sugar moiety, a 2′-α-L-deoxyribosyl sugar moiety, and 2′-β-D-deoxyribosyl sugar moiety.

In certain embodiments, modified oligonucleotides have a sugar motif selected from 5′-kddddddddeeekekee-3′, 5′-kdddddddddeekekee-3′, 5′-kddddddddeeeeeeee-3′, 5′-kdddddddddeeeeeee-3′, 5′-kddddddddeeekekee-3′, 5′-kdddddddddeekekee-3′, 5′-kydddddddeeekekee-3′, 5′-kyddddddddeekekee-3′, 5′-kdyddddddeeekekee-3′, 5′-kdydddddddeekekee-3′, 5′-eddddddddeeekekee-3′, 5′-edddddddddeekekee-3′, 5′-eddddddddeeeeeeee-3′, 5′-edddddddddeeeeeee-3′, 5′-eedddddddddeekeke-3′, 5′-eeddddddddeeekeke-3′, 5′-eedddddddddeeeeee-3′, 5′-eeddddddddeeeeeee-3′, 5′-ekdddddddddeeeeee-3′, 5′-ekddddddddeeekeke-3′, 5′-ekddddddddeeeeeee-3′, 5′-ekyddddddddeekeke-3′, 5′-ekydddddddeeekeke-3′, 5′-ekdydddddddeekeke-3′, 5′-ekdddddddddeekeke-3′, 5′-ekddddddddeeekeke-3′, 5′-ekdyddddddeeekeke-3′, 5′-ekdddddddddeekeke-3′, 5′-ekddddddddeeekeke-3′, 5′-ekdddddddddeekeke-3′, 5′-ekddddddddeeekeke-3′, 5′-ekddddddddeeekeke-3′, 5′-ekdddddddddeekeke-3′, 5′-ekddddddddeeekek-3′, 5′-ekdddddddeeeekek-3′, 5′-eeddddddddeeekek-3′, 5′-eedddddddeeeekek-3′, 5′-ekddddddddeeeeee-3′, 5′-ekdddddddeeeeeee-3′, 5′-ekydddddddeeekek-3′, 5′-eeddddddddeeeeee-3′, 5′-eedddddddeeeeeee-3′, 5′-ekdyddddddeeekek-3′, 5′-ekddddddddeeekek-3′, 5′-ekdddddddeeeekek-3′, 5′-ekddddddddeeekek-3′, 5′-ekdddddddeeeekek-3′, 5′-ekddddddddeeekek-3′, 5′-ekdddddddeeeekek-3′, 5′-eeeeeedddddddkke-3′, 5′-eeeekkdddddddeee-3′, 5′-eeeekkdddddddkke-3′, 5′-eeeeeedddddddeee-3′, 5′-eeeekkdddddddkke-3′, 5′-ekddddddddeeekek-3′, 5′-ekddddddddeeekek-3′, 5′-kdddddddddkeekk-3′, 5′-edddddddddkeekk-3′, 5′-kdddddddddeeeee-3′, 5′-kyddddddddkeekk-3′, 5′-edddddddddeeeee-3′, 5′-kdydddddddkeekk-3′, 5′-kdddddddddkeekk-3′, 5′-eeeekkddd[aLd]dddkke-3′, 5′-ekddd[aLd]ddddeeekek-3′, 5′-ekddd[aLd]dddeeeekek-3′, 5′-kddd[aLd]dddddkeekk-3′, 5′-kddd[aLd]ddddeeekekee-3′, 5′-kddd[aLd]dddddeekekee-3′, 5′-ekddd[aLd]dddddeekeke-3′, and 5′-ekddd[aLd]ddddeeekeke-3′, wherein each “d” represents a 2′-β-D-deoxyribosyl sugar moiety, each “e” represents a 2′-MOE sugar moiety, each “k” represents a cEt sugar moiety, each “y” represents a 2′-O-methyl sugar moiety, and each “[aLd]” represents a 2′-α-L-deoxyribosyl sugar moiety. In certain embodiments, modified oligonucleotides have a sugar motif of 5′-kydddddddeeekekee-3′, 5′-kyddddddddeekekee-3′, 5′-kdyddddddeeekekee-3′, 5′-kdydddddddeekekee-3′, 5′-ekyddddddddeekeke-3′, 5′-ekydddddddeeekeke-3′, 5′-ekdydddddddeekeke-3′, 5′-ekdyddddddeeekeke-3′, 5′-ekydddddddeeekek-3′, 5′-ekdyddddddeeekek-3′, 5′-kyddddddddkeekk-3′, 5′-kdydddddddkeekk-3′, 5′-kddd[aLd]ddddeeekekee-3′, 5′-kddd[aLd]dddddeekekee-3′, 5′-ekddd[aLd]dddddeekeke-3′, 5′-ekddd[aLd]ddddeeekeke-3′, 5′-eeeekkddd[aLd]dddkke-3′, 5′-kddd[aLd]dddddkeekk-3′, 5′-ekddd[aLd]ddddeeekek-3′, and 5′-ekddd[aLd]dddeeeekek-3′, wherein each “e” represents a 2′-MOE sugar moiety, each “d” represents a 2′-β-D-deoxyribosyl sugar moiety, each “k” represents a cEt sugar moiety, each “y” represents a 2′-O-methyl sugar moiety, and each “[aLd]” represents a 2′-α-L-deoxyribosyl sugar moiety. In certain embodiments, modified oligonucleotides have a sugar motif of 5′-kddddddddeeekekee-3′, 5′-kdddddddddeekekee-3′, 5′-eddddddddeeekekee-3′, 5′-edddddddddeekekee-3′, 5′-kddddddddeeeeeeee-3′, 5′-kdddddddddeeeeeee-3′, 5′-eddddddddeeeeeeee-3′, 5′-edddddddddeeeeeee-3′, 5′-ekddddddddeeekeke-3′, 5′-ekdddddddddeekeke-3′, 5′-eedddddddddeekeke-3′, 5′-eeddddddddeeekeke-3′, 5′-ekdddddddddeeeeee-3′, 5′-ekddddddddeeeeeee-3′, 5′-eedddddddddeeeeee-3′, 5′-eeddddddddeeeeeee-3′, 5′-ekddddddddeeekek-3′, 5′-ekdddddddeeeekek-3′, 5′-eeddddddddeeekek-3′, 5′-eedddddddeeeekek-3′, 5′-ekddddddddeeeeee-3′, 5′-ekdddddddeeeeeee-3′, 5′-eeddddddddeeeeee-3′, 5′-eedddddddeeeeeee-3′, 5′-ekddddddddeeekek-3′, 5′-ekdddddddeeeekek-3′, 5′-ekddddddddeeekek-3′, 5′-ekdddddddeeeekek-3′, 5′-eeeeeedddddddkke-3′, 5′-eeeekkdddddddeee-3′, 5′-eeeekkdddddddkke-3′, 5′-eeeeeedddddddeee-3′, 5′-ekddddddddeeekek-3′, 5′-kdddddddddkeekk-3′, 5′-edddddddddkeekk-3′, 5′-kdddddddddeeeee-3′, and 5′-edddddddddeeeee-3′, wherein each “e” represents a 2′-MOE sugar moiety, each “k” represents a cEt sugar moiety, and each “d” represents a 2′-β-D-deoxyribosyl sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-β-D-deoxyribosyl sugar moiety. In certain embodiments, the modified nucleobase is selected from a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

In certain embodiments, the sugar motif of a modified oligonucleotide is a gapmer and the internucleoside linkages within the gap are all modified. In certain embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, or Rp motif. In certain embodiments, populations of modified oligonucleotides are enriched for modified oligonucleotides comprising such internucleoside linkage motifs.

In certain embodiments, modified oligonucleotides have an internucleoside linkage motif comprising one or more mesyl phosphoramidate linking groups. In certain embodiments, one or more phosphorothioate internucleoside linkages or one or more phosphodiester internucleoside linkages of the internucleoside linkage motifs herein is substituted with a mesyl phosphoramidate linking group.

In certain embodiments, modified oligonucleotides have an internucleoside linkage motif of 5′-sssssssssssoss-3′, 5′-sssssssssssooss-3′, 5′-sssoossssssssss-3′, 5′-sssssssssssoooss-3′, 5′-sssssssssssoosss-3′, 5′-ssssssssssssooss-3′, 5′-szzsssssssssss-3′, 5′-zsssssssssssss-3′, 5′-zssssssssssssz-3′, 5′-ssssssssssszss-3′, 5′-sssssssssssssss-3′, 5′-zzsssssssssssss-3′, 5′-zsssssssssssssz-3′, 5′-sssssszzsssssss-3′, 5′-zsssssssssssssz-3′, 5′-ssszzssssssssss-3′, 5′-sszzsssssssssss-3′, 5′-sssssszssssssss-3′, 5′-ssssszzssssssss-3′, 5′-ssssszzszssssss-3′, 5′-ssszsszssssssss-3′, 5′-ssszszzssssssss-3′, 5′-sszzsszssssssss-3′, 5′-sszzszzssssssss-3′, 5′-ssszszszsssssss-3′, 5′-sszszszssssssss-3′, 5′-ssssssssssszzss-3′, 5′-ssssssssssszzzss-3′, 5′-szzsssssssssosss-3′, 5′-zsssssssssssosss-3′, 5′-sszzsssssssssoss-3′, 5′-zzsssssssssssoss-3′, or 5′-zssssssssssssszz-3′, wherein each “s” represents a phosphorothioate internucleoside linkage, each “o” represents a phosphodiester internucleoside linkage, and each “z” represents a mesyl phosphoramidate internucleoside linkage. In certain embodiments, modified oligonucleotides have an internucleoside linkage motif of 5′-ssssssssssszzzss-3′, 5′-szzsssssssssosss-3′, 5′-zsssssssssssosss-3′, 5′-zssssssssssssszz-3′, 5′-sszzsssssssssoss-3′, 5′-zzsssssssssssoss-3′, 5′-zssssssssssssszz-3′, 5′-ssssssssssszzzss-3′, 5′-ssssssssssszzss-3′, 5′-sszzsssssssssss-3′, 5′-zzsssssssssssss-3′, 5′-zsssssssssssssz-3′, 5′-sssssszzsssssss-3′, 5′-ssszzssssssssss-3′, 5′-sssssszssssssss-3′, 5′-ssssszzssssssss-3′, 5′-ssssszzszssssss-3′, 5′-ssszsszssssssss-3′, 5′-ssszszzssssssss-3′, 5′-sszzsszssssssss-3′, 5′-sszzszzssssssss-3′, 5′-ssszszszsssssss-3′, 5′-sszszszssssssss-3′, 5′-ssssssssssszss-3′, 5′-szzsssssssssss-3′, 5′-zsssssssssssss-3′, or 5′-zssssssssssssz-3′, wherein each “s” represents a phosphorothioate internucleoside linkage, each “o” represents a phosphodiester internucleoside linkage, and each “z” represents a mesyl phosphoramidate internucleoside linkage. In certain embodiments, modified oligonucleotides have an internucleoside linkage motif of 5′-sssssssssssoooss-3′, 5′-sssssssssssoosss-3′, 5′-ssssssssssssooss-3′, 5′-sssssssssssooss-3′, 5′-sssssssssssssss-3′, 5′-sssoossssssssss-3′, or 5′-sssssssssssoss-3′, wherein each “s” represents a phosphorothioate internucleoside linkage, and each “o” represents a phosphodiester internucleoside linkage. In certain embodiments, modified oligonucleotides have an internucleoside linkage motif of 5′-sssssssssssoosss-3′, 5′-ssssssssssssooss-3′, 5′-sssssssssssooss-3′, 5′-sssssssssssssss-3′, or 5′-sssssssssssoss-3′, wherein each “s” represents a phosphorothioate internucleoside linkage, and each “o” represents a phosphodiester internucleoside linkage.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides (including modified oligonucleotides) consist of 16 linked nucleosides. In certain embodiments, oligonucleotides (including modified oligonucleotides) consist of 17 linked nucleosides. In certain embodiments, oligonucleotides (including modified oligonucleotides) consist of 18 linked nucleosides. In certain embodiments, oligonucleotides (including modified oligonucleotides) consist of 19 linked nucleosides. In certain embodiments, oligonucleotides (including modified oligonucleotides) consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for R-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (or portions thereof) have a nucleobase sequence that is complementary to a second oligonucleotide or an identified reference nucleic acid (or portion thereof), such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or identified reference nucleic acid (or portion thereof), such as a target nucleic acid.

### II. Certain Oligomeric Compounds

In certain embodiments, provided herein are oligomeric compounds, which comprises an oligonucleotide and optionally one or more conjugate groups and/or terminal groups. A conjugate group consists of a conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups are attached to either or both ends of an oligonucleotide (such conjugate groups are also terminal groups). In certain such embodiments, conjugate groups or terminal groups are attached at the 3′ and/or 5′-end of oligonucleotides.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can alter one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucleic Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucleic Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, a conjugate group consists of a lipid and a conjugate linker. In certain embodiments, a conjugate group is a phosphate linked lipid having the following structure:

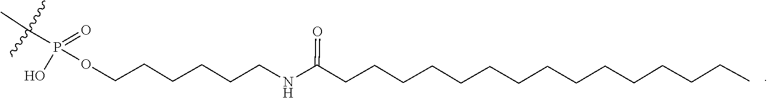

### 1. Conjugate Moieties

Conjugate moieties include, without limitation, intercalators, reporter molecules, polyamines, polyamides, peptides, carbohydrates (e.g., GalNAc), antibodies, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

In certain embodiments, conjugate moieties are selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, conjugate moieties are selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; and 10,550,188. In certain embodiments, a fragment of an anti-TfR1 antibody is F(ab′)₂, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; and U.S. Pat. No. 10,138,483.

In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, a terminal group comprises an inverted abasic sugar moiety. In certain embodiments, the inverted abasic sugar moiety may be further attached to a conjugate group. In certain embodiments, terminal groups comprise one or more 2′-linked nucleosides or sugar moieties. In certain such embodiments, the 2′-linked group is an abasic sugar moiety. Such terminal abasic sugar moieties can be attached to either or both ends of an oligonucleotide.

### III. Antisense Activity

In certain embodiments, oligomeric compounds are capable of hybridizing to a target nucleic acid, resulting in at least one antisense activity. In certain embodiments, an oligomeric compound forms an oligomeric duplex with a second oligomeric compound comprising a complementary nucleobase sequence. Such oligomeric compounds and oligomeric duplexes are antisense compounds. In certain embodiments, antisense compounds are deemed to have antisense activity when they reduce or inhibit the amount or activity of a target nucleic acid by 50% or more in the standard in vitro assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi agents. RNAi agents may be double-stranded (siRNA or dsRNAi) or single-stranded (ssRNAi).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### IV. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain embodiments, the target non-coding RNA is selected from: a long non-coding RNA, a short non-coding RNA, an intronic RNA molecule.

### A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nucleic Acids Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved.

In certain embodiments, a mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

B. HTT SNP rs7685686

In certain embodiments, oligomeric compounds described herein comprise or consist of an oligonucleotide comprising a region that is complementary to a target nucleic acid, wherein the target nucleic acid is HTT comprising SNP rs7685686. In each of the embodiments described above, the oligomeric compounds selectively target the HTT nucleic acid comprising SNP rs7685686. In certain embodiments, the oligomeric compounds described herein selectively target the HTT nucleic acid comprising SNP rs7685686 over wild-type HTT or over a non-target nucleic acid such as BMPR1. In certain embodiments, the difference in selectivity of the oligomeric compounds in targeting the HTT nucleic acid comprising SNP rs7685686 over wild-type HTT is at least 10-fold. In certain embodiments, the difference in selectivity of the oligomeric compounds in targeting the HTT nucleic acid comprising SNP rs7685686 over BMPR1 is at least 10-fold. In certain embodiments, the HTT nucleic acid comprising SNP rs7685686 has the sequence set forth in SEQ ID NO: 1 (GENBANK Accession No. NT_006081.18, truncated from nucleotides 1566000 to 1768000). In certain embodiments, contacting a cell with an oligomeric compound described herein that is complementary to SEQ ID NO: 1 selectively reduces the amount of HTT RNA comprising SNP rs7685686, and in certain embodiments reduces the amount of mHTT protein. In certain embodiments, the oligomeric compound consists of a modified oligonucleotide. In certain embodiments, the oligomeric compound consists of a modified oligonucleotide and a conjugate group. In certain embodiments, the amount of the wild-type HTT nucleic acid is not reduced by an oligomeric compound described herein. In certain embodiments, the amount of wild-type HTT protein is not reduced by an oligomeric compound described herein. In certain embodiments, the amount of a non-target nucleic acid such as BMPR1 is not reduced by an oligomeric compound described herein. In certain embodiments, the amount of BMPR1 RNA is not reduced by an oligomeric compound described herein.

In certain embodiments, contacting a cell with an oligomeric compound described herein that is complementary to SEQ ID NO: 1 selectively reduces the amount of HTT RNA comprising SNP rs7685686 in a cell. In certain embodiments, contacting a cell with an oligomeric compound described herein that is complementary to SEQ ID NO: 1 reduces the amount of mHTT protein in the cell. In certain embodiments, the cell is in vitro. In certain embodiments, the cell is in a subject. In certain embodiments, the oligomeric compound consists of a modified oligonucleotide. In certain embodiments, contacting a cell in a subject with an oligomeric compound described herein that is complementary to SEQ ID NO: 1 ameliorates one or more symptoms or hallmarks of Huntington's disease. In certain embodiments, the one or more symptoms or hallmarks include brain atrophy, muscle atrophy, nerve degeneration, uncontrolled movement, seizure, tremor, anxiety, memory lapse, or depression.

In certain embodiments, an oligomeric compound described herein that is complementary to SEQ ID NO: 1 is capable of reducing the amount of HTT RNA comprising SNP rs7685686 in vitro by at least 10%, at least 20%, at least 30%, at least 40%, at least 50%, at least 60%, at least 70%, at least 80%, or at least 90% when administered according to the standard in vitro assay. In certain embodiments, an oligomeric compound described herein that is complementary to SEQ ID NO: 1 is capable of reducing the amount of HTT RNA comprising SNP rs7685686 in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound described herein that is complementary to SEQ ID NO: 1 is capable of reducing the amount of mHTT protein in vitro by at least 10%, at least 20%, at least 30%, at least 40%, at least 50%, at least 60%, at least 70%, at least 80%, or at least 90% when administered according to the standard in vitro assay. In certain embodiments, an oligomeric compound described herein that is complementary to SEQ ID NO: 1 is capable of reducing the amount of mHTT protein in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound described herein that is complementary to SEQ ID NO: 1 is capable of reducing the amount of HTT RNA comprising SNP rs7685686 in the cell of a subject by at least 10%, at least 20%, at least 30%, at least 40%, at least 50%, at least 60%, at least 70%, at least 80%, or at least 90%. In certain embodiments, an oligomeric compound described herein that is complementary to SEQ ID NO: 1 is capable of reducing the amount of mHTT protein in the cell of a subject by at least 10%, at least 20%, at least 30%, at least 40%, at least 50%, at least 60%, at least 70%, at least 80%, or at least 90%.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissue are the cells and tissues that comprise the central nervous system (CNS). Such tissues include the brain and the spinal cord. In certain embodiments, the pharmacologically relevant tissues include cortex, substantia nigra, striatum including caudate and putamen, globus pallidus, thalamus, cerebellum, amygdala, midbrain, and brainstem. In certain embodiments, the cells are brain cells. In certain embodiments, the cells include neurons and glial cells. In certain embodiments, the glial cells include astrocytes, microglial cells, and oligodendrocytes.

### V. Certain Methods and Uses

Certain embodiments provided herein relate to methods of reducing or inhibiting the expression or activity of the HTT nucleic acid comprising SNP rs7685686, which can be useful for treating, preventing, or ameliorating Huntington's disease in a subject. In certain embodiments, a method comprises administering to a subject an oligomeric compound, a modified oligonucleotide, an oligomeric duplex, or an antisense agent, any of which having a nucleobase sequence complementary to a HTT nucleic acid region comprising SNP rs7685686. In certain embodiments, the modified oligonucleotide, oligomeric duplex, or antisense agent targets SNP rs7685686. In certain embodiments, the subject has or is at risk for developing Huntington's disease. In certain embodiments, the subject has Huntington's disease.

In certain embodiments, a method for treating Huntington's disease comprises administering to a subject an oligomeric compound, a modified oligonucleotide, an oligomeric duplex, or an antisense agent, any of which having a nucleobase sequence complementary to a HTT nucleic acid region comprising SNP rs7685686. In certain embodiments, the modified oligonucleotide, oligomeric duplex, or antisense agent targets SNP rs7685686. In certain embodiments, the subject has or is at risk for developing Huntington's disease. In certain embodiments, the subject has Huntington's disease. In certain embodiments, at least one symptom or hallmark of Huntington's disease is ameliorated. In certain embodiments, the at least one symptom or hallmark is brain atrophy, muscle atrophy, nerve degeneration, uncontrolled movement, seizure, tremor, anxiety, memory lapse, or depression.

In certain embodiments, a method of reducing expression of HTT nucleic acid comprising SNP rs7685686, for example HTT RNA comprising SNP rs7685686, or reducing the expression of mHTT protein in a cell comprises contacting the cell with an oligomeric compound, a modified oligonucleotide, an oligomeric duplex, or an antisense agent, any of which having a nucleobase sequence complementary to a HTT nucleic acid region comprising SNP rs7685686. In certain embodiments, the modified oligonucleotide, oligomeric duplex, or antisense agent targets SNP rs7685686. In certain embodiments, the subject has or is at risk for developing Huntington's disease. In certain embodiments, the subject has Huntington's disease. In certain embodiments, the cell is a brain cell. In certain embodiments, the cell is a neuron. In certain embodiments, the cell is a glial cell, e.g., an astrocyte, a microglial cell, or an oligodendrocyte. In certain embodiments, the cell is a human cell.

Certain embodiments are drawn to an oligomeric compound, a modified oligonucleotide, an oligomeric duplex, or an antisense agent, any of which having a nucleobase sequence complementary to a HTT nucleic acid region comprising SNP rs7685686 for use in treating Huntington's disease or for use in the manufacturing of a medicament for treating Huntington's disease.

In any of the methods or uses described herein, the oligomeric compound, the modified oligonucleotide, the oligomeric duplex, or the antisense agent can be any described herein.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid (aCSF). In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid.

In certain embodiments, where a modified oligonucleotide or an oligomeric compound is in solution comprising sodium (e.g., saline), the modified oligonucleotide or oligomeric compound may be partially or fully de-protonated and in association with sodium ions. However, the mass of the protons is nevertheless counted toward the weight of the dose, and the mass of the sodium ions is not counted toward the weight of the dose. Thus, for example, a dose, or dosage unit, of 10 mg of Compound No. 1625897 equals the number of fully protonated molecules that weighs 10 mg. This would be equivalent to 10.59 mg of solvent-free, sodium acetate-free, anhydrous sodiated Compound No. 1625897.

In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group may be included in calculating the dose of such oligomeric compound. If the conjugate group also has an acid, the conjugate group is likewise assumed to be fully protonated for the purpose of calculating dose.

### VII. Certain Oligomeric Compounds

In certain embodiments, an oligomeric compound disclosed herein comprises a modified oligonucleotide consisting of 12 to 30 linked nucleosides, wherein the nucleobase sequence of the modified oligonucleotide comprises at least 12, at least 13, at least 14, at least 15, at least 16, or at least 17 contiguous nucleobases of any of the nucleobase sequences of SEQ ID NOs: 14, 15, and 18-24. In certain such embodiments, the modified oligonucleotide comprises at least one modification selected from a modified sugar moiety and a modified internucleoside linkage. In certain embodiments, the oligomeric compound comprises a conjugate group. In certain embodiments, the oligomeric compound does not comprise a conjugate group. In certain embodiments, the oligomeric compound comprises a terminal group. In certain embodiments, the oligomeric compound does not comprise a terminal group.

In certain embodiments, an oligomeric compound disclosed herein comprises a modified oligonucleotide consisting of 12 to 30 linked nucleosides, wherein the nucleobase sequence of the modified oligonucleotide comprises at least 12, at least 13, at least 14, at least 15, at least 16, or 17 contiguous nucleobases of 5′-ATTGTCATCACCAGAAA-3′ (SEQ ID NO: 14). In certain embodiments, the modified oligonucleotide comprises at least one modification selected from a modified sugar moiety and a modified internucleoside linkage. In certain embodiments, the modified sugar moiety is a non-bicyclic modified sugar moiety selected from a 2′-MOE sugar moiety, a 2′-OMe sugar moiety, a cEt sugar moiety, and a 2′-α-L-deoxyribosyl sugar moiety. In certain embodiments, the modified internucleoside linkage is selected from a phosphorothioate internucleoside linkage and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, the oligomeric compound comprises a conjugate group. In certain embodiments, the oligomeric compound does not comprise a conjugate group. In certain embodiments, the oligomeric compound comprises a terminal group. In certain embodiments, the oligomeric compound does not comprise a terminal group.

In certain embodiments, the modified oligonucleotide has a nucleobase sequence of SEQ ID NO: 14. In certain embodiments, the modified oligonucleotide has a modified sugar motif of (from 5′ to 3′) ekddddddddeeekeke, wherein each “e” is a 2′-MOE sugar moiety, each “k” is a cEt sugar moiety, and each “d” is a 2′-β-D-deoxyribosyl sugar moiety. In certain embodiments, the modified oligonucleotide comprises a modified internucleoside linkage selected from a phosphorothioate internucleoside linkage and a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, the modified oligonucleotide has a nucleobase sequence of SEQ ID NO: 14. In certain embodiments, the modified oligonucleotide has a modified sugar motif of (from 5′ to 3′) ekddddddddeeekeke, wherein each “e” is a 2′-MOE sugar moiety, each “k” is a cEt sugar moiety, and each “d” is a 2′-β-D-deoxyribosyl sugar moiety. In certain embodiments, the modified oligonucleotide has a modified internucleoside linkage motif of (from 5′ to 3′) sszzsssssssssoss, wherein each “s” is a phosphorothioate internucleoside linkage, each “o” is a phosphodiester internucleoside linkage, and each “z” is a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, the modified oligonucleotide has a nucleobase sequence of SEQ ID NO: 14. In certain embodiments, the modified oligonucleotide has a modified sugar motif of (from 5′ to 3′) ekddddddddeeekeke, wherein each “e” is a 2′-MOE sugar moiety, each “k” is a cEt sugar moiety, and each “d” is a 2′-β-D-deoxyribosyl sugar moiety. In certain embodiments, the modified oligonucleotide has a modified internucleoside linkage motif of (from 5′ to 3′) ssssssssssszzzss, wherein each “s” is a phosphorothioate internucleoside linkage, and each “z” is a mesyl phosphoramidate internucleoside linkage. In certain embodiments, each nucleobase of the modified oligonucleotide is an unmodified nucleobase. In certain embodiments, at least one nucleobase of the modified oligonucleotide is a modified nucleobase. In certain embodiments, at least one cytosine of the modified oligonucleotide is a modified cytosine. In certain embodiments, each cytosine of the modified oligonucleotide is a 5-methylcytosine.

In certain embodiments, an oligomeric compound disclosed herein has the following chemical notation: N¹ₑₛTₖₛT_{dz}G_{dz}T_{ds}^{m}C_{ds}A_{ds}T_{ds}^{m}C_{ds}A_{ds}^{m}Cₑₛ^{m}CₑₛAₑₛGₖₒAₑₛAₖₛN²ₑ (SEQ ID NO: 27), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar is also absent,
e=a 2′-MOE sugar moiety,
k=a cEt sugar moiety,
d=a 2′-β-D-deoxyribosyl sugar moiety,
s=a phosphorothioate internucleoside linkage,
z=a mesyl phosphoramidate internucleoside linkage, and
o=a phosphodiester internucleoside linkage, and
   wherein the oligomeric compound optionally comprises a conjugate group. In certain embodiments, N¹ and N² are each independently an adenine nucleobase. In certain embodiments, N¹ and N² are each independently an unmodified adenine. In certain embodiments, N¹ and N² are each independently a modified adenine. In certain embodiments, N¹ and N² are each independently a hypoxanthine. In certain embodiments, N¹ and N² are each independently an abasic sugar moiety. In certain embodiments, N¹ and N² are each independently a terminal group. In certain embodiments, N¹ and N² are each independently absent. In certain embodiments, N¹ is an adenine nucleobase and N² is an unmodified adenine, a hypoxanthine, an abasic sugar moiety, a terminal group, or is absent. In certain embodiments, N¹ is an adenine nucleobase and N² is an unmodified adenine. In certain embodiments, N¹ is an adenine nucleobase and N² is a hypoxanthine. In certain embodiments, N¹ is an adenine nucleobase and N² is an abasic sugar moiety. In certain embodiments, N¹ is an adenine nucleobase and N² is a terminal group. In certain embodiments, N¹ is an adenine nucleobase and N² is absent. In certain embodiments, N¹ is an unmodified adenine and N² is an adenine nucleobase, an unmodified adenine, a hypoxanthine, an abasic sugar moiety, a terminal group, or is absent. In certain embodiments, N1 is an unmodified adenine and N² is an adenine nucleobase. In certain embodiments, N¹ is an unmodified adenine and N² is an unmodified adenine. In certain embodiments, N¹ is an unmodified adenine and N² is a hypoxanthine. In certain embodiments, N1 is an unmodified adenine and N² is an abasic sugar moiety. In certain embodiments, N¹ is an unmodified adenine and N² is a terminal group. In certain embodiments, N¹ is an unmodified adenine and N² is absent. In certain embodiments, N¹ is a hypoxanthine and N² is an adenine nucleobase, an unmodified adenine, a hypoxanthine, an abasic sugar moiety, a terminal group, or is absent. In certain embodiments, N¹ is a hypoxanthine and N² is an adenine nucleobase. In certain embodiments, N¹ is a hypoxanthine and N² is an unmodified adenine. In certain embodiments, N¹ is a hypoxanthine and N² is a hypoxanthine. In certain embodiments, N¹ is a hypoxanthine and N² is an abasic sugar moiety. In certain embodiments, N¹ is a hypoxanthine and N² is a terminal group. In certain embodiments, N¹ is a hypoxanthine and N² is absent. In certain embodiments, N¹ is an abasic sugar moiety and N² is an adenine nucleobase, an unmodified adenine, a hypoxanthine, an abasic sugar moiety, a terminal group, or is absent. In certain embodiments, N¹ is a terminal group and N² is an adenine nucleobase, an unmodified adenine, a hypoxanthine, an abasic sugar moiety, a terminal group, or is absent. In certain embodiments, N¹ is absent and N² is an adenine nucleobase, an unmodified adenine, a hypoxanthine, an abasic sugar moiety, a terminal group, or is absent. In certain embodiments, N¹ is absent and N² is absent.

In certain embodiments, an oligomeric compound disclosed herein has the following chemical notation: N¹ₑₛTₖₛT_{ds}G_{ds}T_{ds}^{m}C_{ds}A_{ds}T_{ds}^{m}C_{ds}A_{ds}^{m}Cₑₛ^{m}C_{ez}A_{ez}G_{kz}AₑₛAₖₛN²ₑ (SEQ ID NO: 28), wherein:
A=an adenine nucleobase,
^{m}C=a 5-methylcytosine nucleobase,
G=a guanine nucleobase,
T=a thymine nucleobase,
N¹=an adenine nucleobase, a modified adenine, a hypoxanthine, an abasic sugar moiety, a terminal group, or is absent, wherein when N¹ is absent its sugar and internucleoside linkage are also absent,
N²=an adenine nucleobase, a modified adenine, a hypoxanthine, an abasic sugar moiety, a terminal group, or is absent, wherein when N² is absent its sugar is also absent,
e=a 2′-MOE sugar moiety,
k=a cEt sugar moiety,
d=a 2′-β-D-deoxyribosyl sugar moiety,
s=a phosphorothioate internucleoside linkage, and
z=a mesyl phosphoramidate internucleoside linkage, and

wherein the oligomeric compound optionally comprises a conjugate group. In certain embodiments, N¹ and N² are each independently an adenine nucleobase. In certain embodiments, N¹ and N² are each independently an unmodified adenine. In certain embodiments, N¹ and N² are each independently a modified adenine. In certain embodiments, N¹ and N² are each independently a hypoxanthine. In certain embodiments, N¹ and N² are each independently an abasic sugar moiety. In certain embodiments, N¹ and N² are each independently a terminal group. In certain embodiments, N¹ and N² are each independently absent. In certain embodiments, N¹ is an adenine nucleobase and N² is an unmodified adenine, a hypoxanthine, an abasic sugar moiety, a terminal group, or is absent. In certain embodiments, N¹ is an adenine nucleobase and N² is an unmodified adenine. In certain embodiments, N¹ is an adenine nucleobase and N² is a hypoxanthine. In certain embodiments, N¹ is an adenine nucleobase and N² is an abasic sugar moiety. In certain embodiments, N¹ is an adenine nucleobase and N² is a terminal group. In certain embodiments, N¹ is an adenine nucleobase and N² is absent. In certain embodiments, N1 is an unmodified adenine and N² is an adenine nucleobase, an unmodified adenine, a hypoxanthine, an abasic sugar moiety, a terminal group, or is absent. In certain embodiments, N¹ is an unmodified adenine and N² is an adenine nucleobase. In certain embodiments, N¹ is an unmodified adenine and N² is an unmodified adenine. In certain embodiments, N¹ is an unmodified adenine and N² is a hypoxanthine. In certain embodiments, N¹ is an unmodified adenine and N² is an abasic sugar moiety. In certain embodiments, N¹ is an unmodified adenine and N² is a terminal group. In certain embodiments, N¹ is an unmodified adenine and N² is absent. In certain embodiments, N¹ is a hypoxanthine and N² is an adenine nucleobase, an unmodified adenine, a hypoxanthine, an abasic sugar moiety, a terminal group, or is absent. In certain embodiments, N¹ is a hypoxanthine and N² is an adenine nucleobase. In certain embodiments, N¹ is a hypoxanthine and N² is an unmodified adenine. In certain embodiments, N¹ is a hypoxanthine and N² is a hypoxanthine. In certain embodiments, N¹ is a hypoxanthine and N² is an abasic sugar moiety. In certain embodiments, N¹ is a hypoxanthine and N² is a terminal group. In certain embodiments, N¹ is a hypoxanthine and N² is absent. In certain embodiments, N¹ is an abasic sugar moiety and N² is an adenine nucleobase, an unmodified adenine, a hypoxanthine, an abasic sugar moiety, a terminal group, or is absent. In certain embodiments, N¹ is a terminal group and N² is an adenine nucleobase, an unmodified adenine, a hypoxanthine, an abasic sugar moiety, a terminal group, or is absent. In certain embodiments, N¹ is absent and N² is an adenine nucleobase, an unmodified adenine, a hypoxanthine, an abasic sugar moiety, a terminal group, or is absent. In certain embodiments, N¹ is absent and N² is absent.

### VIII. Certain Compositions

### 1. Compound No. 1625961

Compound No. 1625961 is characterized as a mixed cEt/MOE gapmer of linked nucleosides having a nucleobase sequence (from 5′ to 3′) of ATTGTCATCACCAGAAA (SEQ ID NO: 14), wherein each of nucleosides 1, 11-13, 15, and 17 (from 5′ to 3′) are 2′-MOE nucleosides, wherein each of nucleosides 2, 14, and 16 are cEt nucleosides, wherein each of nucleosides 3-10 are 2′-β-D-deoxynucleosides, wherein the internucleoside linkages between nucleosides 3 to 4 and 4 to 5 are mesyl phosphoramidate internucleoside linkages, wherein the internucleoside linkages between nucleosides 1 to 2, 2 to 3, 5 to 6, 6 to 7, 7 to 8, 8 to 9, 9 to 10, 10 to 11, 11 to 12, 12 to 13, 13 to 14, 15 to 16, and 16 to 17 are phosphorothioate internucleoside linkages, wherein the internucleoside linkage between nucleosides 14 to 15 is a phosphodiester internucleoside linkage, and wherein each cytosine is a 5-methylcytosine.

Compound No. 1625961 is represented by the following chemical notation: AₑₛTₖₛT_{dz}G_{dz}T_{ds}^{m}C_{ds}A_{ds}T_{ds}^{m}C_{ds}A_{ds}^{m}Cₑₛ^{m}CₑₛAₑₛGₖₒAₑₛAₖₛAₑ (SEQ ID NO: 25), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
z=a mesyl phosphoramidate internucleoside linkage, and
o=a phosphodiester internucleoside linkage; and the compound does not include a conjugate group or a terminal group.

Compound No. 1625961 is represented by the following chemical structure:

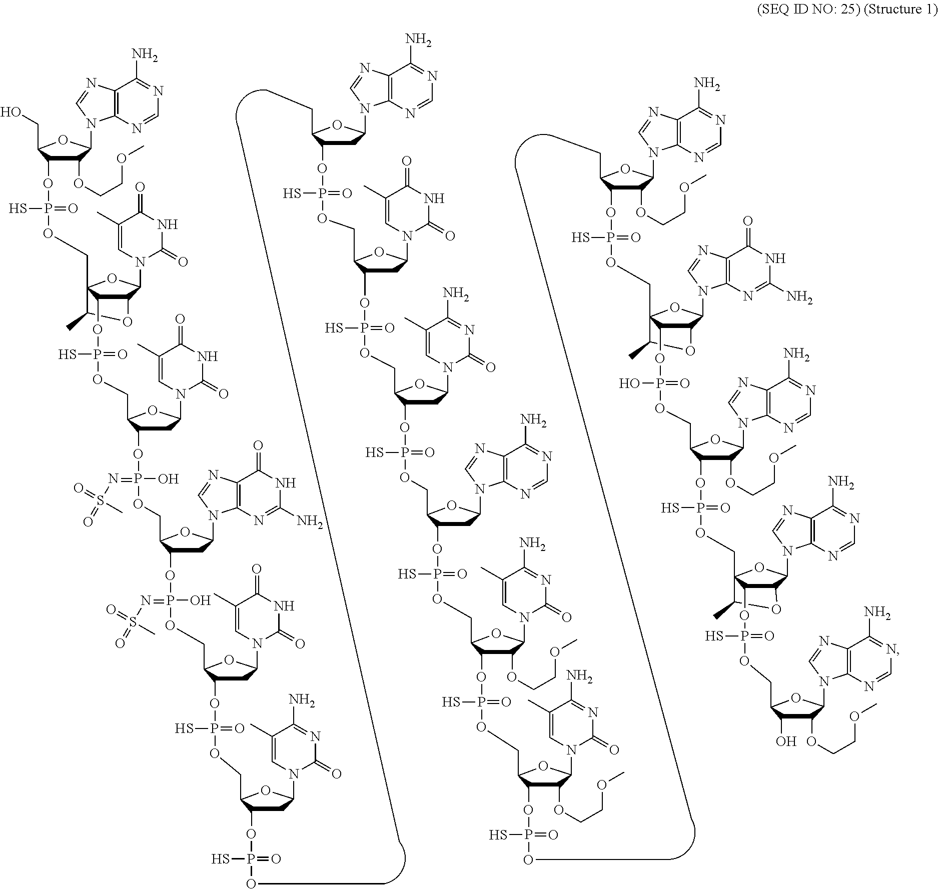

or a pharmaceutically thereof. The pharmaceutically acceptable salt of Compound No. 1625961 comprises one or more cations selected from sodium, potassium, calcium, and magnesium.

The sodium salt of Compound No. 1625961 is represented by the following chemical structure:

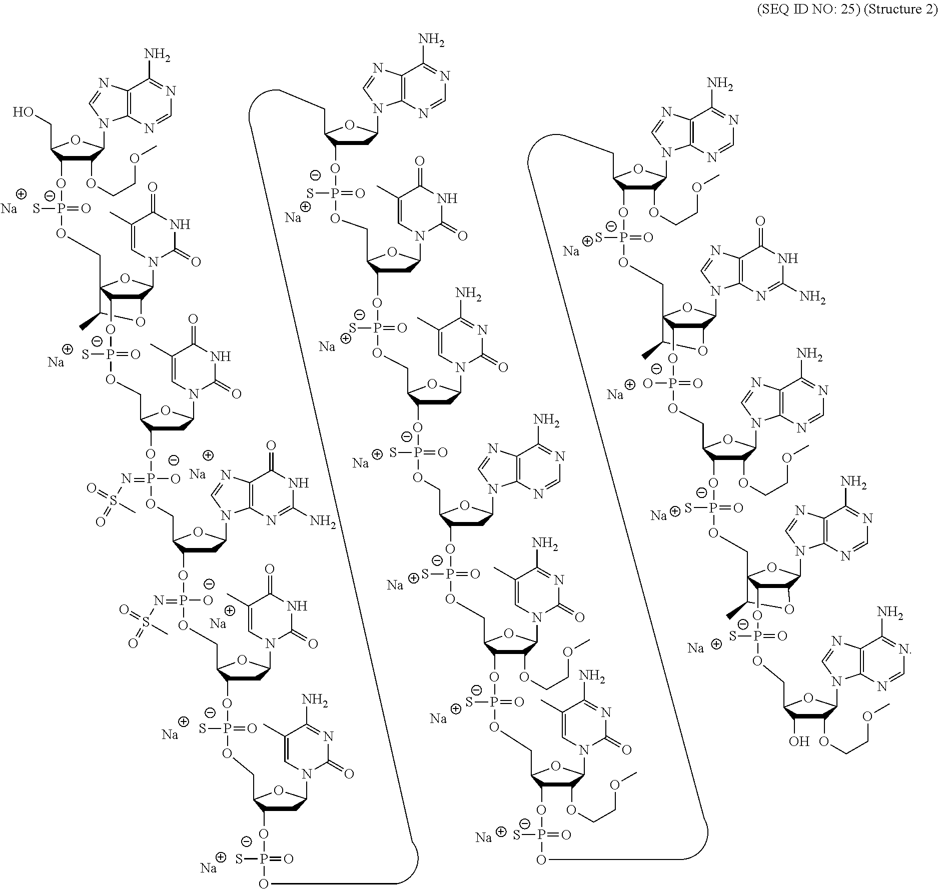

### 2. Compound No. 1637229

Compound No. 1637229 is characterized as a mixed cEt/MOE gapmer of linked nucleosides having a nucleobase sequence (from 5′ to 3′) of ATTGTCATCACCAGAAA (SEQ ID NO: 14), wherein each of nucleosides 1, 11-13, 15, and 17 (from 5′ to 3′) are 2′-MOE nucleosides, wherein each of nucleosides 2, 14, and 16 are cEt nucleosides, wherein each of nucleosides 3-10 are 2′-β-D-deoxynucleosides, wherein the internucleoside linkages between nucleosides 12 to 13, 13 to 14, and 14 to 15 are mesyl phosphoramidate internucleoside linkages, wherein the internucleoside linkages between nucleosides 1 to 2, 2 to 3, 3 to 4, 4 to 5, 5 to 6, 6 to 7, 7 to 8, 8 to 9, 9 to 10, 10 to 11, 11 to 12, 15 to 16, and 16 to 17 are phosphorothioate internucleoside linkages, and wherein each cytosine is a 5-methylcytosine.

Compound No. 1637229 is represented by the following chemical notation: AₑₛTₖₛT_{ds}G_{ds}T_{ds}^{m}C_{ds}A_{ds}T_{ds}^{m}C_{ds}A_{ds}^{m}Cₑₛ^{m}C_{ez}A_{ez}G_{kz}AₑₛAₖₛAₑ (SEQ ID NO: 26), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage, and
z=a mesyl phosphoramidate internucleoside linkage; and the compound does not include a conjugate group or a terminal group.

Compound No. 1637229 is represented by the following chemical structure:

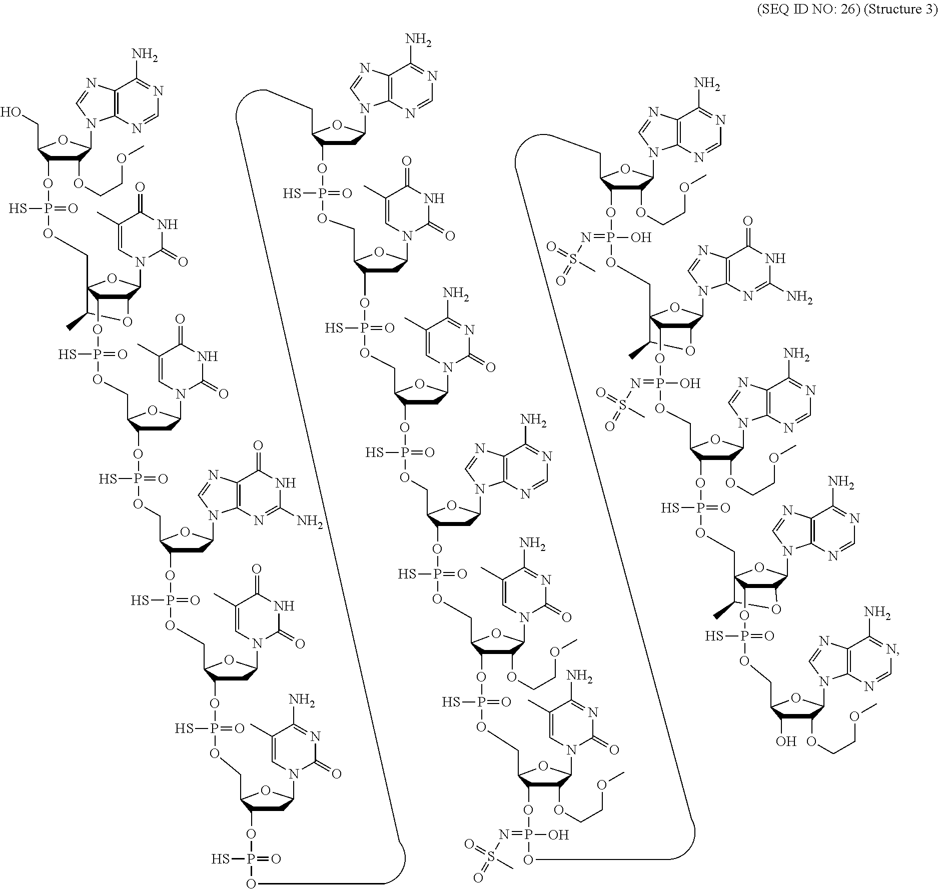

or a pharmaceutically acceptable salt thereof. The pharmaceutically acceptable salt of Compound No. 1637229 comprises one or more cations selected from sodium, potassium, calcium, and magnesium.

The sodium salt of Compound No. 1637229 is represented by the following chemical structure:

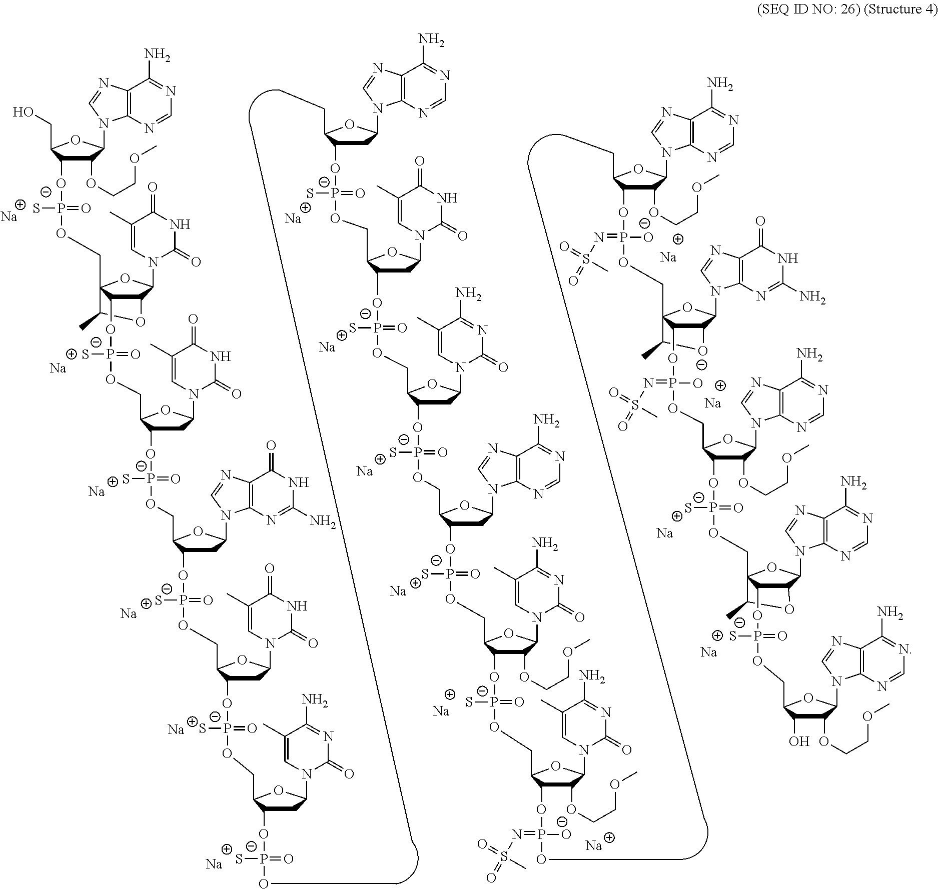

### IX. Certain Comparator Compositions

In certain embodiments, Compound No. 623208 is a comparator compound and is previously described in WO 2014/121287. Compound No. 623208 consists of the nucleobase sequence (from 5′ to 3′): TTGTCATCACCAGAA, designated herein as SEQ ID NO: 15. The sugar motif for Compound No. 623208 is (from 5′ to 3′): kdddddddddkeekk; wherein each “k” represents a cEt sugar moiety, each “e” represents a 2′-MOE sugar moiety, and each “d” represents a 2′-β-D-deoxyribosyl sugar moiety. The internucleoside linkage motif for Compound No. 623208 is (from 5′ to 3′): sssssssssssoss; wherein each “s” represents a phosphorothioate internucleoside linkage and each “o” represents a phosphodiester internucleoside linkage. Each cytosine nucleobase in Compound No. 623208 is a 5-methylcytosine.

Compound No. 623236 is a comparator compound and is previously described in WO 2014/121287. Compound No. 623236 consists of the nucleobase sequence (from 5′ to 3′): ATTGTCATCACCAGAAA, designated herein as SEQ ID NO: 14. The sugar motif for Compound No. 623236 is (from 5′ to 3′): ekddddddddeeekeke; wherein each “k” represents a cEt sugar moiety, each “e” represents a 2′-MOE sugar moiety, and each “d” represents a 2′-β-D-deoxyribosyl sugar moiety. The internucleoside linkage motif for Compound No. 623236 is (from 5′ to 3′): ssssssssssssooss; wherein each “s” represents a phosphorothioate internucleoside linkage and each “o” represents a phosphodiester internucleoside linkage. Each cytosine nucleobase in Compound No. 623236 is a 5-methylcytosine.

Compound No. 443139 is a comparator compound and is previously described in WO 2011/032045. Compound No. 443139 consists of the nucleobase sequence (from 5′ to 3′): CTCAGTAACATTGACACCAC, designated herein as SEQ ID NO: 16. The sugar motif for Compound No. 443139 is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for Compound No. 443139 is (from 5′ to 3′): sooosssssssssssooos; wherein each “s” represents a phosphorothioate internucleoside linkage and each “o” represents a phosphodiester internucleoside linkage. Each cytosine nucleobase in Compound No. 443139 is a 5-methylcytosine.

In certain embodiments, compounds described herein are superior relative to compounds described in WO 2014/121287 and WO 2011/032045, because the compounds described herein demonstrate one or more improved properties, such as tolerability, selectivity, and duration of action.

For example, Compound No. 1625961 and Compound No. 1637229 are each more tolerable in vivo compared to Compound No. 623208 in the assay shown in Example 4. In particular, rats treated with Compound No. 1625961 have a functional observational battery (FOB) score of 2.33 and rats treated with Compound No. 1637229 have a FOB score of 3. In comparison, rats treated with Compound No. 623208 have a FOB score range of 3.75-4. Therefore, Compound No. 1625961 and Compound No. 1637229 are each more tolerable compared to Compound No. 623208 in this assay.

For example, Compound No. 1625961 and Compound No. 1637229 each demonstrated a longer duration of action in vivo as compared to Compound No. 443139 in the assay shown in Example 7. In particular, Compound No. 1625961 and Compound No. 1637229 achieved a 35% and 22% reduction of human HTT RNA in the cortex, respectively, at day 141 post-dose. In comparison, Compound No. 443139 achieved a 17% reduction of human HTT RNA in the cortex at day 84 post-dose. Therefore, Compound No. 1625961 and Compound No. 1637229 each exhibited a longer duration of action compared to Compound No. 443139 in this assay.

For example, Compound No. 1625961 and Compound No. 1637229 each demonstrated more selectivity for SNP rs7685686 than HTT wild-type allele in vitro as compared to Compound No. 623236 in the assay shown in Example 9. In particular, Compound No. 1625961 and Compound No. 1637229 have selectivity values of >73 and >46, respectively. In comparison, Compound No. 623236 has a selectivity value of >43. Therefore, Compound No. 1625961 and Compound No. 1637229 are more selective toward SNP rs7685686 than HTT wild-type allele compared to Compound No. 623236 in this assay.

### NONLIMITING DISCLOSURE AND INCORPORATION BY REFERENCE

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

The sequence listing accompanying this filing identifies each nucleic acid sequence as either “RNA” or “DNA” as required; however, one of skill in the art will readily appreciate that designation of “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (5-methyl uracil)) in place of an uracil of RNA); and certain nucleic acid compounds described herein comprise one or more nucleosides comprising modified sugar moieties having 2′-substituent(s) that are neither OH nor H. One of skill in the art will readily appreciate that labeling such nucleic acid compounds “RNA” or “DNA” does not alter or limit the description of such nucleic acid compounds.

Herein, the description of compounds as having “the nucleobase sequence of” a SEQ ID NO. describes only the nucleobase sequence. Accordingly, absent additional description, such description of compounds by reference to a nucleobase sequence of a SEQ ID NO. does not limit sugar or internucleoside linkage modifications or presence or absence of additional substituents such as a conjugate group. Further, absent additional description, the nucleobases of a compound “having the nucleobase sequence of” a SEQ ID NO. include such compounds having modified forms of the identified nucleobases as described herein.

Herein, the description of compounds by chemical notation (subscripts and/or superscripts to indicate chemical modifications) without reference to a specific Compound No. include only each noted modification, but may include additional substituents, such as a conjugate group, unless otherwise indicated. For example, the chemical notation of “AₑₛTₖₒ^{m}C_{ez}G_{ds}C_{d}” indicates a compound wherein the first nucleoside comprises a 2′-MOE sugar moiety (indicated by the “e” subscript) and an unmodified adenine nucleobase linked to the second nucleoside via a phosphorothioate linkage (indicated by the “s” subscript); the second nucleoside comprises a cEt sugar moiety (indicated by the “k” subscript) and an unmodified thymine nucleobase linked to the third nucleoside via a phosphodiester linkage (indicated by the “o” subscript); the third nucleoside comprises a 2′-MOE sugar moiety and a 5-methyl modified cytosine nucleobase (indicated by the “m” superscript) linked to the fourth nucleoside via a mesylphosphoramidate linkage (indicated by the “z” subscript); the fourth nucleoside comprises a 2′-β-D-deoxyribosyl sugar moiety (indicated by the “d” subscript) and an unmodified guanine nucleobase linked to the fifth nucleoside with a phosphorothioate linkage; and the fifth nucleoside comprises a 2′-β-D-deoxyribosyl sugar moiety and an unmodified cytosine nucleobase; and the compound may include additional substituents, such as a conjugate group.

Herein, where a specific compound (e.g., with reference to a Compound No.) is described (as in the examples) by chemical notation, each nucleobase, sugar, and internucleoside linkage of such specific compound is modified only as indicated. Accordingly, in the context of a description of a specific compound having a particular Compound No., “AₑₛTₖₒ^{m}C_{ez}G_{ds}C_{d}” indicates a compound wherein the first nucleoside comprises a 2′-MOE sugar moiety (indicated by the “e” subscript) and an unmodified adenine nucleobase linked to the second nucleoside via a phosphorothioate linkage (indicated by the “s” subscript); the second nucleoside comprises a cEt sugar moiety (indicated by the “k” subscript) and an unmodified thymine nucleobase linked to the third nucleoside via a phosphodiester linkage (indicated by the “o” subscript); the third nucleoside comprises a 2′-MOE sugar moiety and a 5-methyl modified cytosine nucleobase (indicated by the “m” superscript) linked to the fourth nucleoside via a mesyl phosphoramidate linkage (indicated by the “z” subscript); the fourth nucleoside comprises a 2′-β-D-deoxyribosyl sugar moiety (indicated by the “d” subscript) and an unmodified guanine nucleobase linked to the fifth nucleoside with a phosphorothioate linkage; and the fifth nucleoside comprises a 2′-β-D-deoxyribosyl sugar moiety and an unmodified cytosine nucleobase; and the compound does not include additional substituents.

Herein, sugar, internucleoside linkage, and nucleobase modifications may be indicated within a nucleotide or nucleobase sequence (e.g., by superscript or subscript, as shown above) or may be indicated in text accompanying a sequence (e.g., in separate text that appears within or above or below a table of compounds).

Where a specific compound is described herein by way of a drawn chemical structure, each nucleobase, sugar, and internucleoside linkage of such a specific compound includes only the modifications indicated in the drawn chemical structure. One of skill will appreciate, however, that drawn compounds may exist in equilibrium between tautomeric forms and/or as salts in equilibrium with protonated or ionic forms. Drawn structures are intended to capture all such forms of such compounds.

While effort has been made to accurately describe compounds in the accompanying sequence listing, should there be any discrepancies between a description in this specification and in the accompanying sequence listing, the description in the specification and not in the sequence listing is the accurate description.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of H, ¹³C or ¹⁴C in place of ¹²C, ¹⁵N in place of ¹⁴N, ¹⁷O or ¹⁸O in place of ¹⁶O, and ³³S, ³⁴S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments.

### Example 1: Design of Modified Oligonucleotides Complementary to and Selective for Human HTT SNP rs7685686

Modified oligonucleotides complementary to a human HTT RNA were designed.

The sugar motifs for the modified oligonucleotides are presented in the column labeled “Sugar Motif (5′ to 3′)” in the table below, wherein each “k” represents a cEt sugar moiety, each “y” represents a 2′-OMe sugar moiety, each “e” represents a 2′-MOE sugar moiety, each [aLd] represents a 2′-alpha-L-deoxyribosyl sugar moiety, and each “d” represents a 2′-β-D-deoxyribosyl sugar moiety. The internucleoside linkage motifs for the modified oligonucleotides are presented in the column labeled “Internucleoside Linkages (5′ to 3′)” in the table below, wherein each “s” represents a phosphorothioate internucleoside linkage, each “z” represents a mesyl phosphoramidate internucleoside linkage, and each “o” represents a phosphodiester internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (GENBANK Accession No. NT_006081.18, truncated from nucleotides 1566000 to 1768000).

Compound No. 387916 has been previously disclosed in WO/2007/089611. Compound Nos. 623205, 623206, 623235, 623236, 623208, 623242, 623243, and 572772 have been previously disclosed in WO/2014/121287.

TABLE 1
Modified Oligonucleotides complementary to human HTT

| | | | | SEQ | SEQ | |
|---|---|---|---|---|---|---|
| | | | | ID | ID | |
| | | | Inter- | No: | No: | |
| | | | nucleoside | 1 | 1 | SEQ |
| Compound | Sequence | Sugar Motif | linkages | Start | Stop | ID |
| No. | (5′ to 3′) | (5′ to 3′) | (5′ to 3′) | Site | Site | NO. |
| 387916 | TCTCTATTGC | eeeeeddddd | ssssssssss | 145466 | 145485 | 17 |
| | ACATTCCAAG | dddddeeeee | ssssss | | | |
| | | | sss | | | |
| 623205 | TTGTCATCAC | kddddddddd | ssssssssss | 146782 | 146798 | 18 |
| | CAGAAAA | eekekee | soosss | | | |
| 623206 | TTGTCATCAC | kdddddddde | ssssssssss | 146782 | 146798 | 18 |
| | CAGAAAA | eekekee | soosss | | | |
| 1625897 | TTGTCATCAC | kdddddddde | ssssssssss | 146782 | 146798 | 18 |
| | CAGAAAA | eekekee | soooss | | | |
| 1625898 | TTGTCATCAC | kddddddddd | ssssssssss | 146782 | 146798 | 18 |
| | CAGAAAA | eekekee | soooss | | | |
| 1625904 | TTGTCATCAC | kdddddddde | ssssssssss | 146782 | 146798 | 18 |
| | CAGAAAA | eekekee | szzzss | | | |
| 1625905 | TTGTCATCAC | kddddddddd | ssssssssss | 146782 | 146798 | 18 |
| | CAGAAAA | eekekee | szzzss | | | |
| 1625914 | TTGTCATCAC | edddddddde | ssssssssss | 146782 | 146798 | 18 |
| | CAGAAAA | eekekee | soosss | | | |
| 1625915 | TTGTCATCAC | eddddddddd | ssssssssss | 146782 | 146798 | 18 |
| | CAGAAAA | eekekee | soosss | | | |
| 1625922 | TTGTCATCAC | kdddddddde | ssssssssss | 146782 | 146798 | 18 |
| | CAGAAAA | eeeeeee | soosss | | | |
| 1625923 | TTGTCATCAC | kddddddddd | ssssssssss | 146782 | 146798 | 18 |
| | CAGAAAA | eeeeeee | soosss | | | |
| 1625929 | TUGTCATCAC | kyddddddde | ssssssssss | 146782 | 146798 | 19 |
| | CAGAAAA | eekekee | soosss | | | |
| 1625930 | TUGTCATCAC | kydddddddd | ssssssssss | 146782 | 146798 | 19 |
| | CAGAAAA | eekekee | soosss | | | |
| 1625935 | TTGTCATCAC | kddd[aLd] | ssssssssss | 146782 | 146798 | 18 |
| | CAGAAAA | ddddeeekek | soosss | | | |
| | | ee | | | | |
| 1625937 | TTGTCATCAC | kddd[aLd] | ssssssssss | 146782 | 146798 | 18 |
| | CAGAAAA | dddddeekek | soosss | | | |
| | | ee | | | | |
| 1625940 | TTGTCATCAC | edddddddde | ssssssssss | 146782 | 146798 | 18 |
| | CAGAAAA | eeeeeee | soosss | | | |
| 1625943 | TTGTCATCAC | eddddddddd | ssssssssss | 146782 | 146798 | 18 |
| | CAGAAAA | eeeeeee | soosss | | | |
| 1625951 | TTGTCATCAC | kdydddddde | ssssssssss | 146782 | 146798 | 18 |
| | CAGAAAA | eekekee | soosss | | | |
| 1625953 | TTGTCATCAC | kdyddddddd | ssssssssss | 146782 | 146798 | 18 |
| | CAGAAAA | eekekee | soosss | | | |
| 1625957 | TTGTCATCAC | kdddddddde | szzsssssss | 146782 | 146798 | 18 |
| | CAGAAAA | eekekee | ssosss | | | |
| 1625958 | TTGTCATCAC | kddddddddd | szzsssssss | 146782 | 146798 | 18 |
| | CAGAAAA | eekekee | ssosss | | | |
| 1625973 | TTGTCATCAC | kdddddddde | zsssssssss | 146782 | 146798 | 18 |
| | CAGAAAA | eekekee | ssosss | | | |
| 1625974 | TTGTCATCAC | kddddddddd | zsssssssss | 146782 | 146798 | 18 |
| | CAGAAAA | eekekee | ssosss | | | |
| 1625982 | TTGTCATCAC | kdddddddde | zsssssssss | 146782 | 146798 | 18 |
| | CAGAAAA | eekekee | sssszz | | | |
| 1625983 | TTGTCATCAC | kddddddddd | zsssssssss | 146782 | 146798 | 18 |
| | CAGAAAA | eekekee | sssszz | | | |
| 623235 | ATTGTCATCA | ekdddddddd | ssssssssss | 146783 | 146799 | 14 |
| | CCAGAAA | deekeke | ssooss | | | |
| 623236 | ATTGTCATCA | ekdddddddd | ssssssssss | 146783 | 146799 | 14 |
| | CCAGAAA | eeekeke | ssooss | | | |
| 1625899 | ATTGTCATCA | ekdddddddd | ssssssssss | 146783 | 146799 | 14 |
| | CCAGAAA | eeekeke | soooss | | | |
| 1625917 | ATTGTCATCA | eedddddddd | ssssssssss | 146783 | 146799 | 14 |
| | CCAGAAA | deekeke | ssooss | | | |
| 1625918 | ATTGTCATCA | eedddddddd | ssssssssss | 146783 | 146799 | 14 |
| | CCAGAAA | eeekeke | ssooss | | | |
| 1625925 | ATTGTCATCA | ekdddddddd | ssssssssss | 146783 | 146799 | 14 |
| | CCAGAAA | deeeeee | ssooss | | | |
| 1625926 | ATTGTCATCA | ekdddddddd | ssssssssss | 146783 | 146799 | 14 |
| | CCAGAAA | eeeeeee | ssooss | | | |
| 1625932 | ATUGTCATCA | ekyddddddd | ssssssssss | 146783 | 146799 | 20 |
| | CCAGAAA | deekeke | ssooss | | | |
| 1625933 | ATUGTCATCA | ekyddddddd | ssssssssss | 146783 | 146799 | 20 |
| | CCAGAAA | eeekeke | ssooss | | | |
| 1625941 | ATTGTCATCA | ekddd[aLd] | ssssssssss | 146783 | 146799 | 14 |
| | CCAGAAA | dddddeekek | ssooss | | | |
| | | e | | | | |
| 1625942 | ATTGTCATCA | ekddd[aLd] | ssssssssss | 146783 | 146799 | 14 |
| | CCAGAAA | ddddeeekek | ssooss | | | |
| | | e | | | | |
| 1625946 | ATTGTCATCA | eedddddddd | ssssssssss | 146783 | 146799 | 14 |
| | CCAGAAA | deeeeee | ssooss | | | |
| 1625947 | ATTGTCATCA | eedddddddd | ssssssssss | 146783 | 146799 | 14 |
| | CCAGAAA | eeeeeee | ssooss | | | |
| 1625956 | ATTGTCATCA | ekdydddddd | ssssssssss | 146783 | 146799 | 14 |
| | CCAGAAA | deekeke | ssooss | | | |
| 1625960 | ATTGTCATCA | ekdddddddd | sszzssssss | 146783 | 146799 | 14 |
| | CCAGAAA | deekeke | sssoss | | | |
| 1625961 | ATTGTCATCA | ekdddddddd | sszzssssss | 146783 | 146799 | 14 |
| | CCAGAAA | eeekeke | sssoss | | | |
| 1625962 | ATTGTCATCA | ekdydddddd | ssssssssss | 146783 | 146799 | 14 |
| | CCAGAAA | eeekeke | ssooss | | | |
| 1625976 | ATTGTCATCA | ekdddddddd | zzssssssss | 146783 | 146799 | 14 |
| | CCAGAAA | deekeke | sssoss | | | |
| 1625977 | ATTGTCATCA | ekdddddddd | zzssssssss | 146783 | 146799 | 14 |
| | CCAGAAA | eeekeke | sssoss | | | |
| 1625985 | ATTGTCATCA | ekdddddddd | zsssssssss | 146783 | 146799 | 14 |
| | CCAGAAA | deekeke | sssszz | | | |
| 1625986 | ATTGTCATCA | ekdddddddd | zsssssssss | 146783 | 146799 | 14 |
| | CCAGAAA | eeekeke | sssszz | | | |
| 1637229 | ATTGTCATCA | ekdddddddd | ssssssssss | 146783 | 146799 | 14 |
| | CCAGAAA | eeekeke | szzzss | | | |
| 1637478 | ATTGTCATCA | ekdddddddd | ssssssssss | 146783 | 146799 | 14 |
| | CCAGAAA | deekeke | szzzss | | | |
| 623208 | TTGTCATCAC | kddddddddd | ssssssssss | 146784 | 146798 | 15 |
| | CAGAA | keekk | soss | | | |
| 623242 | ATTGTCATCA | ekdddddddd | ssssssssss | 146784 | 146799 | 21 |
| | CCAGAA | eeekek | sooss | | | |
| 623243 | ATTGTCATCA | ekddddddde | ssssssssss | 146784 | 146799 | 21 |
| | CCAGAA | eeekek | sooss | | | |
| 1625902 | ATTGTCATCA | ekdddddddd | ssssssssss | 146784 | 146799 | 21 |
| | CCAGAA | eeekek | szzss | | | |
| 1625903 | TTGTCATCAC | kddddddddd | ssssssssss | 146784 | 146798 | 15 |
| | CAGAA | keekk | szss | | | |
| 1625909 | ATTGTCATCA | ekddddddde | ssssssssss | 146784 | 146799 | 21 |
| | CCAGAA | eeekek | szzss | | | |
| 1625913 | TTGTCATCAC | eddddddddd | ssssssssss | 146784 | 146798 | 15 |
| | CAGAA | keekk | soss | | | |
| 1625916 | ATTGTCATCA | eedddddddd | ssssssssss | 146784 | 146799 | 21 |
| | CCAGAA | eeekek | sooss | | | |
| 1625919 | ATTGTCATCA | eeddddddde | ssssssssss | 146784 | 146799 | 21 |
| | CCAGAA | eeekek | sooss | | | |
| 1625921 | TTGTCATCAC | kddddddddd | ssssssssss | 146784 | 146798 | 15 |
| | CAGAA | eeeee | soss | | | |
| 1625924 | ATTGTCATCA | ekdddddddd | ssssssssss | 146784 | 146799 | 21 |
| | CCAGAA | eeeeee | sooss | | | |
| 1625927 | TUGTCATCAC | kydddddddd | ssssssssss | 146784 | 146798 | 22 |
| | CAGAA | keekk | soss | | | |
| 1625928 | ATTGTCATCA | ekddddddde | ssssssssss | 146784 | 146799 | 21 |
| | CCAGAA | eeeeee | sooss | | | |
| 1625931 | ATUGTCATCA | ekyddddddd | ssssssssss | 146784 | 146799 | 23 |
| | CCAGAA | eeekek | sooss | | | |
| 1625934 | TTGTCATCAC | kddd[aLd] | ssssssssss | 146784 | 146798 | 15 |
| | CAGAA | dddddkeekk | soss | | | |
| 1625938 | TTGTCATCAC | eddddddddd | ssssssssss | 146784 | 146798 | 15 |
| | CAGAA | eeeee | soss | | | |
| 1625939 | ATTGTCATCA | ekddd[aLd] | ssssssssss | 146784 | 146799 | 21 |
| | CCAGAA | ddddeeekek | sooss | | | |
| 1625944 | ATTGTCATCA | ekddd[aLd] | ssssssssss | 146784 | 146799 | 21 |
| | CCAGAA | dddeeeekek | sooss | | | |
| 1625945 | ATTGTCATCA | eedddddddd | ssssssssss | 146784 | 146799 | 21 |
| | CCAGAA | eeeeee | sooss | | | |
| 1625948 | ATTGTCATCA | eeddddddde | ssssssssss | 146784 | 146799 | 21 |
| | CCAGAA | eeeeee | sooss | | | |
| 1625949 | TTGTCATCAC | kdyddddddd | ssssssssss | 146784 | 146798 | 15 |
| | CAGAA | keekk | soss | | | |
| 1625955 | ATTGTCATCA | ekdydddddd | ssssssssss | 146784 | 146799 | 21 |
| | CCAGAA | eeekek | sooss | | | |
| 1625959 | ATTGTCATCA | ekdddddddd | sszzssssss | 146784 | 146799 | 21 |
| | CCAGAA | eeekek | sssss | | | |
| 1625963 | ATTGTCATCA | ekddddddde | sszzssssss | 146784 | 146799 | 21 |
| | CCAGAA | eeekek | sssss | | | |
| 1625971 | TTGTCATCAC | kddddddddd | szzsssssss | 146784 | 146798 | 15 |
| | CAGAA | keekk | ssss | | | |
| 1625972 | TTGTCATCAC | kddddddddd | zsssssssss | 146784 | 146798 | 15 |
| | CAGAA | keekk | ssss | | | |
| 1625975 | ATTGTCATCA | ekdddddddd | zzssssssss | 146784 | 146799 | 21 |
| | CCAGAA | eeekek | sssss | | | |
| 1625978 | ATTGTCATCA | ekddddddde | zzssssssss | 146784 | 146799 | 21 |
| | CCAGAA | eeekek | sssss | | | |
| 1625981 | TTGTCATCAC | kddddddddd | zsssssssss | 146784 | 146798 | 15 |
| | CAGAA | keekk | sssz | | | |
| 1625984 | ATTGTCATCA | ekdddddddd | zsssssssss | 146784 | 146799 | 21 |
| | CCAGAA | eeekek | ssssz | | | |
| 1625987 | ATTGTCATCA | ekddddddde | zsssssssss | 146784 | 146799 | 21 |
| | CCAGAA | eeekek | ssssz | | | |
| 572772 | ATAAATTGTC | eeeekkdddd | ssssssssss | 146788 | 146803 | 24 |
| | ATCACC | dddkke | sssss | | | |
| 1625911 | ATAAATTGTC | eeeeeedddd | ssssssssss | 146788 | 146803 | 24 |
| | ATCACC | dddkke | sssss | | | |
| 1625920 | ATAAATTGTC | eeeekkdddd | ssssssssss | 146788 | 146803 | 24 |
| | ATCACC | dddeee | sssss | | | |
| 1625970 | ATAAATTGTC | eeeekkdddd | sssssszzss | 146788 | 146803 | 24 |
| | ATCACC | dddkke | sssss | | | |
| 1625980 | ATAAATTGTC | eeeekkdddd | zsssssssss | 146788 | 146803 | 24 |
| | ATCACC | dddkke | ssssz | | | |
| 1626292 | ATAAATTGTC | eeeekkdddd | ssszzsssss | 146788 | 146803 | 24 |
| | ATCACC | dddkke | sssss | | | |
| 1625936 | ATAAATTGTC | eeeeeedddd | ssssssssss | 146788 | 146803 | 24 |
| | ATCACC | dddeee | sssss | | | |
| 1626231 | ATAAATTGTC | eeeekkddd | ssssssssss | 146788 | 146803 | 24 |
| | ATCACC | [aLd]dd | sssss | | | |
| | | dkke | | | | |
| 1626245 | ATAAATTGTC | eeeekkdddd | sssoosssss | 146788 | 146803 | 24 |
| | ATCACC | dddkke | sssss | | | |

TABLE 2
Modified Oligonucleotides complementary
to human HTT

| | | | | SEQ | SEQ | |
|---|---|---|---|---|---|---|
| | | | | ID | ID | |
| | | | Inter- | No: | No: | |
| | | Sugar | nucleoside | 1 | 1 | SEQ |
| Compound | Sequence | Motif | linkages | Start | Stop | ID |
| No. | (5′ to 3′) | (5′ to 3′) | (5′ to 3′) | Site | Site | NO. |
| 1681079 | ATTGTCATCA | ekdddddddd | sssssszsss | 146784 | 146799 | 21 |
| | CCAGAA | eeekek | sssss | | | |
| 1681080 | ATTGTCATCA | ekdddddddd | ssssszzsss | 146784 | 146799 | 21 |
| | CCAGAA | eeekek | sssss | | | |
| 1681081 | ATTGTCATCA | ekdddddddd | ssssszzszs | 146784 | 146799 | 21 |
| | CCAGAA | eeekek | sssss | | | |
| 1681082 | ATTGTCATCA | ekdddddddd | ssszsszsss | 146784 | 146799 | 21 |
| | CCAGAA | eeekek | sssss | | | |
| 1681083 | ATTGTCATCA | ekdddddddd | ssszszzsss | 146784 | 146799 | 21 |
| | CCAGAA | eeekek | sssss | | | |
| 1681084 | ATTGTCATCA | ekdddddddd | sszzsszsss | 146784 | 146799 | 21 |
| | CCAGAA | eeekek | sssss | | | |
| 1681085 | ATTGTCATCA | ekdddddddd | sszzszzsss | 146784 | 146799 | 21 |
| | CCAGAA | eeekek | sssss | | | |
| 1681086 | ATTGTCATCA | ekdddddddd | ssszszszss | 146784 | 146799 | 21 |
| | CCAGAA | eeekek | sssss | | | |
| 1681087 | ATTGTCATCA | ekdddddddd | sszszszsss | 146784 | 146799 | 21 |
| | CCAGAA | eeekek | sssss | | | |

### Example 2: Effect of Modified Oligonucleotides on Human HTT RNA In Vitro

Modified oligonucleotides designed to target SNP rs7685686 as described herein above were tested for allele specific activity in GM04022 fibroblasts,

GM04022 fibroblasts, obtained from the NIGMS Human Genetic Cell Repository at the Coriell Institute for Medical Research, were treated with modified oligonucleotide at concentrations of 15,000 nM, 3750 nM, 937 nM, 234 nM, and 58.6 nM by electroporation at a density of 35,000 cells per well. After a treatment period of approximately 24 hours, total RNA was isolated from the cells and HTT RNA levels were measured by quantitative real-time RT-PCR.

GM04022 fibroblasts are heterozygous with a G and an A nucleotide at SNP rs7685686. The level of knockdown of both A and G alleles were correlated with the level of two alleles at a second site of variation (SNP rs362303) in GM04022 fibroblasts. Specifically, the G allele of SNP rs7685686 was correlated to the A allele of SNP rs362303. The A allele of SNP rs7685686 was correlated to the G allele of SNP rs362303. As such, the level of knockdown of both the A and G alleles of SNP rs7685686 was quantitated based on the level of the two alleles of SNP rs362303 with the C_2229297_10 assay (Thermo Fisher Scientific).

The C_2229297_10 assay uses two different fluorophores to simultaneously measure levels of both alleles of SNP rs7685686 with respect to the alleles of SNP rs362303. 6-carboxyfluorescein (FAM) measures the level of A allele at SNP rs362303 (which corresponds to G at SNP rs7685686) and VIC® measures the level of the G allele at SNP rs362303 (which corresponds to A at SNP rs7685686). HTT levels were normalized to total RNA or to human GAPDH expression levels measured with quantitative RT-PCR using human primer-probe set RTS104 (forward sequence GAAGGTGAAGGTCGGAGTC, designated herein as SEQ ID NO: 2; reverse sequence GAAGATGGTGATGGGATTTC, designated herein as SEQ ID NO: 3; probe sequence CAAGCTTCCCGTTCTCAGCC, designated herein as SEQ ID NO: 4). IC50 values were calculated using GraphPad Prism. Additionally, the modified oligonucleotides above are cross-reactive with human BMPR1. The effect of the modified oligonucleotides on BMPR1 levels were measured using human primer-probe set RTS2623 (forward sequence CACTGCCCCCTGTTGTCATA, designated herein as SEQ ID NO: 5; reverse sequence GAGCAAAACCAGCCATCGA, designated herein as SEQ ID NO: 6; probe sequence TCCGTTTTTTGATGGCAGCA, designated herein as SEQ ID NO: 7).

Selectivity of the modified oligonucleotide for the A allele over the G allele of SNP rs7685686 was measured by determining dividing the IC50s measured by the FAM fluorophore by the IC50s measured by the VIC fluorophore. Selectivity of the modified oligonucleotide for the A allele of SNP rs7685686 over BMPR1 was measured by dividing the IC50s measured by RTS2623 by the IC50s measured by the VIC fluorophore.

TABLE 3
Effect of modified oligonucleotides complementary to human HTT

| | IC50 | IC50 | | HTT | |
|---|---|---|---|---|---|
| Com- | (μM)VIC | (μM) FAM | BMPR1 | Selectivity | Selectivity |
| pound | (measures | (measures | IC50 | for A | for HTT |
| Number | A Allele) | G Allele) | (μM) | Allele | over BMPR1 |
| 387916 | 0.7 | 1 | >15 | 1 | >21 |
| 572772 | 0.08 | 4.94 | 0.68 | 64 | 9 |
| 623205 | 0.26 | 0.36 | 4.91 | 1 | 19 |
| 623206 | 0.16 | >15 | >15 | >92 | >92 |
| 623208 | 0.19 | 1.20 | 10.48 | 6 | 54 |
| 623235 | 0.14 | 0.52 | 3.95 | 4 | 29 |
| 623236 | 0.13 | >15 | 8.56 | >113 | 64 |
| 623242 | 0.10 | 1.47 | 5.27 | 14 | 52 |
| 623243 | 0.05 | >15 | 5.48 | >279 | 102 |
| 1625897 | 0.28 | 3.03 | 12.55 | 11 | 45 |
| 1625898 | 0.22 | 0.68 | 9.70 | 3 | 44 |
| 1625899 | 0.21 | 2.52 | 3.95 | 12 | 19 |
| 1625902 | 0.38 | 7.64 | 5.59 | 20 | 15 |
| 1625903 | 0.52 | 2.97 | 4.81 | 6 | 9 |
| 1625904 | 0.65 | >15 | 14.64 | >23 | 23 |
| 1625905 | 0.77 | 2.73 | 10.79 | 4 | 14 |
| 1625909 | 0.81 | >15 | 6.69 | >18 | 8 |
| 1625911 | 1.78 | >15 | 4.80 | >8 | 3 |
| 1625913 | 0.70 | 4.12 | 7.60 | 6 | 11 |
| 1625914 | 1.21 | >15 | >15 | >12 | >12 |
| 1625915 | 0.43 | 1.29 | >15 | 3 | >35 |
| 1625916 | 0.98 | 8.35 | >15 | 9 | >15 |
| 1625917 | 0.49 | 0.98 | >15 | 2 | >31 |
| 1625918 | 0.94 | >15 | >15 | >17 | >16 |
| 1625919 | 1.36 | >15 | >15 | >11 | >11 |
| 1625920 | 0.87 | 10.49 | 2.67 | 12 | 3 |
| 1625921 | 1.17 | 8.27 | >15 | 7 | >13 |
| 1625922 | 1.77 | >15 | >15 | >8 | >8 |
| 1625923 | 0.77 | 4.02 | 11.49 | 5 | 15 |
| 1625924 | 1.31 | 13.09 | 6.03 | 10 | 5 |
| 1625925 | 0.93 | 4.66 | 7.18 | 5 | 8 |
| 1625926 | 1.87 | >15 | 4.66 | >9 | 2 |
| 1625927 | 0.14 | 0.58 | 1.95 | 4 | 14 |
| 1625928 | 0.36 | >15 | 6.76 | >42 | 19 |
| 1625929 | 0.49 | >15 | 10.26 | >31 | 21 |
| 1625930 | 0.11 | 1.05 | 5.45 | 9 | 48 |
| 1625931 | 0.53 | 1.05 | 6.05 | 2 | 11 |
| 1625932 | 0.05 | 1.90 | 3.08 | 41 | 67 |
| 1625933 | 0.18 | >15 | 8.47 | >84 | 47 |
| 1625935 | 1.77 | >15 | >15 | >8 | >8 |
| 1625936 | 2.16 | >15 | 11.78 | >7 | 5 |
| 1625937 | 0.27 | 0.66 | 11.68 | 2 | 43 |
| 1625938 | 0.92 | 9.88 | >15 | 11 | >16 |
| 1625939 | 0.66 | >15 | 3.61 | >23 | 5 |
| 1625940 | 2.77 | >15 | >15 | >5 | >5 |
| 1625941 | 0.20 | 0.53 | 4.32 | 3 | 22 |
| 1625942 | 0.14 | >15 | 6.90 | >111 | 51 |
| 1625943 | 1.81 | 11.63 | >15 | 6 | >8 |
| 1625944 | 13.85 | >15 | >15 | >1 | >1 |
| 1625945 | 2.49 | >15 | >15 | >6 | >6 |
| 1625946 | 1.14 | >15 | >15 | >13 | >13 |
| 1625947 | 12.28 | >15 | >15 | >1 | >1 |
| 1625948 | 1.75 | >15 | >15 | >9 | >9 |
| 1625949 | 0.26 | 9.61 | 4.08 | 37 | 16 |
| 1625951 | 1.30 | >15 | 10.15 | >12 | 8 |
| 1625953 | 0.29 | 4.55 | 4.56 | 16 | 16 |
| 1625955 | 1.89 | >15 | >15 | >8 | >8 |
| 1625956 | 0.26 | 1.08 | 2.39 | 4 | 9 |
| 1625957 | 0.59 | 14.39 | >15 | 24 | >25 |
| 1625958 | 0.21 | 1.38 | 9.34 | 6 | 44 |
| 1625959 | 0.66 | >15 | 5.24 | >23 | 8 |
| 1625960 | 0.28 | 1.30 | 3.84 | 5 | 14 |
| 1625961 | 0.23 | >15 | 7.30 | >66 | 32 |
| 1625962 | 0.86 | 12.44 | 9.06 | 15 | 11 |
| 1625963 | 0.73 | >15 | 6.83 | >20 | 9 |
| 1625970 | 0.11 | >15 | 0.65 | >141 | 6 |
| 1625971 | 0.05 | 1.35 | 1.85 | 26 | 36 |
| 1625973 | 1.15 | >15 | >15 | >13 | >13 |
| 1625974 | 0.26 | 3.27 | >15 | 12 | >57 |
| 1625980 | 0.20 | 9.33 | 0.44 | 47 | 2 |
| 1625981 | 0.40 | 7.60 | 6.89 | 19 | 17 |
| 1625982 | 0.60 | >15 | >15 | >26 | >26 |
| 1625983 | 0.50 | 5.70 | >15 | 13 | >33 |
| 1625984 | 0.50 | 7.30 | 4.98 | 14 | 9 |
| 1625985 | 0.20 | 1.40 | 5.49 | 6 | 23 |
| 1625986 | 0.20 | >15 | 4.61 | >63 | 19 |
| 1625987 | 0.80 | >15 | 10.33 | >19 | 13 |
| 1626231 | 7.30 | >15 | >15 | >2 | >2 |
| 1626245 | 0.10 | >15 | 0.69 | >107 | 5 |
| 1626292 | 0.20 | 10.30 | 0.49 | 65 | 3 |
| 1637229 | 0.20 | 4.90 | 3.55 | 21 | 15 |
| 1637478 | 0.20 | 0.60 | 3.26 | 4 | 21 |

TABLE 4
Effect of modified oligonucleotides complementary to human HTT

| | IC50 | IC50 | | HTT | |
|---|---|---|---|---|---|
| Com- | (μM)VIC | (μM) FAM | BMPR1 | Selectivity | Selectivity |
| pound | (measures | (measures | IC50 | for A | for HTT |
| Number | A Allele) | G Allele) | (μM) | Allele | over BMPR1 |
| 623242 | 0.41 | 14.73 | 2.96 | 36 | 7 |
| 1681079 | 0.51 | >20 | 4.91 | >39 | 10 |
| 1681080 | 0.31 | >20 | 6.55 | >64 | 21 |
| 1681081 | 0.75 | >20 | 7.10 | >27 | 10 |
| 1681082 | 0.38 | 6.08 | 5.12 | 16 | 13 |
| 1681083 | 1.83 | >20 | 5.87 | >11 | 3 |
| 1681084 | 0.44 | 9.31 | 5.27 | 21 | 12 |
| 1681085 | 1.35 | 17.99 | 6.49 | 13 | 5 |
| 1681086 | 1.14 | >20 | 6.45 | >18 | 6 |
| 1681087 | 1.17 | >20 | 8.81 | >17 | 8 |

### Example 3: Tolerability of Modified Oligonucleotides in Wild-Type Mice, 3-Hour Study

Modified oligonucleotides described above were tested in wild-type female C57BL/6 mice to assess the tolerability of the oligonucleotides. Wild-type female C57BL/6 mice each received a single ICV dose of modified oligonucleotide at 700 μg. Each treatment group consisted of 4 mice, unless indicated otherwise in the tables below. A group of 4 mice received PBS as a negative control for each experiment. Each experiment is identified in separate tables below. At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a sub-score of 0 if it met the criteria and 1 if it did not (the functional observational battery score or FOB). After all 7 criteria were evaluated, the scores were summed for each mouse and averaged within each treatment group. The results are presented in the tables below.

TABLE 5
Tolerability scores in mice

| Compound No. | 3 hr. FOB |
|---|---|
| PBS | 0.00 |
| 623242 | 1.00 |
| 623243 | 3.00 |
| 1625897 | 0.00 |
| 1625899 | 0.00 |
| 1625902 | 1.50 |
| 1625904 | 3.25 |
| 1625909 | 3.75 |
| 1625927 | 3.25 |
| 1625929 | 1.25 |
| 1625932 | 2.00 |
| 1625933 | 1.00 |
| 1625942 | 0.00 |
| 1625949 | 6.00 |
| 1625953 | 1.25 |
| 1625957 | 0.00 |
| 1625961 | 0.00 |
| 1625971 | 1.00 |
| 1625974 | 2.00 |
| 1625984 | 3.75 |
| 1625986 | 4.00 |
| 1625987 | 3.50 |
| 1626245 | 2.00 |
| 1637229 | 1.00 |

TABLE 6
Tolerability scores in mice

| Compound No. | 3 hr. FOB |
|---|---|
| PBS | 0.00 |
| 623242 | 4.50 |
| 1681079 | 6.00 |
| 1681080 | 5.00 |
| 1681081 | 4.75 |
| 1681082 | 4.75 |
| 1681083 | 4.00 |
| 1681084 | 4.00 |
| 1681085 | 2.00 |
| 1681086 | 4.00 |
| 1681087 | 5.00 |

TABLE 7
Tolerability scores in mice

| Compound No. | 3 hr. FOB |
|---|---|
| PBS | 0.00 |
| 572772 | 2.00 |
| 623208 | 1.00 |
| 623242 | 1.50 |

TABLE 8
Tolerability scores in mice

| Compound No. | Group Size | 3 hr. FOB |
|---|---|---|
| PBS | 4 | 0.00 |
| 623208 | 4 | 1.00 |
| 623236 | 4 | 0.00 |
| 1625961 | 6 | 0.00 |

### Example 4: Tolerability of Modified Oligonucleotides in Rats, 3-Hour Study

Modified oligonucleotides described above were tested in rats to assess the tolerability of the oligonucleotides. Sprague Dawley rats each received a single intrathecal (IT) dose of oligonucleotide at dose of modified oligonucleotide at 3 mg. Each treatment group consisted of 4-6 rats, unless indicated otherwise in the tables below. A group of 4 rats received PBS as a negative control. Each experiment is identified in separate tables below. At 3 hours post-injection, movement in 7 different parts of the body were evaluated for each rat. The 7 body parts are (1) the rat's tail; (2) the rat's posterior posture; (3) the rat's hind limbs; (4) the rat's hind paws; (5) the rat's forepaws; (6) the rat's anterior posture; (7) the rat's head. For each of the 7 different body parts, each rat was given a sub-score of 0 if the body part was moving or 1 if the body part was paralyzed (the functional observational battery score or FOB). For each of the 7 criteria, a rat was given a sub-score of 0 if it met the criteria and 1 if it did not (the functional observational battery score or FOB). After all 7 criteria were evaluated, the scores were summed for each rat and averaged within each treatment group. The results are presented in the tables below.

TABLE 9
Tolerability scores in rats

| Compound No. | 3 hr. FOB |
|---|---|
| PBS | 0.00 |
| 623242 | 3.00 |
| 623243 | 3.00 |
| 1625897 | 3.00 |
| 1625899 | 0.75 |
| 1625902 | 3.00 |
| 1625904 | 3.00‡ |
| 1625909 | 3.00 |
| 1625927 | 4.50 |
| 1625929 | 3.00 |
| 1625932 | 3.00 |
| 1625933 | 3.00‡ |
| 1625942 | 2.00 |
| 1625949 | 4.50 |
| 1625953 | 2.50 |
| 1625957 | 2.00 |
| 1625961 | 2.00‡ |
| 1625971 | 3.75 |
| 1625974 | 3.00 |
| 1625984 | 3.50 |
| 1625986 | 3.75 |
| 1625987 | 4.00‡ |
| 1626245 | 2.33‡ |
| 1637229 | 3.00 |

‡indicates fewer than 4 samples available

TABLE 10
Tolerability scores in rats

| Compound No. | 3 hr. FOB |
|---|---|
| PBS | 0.00 |
| 623242 | 1.50 |
| 1681079 | 4.00 |
| 1681080 | 4.00 |
| 1681081 | 4.00 |
| 1681082 | 2.25 |
| 1681083 | 3.00 |
| 1681084 | 3.00 |
| 1681085 | 2.00 |
| 1681086 | 2.75 |
| 1681087 | 3.00 |

TABLE 11
Tolerability scores in rats

| Compound No. | 3 hr. FOB |
|---|---|
| PBS | 0.00 |
| 572772 | 3.25 |
| 623208 | 4.00 |
| 623242 | 2.75 |

TABLE 12
Tolerability scores in rats

| Compound No. | Group Size | 3 hr. FOB |
|---|---|---|
| PBS | 4 | 0.00 |
| 623208 | 4 | 3.75 |
| 623236 | 4 | 1.25 |
| 1625961 | 6 | 2.40‡ |

‡indicates fewer than 6 samples available

### Example 5: Potency of Modified Oligonucleotides in BACHD Mice Expressing SNP rs7685686, 2-Weeks

Transgenic mice expressing human HTT comprising SNP rs7685686 (The Jackson Laboratory, Stock No: 008197) were used to test activity of modified oligonucleotides described above.

### Treatment

The HTT transgenic mice were divided into groups of 3 mice each. Each mouse received a single ICV bolus of modified oligonucleotide at doses indicated in the table below. A group of 4 mice received a single ICV bolus with PBS as a negative control.

### RNA Analysis

Two weeks post treatment, mice were sacrificed, and RNA was extracted from cortical brain tissue and spinal cord for RT-PCR analysis to measure amount of HTT RNA using human primer probe set RTS2617 (forward sequence CTCCGTCCGGTAGACATGCT, designated herein as SEQ ID NO: 8; reverse sequence GGAAATCAGAACCCTCAAAATGG, designated herein as SEQ ID NO: 9; probe sequence TGAGCACTGTTCAACTGTGGATATCGGGA, designated herein as SEQ ID NO: 10). Results are presented as percent human HTT RNA relative to the amount of HTT in PBS treated control animals, normalized to mouse PPIA (% control). Mouse PPIA was amplified using primer probe set m_cyclo24 (forward sequence TCGCCGCTTGCTGCA, designated herein as SEQ ID NO: 11; reverse sequence ATCGGCCGTGATGTCGA, designated herein as SEQ ID NO: 12; probe sequence CCATGGTCAACCCCACCGTGTTC, designated herein as SEQ ID NO: 13).

The half maximal effective dose (ED₅₀) of each modified oligonucleotide was calculated using GraphPad Prism 7 software (GraphPad Software, San Diego, CA).

TABLE 13
Dose-dependent percent reduction of
human HTT mRNA in HTT transgenic mice

| | | HTT RNA (% control) | | | |
|---|---|---|---|---|---|
| Compound | Dose | Spinal | ED 50 | | ED 50 |
| No. | (μg) | Cord | (μg) | Cortex | (μg) |
| PBS | — | 100 | — | 100 | — |
| 1625902 | 10 | 86 | 195.4 | 97 | 350.4 |
| | 30 | 70 | | 85 | |
| | 100 | 56 | | 59 | |
| | 300 | 42 | | 57 | |
| | 700 | 41‡ | | 40‡ | |
| 1625904 | 10 | 82 | >700 | 88 | >700 |
| | 30 | 88 | | 81 | |
| | 100 | 81 | | 75 | |
| | 300 | 82 | | 66 | |
| | 700 | 52 | | 52 | |
| 1625909 | 10 | 98 | 439.6 | 96 | 465 |
| | 30 | 93 | | 92 | |
| | 100 | 88‡ | | 63‡ | |
| | 300 | 53 | | 56 | |
| | 700 | 42 | | 46 | |
| 1625942 | 10 | 100 | 408.6 | 77 | 206.8 |
| | 30 | 94 | | 64 | |
| | 100 | 72 | | 49 | |
| | 300 | 52 | | 49 | |
| | 700 | 44 | | 42 | |
| 1625961 | 10 | 107 | 172.8 | 71 | 70.1 |
| | 30 | 91 | | 55 | |
| | 100 | 55 | | 45 | |
| | 300 | 37 | | 37 | |
| | 700 | 24 | | 29 | |
| 1637229 | 10 | 86 | 164.1 | 73 | 108.7 |
| | 30 | 96 | | 64 | |
| | 100 | 50 | | 38 | |
| | 300 | 33 | | 50 | |
| | 700 | 31 | | 33 | |

‡indicates fewer than 3 samples available

### Example 6: Potency of Modified Oligonucleotides in BACHD Mice Expressing SNP rs7685686, 4-Weeks

Transgenic mice expressing human HTT comprising SNP rs7685686 (described herein above) were used to test activity of modified oligonucleotides described above.

### Treatment

The HTT transgenic mice were divided into groups of 4 mice each. Each mouse received a single ICV bolus of modified oligonucleotide at doses indicated in the tables below. A group of 4 mice received a single ICV bolus with PBS as a negative control.

### RNA Analysis

Four weeks post treatment, mice were sacrificed, and RNA was extracted from cortical brain tissue and spinal cord for RT-PCR analysis to measure amount of HTT RNA using human primer probe set RTS2617 (described herein above). Results are presented as percent human HTT RNA relative to the amount of HTT in PBS treated control animals, normalized to mouse PPIA (% control). Mouse PPIA was amplified using primer probe set m_cyclo24 (described herein above).

The half maximal effective dose (ED₅₀) of each modified oligonucleotide was calculated using GraphPad Prism 7 software (GraphPad Software, San Diego, CA).

TABLE 14
Dose-dependent percent reduction of
human HTT mRNA in HTT transgenic mice

| | | HTT RNA (% control) | | | |
|---|---|---|---|---|---|
| Compound | Dose | Spinal | ED 50 | | ED 50 |
| No. | (μg) | Cord | (μg) | Cortex | (μg) |
| PBS | — | 100 | — | 100 | — |
| 1625961 | 10 | 99 | 135.2 | 102 | 123 |
| | 30 | 78 | | 73 | |
| | 100 | 47 | | 48 | |
| | 300 | 34 | | 34 | |
| | 700 | 28 | | 22 | |
| 1637229 | 30 | 69 | 206.8 | 84 | 250.8 |
| | 100 | 53 | | 64 | |
| | 300 | 47 | | 44 | |
| | 700 | 41 | | 36 | |

### Example 7: Duration of Action Study of Modified Oligonucleotides in BACHD Mice Comprising SNP rs7685686, Single Dose

Modified oligonucleotides described above were tested in HTT transgenic mice (described herein above).

The HTT transgenic mice were divided into groups of 4 mice each. Each mouse received a single ICV bolus of either 200 or 300 g of modified oligonucleotide as indicated in the tables below. A group of 2-4 mice received a single ICV bolus with PBS as a negative control.

Mice were sacrificed at various timepoints as indicated in the tables below and RNA was extracted from cortical brain tissue, spinal cord, striatum, and hippocampus for RT-PCR analysis to measure amount of HTT RNA using human primer probe set RTS2617 (described herein above). Results are presented as percent human HTT RNA relative to the amount of HTT in PBS treated control animals, normalized to mouse cyclophilin A (% control). Mouse PPIA was amplified using primer probe set m_cyclo24 (described herein above).

Compound 443139 is a comparator compound and is previously described in WO 2011/032045.

TABLE 15
Reduction of human HTT RNA in HTT
transgenic mice at a dose of 300 μg

| | | |
|---|---|---|
| | Timepoint | |
| Compound | (Days | HTT RNA (% control) |

| No. | post-dose) | Spinal Cord | Cortex |
|---|---|---|---|
| PBS | 28 | 100 | 100 |
| | 56 | 100 | 100 |
| | 84 | 100 | 100 |
| | 112 | 100 | 100 |
| | 141 | 100 | 100 |
| 623242 | 28 | 44 | 33 |
| | 56 | 57 | 50 |
| | 84 | 66‡ | 59‡ |
| | 112 | 61 | 51 |
| | 141 | 89 | 78 |
| 623243 | 28 | 42 | 37 |
| | 56 | 61 | 54 |
| | 84 | 60 | 67 |
| | 112 | 55 | 52 |
| | 141 | 78 | 72 |
| 1625902 | 28 | 45 | 34 |
| | 56 | 60 | 55 |
| | 84 | 54‡ | 64‡ |
| | 112 | 71 | 69 |
| | 141 | 74 | 82 |
| 1625904 | 28 | 58 | 53 |
| | 56 | 69 | 71 |
| | 84 | 80 | 72 |
| 1625909 | 28 | 59 | 48 |
| | 56 | 72 | 66 |
| | 84 | 72 | 61 |
| 1625929 | 28 | 56 | 49 |
| | 56 | 70 | 74 |
| | 84 | 69 | 84 |
| 1625933 | 28 | 43 | 36 |
| | 56 | 53 | 53 |
| | 84 | 56 | 48 |
| 1625942 | 28 | 52 | 45 |
| | 56 | 63 | 66 |
| | 84 | 71 | 64 |
| 1625957 | 28 | 47‡ | 28‡ |
| | 56 | 59 | 55 |
| | 84 | 60 | 59 |
| 1625957 | 28 | 45‡ | 43‡ |
| | 56 | 60 | 59 |
| | 84 | 63 | 65 |
| 1625961 | 28 | 34 | 23 |
| | 56 | 52 | 40 |
| | 84 | 51 | 56 |
| | 112 | 46 | 51 |
| | 141 | 75 | 65 |
| 1625974 | 28 | 31 | 26 |
| | 56 | 48 | 38 |
| | 84 | 42 | 46 |
| 1625984 | 28 | 49 | 33 |
| | 56 | 71 | 56 |
| | 84 | 68 | 59 |
| 1637229 | 28 | 43 | 34 |
| | 56 | 84 | 52 |
| | 84 | 56 | 63 |
| | 112 | 67 | 68 |
| | 141 | 88 | 78 |
| 1681082 | 28 | 44‡ | 35‡ |
| | 56 | 67 | 61 |
| | 84 | 55 | 68 |
| 1681083 | 28 | 48 | 40 |
| | 56 | 76 | 56 |
| | 84 | 58 | 61 |
| 1681084 | 28 | 40 | 30 |
| | 56 | 57 | 43 |
| | 84 | 53 | 49 |
| 1681085 | 28 | 44 | 39 |
| | 56 | 71 | 54 |
| | 84 | 65‡ | 85‡ |
| 1681086 | 28 | 44 | 49 |
| | 56 | 87 | 57 |
| | 84 | 77 | 92 |
| 1681087 | 28 | 50 | 50 |
| | 56 | 95 | 78 |
| | 84 | 78 | 103 |

‡indicates fewer than 4 samples available

TABLE 16
Reduction of human HTT RNA in HTT
transgenic mice at a dose of 200 μg

| | Timepoint | HTT RNA (% control) | | | |
|---|---|---|---|---|---|
| Compound | (Days | Spinal | | | |
| No. | post-dose) | Cord | Cortex | Striatum | Hippocampus |
| PBS | 7 | 100 | 100 | 100 | 100 |
| | 14 | 100 | 100 | 100 | 100 |
| | 28 | 100 | 100 | 100 | 100 |
| | 84 | 100 | 100 | 100 | 100 |
| 443139 | 7 | 50 | 55 | 39 | 53 |
| | 14 | 59 | 23 | 46 | 32 |
| | 28 | 49 | 47 | 38 | 44 |
| | 84 | 94 | 83 | 123 | 104 |

### Example 8: Duration of Action Study of Select Modified Oligonucleotides in BACHD Mice Comprising SNP rs7685686, Single Dose

Modified oligonucleotides with the following Compound Nos. 1625961, 1637229, and 623236 will be tested in HTT transgenic mice (described herein above).

The HTT transgenic mice will be divided into groups of 4 mice each. Each mouse will receive a single ICV bolus of either 200 or 300 g of a modified oligonucleotide. A group of 2-4 mice will receive a single ICV bolus with PBS as a negative control.

Mice will be sacrificed at various timepoints and RNA will be extracted from cortical brain tissue, spinal cord, striatum, and hippocampus for RT-PCR analysis to measure the amount of HTT RNA using human primer probe set RTS2617 (described herein above). Results will be presented as percent human HTT RNA relative to the amount of HTT in PBS treated control animals, normalized to mouse PPIA (% control). Mouse PPIA will be amplified using primer probe set m_cyclo24 (described herein above).

### Example 9: Effect of Modified Oligonucleotides on Human HTT RNA In Vitro

Modified oligonucleotides designed to target SNP rs7685686 as described herein above were tested for allele specific activity in GM04022 fibroblasts.

GM04022 fibroblasts (described herein above), were electroporated at a density of 35,000 cells per well with modified oligonucleotide in a 10-point dose response ranging from 0.005 to 20 μM. After a treatment period of approximately 24 hours, total RNA was isolated from the cells and HTT RNA levels were measured by quantitative real-time RT-PCR.

The C_2229297_10 assay (described herein above) was used to measure the level of A allele at SNP rs362303 (which corresponds to G at SNP rs7685686) and to measure the level of the G allele at SNP rs362303 (which corresponds to A at SNP rs7685686). HTT levels were normalized to total RNA or to human GAPDH expression levels measured with quantitative RT-PCR using human primer-probe set RTS104 (described herein above). IC₅₀ values were calculated using GraphPad Prism (San Diego, CA).

Selectivity of the modified oligonucleotide for the A allele over the G allele of SNP rs7685686 was determined by dividing the IC₅₀ values measured by the FAM fluorophore by the IC₅₀ values measured by the VIC fluorophore.

TABLE 17
Allele selectivity of modified oligonucleotides
complementary to human HTT

| | | | HTT |
|---|---|---|---|
| | VIC | FAM | Selectivity |
| Compound | IC₅₀ (μM) | IC₅₀ (μM) | for A |
| No. | (A Allele) | (G Allele) | Allele |
| 1625934 | 1.455 | >20 | >14 |
| 1625961 | 0.275 | >20 | >73 |
| 1625972 | 0.633 | 19.59 | 31 |
| 1625975 | 0.961 | >20 | >21 |
| 1625976 | 0.682 | 5.86 | 8.6 |
| 1625977 | 0.471 | >20 | >42 |
| 1625978 | 0.902 | >20 | >22 |
| 1637229 | 0.435 | >20 | >46 |
| 623208 | 0.638 | 5.54 | 8.7 |
| 623236 | 0.466 | >20 | >43 |

1. A modified oligonucleotide according to the following chemical structure: 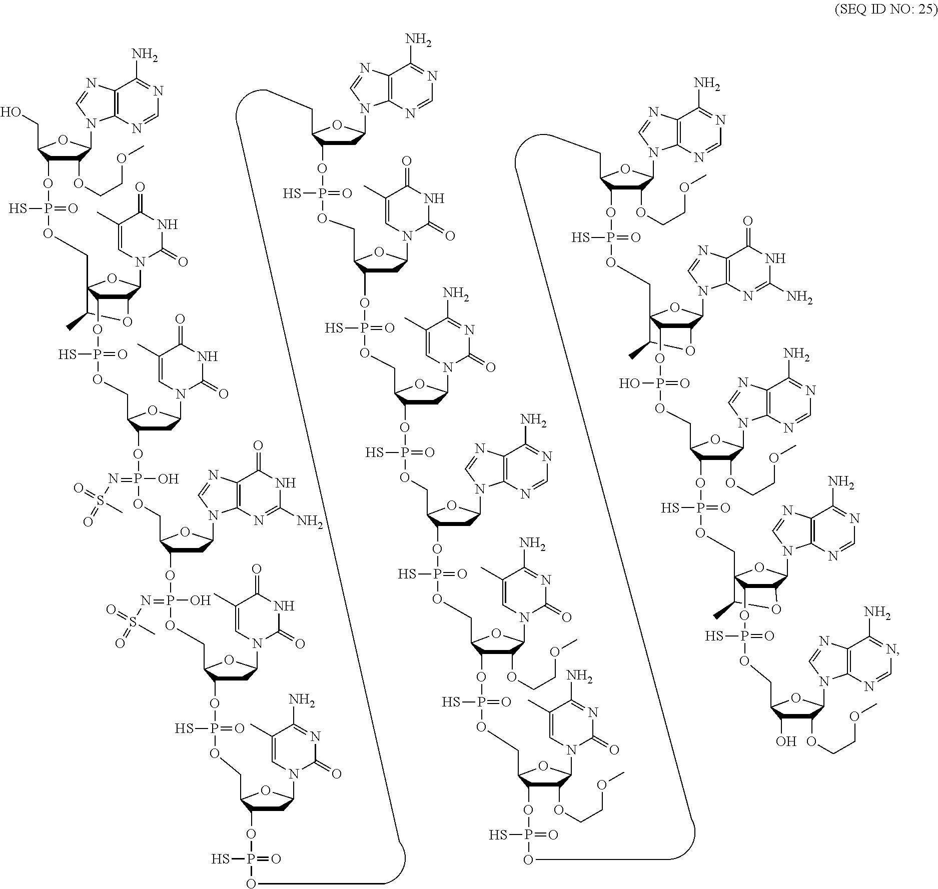
or a pharmaceutically acceptable salt thereof.

2. (canceled)

3. The modified oligonucleotide of claim 1, which is a pharmaceutically acceptable salt comprising one or more cations selected from sodium, potassium, calcium, and magnesium.

4. A modified oligonucleotide according to the following chemical structure:

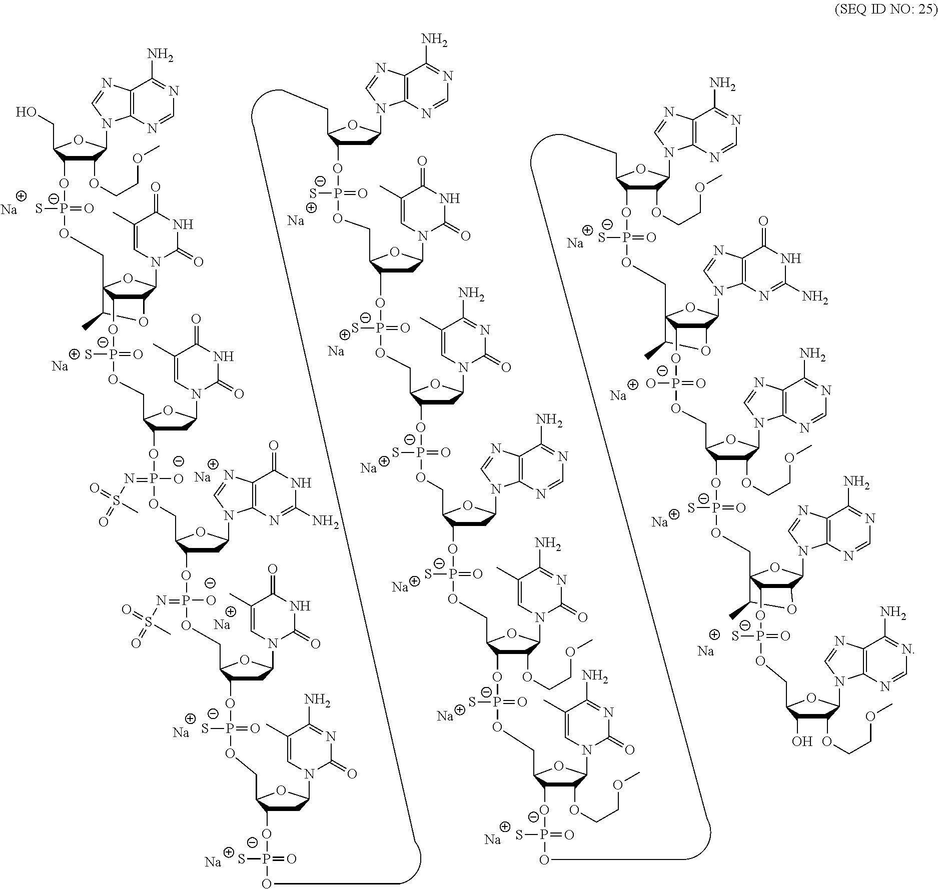

5. (canceled)

6. An oligomeric compound comprising a modified oligonucleotide according to the following chemical notation: AₑₛTₖₛT_{dz}G_{dz}T_{ds}^{m}C_{ds}A_{ds}T_{ds}^{m}C_{ds}A_{ds}^{m}Cₑₛ^{m}CₑₛAₑₛGₖₒAₑₛAₖₛAₑ (SEQ ID NO: 25), wherein:
A=an adenine nucleobase,
^{m}C=a 5-methylcytosine nucleobase,
G=a guanine nucleobase,
T=a thymine nucleobase,
e=a 2′-MOE sugar moiety,
k=a cEt sugar moiety,
d=a 2′-β-D-deoxyribosyl sugar moiety,
s=a phosphorothioate internucleoside linkage,
z=a mesyl phosphoramidate internucleoside linkage, and
o=a phosphodiester internucleoside linkage, and
wherein the oligomeric compound optionally comprises a conjugate group or a terminal group.

7. (canceled)

8. The oligomeric compound of claim 6, wherein the modified oligonucleotide is a pharmaceutically acceptable salt.

9. The oligomeric compound of claim 8, wherein the modified oligonucleotide is a pharmaceutically acceptable salt comprising one or more cations selected from sodium, potassium, calcium, and magnesium.

10. A population of modified oligonucleotides of claim 1, wherein all of the phosphorothioate internucleoside linkages of the modified oligonucleotide are stereorandom.

11. A population of modified oligonucleotides of claim 1, wherein all of the mesyl phosphoramidate internucleoside linkages of the modified oligonucleotide are stereorandom.

12. A pharmaceutical composition comprising a modified oligonucleotide of claim 1 and a pharmaceutically acceptable diluent.

13. The pharmaceutical composition of claim 12, wherein the pharmaceutically acceptable diluent is artificial cerebrospinal fluid (aCSF) or phosphate-buffered saline (PBS).

14. The pharmaceutical composition of claim 12, wherein the pharmaceutical composition consists essentially of the modified oligonucleotide and aCSF.

15. The pharmaceutical composition of claim 12, wherein the pharmaceutical composition consists essentially of the modified oligonucleotide and PBS.

16. The pharmaceutical composition of claim 12, wherein the pharmaceutical composition consists of the modified oligonucleotide and aCSF.

17. The pharmaceutical composition of claim 12, wherein the pharmaceutical composition consists of the modified oligonucleotide and PBS.

18.-134. (canceled)

135. A pharmaceutical composition comprising a modified oligonucleotide of claim 3 and a pharmaceutically acceptable diluent.

136. The pharmaceutical composition of claim 135, wherein the pharmaceutically acceptable diluent is artificial cerebrospinal fluid (aCSF) or phosphate-buffered saline (PBS).

137. The pharmaceutical composition of claim 136, wherein the pharmaceutical composition consists essentially of the modified oligonucleotide and aCSF.

138. The pharmaceutical composition of claim 136, wherein the pharmaceutical composition consists essentially of the modified oligonucleotide and PBS.

139. A pharmaceutical composition comprising a modified oligonucleotide of claim 4 and a pharmaceutically acceptable diluent.

140. The pharmaceutical composition of claim 139, wherein the pharmaceutically acceptable diluent is artificial cerebrospinal fluid (aCSF) or phosphate-buffered saline (PBS).

141. The pharmaceutical composition of claim 140, wherein the pharmaceutical composition consists essentially of the modified oligonucleotide and aCSF.

142. The pharmaceutical composition of claim 140, wherein the pharmaceutical composition consists essentially of the modified oligonucleotide and PBS.

143. A pharmaceutical composition comprising an oligomeric compound of claim 6 and a pharmaceutically acceptable diluent.

144. The pharmaceutical composition of claim 143, wherein the pharmaceutically acceptable diluent is artificial cerebrospinal fluid (aCSF) or phosphate-buffered saline (PBS).

145. The pharmaceutical composition of claim 144, wherein the pharmaceutical composition consists essentially of the oligomeric compound and aCSF.

146. The pharmaceutical composition of claim 144, wherein the pharmaceutical composition consists essentially of the oligomeric compound and PBS.

147. A population of modified oligonucleotides of claim 3, wherein all of the phosphorothioate internucleoside linkages of the modified oligonucleotide are stereorandom.

148. A population of modified oligonucleotides of claim 3, wherein all of the mesyl phosphoramidate internucleoside linkages of the modified oligonucleotide are stereorandom.

149. A population of modified oligonucleotides of claim 4, wherein all of the phosphorothioate internucleoside linkages of the modified oligonucleotide are stereorandom.

150. A population of modified oligonucleotides of claim 4, wherein all of the mesyl phosphoramidate internucleoside linkages of the modified oligonucleotide are stereorandom.

151. A population of oligomeric compounds of claim 6, wherein all of the phosphorothioate internucleoside linkages of the modified oligonucleotide are stereorandom.

152. A population of oligomeric compounds of claim 6, wherein all of the mesyl phosphoramidate internucleoside linkages of the modified oligonucleotide are stereorandom.

153. A pharmaceutical composition comprising a population of modified oligonucleotides of claim 147 and a pharmaceutically acceptable diluent.

154. The pharmaceutical composition of claim 153, wherein the pharmaceutically acceptable diluent is artificial cerebrospinal fluid (aCSF) or phosphate-buffered saline (PBS).

155. A pharmaceutical composition comprising a population of modified oligonucleotides of claim 148 and a pharmaceutically acceptable diluent.

156. The pharmaceutical composition of claim 155, wherein the pharmaceutically acceptable diluent is artificial cerebrospinal fluid (aCSF) or phosphate-buffered saline (PBS).

157. A pharmaceutical composition comprising a population of modified oligonucleotides of claim 149 and a pharmaceutically acceptable diluent.

158. The pharmaceutical composition of claim 157, wherein the pharmaceutically acceptable diluent is artificial cerebrospinal fluid (aCSF) or phosphate-buffered saline (PBS).

159. A pharmaceutical composition comprising a population of modified oligonucleotides of claim 150 and a pharmaceutically acceptable diluent.

160. The pharmaceutical composition of claim 159, wherein the pharmaceutically acceptable diluent is artificial cerebrospinal fluid (aCSF) or phosphate-buffered saline (PBS).

161. A pharmaceutical composition comprising a population of oligomeric compounds of claim 151 and a pharmaceutically acceptable diluent.

162. The pharmaceutical composition of claim 161, wherein the pharmaceutically acceptable diluent is artificial cerebrospinal fluid (aCSF) or phosphate-buffered saline (PBS).

163. A pharmaceutical composition comprising a population of oligomeric compounds of claim 152 and a pharmaceutically acceptable diluent.

164. The pharmaceutical composition of claim 163, wherein the pharmaceutically acceptable diluent is artificial cerebrospinal fluid (aCSF) or phosphate-buffered saline (PBS).
